SEQUENCE LISTING (182 sequences)

SEQ ID NO: 1 - DNA, 990 bp, Artificial Sequence
  ST73 E.coli_CFT073 AE014075
tcgtaattctttttaatatccttataaaataattgttttaaatggtggtatttaaaccaccattttgttcttaatttttgtataaagaccaggtacgttctagtatttttaatgcagcaggtaactgctcaatcattttaccagaaacttcataatcgctttctttgttgtttaatggattaacagcaacgtaggaaatcgttgatgttggcttaattattttaaaaacttcctttgcagattcaccatagatgaaaggtaactttgaattaatagagatttcttgtttatcgaccataaacctggtaatagcagagaaatgagtctccccatgacttataacatagcctttcggaacgttaatttctttcagctcaggaatatactgaacataaaactgtaaatctttcacatagcaaaattttgcactactatcattattcttcgatttacggttttcgcattccatttcccatgaaaatagtccgtctccgatgtaacctgcaactacgcgtccatgacttaaatggtatgcaacgccgtcaacagtgccttgctcttctgaagaaaaactgaattctttattttcttcattatacggactgatagaacaaactatctttccttgaggaaaaattggtcttttggtgaataacatatcctccgacaaaatggtaaaaccatcaatggtggtaattgtttttttaattaaatactcactgcaattgacttcctttaaggcttcttgtttttgagcccatgataattctgcagttgttaatttacgataaaatttttcagtgtaactttcagccgccgcaaagtgtgacagtgacgataaaacaataattcccatgaatgtttttttcatgtatatatccttataggtatgcttatcaaataattcaatggaagtatatcgataaaaataaagccgaacatgttaataaacatactcggctttattttcggaattacacccctattgtt

SEQ ID NO: 2 - DNA, 10734 bp, Artificial Sequence
  ST73 E.coli_CFT073 AE014075
ttacttcaccacttccatcagcttactgaagctatccactacgtcatacttcacgttttctggagcaaacgtcttgttaaccgcatcgaagaacttacgggcacatttaatcttggtggcttcaataggccgtaaatccagtgacgacatcgagcccttggtctcagcaacgaaatacacatgcttgaccgagccttctttgaaggaaatagcccagtcggggttgtaatcgccgacaggggttgggatcagaaaaccacgtggcaacttggcatataccaccacatccttgctggtatccaactcttcgacgaacttgcgctcaattgcagagtcggtaagcacgtaatcatagatgtgattttgcaatttattcccagccttactgagatcctgtttgctctgacccgtggtaaagatgtccaaatcaaacttgtcttctagcgtgtcataggccagatgctcgatgatgacattggccttttgttcgttgaccaagcgaatgacttcggcaatgaaactttccgggttgctcttgaattgcgcgaagacgtcagcatggagaccttttagaatttcggcaatggttcgtcttgtaagcaaagtcccctctgataacttaccaatcaagtcgtactgcacagcggagtggatagaggcggtgtgcgattctgtttcggtaactttcagctcgaaggcgataccctttttgaggtcatcgtaagttgccgtatcagcctgcttgccacggtgaatggtgtattgtaacggcgttactcgtaacccagcattcttatcattgagcgccttgatcgcattgttcactagctcaacatcatcaaacagtacgctgtaggtagctttgcgattgattcgcccccacagcgctttgaactcctgcttttcaaagttggcgttggtctgcagtcttttgggcttgcgtccatcttctacctgcatgagctggctttcgctgaatacgctgttaatcagctggaatacctgctctgcatagggctgcagctcgtccggcaatgcctctaatgtgccgtctttcttagcttggtggtatttctctgtgatattgcgcttcttatcgacgtagccattctggatcagataaaactcgatgtccgtggcttggtcgtccgagaccgtaatcgggccttccttcgtgttcagcatcttgcccgtgaagtaggccttgtccgccacacgcgggcgagcagacaaggagtcgctaatgtccttctgcaaggcggtcacgaagtccttgtaactttcgctggccacaactgtcagtacgttaatgtcatgaacaatggatgggtgatccatgcgttcaccattttgattgacggacaaacgcaggccgcgcccgacctcttggcggcgtgaaatggtgttatcactatgcttgagtgtgcagataacaaacacgttggggttatcccagccctcacgtaaggccgagtgtgagaagataaaacgcaccggctcagctagtgataataaccgttccttgtctttgaggattagatcataagcatctacgtcatccgacaagcccgcattttcaccgcgcgcagcggctttgggatctgtgaggcgcttgctcttcttgtcgatggaaaagtagccgctatgcgtcttatcggccgcaatgcggcgtagatatttagcataaggggcatccccctccataaacccattatcaaacacttcttgaatgtgcttagcgtactcctcttcaaatatttttgcgtactctcccttttcatcgacagcggagtagtcccggtacctggccacttcgtcaataaagaacaacgagagaaccttcacccctttatcgaagagctccatctccttatcaaagtgcgccttgatcgtctcacggatctgaatgcggcgcagcgctatctcggaaatatcaccaatcacatcacccaccgtcagttctacgccattggtaaagctcaaggtgtccgtattggcattgatgtccgataccacaaacccatcgcggtattgatccagctcgttcgatagcacaaacagattgtcacccttacccagtttgcgagtgacgcgcttgatgtttccaccgctgagtttctgttcgaactctacaaccgctactggcggttttttgtttgagatttcgatggattgtaaatagagataggcattggttcccgccagtcccttcaccgagataccacgcacctcaatcttcttcaccagcttctggttgtaggcatcgagcgcatctaggcggtgaattttgttgtgtgtggtcttgtgtgtagcggaatagcgcaacaccatcaacgggttgaaattagccaaggaatctagggtcttgcccccctccatccgctgcggctcatcgaggatcaagatgggtcggtttgcgctgatcacatcgatcgggcgacgtgactgaaaatcatcgagcgcttcatagatacggcggttatctttgccagtggcgttaaacgcttgtacgttgatcaccatgacattgataccggcatctgaggagaaactctccagatggtgcagctgcttagagttgtaaataaagaagcgtgccttcttgtgataggtctcctgaaagtgctcggccgtgatttccaacgacttggctacgccctcgcggatggcaatgctcggcaccactatgacgaacttgctccagccgtactgtttgttcagttcgaagactgtcttgatgtagcaataggtcttgccagtacccgtttccatctcgatatcaagatttagcttggcgaccttggtcttgaccaagctctccgacaacggcagattttgctggcgctgcacctgctggatgttatcaagcaatgcaacctcactaagggttagctcagcatttttaaaaccagtatctaccgcaaacagatcacccataccgtttttggtattaccagggtcaaggcgatagctcatggccgcatcagaggccggtggctgcccctgaaaaatatcgacgaccgcctgcactgcggcagcctgatagggttgatgtttgaatttaagcttcattgcaccgtccccctcagatacattttacttcggtgacaggcgacatgagcttgaaaatctgctcgacattgatcttgaccgcactgttcttaaagcccgcgtcgcggaacacaacgcgcagcggctgcagtttggccagttccttcacgaaggtttcgtcaatgctgccactggcgtcgaagcaggcggtaagcacattgccatcgacaaagaacacgtctttaccttggatggactgcttggcgataggcagggccagatcgacgccccaatccagcattacttggaacagcagatcctcaggcgtgcgatcaggcttgatgttgtcaacaaacaagtcgagattggctttttccagcacatcggggctgtagtagacgtcagccatgttcgatgtatcgactttaagcaggcgaaatcctaagtcaagttccgtatttcttgtagccaatatttctgaaaattcttctctaattttttttgcagctctccgaatgcgattttttccaatctccgaaactactgagtatccaagttttttggcttcagacttctcgtcacattcctcgggcaattgaaccattatgaatcgacgatttgccccatcttccgcattcaaaagcataagtgcatgcgccgccgtgcacgagccagcgaagaagtcgatcaatatatcgtcattcttgcaaattgattgaattagtctctttgctacctgaaccggtttcggtgtatcaaaaggaattttaaggtcagcaagcaaagcgtcgtcactgccaccgtaataaagtagagaagggatattttcgaataaattgtcattcagccgatattttcttgttggctgcgtcttctcgtctggaccgaaaataatcaaccctgacttcaagagttcttgcattgttgcaggagggtttcgccacccacgctcaggaacagggcactccttccctgttacaggatgaattagcggaatgaaataatcttccggtgcttttttcttattcggccacgccatagaaaccggacggaagacgtcgccattgtcatcgatttgattatatgccttttcccccccagttagatcttgctggtttacccaatctttgtattctttccttgctttttcagttaccccgccctccttgctaagaatctgcttggcttttgcaagcatttttcctgcattctcttttggtcgctggaactcgcaggttgtcttgaagaattctcgatctttgcagtaaatacaaattagctcatgctgctgcgcgacacctgtggcatcaccttttggatttctcttatcccacacaatagttccgagattattcttctcaccatatatttctgccagaagcttctccaggttgggatattcattctcatcaatatgaattacgattaatccatcatctcgtagcaagttgcgcgataactttagtctcgaatacatcatggacagccagtctgagtgaaagcgcccgcttgtttcggggttggctgttaagcggtttccttctctatccacctgatttgaacgaagaaaaaactcagagacctcgtctacaaaatcgtcggcgtaaacaaaatcgtttcccgtgttgtacggcgggtcgatatagaccatcttgattttgccaagataattttcctgaagcagcttcagcgcgtcgagattatctccctcgataaagatattcttggtagtgtcgaagttgacgctctcttcaatgtgttcgccttttgaattttttgtcgtacgtactggacggagcgtctttgcaatcggtgcatttgcagtgagcagtgcctcgcgcttaccaggccaattcagctggtagcgttcctgcggcccttctacaatcgaatcagacagttcctggcgcaattggtcaaaatccactgccagcttcacgctaccatcctcgcccttagcttcggtgacgcagcctggaaacagatcgcgaatgcgagcgatgttgtcctgcgtgagattgggtgaatgcatttttagcttttccatattcttttcctctggccgttatgctgcttattgaaaaggcatagcggctcacaattcaagttcgtattggtatggttagttgccttgctgccagtcctggcaaaagtgcaggcgcaggctctcgagtgcagatggcaagctgccctagcaggtcacgtccaggtaggttgccgcacgggtgatgccgacgtacaggtatttatcgaacagcgccgggtgcagagtggcgagctggtcaatgctgacaaagaacaccgcctcgaactccatgcctttgatgtgctggatgtcgaacacgcgcacgttactttctcttctccctcttcatcgtctaccacaacacgagcaatggcaggctccgccaccagtacctgatggttttcgcggttcgcctgactgatcttacccagcctttggatggctgaactggacgtcattgcgttgatgcttgccagcgcatcggaaccaagtgtccgcccatcgtacagcttgcccctttcggtttcagcgatgctttcgagttgcgccaatgcttcaccagcgatctcctcgatatgtttccttgactcgtcatttaggctctgcgtcatgcattctctctccctgtcaattcttcaagttcgtttttaagctgtcgcaattgcgcgttgatctctactttgcggttaaattgtttttctttggcaagtctactggcagctttctcaacctcacgctgcttggctgcaaccatttcgacacgtgcgaccaagtccgcgagactttcttgtgggcgcgcgggcatggggatcaggcggtgaagcacctgctcatacaagccgcctagatcgagtgctaagggtattgcggcacgctcactgtcactcggcaaccaggctgtcgcaaagtagctactcaagacccaacgactggcgtctgactcattggggcgtttgtatgcagcgatcacctttgttcggccatcaaagctcagctcgaatatgatgggaaactgcactgcgccatcgatacaatgtaaaacgtccagattcagctccgatgtcttgagctggatcgagaaaatctgaagctctggtactcccggcctagcgggcaggttaatcgtttctggggccaacttgtatcgccagacgatcagctccacctgctcgatgaacaagtccttcagccttgtattggcgccgctatgttcgtagatcttattctttggcagggtacgaccaaaggcggcctgcttcgggtagtttatcaacgcgatattcgactcaaattggataggctgactcatccggcctcctgaatcactaggaaggtgatcaattcgaagtcgtctagcccgacgatagtattgaccagcgcggtagtcttaccggcgaagaagaggctgtccagatccttctcttccttcacttcgatcattgagcggatagctttgccaagcagatctgaatacacttgcatcttgctgccatcggccgtttccttgttgaacaagagacaggcgtctgtaataggctgggtttgccccttgcagcagctacgtaccagatccagcaagcgtttgacctccgtgtgatcgtgaatgacttcaccctcgcgattaatgtagacgaggtaataagggtgtagccggttgtgctgactgacgttgacgcttggattccggttgcgcagcgtgaagatcacacccgggcgcaggcccatctccggcttggcagacactacggcgtgcattccgtttggcacgttgctcaattcgccattagccttcacatagttgagcaaatccattcggaagtcattgagcccgagatcagtgatcgacactccggttttcaggtcttccagctcaatcacttcctcttgcaaacgtcgcaattgctctttgcgataagacacgtcgttggcttgggcgctcagcacattgtcgtcaccggtggctgtgacatcagcaatcatcatccggctctcaacccgttccttgaggttgatgtattcgtcgagagagatgtctggccagtagttaaccagttggatgctgctgttgggtgaaccgatgcgatccacacggccgaagcgctggatgatgcgcacgggattccagtggatgtcgtagttgatgaggtagtcgcagtcctgcaggttctgaccttcagagatacaatcggtgccgatcaacaagtctatctctgccgcctcatttggcagcacgatggctttctccttcgagtgtggtgagaaaagggtgagcagttcttggaagtcatagctcttcttaagcgtgctcttcggcacccctttgccagtgacttttgcactgtgcaaggtttgtgtggccagcaattccggagccaaattggcatataggtagtcagcggtgtcggcgaaggctgtgaagatcagcaccttcttgtttccaggattcagcggtgctgcgactttttcctgaaccagtgctttgagatgctgtagtttggcatcatcggcagccgtgatcttgttcatcgacgtcagtaaggcatcaatgatttccagatcaatcttcaggtcgtgctcccaagaaggcaagtccatatctgcgaggctgattttgaccttgccaccgatctcgctgtcaccgatgcagggcaggtcgtcgtccgcatctaggttttctaactggtcggtgaggtcattgattctactgtcaatgccactgaggtcgctagtctcattgaaagtgctgatcttggccagcgtattcatgtggttcgcgcgcagggacttgagcgtcaagcggaaggactcaatcgaactttccagacgcttgagcaggttggtcgtcatcaatgcctgcaggctcttttcgcggtcgacttgtttaagcttgcctttccctgcaacctgagtgtcatacatctcttcgtacttcttgagtcgactgggcaggatgtagctgattggggcgtaaacagcgagcttgagtaaagagagccgctcgaaaatttcgttaaaactcatcacatccgttcgctgcgtgagcgagcagtggaacgacaagggtttgcggcgctcagggaattggccgatttccttggtatcgtagaaggtctgaatatgcttgcgcgaacgtgcgatggtgacgctgtcgagcaattcgaagaagtcgaagtccaacgaatctaggatcgcccgcgctgtgcgatcttccgatggaagctttgcccatgcattgaagctagcctgtgcgccacggaaaatgtcctctaccgtcttgccagtacgcagcttcttgctgaggttttcggaatcgccttcgtaggccaacgccagttggtttcgcaggtcgttgaagcggttgttcaccggggtggccgaaagcatcagcaccttggttttcacgccctccttgataaccttgttcatcagcttttggtagcgggtttccttgtccttataagcatcattgttgcggaagttgtgcgactcgtcgatgataacaaggtcatagttaccccagttgatgcgattcagcggtgtgccgaacgactcaccgctcgtacggctgaggtcggtatgacaaagcacgtcgtagttgaaccggtcgcgagcaaatatgttggtcttgaggttacggctgtagttcagccagttgtccgccagcttcttggggcataacaccagaactgacttgttgcgcagttcgtaatactttatcacggccagcgcggtgaaagtcttacccaagcccacgctgtcggccaagatgcaaccgttgtagctctcaagtttgttgatgatcccggtggcagcatctttctggtaattgaagagtttgttccagatgagtgtgtcttgatagcccgtgcggtcgttcggcagagcatcctcgttaatgtcgtccagaaactcattgaagatattgtagagcatcaggaagtagatgctctcgggcgagttttcttggtagacggacgtaatgtggtcgcagatctgtgccgttacatcctccagcttctcggggtcttgccagatctggttaaacagactgaggtaggtcgccgtaaacgtcgactcgtccattttgttgacgaggtttgagacagcattgccctgctggtagccaagatcgacggcggtaaatccgtgcagtggcatgtaggcagtctctgtggcggcggcctgaacacaagcaaactgctgcatcggagccttgctgcgattggacttgaatgttgccttgcgccgcatccagtccgcgcactcttttgcaattgcgcgctgggtcagtttgttacgcagctgaatttcgaactcactcccgtacaagctccgttcgcggtcagccttcagtatgtggaattctttgcgttccttgcgaatcttgtcggtaacctcgttagcggtaaacgtcggcgaggtaaagatgaattgcagctcgtcgattttttccagctctgctttgagcgcttcgaacgcgtatatcgaaaagcaggaggcagcgatcttcaggcgagcactaggtttgagcgtctgctttaggtcgtctccgagcaggcggttgatattgtcgatcagttccattaatagtcagccttcttagtggctctatagcctggtgccagtaaggcattttttacgccgtaaattgcgagatgatctttgagccataaccggtactccggtccacgaaggcaatgatcaggtgagcagtctacccgccagcgctgcaacatatagcccaccaccgcagcgcgaacgcgcattctgatcgaaccatctgtcatcccgtagtccatcttgataatatcgggtcgctcaacgctcgggtgcggcacgaaatccagctcgacgatgcgcgtccactggatatcattatgcggtcgttcgttggcctgtggctcctcgtcgagcaatgttggttcttcgatgcgggtgacgacgaaatcccgaaactctctactcttgcgatcaaaggcccgtacgtgccagcgcaggccggtatcaaccaaggcaaagggcacaattacccgcttggactcgccgctgctcatcgagtggtagcggatggcgacgggtcgcttagcgtggattcctcggcaaatcgtggctaacacatccattttgggaatgcttagggctgcgggggattcgcatggtagcagtgcctgcattgagccattcactccatcgccaaaaccgagagacagcgccgacagaatgcgcagcgatgcgtgctcgaataacggggagaactcctgaccgatgcgatagatcttgttgctaccatcgaaggtgatgttatgcggcacaatttcccggtataaagccaaatcgcgagtcgcccctgccggagccacaccgaagcgctcaatcagatctgggcgaccgatctcaccgaagaagtagagccgaaaatcgatgtaggccagccgatcgcgctggaccagactcaagtcttcaagccgcttaggaggggaactagacatgaataatcactatataggaaaatgaaaattgaatacggtaatccataaagagccatggttgtaatcaaattgatgacaatcatgcattaatacttatgtaacagcaagaaatgatgtcttaaacgtagacaataatcgtattggccgtgtttctatgattgtaaaagc

SEQ ID NO: 3 - DNA, 2221 bp, Artificial Sequence
  ST95 E.coli_UTI89 CP000243
gtggttgtccgctttgtgccaatagcggacattgctggctcatggtatgttagtttaaggaggcaggttatgctctgtatttcgctgtggtataatagttgcgcaattgatacaagggagcgttacatcgtgaaaattgttttcgaggttaatggaaagaaagttccactaaaatctttaaaatacataagcaaaaaagatcaactcgaagtaatgaaaaactggttctttgaaaactttgaagatccggcaaatgcttgtccgtatgaaagtagagaaggtggctatgcttacatatatggcgggccgtatgatgcgagcgaagaactacaatccatcttcggtcaatatgttaaatctgagtatattgaggaattagtagatgaattacaaacacaatgttttgactggtctggaaactcaaataatatagatgattggtacgatgatgatatctacgacgctgtaacgtcttccggtaatccgtatttaaaattcattgataatatcgataaaataaaaaaacttgcaaaagataaaactgaacaacagcaaaaaaaccacttgcttagtttgctctacacgaatgttattacagctttggaaacgctttatgttgagctattcattaattctatagagaaagatgatgtctacatagccaattgtatagaaaaaggtaagactgaatttaaagtaagcaaagatattgctgcgttgcctttcaagggtgaacccatcgagaaaattaggggagagctaatcaggtcaatcaaggaacacttgatcagtgcaagctggcatagcactaaaaaggtaatcgaccgctatgaagctacattcgatataaaagtacaaaaagactgtccgatagaagctattgaattggcaaccctcaatcgaaatcacttagtgcatcgtggtggaaaggataaagaagggaacctcgtagtgattacagaccaggatcttgaaacgctgattgaaaatgcatcgaacctggcaattatgctctacaacagtttaaatgtagcaacgaataaaaccaccattttgcaaccagatgacaaaccgttcattcatgaattttaatagcgcaggtcatacatggatattcgcacacgaaagacaaaatttttagagtcgttagattcaactgaagtgatacgcaaagcagtaagtctcgctatagattgcattattgataatcataatagtaatgaagatacccccttagttattaccagctatgatgatctctgtagaatccaggtgctaaattatgtacaggagttttgcgaagctgcattccctgatatggatgaatattactttagtcctaacatacttcgcatcaacggcaagactagtgaagaagcttgtattaacttaattaaactcttaagaagtacaaaaggcatgctattctggtccgatgccccatcgtggttcgctagcctccccgacggattattccatgttgttaacattgatcaaaaaatcgttactcgcggccttaataagaaaaactctaagccgacaataatcaataaagattacagtgtagatacactactatcagagctatttttgaatggcgcacacatggagcaacccaacgtacacaacgtttctgagggaaacatgaagttctatgatgaatgccatgctggattaatcagaccaatacccgctccaataggcgcgtcatacgatgaagagatcacaattaattctccagattggcaaaagctcgcgtgtgtagctttacgccgctatcagtcgaaggaatgtcatgacggaatgcaatgggatacaaccgatcatggttggactgatgtaatcgcgtatccattcgtcgaagaaatacaatcaatggataatagtggttatcgacaatgtttagttgggctggtcacaataaacaattcaaatgcaaattctccgtacctatctaccgtttggatacatccattttatcgcaggagagggttattaagtaagttatggccaaaattacaagagttatatggaagtaacttcgaaattgagcgacctaatgaaaatatgaaagcatttctgaaaagtgcgaaacacgcagattactaatttatgaatcagtctgtggagttagggttggtgtcaacaagtagggtaagagcctcaacgtgatgctatgccgacctgctccctgagaattcacacagaaggctgctagcaa

SEQ ID NO: 4 - DNA, 336 bp, Artificial Sequence
  ST95 E.coli_UTI89 CP000243
ttattgcgtgagcttcgcatgatagctggagcatctcttgtcatcactaattagctccgctttatccgttcttgcccatattatatttctttttattatttttgctggattaccggctgctgcacacatagatggcacatctttagttacaatactcccatacccaatgacagatccgcttccaacagatactcctttcattatagagacatttctccctacccatacgtaactagatataatgatatcttttgcccaattaatcctttttttgctatgaatatcaaatataggatgcccatctgatgcacgcaaaataacatctcttgctatcat

SEQ ID NO: 5 - DNA, 1705 bp, Artificial Sequence
  ST95 E.coli_UTI89 CP000243
taacagatgtgagcgtttgctatgagcgatatatagtcattcgtaccctccgatactaatcatcacagaaattaatatattcattccttactgaaaagctatgaagtagatatataggattggggcgcgtatggcactggacagttatgcatctccgaatattattccggagctaagacaaccgaatgccgcagcaggtaagatcacgctaggggggctggacagaatggctgttacgcccagtgtgaagaaaagaaggctaagtatggtagaatttccatacgcacagaccttgttatccgactatacagcctggaaggcactatgacaaatctacaactggaagaaagatttagggcgcttgagaaagattacgatgcgctcatttccacaaaatatactgcgcagaacgttctcactcacactatggaaacctatgttgattctggtaaatataaaaactggatcgccagagtgaaaaagttaatagaggacagctacggaaaagagtctgattattacaatgacttcaatactgtaaacagcaggtggtcttctaattacaatacactgatcaaaagttataaaccgttgtttgatgcggcgagagatgatcttgcgcactctgctgtcagtactaatactcctcaagaaggttcgccgctgagcctcgtacttaatattcttaacaggtttccgacctttgttcgccagttaaagaggcggtacaacggccgtgcgccgcttgaagttaatgatgagtatgacgtacaggatttgatttatgcactgctgacgcttcattttaatgatattagggcggaagagtatacccccagctttgcgggcgcggcctcaagacaggactttcttttgaaaaaagagaaaattgtgatagaggtgaagaaaacccgtgaatctctgggagcgggtaaagttggtggagagttgcttattgatatggcgcgctaccgcgctcaccaagactgcgatacgttgatcttgtttgtatatgacccggattgctacataaataatcctttgggcgtgaaaaccgatctggaatctaaagacgcggaaggaaaagtgaaggttgttattgcgcagttctgacacgcttaagtgtcaccttacaggattaggtttgcggccgtgccagcaggacgagcatgaaaaaggccgctggcacggccggatttgtcatctttgccaggtcaaatcaaaatctacgtgaaaatccgcaggcttaagctcgcaactgccaccacgcccaattaacgacgttatgtttacaacgtttgaacctgttgagatggcaagttgagtgtcatctgtaaacgtgatgattagcatttcagtctgatgaacctgtactccagttttctgaaaaccaatctggacagatttcaccgttttcccaaccatttcacgggcttgattgatagagtaattagcttcaacttgtaggtcattcctgaacttgggtagtgtcattttacatcctctttagtgaagttttcgctatttaaatcagcagcattccttttaaatcactggcatgtcagcagtggatagtctgagtgtaacgtccagtgaggggaataaatattggtcagactttgcacaaaagagccaactatcgcgggctctctctcatcatgtgcgaaaattgaccacaatatgaaatccagttccattccgaaat

SEQ ID NO: 6 - DNA, 2661 bp, Artificial Sequence
  ST95 E.coli_UTI89 CP000243
tttaaagtgggatctaactctaataatttaaaaaatttaattgggtcttgatattaatcacatagactcaatgctctttagttcttacataggtggttgtattatgagtatccccagtttagatgttcataaagttgttaagtgcgcagagcataaaaaggctttagatgcaatatctctcaaacttaagaataacattattgactcaagctctgaatacgattttcttggcgacacaataattttcaataaagataaaaaaataggttaaccctcaaactttcaacatatgaactagaacttattttcaaagaaacactgctagtcattgatgaagctcctgtattgaagtttgctgccattgacccaaccgatgaaaataacaacgagctgattgatttatttttatccaaaggtggatttatttatattagagaatacaaatccgagccacattatgagtatggcgacacttctctatttttacatatacttgacgctactcttaaagagcttaaatctgcaaacaaaatatcctattaaataaatttttgctaacatagccccgcgccatgacaagattttctgttctagatgcaagggctatgtgtgaatatctataaacaaaaaccccatccggtgatttttgtcattttttagccatctcaattgcggcatcaaaaagtcgttcaagaataaaatcagggtactgtaatttttcttgctcgatatgaagctcataaattttcccttcccactcccatttaaaattaaatccagaagcagaaggtgcgaaatgtgttttagctttagggttagcgtaaccagcaacagtcaacacatcaaacttaaaatcttgaggtttaatacgcataatcatatcctttgccaagttcatcattattcttcgcgattataaaacaatgaataaaatcattaaagcatagacgttaaatatcctcaaaatgtgattagttgggacattgcgtcctgatatactcctgcaagtaattgacctgtgaggttatcttgtcgattccacttcggagacggtaataatggagttcagcatctgctgtaagtctagggctttctccatcgcccatgccgctggctccggtcgttgactttgtacaggtggcggcgacttgcaggcgcttatgcccagcagaaacatcagcacgaagactttcgatagtcgcattagcatcagcaagctcctttgtatatctggcatcgagttcagctacattacgttgacgcttctgcatatcagcgatgatggatgtggctttatcgcgctgtactttgtagacgatggcgtcatcacggtaattatcaacagcccatgacaagcagacgatgatgcagataaccagagcggatataatcaccgttactctgctcatacctgaatctctttgaccgttccgccggcttctttgaattttgcaatcaggctgtcagccttatgctcaaactgaccataaccagctcccggcagtgaagcccagatattgctgcaacggtcgatagcctgacgaatatcaccgcgatcaatcatcggtaaagcaccacgctctttaatctgctgcagtgcaacagcatcctggctttttggagagaagtctttcagtccaagctgtttacggtatgcatcccaccagcgggaaagaagctggtagcgcccggcagccgttgatttgagttttgggtttagcgtgacaagtctgcgagggtgatcggagtaatcagtgaatatttctccgcccacaatgacgtcataaccatgattacgtgtcggttgtagcccattatccgttccttctgaccaggccaccatatcgaggaaagctttacgctgaggattaagattttgcatttttcacccctgtcagtcgttcccagaagtacgtcagtgcaaccgaacccatcgcaccactaatccccgcagttgcgagaatcatgtaaatactgaatccactttcgatactgattaggccaccaataacaccggtgaatcctgatacaactatctgagcgagggcatttatccagctccacgttgctttactctgcttcacatcaatcaggtaacggacaagccccccccaacctgcgatgatcagcaaaacaagccagaacgctccggcaagattctctttttcgtgcatatgaatagccgcagtttcgcctccgacagaaaatcggagcgtgaaatgatatgttgatttattattaattcgaaaattacattctaattcatcaggcattgatgattttataataataaaggccgttcgttacggcctttattattgatatttattttacatcccaaggtagttcctcaagagagaagactaattcattatcaataatttcgtattctgcataaccttcaacctctcctgagaagttattgtcaacgcatccttcataatagctatatggtgctataaagtgaaacccatattcatctatctctgtgatttcaaaattgtagtaatcgtaagaatatcgaatatcatcatcagaaatagaactaagtacatagttattaaatttatcggaatgcttaacgataagttctgacaacgcgtcaagattctctggagagatatttcctaacggtaatctgtactcaacggcattaatcat

SEQ ID NO: 7 - DNA, 1871 bp, Artificial Sequence
  ST95 E. coli_UTI89 CP000243
ttattttaagtcattatgccaatcgtctactgtttgaaaccaaatttttaacctttgcataaaggtacgatcctttttgggagggtggtacctttgaaaagaagtatgtaacgcttcgataactcccatacaagcatcaattacttcttgtgctttaaccgggcgatcggcatctgaaagcttgtgagcgacctcacaatccagagttgctttctctagtacagatgagaacacctcgtcaggtatatacagtcctctgctttccaaaatgcaaatcagtttggaaacttgttgagctttagccaagatagttaacaagtatgaaacagaagcctttgttctatccttttgcaaccttaacaatcgatgctttgatgctctaattttaggttgagttgtctgactgagccaatagttagaagaaatatcagaaatttcaataactaactttgaaatagcatcaataattgatttcgattcagaacgtgtcgccagttttacagaattattgtacgcaactcgccatccaatgaaaactaagcagacgctaactactgtgatacaccacgaatactgtcctatttgtgctactaatgacattggttccgtactgatcaactatcgcctcttatactctttctggttgtgctaaatcaataaatttatcaatctgttctttgtagaacggaacatcactttttataatcaaacgtgctttaacgttggctttaggtatcccttccttacggatgagacctccaaacgcctcttctaagaaagaggaaccaacacccagtgctataccgcgaaaatcaaccacaatctcctgttcagtacccttcagcgcaggcaccaaaaactctctacgaaatctttccgcactgtgtgggctatccgttgtgtaacgtccgaaaggtgttctggagaactccttcgctatgacgatatttctcataaaatcatctcctgggaaccagcgaccattgtagtaaagtacccggtatgtactcagctagcttctcacatcgaggactactgtcattagaattatagctataacgagcccttcctgttagtatcagaagtgtctcattatcctcacaacctgccccaataggtctcttgatatcttcagagccatttccccgtccggaaccaacaaagcgggattcccctactaacatagctctttccacagaagatacttcgttttgaatgctatgaccatcaccattaggcacgaaacttctataaatccccaatccaaggtcacaaacgataaaaacgactttattttcatcacgattgaaccaagcgcactgccaccatcgtttacctttgagaagctgaatatctgactcgaacccctcatcttcatacgcatgatgagaaacattgagcaaagcttcactaatcgctgtcagcagtaagcctaactgttcattattaagcagagcacttttctgcagcatgagtactgtttcaacaatatgctcgtaaggttcgacagctgactgaaagtatcgttcctccctcgtaagtgcattcagtttctcttctgtgccagcaagcaaagcacgagcaagtccagtaccaacgatccatctatgcccagatgggttatcatcctttttaggccatttaaaacgtataaaattagggtcccgggttaaaaattgagctctatttactatggcaaagaacagtaccgatgccgcagctgaggcaaattttaccttggacaaatcaactataactcgacctttattttttactccaatcgattcaatcgagttgatgaagttcaatgtcccagccctagaatcatctgagtaaatgcaaaatttgcttgggggtaccagtatggtcat

SEQ ID NO: 8 - DNA, 3304 bp, Artificial Sequence
  ST95 E. coli_UTI89 CP000243
atgacatcgacgatgcatatggacagggccgataagaataaagttggcaccgttgaaattgaggatctacgggttattccggttatttttctgcccggagtcatgggttcaaaccttatggataagaaaggaaaatccatctggcgatatgatgactctatgagtctgatgggctggtcactacctacatcaggaccaaaagagaggaaaagattattgcatcctgacagggtggaggtcgataatcgtgggagaattcccgcacctccggatgcccaagaaaaacttattcagttaggtcaacaatatccggaggacccatccgataaagaagcaatggataattacactcaggcagtacgtgacattttggataatatagagccagaagcgaaattatttggttcaagaaaagatcgcggctggggagaggttgcaaatgctagctacggttcctttctcgatgtattacaaacagcattgtaccgggacaaacctacaaagaaaggtgagacgttaagtgcgacctaccagcaactactcgatgtacctttgggtttggagtatggtcctgactcacttgatgaagagtatcttgaagtcataaggttgtatcaatttccggtccatgtcgttggctataactggcttggttctaatatgctctcagcaatcagactacaagaacagatcaagaaaattgttggaggctatcaaaaacgaggaatgaaatgccataaagttattctggttacgcattcaatgggaggactagtggcaaggtacttttcagagtgtctttcggggaatacggatgtctacggtattgttcatggcgtattaccatcaattggtgctgcggcgacatatacccgaatgaaacgtggaactgaaaacccagagtcaaatccggaagggtatgttatcagtcatatcttaggtcgtaatgcagctgaaatggtagctgttttttcccagtcgccaggaccaatggaattactacccatgaatgattatggtgaagagtggcttaatattgtcgatcgtgatggtagtacattaacgctccctaaaaatcttcccattaaagaaggtattgcccccgaagaacgcacctacgctgaactttatctaaatagagaaaattggtggaaattggtggatgagaatctattgaatccttttaatacttcattaaatcaaaagcaaattgatactgattggaatatatatgaaaatttaattactgagagtgtcaatccatttcataagcaaattgctggaaaatatcatatcaatacttactctttctatgggagagcgaaattgggagatattcctgaagcgcatttaacgcaggaaaatgttctttggaaaggctcattatcaatgggaaaaaaaagtgatatatctttagaaccaaaattcattgatggtcgtcttgatttaaatgaagttggtaacattagaactattaaggatgaattttctcctgaagaacaggcgtgggaaataaatactgacgacggtgatacatatgttaagataggacagcgttttacattacgagactcttgtgaaaacggggatggtacagtacctcttcgtgcaggacaaatcgtacacaagaacatcctagagcgtttggctgtacaggtttctcacgaggctgcttatcgaaatcccgttagtcaggcatttgcattacgttctattataaaaatagctcaggaggtaaaaaaggatggtaaaatgtcatactctgattaaccgtagaaataaatgtctgctgattgtttttatagtccttattggatggattatattcagacctaaagcatatacttattcactaaatgataaagaaaaagagatgctcataatgttatcacaacatcctgaaactcggtactttggattttattccatagaacttccggctgattacaaaccaacaggaatggttatgttcatacaaggatcggcgatgatccctgtagaaacaaagctacaatattatcctccttttctgcaatatatgacacgatatgaggcagaactaaaaaacacctcagcattagatccactggatacgccttatttgaagcaagttcacccactaagtccacctatgaatggagtcatttttgaacgaatgaaagcgaaatacaccccagattttgcacgagtattggatgcatggaaatgggaaaatggcgttacgttttcagtaaaaatagaagctaaagatggtagagcaacccgctatgatggaattagtaagattgccgaatacagttatggatataatattccagaaaaaaaagtacagttacttactattctttcaggactacaacctcgtgcagataaccaacccccatcagaaaataaattggcgatacaatatgcacaggttgacgcttcactacttggagagtatgaattatctgtagattataaaaatagcaataatattaaaataagtttgcagacggataataatagttatattgactcattattagatataagatatccgagtaatggaaacagagcatggtataactctatataaaggtcgtaagaaaattaatggtctggagatatctgaatggtgggttcgtaagcaaataatgttgaactcagaaccatcacgagattatgaattcaatctggcaatccatgaggataagaaaaacaacaaacagttattaaaacttgttatgaattacagtgttgacatcttacacgctgacaatgcattaactgaacatgaactgatggcgctgtgggaaagtataacagggacaattaaatatcatcccacgcagtggtaaagcagataatcagaacagtctggagatatcggaatatactcgtggtacttcaagttgcatggtacgcacaaggcccttgtcggaaagggcttgggctacagaactcatttacgtgcatcttcaagtttatcgggtatagtagattaaacagcagatcacgctaagtactgacccgctgcaaaattacaggttttatctgacagtccatgatgataaaaagaatggtggtcaacggttaatacttacgatgacttatagcatcagaccttctgaattcaaaagaagtcttaaccgaacatgaactggttgccgtgttgcagcaaatgaccagtacgctgaaataccccctctcacaaaacataaagataatggtatctcatttatatcaatgaccgggaataggcaactatactttcattttttaaattattttcctgttgtaaataaatcagaggtaaaaatta

SEQ ID NO: 9 - DNA, 2494 bp, Artificial Sequence
  ST95 E. coli_UTI89 CP000243
atggcaatgggaacaggctactttctggtcaggggggacaaaaccacctgcggcgggaagattattgaaggcgcggacgaccatacgatcatgggtatcccacaggcacgggatatggatcgggtcacctgcggcagatatccgggaatgtttattattgtgggcggcgtgccggaaacggatattcacggcaggctgatggcgggatctctggacagccagagcagttgcccgtgcaaggcgcggtttattgcgtcaatgatggatgatacctatgaaacggatgacgggggaagcgagccggaacaacatgcgcagtcggcgaggaaaaatctgacttccggtaatccagataaaaaatacagtcatcagataaaacttcaacatggcgaaaataatgttagtgtacaggacataccttatgtctttatcctaaataataacatgtctctcagtggcaagactaatcaggatggcgagactgagagaatatatacagatactgctcaaaaggtaattgcacttacaggaaaactggctgattcctggctgaaaagagggaaaaattttggttcattaaaagaaattgacaatagaaagatcgaattaacaactgaagagaatgaacctgtaaaatatgtcaactggattaatgggcgtgattatattgtgattgtggcggccagaacagccgtaacaaactggataggaatggaggactcaaaaggaaatcagtatcgatttataaattgtggattagaacaattacaacagtttcctccagccagtaaacaggattccagttcgcagcgaattatggtggttttttcgcttgggtatacgcaaaaagatattgatagaattaatgattatactaaagcgcacgatggaagaattatctatgtcaagaataaggatgaattagtttcattcctgaatcaacgcaaagaaaaaggaagagtgattaaggaactggttattctgtgccacggtgttattaaaacagcttcatatcattatcatcatgaagacaaggatattgaaaaaaatggaatgtttaaacatgaagatattgcagcagttcacgagtcagtttttgattatgatgcccatgtgacaacatatgcctgtcgggccggtatctcagatggtgataaagatttttccgggaaggatgatgcagggcaaaaggacagcccggcacagaaaatggctgataactgggatgtcatggttaaagcgtttgagatgagatcggactacagtctggcttatgggacaggcaaggaaattaaagaagctcaggaatatggtagtgtggtagaaaaatataaaaaagatatagatatgtataataaagaaaaagcaaaggggaatactgaagtttcccctccagtgaaaccagagggatatgatgagaaatcaaaacgccatgcagatgttactaccagagacaaaaatgaaaagtctggcggaggaccaatagctcctaatggtgcatggcatatgccaagaacaggggattcgccgaaagggctgaaaagcgggttacaggattatcagcctgaggagtgggttcaataatgttcttaaaaagaaaatggtattacgcagtgacgacatctgtcgtcattactttgtgtggtggaggatattatatgtacaggcaagaatatcagatggttgtcactgtaccaactgctgacgcgaacgatcccaactggccaaataaaaggatacagtttgataccagcgaatggctacagcaacttcaatatattaaaatagatgatcattatatattgaatactcaatatactccaattgctaatttggatgactttggtattacattaaaattacagaacgcattaaatgggtcggataaaagacttcctgcactatatggccttgctgagatggatgctcagaaatttaaagacctgatgcgcggtaaaattaaatgtgaatatctgaggacgacatttgatgcggaaacattaaagcctgtcaatgattatttccttatttcttttacttataaagataagtggtatgaatttgagacagaaagaaaaatatctaaaacaagtgatgatgggtattttttgtgggcatttgataatactgtccacgaagcaggctattggcataacacagatccggctgcgtattcctatagagattaccagaatggtaaggctgtgaaataagtgtcaggatactcaggcggcggtactgtgcgatacgcagcttagtttggattactgattgtattgtggcctatattgaagcagctcagcaggaatttgttgagaaagccagagagactgcagatgatatctgggacttgggaactatgtgggcaaaaaatgtgctaattaaaggaagggaggtactggagcatgaggtgaagtaatatctaagagaaaatcttgataatataattaagtggggactaaattagagatgaatattcaggcaataaaagaaa

SEQ ID NO: 10 - DNA, 4345 bp, Artificial Sequence
  ST95 E. coli_UTI89 CP000243
atgattgtaggtaaaacaccgcatactacagctatttctctgatgccgggaatgtcacctgaagcacttatgcaacaagtaaaaaccatactcaatcctgatgtagaaacaatcatatttacggatatttatggcggcacaccttcaaatatcgcctaccttctttcacgagaatttcccattcgctgcatcagcggtgtcaatctggcaatgttaattgaagccaacatgttacaggattctgacgaagaacagtcctttgatgaatatattcagcatatacatgatgctggtaaagagatgattcaaacttattgttttaataaaggatgataaactatgagtatagtacatgcccgtattgattaccgtttaattcatggacaagtcataacaaaatggctaaaaaggagtgatgcaaataaaataattgttattgatgatcccttgtcacgcgatcctttcctggctgaagtctataagatggcagcgccaagtggtgttgaagtcataatgacctccattgaagatacgttacaacgctggaacagcaatagtttctatgaaggtaaattactaattcttttcaaatcgatcgattctgcattaaaaactatacaaggtggtttgatgttagaggaattacaagtcggtggtgttgaaaatactccgggaagaaaaattgtctttaaccaaatatcactcaaccacgaagatgccgacaaacttcaaatcatcgaagataagaatatcaaagtttatttccaaactattcctgaagaagatcctgccagcctacaaaaaatcaaaaataagttaccttaattaaggagaggagcttatgtctatttttacagcagcaatgattgccctggtatattggatatcccaagcaaaagtatggtacggcttttctattatgcgtatgccattatcgatagcacctattatgggactcatctttaatgatatgccaactgctttatctgttggggccaccttacagatgatctatatcgggagtattgccccgggaggaaatcctcctgcagatgaaggtctagcctcgtgtatagcaattccaattgcacttaccgcaggtattaaaccagaaattgctatctctttggctattccactgggattgctcggcgttgttctggaaaacgtacgtaaaacactgaataccacatttatccacatggctgatcgctacgcagaaaaaggtgatataaaaggcatccaacgcgcagccacaatatatcctctcctgttagcctttccgatgcgttttgtaccggtattcatcgcctgcctgtatggcccggatgccattgcctcttttgtaaacctgcttcccgcctggtcgacgaatggcctggcaattgccggtaatattctgcctgcactcggttttgcgataaccataattgttatcggtaagaaacaatacatccctctgttcattattggctttttcctcgtgacctattcaggcttgaataccattggcatctctatttttggactctgtgccgtcctcttatatatgcagtcacaaaatacaaaaggaactaaaaatggataatataattgagtcaggatactcagaatcctcccctattacgaaaagagacgttcagattgtttggctaaaatggcacgcgttctctgaaacatcattgaactttgaacgtttacaagcactggcattttgcaatgcaatgacaggcgtattggcgaagctatacccagatgaaaaagatctcgccaaagccttacagcgccatttaaccatgttcaacacacaggctaactggggctcggttatcgctggtattagtattgcactggaggaaaaagccgcccaggaatcagaagaacaacgtgaatcaacaacacaacttgttacgggtttaaaaactggtcttatggggcctgtatcgggtattggtgacacgttagactttggtacattgcgtcccattgttattggtgtctgtatcccgtttgttattcagggatatgttattgctgcacttctccccctgatttaccaggttagttatatgttcttcgccagtcgatttgctcttaatatcggctataaaaaagggaaagagtcaattctggaaatcttgcattccgggagcatacatcgaattatcgaaggtgctggaatgttcgggctgttgatgatgggggcgctttcagcaacgtatgtaaaaattaagacgccattgcatattacaaccggcggtggcaataccattgtcttacaggatatgctagataaaattgtccctaacatgttgccacttattgccgtgttgggtatttatttttacatacaaaaatgtggccctcatttcttacgtattctggtcactattcttgttttgagtcttgcattttctttctttggccttttataaaatgaaaaagataattatcactccccggccttttgtgggtaaaggcaaggtctatattgataagttaaaatccgcaggttatcaagttgaatgtaataacagtggaggtcgatatagcaaggaagaactcatcgagaaaattaaagacgcgaatgccattatcaccggaaacgatcctctgagcagagaagttattgatcaggcaaaaaacctgaaggttatttcaaaatatggcgtagggttagataacatagatgtcgattacgcaaatagtaaagacatcgtggtacataaagctcttaatgctaactctatttctgttgcagaaatgaccattctgatgatgctctccagttcccgaaaatatgttgaaatcgagagtcaggcaagaaatggtaaggacatcaggcttgtcgggtatgagctatatcaaaaaaatctgggattaattggacttggtgcaattggtcaacatgttgctcatattgctcattctatgggaatgacgattactgctcatgatcctcatattgataaaagcaaggttccatcctatatagaacttaaatcacctgatgaaatatatcagtacagcgatgtaatttctctgcatttacctttactggacagtactcgcaatataattaacgactctgtatttgagaaaatgaaatcttcggctattttaatcaatacagccaggggtggattggtcgatgagaaaagtttatataccgcacttagcaatcagaaaattgcttttgccagtgaagatattgaactgagagaacgttcaaaagaactcaccgaacttaaaaattattctattacccctcatgccgcttcatttaccgatgaggccgaccataacacaatgcaaatttctattaaaaacgtattacaggaactggagaaagagtaatgaaagagctcaaagggattattaccgcaatggtaacgcctttcgatgaaacggaaaaaatggatatcactgcggcgaaaaaaatggcgcgctggttagtagataatggtgttcatggtttattcatttgcggaactaatggtgaattccatttgctttccgatgacgaaaaagttgagttaacaaaagccgtcgttgaagaggtcggtaatgaagttaccatttgtgcaggtgctgggtgctgcagcacaaaacaaaccatcgaattgactaaacggttagttgatgcgggtgccgattatatttctgttgtcacgccgtactatctggtaccaaatcaggaagatttataccgtcactattacgatattgcctttagcacaacagcccccatcattctctataacctgccgggccagacaggattaagcatcgaatacgaaacggcaaatcgtttagccgatatcaaaaatatcgtagcgataaaagacagtagcggtaaattcgaagtccagaaacagtatctggaaatcgctaaacataaaaactttaaggtattaaacggctctgactcgttaatgctggatgcatttaaagaaggttctgtggctgctgtcgctgcaacgtcgaatgttctgcctacgattgaagtcgatttatataattacttcatggccggtgaaatggagaaggcgcaggaagagcgtaataaaatggatgcactgcgcatgacgatcaagaaaatgaccgcgcctgcagtaatgaaggaagcattaaacctgatgggtgtgaaggcaggtattacacgtagaccaattcatatgccaaacgacgcgatcgttgaagacatcaaggaaatgttgaaaggctataatttcctgtaaaggacaaatgccggatgtatctcttgtcctacaagatagcgtaaacaaaaacccccggactcaaatccaggggttatctgtggcaacttaacgcgctgcgcgat

SEQ ID NO: 11 - DNA, 41 bp, Artificial Sequence
  ST95 E. coli_UTI89 CP000243
actttgaacatccactcgatcttcgtctacttccggtttat

SEQ ID NO: 12 - DNA, 1206 bp, Artificial Sequence
  ST131 E. coli_NA114 CP002797
ttatttgaccgtgacctgtccgttcataagtagagggaggagccagtcgcggagttggattaaatgactattctcttgagtgtttgatgccagtttattataaattggcatcattttattctcgaataacgccacaacatcaggagtggggcatatgatttcacaattagtgatatgcgtattggtcaggttattaatattagttcccaagcactgattgtttatataagacttaaaccatttcgattgcaggaatgtatttataaaataaaatgcatgttttttttcgaaataatacacgccattcttccctacatgctccttactaccacttgacatcacgatcaatatgtcaaatttatttagcatttgattaacgtttactctactagaaggaatgaagactaaatcatcaatatcaacattatttcccgtcacatttgtagcgcgaagaatgcctatcgtatctttatcatcctgagttcgaacatcctctttgctataagtaacgccacgatctaactcaataaacttaccaagaatggagtcattccaacctgccggaatttcccgtttcagcgtggcgttatactccatcttcccgccggaggttttatacggttttccgttggcatcagggaaatcaaactgtacaaaccagtagtcatacagtgttttcgccatcgcttccagttccgtgttgatgcggttgttgagggcgatttttttatcaataacactaagaatagtcaccaatttttcttgacattcaatttcttcaggtatcctgactggatattcttttaaagaaactagatcaactccagtttgtcctccggttctggcagataatttatcaacatattggaaaacagagtcttgcttcaaaaaatagtataaaaaatcacgacttacaccttctttaggtataaccttcagcaaggcaacgttatatgctcctttcaatccgcgacatatttgaaaaattgggggaccatacctaccaatcataatatcgaattcatcacaaaatttcttcgtcgacgatattggtatatacgtgggaaatgcatccgttttgtaatcacgcgtctggatcaacctgacatatccaggtttagatacataaataaattgagattttggaggttgacttcctccgacaaaatcacatacatctttaattttcagataattaaatttcat

SEQ ID NO: 13 - DNA, 2737 bp, Artificial Sequence
  ST131 E. coli_NA114 CP002797
ttaccagcagacaaagccaccatcgaccaccagatccacacccgtgcagaacgacgccgcatcgctggcaaggaacagcgctggacccgccatctcttccactttcgccatccgttgaatcggcgtctggctttcaaactcccgagtctgatgtaccatttcaggtcgtgtattcattggtgtcgcggtatatcctgggctgattgagttcacgcggatccccttactcacccattccatcgcaagacttttagataaatgaatcacgccggctttggaactgttgtagtgggcctgttccagaccacgattgacaataatcccggacattgacgcaatatttataatcgaaccgtctccgttctctaccattaactcagcttccgccttacaggaattccacacaccagtcaggttgatatcgataacgcgttgccattgctcgctttccatctccagcgccgggtttgcgttagcgattccagcggcgtttacggcaatatccagacgaccaaaatgtgtttttgccagagcgactgctgcgcgaagatctctcaattcccgaacatcgcccgtgtaatacaaggcttgtccgccaagtgcttcgatatgattaaccgtctctttgagtccgccatcttcccgtagatcaaaacacaccactcgtgcgcctgcgctggctaaagccagagcgatcatttgtccgataccgctacctgcgccagtgacaaaggcgacgcgatcgtggagatcaaacaattgttgagcaaaatcttttgcgatcattttgatatctctcttagtagcccgcaggggcattacgacataatcaggccgccgtcgatgttgatggtttgaccggtgagataacgcgcatcgtcagaggcgaggaaggcaaccagtccggcaacatcttcaggtttaccggcacgcttcatcggaataccttccacccattccgccataagctcgcctttgccataacgcttgtcatcggtactgagaatttctccccatacgcggtcgttgtagtcccacatttcgctttcgataatacccgggcagaaggcgttaacagtgatattccactgcgctaactcgtgcaccaggctttgcgtaatgccgatcacacccattttgctggctgcatagtgtggggtatagataaacccctgacgtccctggccagaagaggtgttgatcaggcagccgtgattctgtttcaccatatatttggctgcttcccggcagcagagccagactgccgtagtgttcaccgccagcactttttcgaaatcagcagccggcattcgatcaaaataatcgatggtgataatgcctgcattctgaatggaaacatcgatgctgccaaaacgtgcagccgcttgttcatagagtgcctgtacttgcgcttcatctgtcacatcgacttctaatgacaaaatgtcagcctgataacattggcgcagttgctctgcagtttcatgtacgcgttcagcgttagaaaccatcaccagatttgcgccatcgcgggcgaagcgttcagcgatcccggccccaattccccggcatgccccggtaataactacagtcttaccctgaaaattacgttgcatattttttcccctgtataagccgggcaatttaatgcccggtggaatgatgttaattaactggaaagcggttttacggcaggtgcggcggccatcttttcttcatggcgacgcatcaaaatgactttgttttgcaatttttgctggaactgatcgaccaccacggcggtgacgataacgaggcctttaatgaccatttgccagaagtcactgacacccatcatcaccatgccatcggcgaggaagacaatgacaaaggcaccgataatcgagccagagacacgtccccgaccacccgccagtgcagttccaccaagaaccgtggcgccaatagcatccatttcgaacatattgccggtcattgggtgagcagtttgcagctgcgaggcaacaatcaaaccgacaaatgcggcgcataacccggagaacgcatagacgaacactttaacttttacgatggggacgcctgccagtcgcgcggcagattcattgccaccaatggcatagatataacgaccgagcggcgttttagtcgtgatccagtagcctaaaatcaaaaagccaatcatcagccagattggtagataaatgcctaaaaacgtgccagaaccaagggtggcgaaacccgtgttgcccagcgtttccatgccgttgagattgggataagtgctgccgtcattaaacagcagggcagagccacgagcgacgtacatcataccgagggtgcagataaagggcgcgacaccaaagcgggtgattaccgcgccattcaccagtcccactaaaatgccaaataccgccacgcagaggatcacttccggcacgttgaagaaaataatattgccgctccacaatggcaggccgtttgttagaagagccccggcaaccattccgcagatccccgctaccgcgccgacggaaagatcgatgccgccggtcaggatcaccagcgtcattccgattgccagcagcccggtaatcgcaacgtgttgagtcatgatcaacaggttagaggctgtcaggaagttaggcaccatcacgctaaagaaggcgatcactaatagcagagcgataaacgttctggccttcaacaggtacatatagatcatatatttctgattcat

SEQ ID NO: 14 - DNA, 6610 bp, Artificial Sequence
  ST131 E. coli_NA114 CP002797
ttactcgcctcccgccctggctattttcgcaggcgttatatccgggatagtttgcggaatatcccatgcagaaaatacgccataaacctgaaccgtttccccggttttaagctttgccgactgaatcatattgatggcttgctcattgatagcacgaccatattcaccaaataaaacctgatcgttaaaatgctcatagcttgcacctttatatgcatctcgcagaagcgtgccgcgaagagttggccccgtctgcacgacaatatttgtgccgctgatatctgtgatggtcatttttccgctacgggatttcgcatcaatattggtgatgctaccttcgaccttaacgtagaaaatacacgggttttcttcctgagaacgatagcccagcgttttacaggcagcatcgacatctttttctgcctgtagcgccgccattaactccgcaaccggacgcgcattttctaccacctgcggcacgatgtttttctgccaggtctgatcgatatttgccatatgaggattaggcggggatttgagatcggcaagttcctgctgcgagacgatgcgacatccacccaggctcagaattgccagcgccagccagacttttttatccataatattctcctctgcgccctcattgtgagggcgcaacgacctgggatcaggatttgatattgaaatcctggactttatctgcgttatctttggttatcagaatgccgcggaacatcacccgctgtttggctggtttaactcctttttgcaggtaattatccagatcggtaacaccctgcgcggcaatggcttgtgcttgtaacatcacggtagcctgcaatgattcggctttcacggcatcgcgctcatcgttactgccatcaatgccgacaacaataacgtcattgcgattggcggctttcagtgctgcgatagctcccagtgcaaccggaccattaccacaaatcacccctttaacatcgggatgggcttgcagaatactgtccattattcgtttgccatcaattaacgtgcctttggcatcttgcttcgccacgctgaccatttcagggaactggtcaataacctggtggaaagatttcgagcgagttacgcaattattatcagcaagattacaggtgagttcggcatattttcctttctctcccattttctcgacaaatgcgttggcaacttccgagccagcctggaagttattatgggtaatttgcaccagtgcaacatcatccacaggtatttcgcgattaattaatataacgggaatacctgctttttttgctttttcaatggctgcaacactggcagtggagtcggcattatctaaaataatgccctgaacttttttaccaattgcagtatcgatcagttcattctgttttttgacatcttcaccatgagaaagaatggtcgttttatatcccagttgctgggctttttcactggcacctttcgcttccgaggcgtaataaggattatccagtgaattgaccataatcataatggttcctttttctaccgcatgagcagaaaaagaaagagcagtcagggtcgcagcggttaacagtgttaaacgtaatttcagaaacaacgtaaagcctgaattatttatgggccggtattctccttacgttggttagtcttgttcgtcaaattgacgatgccggtttccataatgttctgtttaccgcatgacgccactcctgccatctcttttgcagtcgctgatgacgcgccatgtccggtttaaattcactgtgttcagttaataacgcttttaaatctgcaacggtaccggggataagcgctttgcgggcgagcaggcccgcaccaatggctgaaagctctggcacatcactgcacatcaccgggcaacccagcagatccgcctgatattgcatcaaccagtcatttttggtcgggccgccatccaccattaacgccgataagtgaaaatcatcatgctgttgcattgcctccactacatctgcaatttgataagtgattgattccagtgcggcacgaatcaaatgcgcgcgctttacgccacggcttaagccacacaccacgccgcgagcgctgtcatcccaccatggcgcgcccaggccagtgagagcaggaacaaaataaacacccatagtcgaatccactgaggctggcagggtattcagttcgagggccagctccgcgtctgtcagttcacttaaaccggtgctgtcagccatccatgccacggcatcgccggtgtgggggatatttccttccagaccgtaaataatattttcgccatcatgccaggcaatggttgtggcaagtgtggcgatatcacactgagcagagttgacgggggccatgacggaagaaccggtcccataggtcgctttcacacatcccgcttctcccaacgcatgaccaaaaagagcggcgtgagaatcgcccaccatcgccatgacgggaataccatccggaattgttttaagccctcgggtataaccgaacaggccactggaaggtttaatttctggcaatgcagcgcgtggaatgtgaaacaacgccagcatttcctcatcccactcgcctgttttcaggttaagtaattgagttcgcgcggcgttagagtagtcgcagcaaaatgaatcgccagcagtcaagttccatagcaaccaggcatcaatggtgccgagacaaatttcaccctggtcggcaagttgcacaccctcaggtgtagattccagtagccaacgcatcttggacgcggaaaatagcggggcgataggaagacccgtagtcgttttaatttgctgctctttgccctcgcgacgcagtgtttcacaaaactcggcactgcgcgtgcattgccaggtaatcgccgcattgagtggttttccactctgacgataccagccgatagccgtttcacgctgattactgatagccagagccgctacattttctgcccccacgtggttgatggcctgggcaataacatcaagcgaggcagagattaacgcttccccggattgctcaacccagccttcacgtggcgtctgaatagccagtggttgagaaaacttcgccaccacgttaccgaaaccatcaagtgcgatggcttttgcattggtggtaccctcatccaacgcaataacgaactctttatttcctgcaaacataggtagtctcctgagtcacattgcatcctggcgtggaaggagatgaggcgttcctcatggtcacgttattacttcatcaggccctgctgtgacattttgtgttaaggcatgaatatatattcgatactgattatttatttataccttagccagattattcttcctgagctagagaaaaaatcatcaaatgtgctggctatcacacttagaagcgtggtgacagggtgagaaagcctcgagaaacaggattgttgaatcctttcagtttattgatattaaaggctttttaagcaatcacctttgcgagaagattttttcgcaaaggcgggcagaagaaagggaggagaggattaatccgcaatcagcattctggcggtggggaaatcagtgatcaaaacatcaatataaccaccctgcatggcgcctttgatggcggcaactttgtctttgccgcccgccagtgccaccacattcgggcattttttgattttatccagtgccataccaatcactggatcttcatcgtcctgcaacacgggctgaccatgtttatcgtagtagtgcaggcagatatcgcccacagcaccgcgatccgctagtacctgtaacatatcctcgtgataatagttgcccgaggtttttagcaactgcgagggttcaaggataccgatgccgacaatggcgatatcgacgtcgtcaaatctggcgataacgtcagccacatcctggcttgccaccaggcgttttttctcttctaccgaaccttcaatgctttgagaaggaagcaaccaggcttcgcaattcagatgctgggccagggtctgtgtcaaaatagttgcctgcacattgccgtttggccccacgccgcccagcagctgaataacgccgctggcttttaagttttgggcgtgaacttcatcgaccatcgcgcgaatcgtagaactccacgaagagacgccaatgaaatcttttggccttaaacgcgtttccagatagtgcgcggcggcagagccgatggcgcgtttaatggtgtgatcactggcatcatcttcggtatcgacaacaatcgcttgtttaatgccgtaacgctcttcaatgcctttttccagattgagaaagatatttgacggctggacaacgctgattttaaccacgccttctttctggcaacgggttagcgctctggatataaatgactgagaaagcgagagaagttgggcgatgtcagactgttttcgcccctcaagatagtaaagagtggcgatcttcaccagtagccgctgttcatcctgcttagccatatatttccccgtaaatcattaacgtaccgcccttatcttagaggcttttatcatacgcgaaaaaagaagacagttaagtgtgatggagttctaactttaaccaatccttaaaatcagcgatggacaaaaataaaaaacaggcggcataattgaataaagaatcattattgaatatatattcaagatgatatcaggtcaactgatgattacccggacatgtaactgacggtgtaaggaggcagaatgttcctgagtatgatgcagcagcgtaagtcttgccgcgcaacgccctgggatttctggtttttttagtccccggtggaataaatattccatgttgaaaataaattcagaggtattaaatgaatccctatagctatgatatccagacccttgagcgtaaagcgcgggcggtacgccgccatatcgtgcgcttaaatgccaatagcccggcaggtggacacaccggggccgatctctctcaggttgaattactgaccgcactctattttcgtattctcaatgtcgcaccggatcgccttcacgacgatgaccgggatatatacattcagtcaaaaggccatgctgttggatgttactactgcgtgctggcggaagcaggattcttcccactggaatggctttccacttatcagcagccaaattcacacttacccggccatccggttcggcaaaaaacgccaggtattgaactgaataccggagcactggggcatggtttccctgtggctgtaggacttgcgctggcggcgaaaaaaagtcaaagccgtcgtcgtatcttcttaattactggcgatggtgagctggctgagggcagtaactgggaagctgcactggcggcggcgcattacgggcttgataacctggtcattattaatgacaaaaataatctgcaattagccgggccaacgcgcgaaatcatgaacaccgatcctctggcagagaaatggctggcattcggtatgcaggttactgaatgtcagggcaatgatatggcttcggttgtcgcgacgcttgaagagcttaaacaggaaggcaaaccgaatgtggtgattgcccacaccactaaaggggcaggcatttcctttatacaagggcgtgcggaatggcatcaccgggtgccgaaaggcgatgaaattgaacaggcactggaggaactcaaagatgcgtaacagtgaacatctggcaaatgtgatggttcaggcgtttatcgatgccgtaaatgaggggatcgatttggtgcctgtggtggctgattcaacgtctacggcaaaaattgctccctttattaagcaattccctgaccgactggtgaatgtgggtattgccgaacagagcatggttggtacagctgccgggctggcgctgggcggcaaggtggctgttacctgcaatgccgcgccgtttcttatcgctcgcgctaacgagcaaatcaaggttgatgtctgctacaactttaccaacgtaaaattgttcggtctgaatgccggggcgagttatggaccgctggcgagcacgcatcatgcgattgatgacattgcggttatgcgtggtttcggcaatattcaaatctttgccccgtcttcaccgcgggagtgccgtcaggtgattgattatgctcttcgctatcaggggcctgtttatatccgtatggacggtaaagccctgccggaaatccacgatgaggactaccgatttgtaccaggtgccgtggtgactttgcgcgagggcagcgaactggcgctggtagccacaggctctaccgttcatgaagtggtcgatgccgcagaacttttagcgcagtctggtatcgcggctacagttgtcagcgtaccgtcgattcgcccatgcgacaccaaagcattgctggcagcgttaaaaggatgcaaagcagtgatgacggttgaagagcataacgtcaatggcggccttggtagcctggtcgcggaagtgttggcagaggccggaacggggatcacattaaaacgcgcaggcattatggatggagaatatgccgcagcggcggatcgtgggtggttgcgtcagcatcatggatttgatgctgccggaattgcagctcaggcacgggaaatgctttga

SEQ ID NO: 15 - DNA, 201 bp, Artificial Sequence
  ST131 E. coli_NA114 CP002797
aagcatttcgatggcatataaggagtattacgcgctacatattacggaaaccaattctttcttataaaaactatagaaaaattacatccgaactggtaaagtgattaaataaagtcctaattttactactgaaatggtatctttatttaaagatctttgctttatataccaattttttaacgccctgacaggagtaaacaa

SEQ ID NO: 16 - DNA, 17304 bp, Artificial Sequence
  ST131 E. coli_NA114 CP002797
attggtagagaattttgcaaatctggttttgtaaacgttaaatcagcagcgcactgataaacatccagatctgattcatggctaattgttctgacattttctacagaaatacgcacatccttatccagtttcatccctttctcgattgtctttcttgcaatatccccgaccagattttttgcatcatcactactacattttggcgttttctcgccgcagccagccagcaaaccagcagcaattaataccagaagtattgctcttttcatctgtagttccttcgtaaattaatgatacttttatttcttattaaatagaccatccattgcgcctgccgcagaagccccgcagtatccctcgcaaccacgtgtcataatgctggccttaccgtttgtcagttcactaattactgcaacacactcgctggcattgtcattccagacccagcgacgcttctgactttctccggcagctatttgctgtgctgtcccctccatattgcatacgcccgaaccatcccccgttgaggcattaatgttgaattcagcgttattctgtcccggatgcagggttagaaccgctccgggttttacgtactgcgctgctgcgactggtagcgttatggctgccatgaataaggccaacagttttctgttcattaattaattccctgttaagtgatacggatgtttttggtgattttccccagtgccagcggtctgcgtttttccacaaagaaataaacgacgttgaatgcagacttgcgatgattccctcgaccaagagagggccagttgcggaaggattcaatctgttcttctgaatagggtgtgtcagagaaatcaaacagcacaagcaaggctttgccgtcatgtttcatcaatttctttacttctgtactgttgacggtcgcagagacattggaacgtgccgcagttggtttacgtactgcaaactcaacggccacatcgtcaatgacaaaatcaatgtaaccgtgcccggatacagttcctggcagtttgctttttacttccggtgagacgttatctgcaaaccaacccagcaaatagcaacgtaccagaggcagtaattcccgctcgctacattcgttcagacgcagggttttgacgaactccctgcgataaagagaactgaaaaaatactcaaagcattccgttatttcagtgatggtggttgccatggttgcatcctttacctgctattaggtccatttttaagggcatacctttccggccataatgtttccggtgtcgtatccagagccccggcaataatgcgctcagctctgggataatgacgctctaaggcgttatttaatgtccgtggcgcaaggccgtgactgcgtgacagtaacgccagcgttaaccctcgcttgtgaagtgctgccacaatatcggcacgatgccagttccctgaacttcctttgatttgctttttctgcgatgctgttgagttacgcacagtattaactccatggaatcagtacatggatattaaatgcgaataaaagatacctttcaagttcttttggtatctatttattggttttcatattaacgcattgatatctggtgaaatttatatgagcagtaagagaacagtttcggctatacctactcatcctgaagcttctgttgaagaggctaaagactggctgacagcagaggaattcactcatattgaggggatgccgggcactgcgcgtggcgccaggatgcgcttggaaaagctggcatccctgcacccggaaatacgtcgtaaaaggactgccgggaaaggtgtggtttaccatattagcgcagccacaatgtcgcttgatgatgagagaaacagggctaagggcgaagcgtttactctcaacgacagtgatcgccagctaagcctctgggtgcagctgtatcgcaatatgtcgccagcttcgcgggcgcaactgatggagcaggccctcaggctggtaaaggacgatctgatgtctcaggggcccaatgtttctgaactggcggtacaaatcatgaagctgcccgaagatgagcgtaagcgcctctttgaaaagctgaacgggaaggtgtgatagcgtcagttttccgccatgccagacagaaggaaccaccccggaatcctgacctgtctgacatggcagaatggaaagtatgccatcacttaataatgaataaaaacctattgccagttatttcattgctgaaccacatattagtttctctttgttactatttgcataaaaagtatatttttgatataataatattgctggtgaactatattttacccagtcagtacgttgtcatgcacttaccatttattaggtgattaaaccccacctgtgctggtgcagacataacgtcttatcagaatatattgaacaggaaaatcactgatgaatattgtccatcaggaagggctagctatgcccgtcgatcaggaattaataaatatcatattaaatgaagcgggaaatccaccgccgcataaagccaaaattacagccgtcagtctgttatttaaagacctgagttacagcgctgaaaagggtggttaccatcctgtcgaaatacggattatttcacgcaatgatgaatggtattttgattacattaccgacttcagttatatggggacggtataccccgaacttgaaaaggaaattgatatcagctggagtcagcaatatgtctttcttgcttatgttggtgacctgcctgttaacgccggaagggagctgtttgaactctggcaatccaactttattcagtatcacggcatgaacatctacaccattaccgttctctgggaaagctaatccccctgtactaaccatcactaatttatggggaattatctgtgacgactgacagagggatacgtgcgtctcaacagggcgaactggactgtctttgcggtgcttattgcgtggttaacatgctgagctggctgtatgacggcagggtgaaacgacgcccgttaatgaactggcttattcagacttatcagcagcgctggcctttgcatgaatggctgactgaaggaattgaagaagaccgcttagactggttaatagctcaggtgttgcagaaagggcattaccgccgccagttcccggttgaaatcaccagaccctttgcagggaagcgggggttaacagacgggcggctgttcagggaaatgcagcggtttcttgacgtcactgaccactctcgcctgattatgctcagcgaccagtttcactggtcccttctggtcaaaatggacgaagaaatgctctgcttcttcgacagcaacgggcgaaccaccatgccccgtaaagctttctccctgcgtactggcgtaacccgccgccagctgtttccagacgccatttacttcattgaacgggagttctgacatgaccatgtttacccagcgtgaaacccgtatccttgaccaggcgcgtgacattattagccggtactaccagcgtggggttcaactctgttcccctgatgatgttcgacggtgcgtgatggttgagctggcaccgcttgaacatgaagagtttggcatcatcctactcgataatcataatcagctgctgcaccgcgaaatcctgttcaggggtacgctaaactcggtcagcgttcacccccgtgaagttattaagcgtgtgctgaaacataacgccgccgccgtcatccttgtccataatcaccccagcggcgagcctgaacccagccggtgtgacatccagttgaccaaaaagcttcaggagctgctggaaatactggatgtgcggttacttgatcactttatcgtggcaggaactgaaacggtgtcgatggctgagcgaggactggtgtgatgctgaaagttacttggtttaatgtacgttattgcgggttacgcgcatgagcaggacagcgttgcgtcaggtcgtgccggtggtgcaaaccaccgtgttcgatgcgtatattgtcggtctggccccctcgggacggcgaggtatcaccagcctgttaaaccgcagcgccagcatacttaaacacggtgcggatgcagcagactatccgtgggaacaacttaactacgccgccgttgccaaagtgcgcgcggcactgctggatgacggttacgccgtgtcatcggtcaacatggcgctgtcagcactccggggcgtagcgcagacggcctttaacctcaattacatggatgccgaaacgctggcacgtatccggtcagttaagcgcgtgagcggcgatgtccagcgcaagggaagggcgctgggcaggcaggaaatccgggtactgattcaggctgcaaaacagcatccgcaatctgtacggcgctgtcgcgatgtcgccattgtgctgatgctttgcggaacgggcctgcgggccggggaactggttaaacttgagcgccgggactatgacaacggcatcctgacggtgaggcaagggaaggggcgtaaataccgtgaaattcacgtggcggatgcggtggacaaggctattcgtgcgtggctgaaagttggcgcagacgaggctgacagtgcgttgtttaggcgaattcagcgcagtggtaaggtcgcaagccaaccgctgactacgaccgggttaacgggtatcctggctgagttacagcaaacggcaggtatagcacgttttaccccgcatgatatgcggcgcaccttcatcacccgtttgctggagcagggcgtggatatcaataccgtccgccagttggcaggccacagcgacatatccacgacgacgcgctatgactgtcgtggtgatactatgaagatcagtgccagcaagcgcatcagatgtttttagcagttgaggcgttgccagactcggtttcagtttcatcttcagcactcaaccgcgcgtacttttgattctggatgttaatttgtgcattgatattttcaggctgaagcatccacgcaacatcctcacgcgcccattctttaccgtcagagagtcgttttaaccggggtttccatattccatactcattaagtttttctgctaccgaagcatatttctcacgatctgactcccccgtatgaagcttgtcaaaacgaccttttacaagctggtaaacagtatagatttccagtgccttaggggacctggccgggactatcatggggtgttcctccggcctataaatcccgtttggctctccctcattgagcaggtcattaaacatgtggttcacgcaggcactcagcacatctgccgctttctccgcagcgaatttatccgggctaaaacccgctcccgtcagtttccgggcaatcagtgccagtttttgccgggtatcccgtcccagataaagcttcttctccagaaattcaaagacctcgttactgcttttccgttcccggtggcatctggcgtcagccttgttgctttggttaccaatcatagtcacctccatgacagtgttttgtacaaaatatcaccattgagcctcagtgaaaattgcttctttatatgaccgttctggagacagaaatagatgatctcgaatgacatccacattaaagatgcccttaacgtgctcaacgaaggcatcgttgagtgaatccttgcgttttttggtggttgtaaataacccgctcctgaaacagctcaaaggagcacaagcatgaacaccaaccgtattttgcgtaaaaaagaggtacttcatctgacggggaactcgtcagcgacgttgtaccgactcatctcgaaaggattctttcctttgtcaaagaggctaacgggcgacaatggccgggcggtaggctggctggagagtgatatcaataactgggttaacagccgtatgcaggcgggagaatgaaatgaaagagataaaatcgaaaaccagttctgcgaaaaaaacgctggttccttggttcgaaactatggggaaacaggttggtttatcacaagaaacgatatcagcagtgttaaaggagcagcgtttacggatacgtaagcttaactacgcatctgcatatacagtgtccaatattctcatacaggattgcggcggggccgatgacatgtggagcaactacgctaaactggcgcttatccttgagcgttgtaaatacattccctgcgggagcagggcctgtgcatattgttctaccgctcagcaattacattatcagggggtggatgtaaccggggaatacgctttatatcagttgcctgagttatttttagattttttagaaacaaacgacaaaaattcctcttatcattgggattacaataagttagttgagcggcatgaagatgctcaggcgaggtcattcgggtaatagccatttctgtatggtgaaaaactgacatattaccctgccactgcattttggtggcagggtaatcaatacagctaggaatattaaatgaacgattcttattacttcagaacatctgacccggattacattcgtcacgcaccgtcaatgcttgagattttaagtaaagcattccccactattattacccagcaggaaagtgagtatgaactgcagcgtcttaaccatttactaaataaactgaaaggcagcaatcgctgctacaacatcattgctcaaaaactaaggcagtgcagaaacggatctccatgcaacagtcttatatgcccgcattgccagcgggaacgcattctggcgcaactggctatgttgcatgtcttgccggggaattcagcagaatacgtgggagtggtcctgtttttcaacaaggatacgcaaactccgccgccatggaagaacattggggcgctcagggcgcaaatcggtcgttataagcagaggataagccgggtacttaaccgcctggggtacgccggtccggccacaggcaccttcagtatgatgcgacatatgcctgatggcccagaagagcgtattttctgggttccacagttgtgtttgttcctgcccaatgacagtacgttgattaaggggctaaaagcgcatatgtcgcgaagtggtggggcattcattgatgcatcgacgcttaacacaccagtgatcgtgctcaggtataaagatcccgcgagactgatttcatgcgccctgaatccagtctggcacacagcagattacacactgactgataaagacgctcttgttaagtcaagaatggagctactgaaaggccggacactgttaaaaagtctgcttaccctggactcactcggaacaggtgtcgtttctttttcgtttgggcaacccccagggaaagtgcactgatttaccggtaatgcagcaactgcaatgcacttttgctgttgattaaattgcagtggaacgacgaataacagcggcaaccactgcattttcgttgttgtaaaactgcaaagcttgccagtgattttgatggcgctgatgcactttaacattccaactgtctcgattttaacgcaaccgtctgttttctgcaggggctcacggatgcctctgcacacccgatccttaccatcaactgacgagttagcgcatctgtgagggctttctgtgtttctgtcagtgaatattctacacacaatggggggagcggtcagcgagcaccattgccattgagcgcaacggagagcttggggacatgcagcactacggaggcttcatctccttctcatgtcattaaacgatacggcgaacgagcgcagcgagtgagggaaggcgtcagcctgaacagggggaaagatttgcgaccggagtggagcaaatcatgggcggagcccatcctcgcgactggggttttgactttcgaattgcccagcgttgagtttggttaatgtagtgagacgaggatgctgagaggggaccgtgagtactgaaaaggattatccagtgaagatccctaagtgggtttatgaccgtattactgaaataacggattatgtcgtgggggatacgcaatgggctgttacccggcggcagacagtacggtggttcctggctcacatatggctggaaacagacgatgatggctggactatatgcactgtccgggatatccgtagcagctatgccagtctcctgggacagtgtgaaattagttaccagggggagtgctttatgtccacgctaactgatttcctgccgacactttcggatattgaatttcgtgcaggaaaagcgagcaaagacccggaaaagcgtagagccagtgcatggcggttcaaccccctgctgcctgtgtgtgatgagtctgccaggggtaagttaaatctggttgacttggatactggtgagtctgttagttttagagctctgcttcaggggaacgggaaagctcctgggcatgcgatagatgtagagaaacgccagacggccctaaaacaaacggaaaaggacttcttggccaaagttgggcgggggcgcatgagtatacagtttgtgaaggaattgcgtagccgtgagccggatcactattaccgcgcaggaatacgcagccttaaccatctctacaatggcaggattgagggacaatatgtcacttacgaccatcattatcgtctgacgtttggagggcggtattatgatcaagctttccagaatctgcccaatgagttcaaagctaaatttcggaccgggttactcaactatgacatcgaagcgtgcaatctggcctgtctgaaccatctgttcagacagtatgacgtcgattatcgtgtgaagtcatcgatatataaaaccatgatggaacataccgggctgacccgtaaacagtgcaaacagatggtccataccaccacctacagaattgggcgtgtgactattggtgttaatgatggccttggggcaaaagtgtatggatggtgcggtaacagcaaaaaaaaggcgctaaagattttgcgttggtggaaccgttatatcagtccattgaaatctgcgcttgagtcattgctggaacgtgtgcatggcgcacaccgcaagagttgttcatcaccgcgtaactatcacaggtacgctaatgaagtcggattaatactggatcttaatagtgaggtgtatcagcgtgaaaagactcaccatcatcaatatgcgcggaataaggcactgttggcgtttatgatctgtggtgtggagcaggcgtatatccgtgaggttgtcagtatgaaccctggtcgtgtctgcatgctggatcatgatggggtagtggcaacgggcgcgctttcactaccggactggcgtggtttcacgatgaaagtgaaggactagcttactttcttgaatttaactatctgcgatctctgataagggcgtattacgttgtcaatcataatgggatatgcgcattggaacgatgtgcatatccagtaagtgatccatgtccgctttgagctgtgagttcaacgggtcgatgcaacgctgtccctaatcaagggggacgttgccatgaaacgaagaactcgtattaactacacgccagagcaaaaggtgattatttgggacagatataagcaaggtgattccctgcatgatatcccccagaatgttcgacagatttcactctactatcatgcctacaatccaccaggccggtggctaccgtcctcccgtgcgaaagcggcaccggttagcgttttcgcttgatgaaaaagaggagatatccagaggactggtagcaaaacgcatcatcagggacatcgctgctaaattatcaatagctccctcaacgattagccgcgagatcaggaggcacggcggtgcaaaacaataccgtgcagcaaaagccgatcctgctgcgtgggaaaatgctctgagaccaaaaccttgcaagctaattgaaagccccacattgtgtaaaatcattgcagagaagatgcatcaggactggtcgccggaacaaattggcggttggctaaaacgctgttatcccgataatcaggaaatgcatgtgtcacatgaaatgatttataaaacgctttttataccaacccggggagctttaaaaaaagagctgcagcaatgcctcagaagcggaagagtagtgcgtagatccaaaacctcatcacttaaagtaaacagagatacattctggtcacgaatgcccggttgttcgtgctttgcgttttaatcgcattttacagtttctgcctgcgtgcagaggttgatggtattttctgaaaaagcggctcgctttgctaatgcatatggcacaaatacaacagtttgtgtcattctgacccatctttataccgcgacggtggaaacatggatgagctgctaacccttggctgcattgctttattcttcgcactggtggtggttcccatcctcgcgattatcgcgctcagccgcagcacggctgttcgttctgagctggcgaccctgcgtcgccgggtggaggtgcttgagcagcgcggtgtgactgaagcacccgccacagtggtaacaactgtgccgcagagtgtttcgcagatggttgtgcctgttgaaacgtcttcgcctgcggttaacatcgtgctgtcggagcctgtggctgatgtcacgcctgagtccgttgacccctggcgtccacaaccgaagccgcaaaacgaggcgctacccacaccagcggtcgaacaatcttccgcttttggtggggtgatgtcgtccctggtgcgctggtttatgcagggcaacccgctggcaaagctgggtattctgcttctcttccttggcttctccttcctgctgcgttatacggttgaacactccctgttcccgcttgagctacgtctggtggcgacttcgctgtttgcgattgccctgctggtagtgggctggcgattgcggcataaacagctggtttatgcgctgatcatacagggtggagctacggggacgctctatctcaccgtttttggcgcattctggctctggcagatgctgccgatgacgctggcgtttgcattactggtggcgatttgcgcggcgagcgtgggactggcgattatgcaaaaagcgctcagcctcgcgatgctggcaagtatcggtggctatctggcaccgctgttgctttccaccggcggcggtagtcatgtggcgctcttctctttctaccttctgctttccgtcggtattctcgccatcagcatctggcaacactggcgcgagcttaatctgcttggcctgttatttacctttggtatcggcggtctttggggactcgacggctatcgatcagagtattacctgagcagtcagcttttcctgatagccaatacgatcatttttggtgtgctgagcgtggcgctatcgctacgagcacaggagaaaggtaagcagattattgatggcgtgctgttgtttgcatcgccgctggtcggttttggcatgcagtacgccataacccggcatatggaatatggtccggcgcttagcgcgctgggttacggcggtttctatctggcgttagcatggcttgcattgcgtcgctatccctctttggggaggccgctggtgctggcggcgctggcgctgggtggggcatttaccacgctcgctatcccactggcgctttcggcccgctggacggcgatggcctgggcgctggaaggtctcggcatcctctggctgggcgtgcaacagcagcagcggcgtatgagctatagcggcactgcgctgctggtgctggcagtgtgcagcgcgctgtgggcgcagatgaacgggatgagcgcgctttctctggtgctaatctttgccgtactcagcctcagctggctggctgccgcctggctgtggcgcaatattcagttgcagggtagttgggttctgttggctggcggtttgatcttctggataatcgcgctcattggggcatcgcaactggtgctgaaaaaggactcgctggtgctgtctggcgtgctggcgttaatggcgatatcggtctggggctggcggattgtctctggtcgtctggcctggtgggagctggatgtcagcaaatggttgctgtggccgacgatgctggtgatgctgctgtctcaaatttcacaacatgagatttttgctgcgggctggcagaatctggcctggtgtctggcgttacccgctgcgggggcattgctctggcgtgatgctgagacgttgccgccgcgcctctccagactggcgcacctttcactcttctggatgattctgctcgcgctggcggcggagctattctggtttgcgcaagatctgccatggggcatggcggcgtggggctccggcctgatgatggccgcggggggcctgctcatctttctcgttcatgaggcggttcatcgtcagctatggccattccgaagctggcccgcactctacgcttctcaggcgatgataccggtagctgcggtattggttgttctgctagtactgacgaatttgcaggatggcgtagtttatcgccagacctggctgccgctggtgaatccactggaggagggggccgcttttgcgctactgggactcattgtattttatcgcgtttcgcagcgctttttcccggtgcagatttcggtatgccgtccctggccgctcattgcactgctggcgcttagcttctggtggctgaatggcatcctgctgcgcgcattggcctggtacggcgaagtgccctggagcgcggaaacactgtggcattcgcggctgattcagacctgttttgccctgttctggatgctggcggcattagtggtgatgctacgtgcgacccggcgttgctcacgtcgcgaatggctttgtggatccgtattgttagggattgttattgtgaaactgatgttggtagacagtgcgcgtggcggcggcctggcacgcgcggtcacgtttatcggcgtggcaattctggtgctaatcgtcgggtatttttcaccgttaccgcccaaagcgggagaagaaaaatgaaatggatgaaagcggtgttatgcagcgtgctactgagtactgcgggcgcggcggtcagcagcgatgaagtgaaggaagcgccaacggactacgcaacaggcgtcgcgctggaaaccacgggggcattcgccctggtatcgcatttcgctgccgcaggtggtgtatcagagcactgcatggccggatttgcgcgatgtgcgggtttttaaccatctgggggatatggtgccgtttgcgctggtggcgcagaaaacccagaccgttacgccagagacagtggctctgcgtcttttcccgcttgatatgtcgccggttccgccgcgtgatgagggtcaacgtagtggggatgctgttgttttacgttcaaaaaacggtattgaaattcatttgcaaagcgatgatgtcactgcgctcgggcagagctatttactgacgttgccggaagagaggaaagactctctttcactgacgcaattgcgtctgaactgggggacgccgacgggcaactggcaggggaaagcctcggtttatgtcagccgtgatttacgctactggcgacccgtgcaggaagatgcgccgttaatggatctgacgcgggatagcgatcgtctgaaaatggacgccatcagcaccaacctgacattgtcacttgaaggaaatcgctacctgctggtgatcctcaactcgcaaagcccggcactgacgttaaacagcgtgagcgctattgctgacagtaatgagccggagtccgaacgtattgtgattggcgctcgggcggataaggtgtcggatgatgaagccgtctggcgatggacacagccgcagcccctgacatcgttgcggatcgatctggaaaatgaaggcgttttaccggtggaactggtctggcgcagcggcgaaaaagaaccctggcagtcactgacaaaaacggtgttgtatcgcctggacgggaagcgttcagaagatattcgtcttcctggccagttagttgaggctgttcgaatacgtacaattaatgcgcgattgccggaggcattgccagcattgagcggtgcgcgcgacagttatcaactggtttttaacacgcaaggcaaaggtccttacatgctggcgtggggaaaccgggcggcgaaaaaggccgatgttggactcgatatgttgatccctgcgtcgctgcgtaaaacgcaggagatagataatctgccgtgggctataccgcaggagagcgtaactctcggcggtgaattgcgactaacggcgacgtcggcagccgaacaacaaagtcagtggaaaacgctgctggtgtggggggcgttaatacttggcgttgccgttctggcgtttatggcatggcgaatctggcgagaagttaaaaaggacggtgcggcgtaagccataaaaaagcacagttgcatagttcacggtgttgataaactgttacaacagaggcgtttaagctgaggcggggaaataatctatttgtgaataatatgttacttcactggggcgtgagtcatgggaatgattttgatacctgcgttttttattttagccatcctgattatcgctatcttcatttcagttaagaatggccgtcacgtatccgttgtatgccttgttgccgtgggtgagattttcgtattcccattctattttacggcccttaagcgtttccggagcatcaaaacttggtaaagatttaccatgttgacccattatgcggcccctctggagaccactttcaggtattttctctactgatatgtacacatggagagatgtgattgcaaattcccatcagcattactaccaaaagaatcagacaggaagcagcatagtattaataagaatattttcaaacataaaggatgattatgagtaacccaggcccacttgcagcgttagtgtatggttttattttgatagttttgctatcggtatctttaatcctttattcaattggacgtaccataaaaaacgagattggaaaaggtatagcatacgcgcttactggtattttattaattctgtcattattagttggcgctttcattttgacgcttcttacataaattcctcgaaaaatacgctgacagcatcagctaatggacccgactctccgttgaacgcatgctcgcctgcggcacctgtgccatgggcgtccgccgctacgggcaggcatcaaggttcgcttcacaacagcggacctgctgctacagctatccacttcatagcgccagggccgttacaaaacgacactcaatcgcggtgtcatggccccgaagctacttatcatcgatgaaataggttatctgccgttcagtctggaagaagccaagctgttcttccaggtcatcgctaaacgttacgagaggagcgcgatgatcctggactccaacctgccgttcgggcagtgggatcagacgttcgccagggatgcagccctgacatcggcgatgctggaccgtatcttacatcactcacacgtagtccagataaaaggagagagctatcgactgaagtagaaacgaaaggccggggttatacctgaagctaatcctgagtaaacaaggtggatcaatattaaaccgttggtggtgacggttagtggatcactttattacccgttgttgacagtaaacgcaggaagtcagatgtgatgcgccgaaagctgctttcgcacatgttacagggactggaaagccgggctgtttacgattacgttgccagtcataagggcgtttgcgcactggagcacgatgggtttttttcatataaagaggtcactgactggtcccatccatatctgttgattgagaaaaagcattaaggggagccactcagtcaattacagcttatctcggttgataaacaagacgggacatttagcactttcatgatatgcatagccggcaggtgatcttagcctgctttcagcgatgaaccacacaatattccccttttgttactttgatatgattgctcaaaatgggtggtaacactgagatcgttgaaatgactgcatgttatattgttacttttgttaccgttgaaaacatcacgttggagagctaaatggattggccaaaggagtacagcaagacgactcaagtggagtgaccacattccggtagacagtttctgtcctcacggtgaggccttttcgaaggcctccgggctgacgccacccagatgactgtggcgccgggtccggttgtagaacacttcaatgtaatcgaagatatccgcccgggccatgtcccgggttttatatatccgctttctgatgcgctcttttttcagtgaactgaagaacgattctgccaccgcattatcccagcagttgccacgacggctcatactcggcgccagattgttggccctgcagaaccgctgccagtcatcactgccatactgactgccctgatcgctgtgcactatgacgttttctgcaggcttacgccgccagactgccatcagcagcgcatccagtgccagctcgcgcgacagcgttggtttcatcgaccagcccaccacattacgggcaaaaagatcgataaccaccgccagatacagccaaccctgccaggtgcggatgtaggtaatatcagtcacccacacctggttgggttttaccatcgtaaattccctctgtacgcgattaggagctatcagtgacggccttcccctgacgccacgcgggaccttatagccatgtacggcctgtatcctgttccgcttcatgattctggcaacacggtttctgctgcatacttcaccgatttcacgaagatcgccgtgaaccctgcgataaccgtagactccgccgctcagtgcataagcatcgcggatgagctccagcaaccgctgattatccttttctccggcagaaacagggcgatgaagccagacgtaaaatcccgcacgggcgaccttcagaacccgacacattgttacgatcgaccagatttcacggtgatcgttgatgaagcggtacttcagtcgggctcccttgcaaagtaccgcgctgcctttttcagaatatcccgttcttcctcagtacgtttaagctgcgctttaagcctgagaatctccgtccgcgcatccagtaaatcctgcgcctgatgcccgctgttatccggtctgactgaccggacccacttataaaggctgtgagccgatacgccgaggcgttcagaaacatcggcaacggaatagccccgttcagtgacctgacggacagcttcttccttaaactctggggtaaatcgtggtgtgcccatagactcctcctatgctcaaactatagggcagatttgtctacgggcttgggggcactccagaaccttgaggaataagttctatcctcgtggttcactttgtgcagcaagttc

SEQ ID NO: 17 - DNA, 23818 bp, Artificial Sequence
  ST131 E. coli_NA114 CP002797
ttaaaacgcggttgggtgaattccatattcaccgtttgatccataccctatgcgatacatcaaagtacgatctgttgtcaccttgcccgcttgttcgctcatgccaccaccacatactccttttggccacgcgctgaagacatgatcaccaatcgttgggtatacaacgactttctgagctgtacctaaatctgccacctctttaccatcaacataaacacgggaaaaacacgcgcttccaaagaaacccgagtcacgcttgatgatcacctctcccgtaccgtctcgtttttcgagtaacgcggtactgataatttgtttttcaggaacaggagtggcttgttcatttgagacaggttttgtagcacaaccgacaagagcaagcataatcccacaggcaagaccatttttaacgaccatgtttgaaaactcccaatattaaaaatatgtcagcatctaacgataacaacgttcagcaccagagactcttcgttctccggcttacgcctaaaaatatcgcaggtgccgtttgcagtcgacatctgttaatactaaattcataccgccaccggctcgccgagcccatttactccagagacagccgaaccgaaaaaccgttttcagcgagcgtcagactcagtgtgttgatagtccagtcccggtcctcctcggtcccgaaaccttgagttgtgactttgcattctgctgtcagcgacactaaatcgagcgtcgctggcgtcgtgtacgtcatagagaactcttttttagctgcaccgcccttgagccctggtgcagcttctttcgctgcgtcgagcgacggctcaactgccggccattcaatgacaggatcgccgccactaccttgtccgcaattctttgatctgcccagttgctgtgtcgtgatatttaatcagatacgtcggttccggattttcgccaccgctaccactatcgccactttgtccatttctcgaatagccccagctggagttatcacgacgagttagcgtgtagtgctggagcggagcaccgctaacagattcgcccgcgccgcgcggcaggaatatccagtacccgcccgcgggtttactcaccgcatcgaatctcgttgcgagccgcgacatcaggtgcatatctgactcgccgacctgatcgagttgcgtgataacattgtctgcaaaccgctttgatacgcgtggttttagcccattatcgctcgctattgtcgcaacgatatcgctgacagtgacgtcgctccaggaacgagtctttttgctctgaactgtcggagatccttttgctgcgttcatcggagccgcttttgctgtgaaatcaactacccggggttcaccgccactgctagcaccgtcaacgatgtatatccctttatcaattatctgattaccgaaaccgagcccaatactgatttttactccgcgggccggaattttcagagtttcagaaactattgctacgcggagttcgtctgtctgtttactggagccgccgtaatctgtcagcgtgatattgatcaaaccttttcttattttatccgtgatgtttttattatcagcagatatataaaaattaggtttccacgcctccacgccagtatttacgtaatcactcattcatcaatcccataattgcgtttcttttttaaccggctctggttcgatatctggtaaaacaattttttctccagcacgaaacacggcacacatatccgttacgccgtaattcgaaacctgatataaaacagtctctgtcgtttttgacgctctcccgtagaccgcaaggcagatctgatcgagacggtcaccgtcccgtgtagtgtatgtaatcatttggggccccattaattaaaaggatttcataatgagaattaattttagtagtcggagaggtattagtatttcggcgtcaaaaaaaataccatcaggttgttttacatttatgttcgtagttttttttattatttttatacttgttaagtgctctggtagcgcttgattttgtattaatctagcgaagctccataatattcgagcgtgagtgaaaagtcctgcgatttaggtgttccgcccatcaaaaagctgttttgcgtttcagttagttctcgcagcacccagtaccctttcacctccccggtccccagcactagctgctggggttctcccgtgttcccgagcgctcgcaaatctgtcaacaggccgacgccgcacccgtcaagaaataaagcgtttatctttccatcgatagtaattgtggggttcggtcgtccggttatctgcaacgcgtcgtttttcccgaatcgtgtttgctgagcccagcgccaggcgtcactacgttgcagtttgttgtacgctactgttgatatagcaaaaatgaaatcacccaggccgagcatgatatcctcagcaccgtttattgcatcactcattgtcaccccgccgcagctggtttatcaaacatcgcgccacgattgtacgaactcgcagcctgtctcagtccatcgagcacccccgacgctgcagattgttgtattgactgcggatctgtcgcaccgacaatattgatttcgattttattgttttgctccggcgcaggtgtcgagactgcctgcttcaacgtatcaatatttaacgtcgacgaatccagtgctgcaggcgcggtatttttatttaccatcccggataatggattaacaaacgcgagcattgattctgcctgagattttgcctgttcttgccgttctggatgccgcatattttcgacagcctcttcggcttttgccttcgcaatattatcagcagtcaatccagcgttcggtgaatatttacctcccgaatccataaactcttgatatgcccgattatattcgtcattataagttttctgttcttcatctgatttttcatccggaataagccactccagtttctttacgacagcccatataatcttcccgaatttgacgcaaccttctccaaaattgacgatgccatcccacatttttttcatattttctggttttacccactcagtaattgtgctgacaaaaccaccctgattatccttaaaccaggatgtaaatgtttcacgtactgagtcaaacgtcggcgcgagttcccccccgattttaccgagtgtatctgcaagtcccgacgtaatagagccccacagatttgtcactgatacatgcgcacgcgccgccccttccgcaccctcttttgttaacaaattcgacttttgcgcgtcagacattgtctgttcccagcttttacccgtcgccttcatatacgtcatgatcttattagcctcgccccccatgagctggtcagcgaggctcgccgcttgcttctcatctttcatttcagagatgcgacgcataacctcattaaactgtttttcccgatcccagccagccattcgttttttcgtcaggccaatttgaaataacatgggattcaggcttttttcgtttccgatttcgccgattttatttgtaagttcttcagacaaatccccgatattttcgccgttcaggcccgcggctttaccaatattttcccacgcagcgtatttttcgacgccaacgccgtaggacttcgccagaccgagtttttctgacgtctctgcattcattgccagcgcaccagctgcagcaccgccaatgagccctgccgcgccaagtgtcccccatttgataccccgccccgtagccctcaccgcacctgcggcggctcctccgccccattttgcagctccttttgcgcgacccgtccacttctccagccgctgttttttagcaagttgagtattcagcgcctcctggtcctgcgttgtcgcttttattcgacggtccagcttttgatactgagcagataaatcagaaacgtctttacctgccagtttcattgctgcgattttgtcttttaatttatcctgacttttcgttaatttcccgacagtagacgacgtctgccgaatcgcatcattgagttctgttgtaccgcgacgaaacgacgggtctattcgcccaccgaatacaacctgcgttttaaagttttgagaaatagccatcgattttcatgacctcgtttacatagaaattaaaaacccggtacggcaggtccatttgcgtatcgggcgggatggcaagttttttcgcaataaacggaatcagaataactatatctgatgtcggtctttcggaggctttaccagttcgttaaatgcggcctccatttgatcgtaatcgcacgcaggcagtgcatataaatcctcacgctccagatttgccaggcgcgccagcattgtcgctgtccgctcttcaagaccacttttatcgttactgaacataatgcggtcgcgtaattttggttcgcgaatagtaattgtatcgcggctctcccccttaataataaacgggcgagagagcgtaatagtcatactgtctaataatgattccattttatttgccttttaatatacatggagaatacgagcaacgcccgcaagaacgtcaacaccattaattactcgtttcatttttgcaggatgaatctcgtaaataatttttccattttgaactcgtttataatagctcggcgcaatcgttactgattgcccaactgctgattttgaatcagtcccctgcgcgtcatcagtaatcgccgtaataagcccctcatatgtatcaatacgttcaaaattgacaccgtaattgtcagtatatgcacttcgaatttcgaaccttgttttagcacctggtattaatcccaaataccccatcacaccagaatcggagccagagactttaaaactggctgacattttctccattcccccgtccagagtgatagctgtatccatagcgcccgtcttaaaatcatcttcgattattttcatatcgacgggagtgtattcagtgcaatttaaaactcgttctccctggacgaacaacgcaaatgcacgataggtgttactatctgccatatcaattctccagattattagactgtttctttaaaaagagaggccagagcttcaactgtgtaatcattattaatgcggtacattaacgtaattgtttgtgccggggatttcggtccaaagtcaacattaatataaagattaccagcggccagactttcttttgtatttaactcttcatctaaccacgcccgtcctccgttaatcgcgccctgctcctctaatttatgcagatacgcgtttatcgactctacaacgtctgttccgaggtgcaggtctatcgggcgatcaataaaatcggtcatcattgcccgggctattgaatcctcaattacgtcagctgtccgacgaacgcattcaaaggaccattggggactgaggctacacagatagttaccccagtgtctgaatccatcatagcgaataattgtgctgacctggctggcgttcagcagatttgctgtgcagttcgtttcaccgattataaaatcatcgacttgttcgagtcctgtaattccgtagatattttgattcgatttgctccaccaaaaccctttttcgttatcaattctggctcgtaaaccagcagcgatagctgaatacggacgagataaaccactaacatcactcgtagaataaacacgagggcgcagaatttccattctgccgccgtacctctgacgacgatttacaacgtcagcaggcgtagcgcctcgcggtgaatcgatatatccaacgcctcgtaatttatttgtcaccacttcaagttgttctgcaatataatcgttagcactataatctggagcgattaaaattctcggcgttacttgatttatttgcgcagacgtcaacaatgcgttaatcccgttaacgacgttctgccgcatttttgtttcgtcgctgtcctcttcgacgcggacaacaacaataatggcatcctcctgatttagtatttcagatacagcgggaggaagggttccagacccaccgagcaatccagcttttttttgactgcctgcaattaacgtcggaacattatatttgaacgattcgtctttaccaccggataaatttgttggggggagcgcgaatacagtccccgttcctgcgtgatcaccaccgaatgtgacgttaatatattctccaatctggagtccttctttatactgctggttctgatcgtacatttttgacggggtcatggcaccgtccgtgatcattgttaattttctcgaaccgtcgggcagtgtgctgtatcctggagttgctacattcccgttaattcccaaatcaacaatttcaacaatccagttgtttccgtcggcccctggcatgacagtcgaaaactcaacaagattgtctgcgagctctgatccccataatagtgatgcacaagttgggtagctggaatcgggagcagtaccaacaatcccaatcacggaaatatctactgtgttgatctcttttgtgccgtcgtcgtattcgagcgtgcgaataccgtgtaaaaaattagttacgctcattttattccttctctattttcaccaattcgcgtcgctattgcttctttaagctcggccaaattttcgatttgcgcgtcagatgcggttccggcatctactgcacactgaattgagaatgcctcaattgacagatcgccaacagtccgtaatgtagtctggagtttttgcgtctgctctttttttacaaatttagaaatgctcccataatccccgctcagcgctctttcatataaaaccctaccgtgttcctcgcaatcatttttcattgcggtgaatggaatgtcgtttccaatcccgtcaatatcgcaaataatatttatagataacccatgctcgtcattaaaaacagggtttgttatttttctaaatgaatcaacgcttatcattacgcaatcctaatagctaaaaaacaggcagtcccaatatcaccgactccaccgggtgaatacgccatggctcgccatgagccaataagtggtgtgtcataaaatggttttgtggcaatatccgtgttatatgaaccggtattatttatactagcgtaatataaatcccctccagcccatatggagcccgggagtatttgtggtgcatgattaatagcgacaagcgcataagtaccaaccgcgccaacaacacctatttccggtgatgggtccggtgaaagacttatatctatgttgccattttcatccggcgactccccgttaatcgttttaaccggtttaagattagcctcattccagtatttatacgtctgcccgccgcacagtaacaccaggccatcatagtgaactccgcgtaatctataaaaaaccttctgattgttttctccacgggtcgaaaaatctaaataatcgcccgacacatctgataaaccagcaaatgaaaacaacgtcaaatcagtgcccggtttgtttatatttatttgttttttagaggataaaacgtcttcaaaaatcacatcatctttaaaagtatttggatgagtaaatgcattacccttatctctatatgcaacgtccccatctataccaatatttttccgcgcagcctgctgagcagtttcaccatttgcgggaatttcagataagtttttacttatctgcaaatattttttgtcagcttcctcgggagttacgtagttgtcatatactacaacattaaccgtagatgtgctggacaccgcgagtgtgtatttatacgtgattgaaatcagcgcaccgttgctgtccgtaggttttaaaatgtccggcgagcgcgccacagagtacagctcgcctttatctgttaatatccccacctcacggatagtatagcccccagtgtctggcggtacgtaaagtgtaaagataacctggttaccgctattttctgcggatttaatatccccacgatacgtctcgtgaattaatgcagttcgtgccgggtccggtgttacgtcgtcgtttccgtttgcgtcgccaataacgaatttattcagcaccacgggtacacccgatgccagcgcagcggcttcaagttgtgcgccacggtctgtcagtactgcgtaaattttattttcagccatttggccctatctccacatttaaactaacaattggtgagcccgatatatatagcccgccgttaatttttacaccgccgtcgccgtctgctgggacgtttacgcctattttcgcgtaaggtgtaccggctataaaaaactcaccatcgacacccgtttcgaaatttatttcttttaataaggaacgacaattttttgcatcaaatatttgtgcaataaatgtattcaatagttctaaatctaaccccatgctttttagcagatatattttaattgcgaatgtatacgggtcgtccctgggcgtggcttcaaaccactctctcattttgctctcaaagccaacagttaataacgctcgttcaacagctccacgcgttccgcggtgcttattctgccacgcagctgctttaataatttcgcgtttttgctgctcactccagtcggggttccagtaggtgaccgcgtactcccacgccagccacggcaacagatttgcagggcacaaatcgggatttttaataattcgaatatcgccaggcagatcgccgacgtgagataaaacaacctcaagcgccctctcaggggttattgcgttcggaggcagtacgctttgacttgtcatggctaccccatttgcacgctgagtttgatttctgtgcagtacggagcctgcccgacgccagcgatcacatcctctgttggcgatatcagaatcacacgttcagtgccgtcacgatgtatcgcgtcgtaaattcctgacagagctgccgtcgctccgattttatgttgcgtatccacatatatcgccaaatcgctctgagcaccgttaagcaaaacgttctggtcgggtccgagcccggctacgataacggcctcaacccggtaattcaggttcgacgcaggttttacagtcacgaaatcagtcagcggtcgagtatcgtcaggagacagggccgcattcactgtattcagtagtgattgaggcggagtgccgtcgccggttcgcgacaagacatacatatcgacgtatccaggctgggtcaccggcgggccgtacgcctgagctgacagaacgtcgggatcactcgacagcgcgaaatagttataggcgttcgtactgccggcagtattccgcgcataccatgatagctgtattcggtgtcgatactcgtcgtcactctcgtaaacagcctcagtcgggggaattacatcgggattagctggcgttatcaacagccgcaggcagtcaaaattagcgccaatctggtcgaggtcactcccctttgcaaacgcgagcaatacagcgagcatcccctgatttacccgagctgtaataatgatttctctgtatgcaactatttccagcagtttaacggccgggtctgactccaggagtgcgttaaattgtggatataatatctgcagctctgacagtagattagatttaacatctgaaaataacggggttttaacgaatgctggcggcggcagcagactcatatcaatagttttcactatcgaattctgaactgttgatgttatcaaaatatcagctccatattattaaactctaatcgctgttgtgtttcgatatctgttgcgataattaatattgttattttcccgatatccactgctaacacatttattctgtcaattctgacgcgtggttcccatcgtgctattgcgccagcgctttccatgattattcgcatagcaagcgacggatctgtgggattatcaacaagatcaaataatttgctgccatattccggcagcattacacgcgtccccagcggggtcgttaaaatatcaacgatagactgacgaatgtgatcagtccccgataaaagtttaccggtgttacggtccattccaagcatctttacgccattggttggttcggttgttgagtttccccgtccggacatatatgagtgtgtccgttatacgttgtgcggacttcagttattgtgccttgtcggtcctgcacctcttgccctgctaacacactaccgcgagttgtgatattacctgttccaccgttatctccagtgacagaaatatcgttacggaatgtcgctaatttatctactactaatgtctcggtaatatgcgttggaccagctagtgttataccacccggagcccacgcgctgattgtttgagctgtcagcgatattctgttcgcagcagtaacactaacagaactcgttgctataacgttcactaaatcagcatgtgcgttaattgtatctgtcgctgtgacattaataatatcagcgtctgcgttaattaatttcccaccatggatattaactacattttcgctattaatagtaacatctgcgcgtccaaatatttctatttcagactcaccgagtatttttaccacgccgtccggtacgccctgccatgtcagagtgtgattttttgtgttatacgtcatcgtagccccgtcacagtatgcagtgacatgatcatgggggtcattgcttggcggagctttgtcgtcaatgtgcagcccgagcataactacaccgttccgcgtatccccaccctcagaaacaacagtaacgggatcgcccactgatggtgcgctccagtcgacgcgttctgaggtggcgtgagtaaaccactgcagccagcctgactggtgttcgccaccaaatgagactcggcagcgtggaggattcattttaactgcagctatcgtgccgcgtttaactgcgtcacgcagcctgcgcgcgttctccgctgcagcatattcatcgtccgattgggccataatagtcgccctcatgttctgcgccgatatctggtgctattccgacgtatatatttttcagggtcccctgtgagggataatcgaaaatatctggaccaacgcctacaacctgcgtgaacgacacgcactgaactgaatgcgaaccgactgactgcccgtttttaatccagtcgcacggttccgcgtcgctgaattttgccggtttagttccgggaccgaacattcgcccattgatccagctcgtcatatagagcgctgcattctgcgatttcagtccgtactgatcagctgcaaattcacgcagcaaatacaaattacacgacagctcgactgactgaattgagcctggaaccgattcgtcactctgtgaccagttgctgatttccaggaacagagctggcgtttcaaatccagccggaatttccggataaattccgaatgttttgatgaacggtatttgtaaaaccgcgcctttaacgcgatcaagatattcgtcaaatgcatccagtccgctcataatcgtttacctgtttttgggtctacgtggacattacctttcacgcgaccgcgcaaatctttctcaaaaaagcccataaaaacagggccaatgttttcaaaaatataatcatcgatagcatcctccagcgcgtcgtgcacaggtacgcgagcctcttcgattcctccgcgatccagtcgaatccagacacttttcgcaccgtagcgtttcgctacgaatgaatcgggccaactcatcatcgccagccctgcacttttgggtgtaaaagttgcccctctagcgccttttttggttttcaaaaacaggccggtctcaagatcgcgaggctgtttttgcctccgtggattttgtaatcgcccctttagctcagatacccggaaatcattcagaccgtaccagagcttggcgctgctcaggtctcctgccccttcttttgtaaaattacggcgcttaatgaacggcttgattcgtttatctacggccttatgatttttagctgccagtgcagtcaaaatcattcctgctgaaattcggtgcatatgttttgcagttcggttcagcgccctgttgtataccattaacatttgatgttgcgtcgcgctgatttcgactctgagctgctccagcgccgaaacatcgatatcgaacatctgcgcgttactacgtaaatcggccacggttcccccttaagtgggaccgaggtcccacttagtattttgaatactcgctgttttcgctggacgtgtgaggctctaaaaaaatattcgtgagccctgtgccatcaggttgaggctctttaaccacgtaatcggtccagccagaccaggtaacagagccgtcaggttcgatgctgcgctgctttggaacctggacgacgtcacgcgcaacaacgccagcgacgtcgtccgagagcgccgtcaggctcgttactgtaccggtaataaaaccagcgtgaggaacgtcaacgcgcgcgtaaggctcgttgaaaatagcgataatggggtcggttttaccgcgtaatttaacgggccgtccccactcacggatcatctcctcgtcgccggcccggaggtcatcgtagtagctcatagagcgtaaacgtgatcagctgcgataaggtctgcgacctccggtgaatcaacaacaatttcacggcctgcggcaacaatctcgcgaacgcgacgcccgttcacgtagtgataaacgtccagcgtgttacgcaacttgacgcggcgcgtagccggtgcgagctcctgctcttgtgactgtgaaacgatagcgctcgcgattgattgagcgagttcattatcggtcccggacgcagtattcactgtaatctcgatgcctcccgcgtcaccggattgctcgttcagaatatcgagctctgactcagcgccggcaacgattgattccagttccgcaatagtcccgctgcgcggtaattcgcgaccgagctgagcgctcagatcgtcaatacgcgccagcaattctgtttttgtgctcatgttgattccttaacaaaaaagagcctttcgactcttacgcgatttttacgacaacgaacgcgtcggcgtccgtgaggaccattgctggcgcggattgcgtcattgtttgcgtcactgccggatcaccggtagtggtccagacttttggatagcgcgtcgcttcggaaataccctctttcagcgcgtcttcgtcctgaattgcgccataagtgcggagcccgcggttttttgtgttgccaaggatcatcgtgttatccggcatataacgcgtttcagtgtctgttaccgggtcgaggtattgccctttgtagacaaacagcgcagtgtcgccgtagtagcctttaaagctgacaacgtcgcccaggtttttcagcgcgacctcgagctggctgtttgagccacggcgcgtatccagtgccgtccagaattttttaaaacgcttcaactgcttccaggctttaccatccatgatgatcacgttaaccgcaccggaggcgaggtccgcgtaggattcaatatcgtcgctcgggtcatacgtttccgcgtcctgagtcgaccaggctgtcgaaccggactgagtgacgttattcgtcgcgctgcggctcatgtcgatctcataggtctcgatattgctgccggaaacagtgtatttaccgtacagaacagcctgtaccgcctggaactcttcgagctggctgatgctcagttcctcatcaagcaggttctgcatgatgatttttgcacgacgttcgaccggcgtttccggctggccaatctgctcaccggctgcgcgcttgatgctcatattcggattgatcgtgtgcttgggtttcgtgtaacccggtttgaaatggttcgttgaatagccgcgagtgcggtcaactttgccggtgatcatcggcgcgcagtacacggccatgttcacttttcccgggattttatccagaaaaacctcttcgctcgtaaacgtgtaggtctcacggaaaaacagctttaaaaacaacggattgaacttaaatacctgctgtgtcgcggtaatcagttctgacgtagtaaacgaatcgctcatttatatatcctgttataaaaaaaccgcccggaggcggcttgttttgagttttttgctatcagccgacgctgatcggcgtacccgcaaatgctgatttgcgtttagctacgtcggtaaccgtaccccagttaatcgccgtgtaacggaacgagcctgatttatagtacgagcagtctgcagaactggagccagtattaactgcggtggcagtgaggccaatagctttaccgactgagccgtcccaaaccttaaacgtcccgacagtagcgtcgagcataatcggtgtcagccggggtacgttgatcccgccctgaaaatgaccgacggtcgtgacaacaaggtcaggcccaagaataaaatcgtccgggctatacgtttctgtagtcattattcatcccctaaaattgaacgccctgcagcaaccagcatcgagacgtttgcggttgttcctgttgccgtagtactgccagcatcctgaatagtcgctggtgattcagtgctcattagtgtatcgagcgccgtttctgtccgcgcctgcgccgttttaggtgctgcagcgaggactacctgcgcctgcccgaccgtcatcccggggacgccagcgagcgcttttgcctgcgcctcgcgcccaacagcttcgggacaattgatgatggccatcacacgcgccagttcgcttgatgcggcgtccgcgcgaatctgctccgtgttaaactcaacctgcgcgacagtagtcgtggtcgcggtttgttctgtggtcgtggtctccgccgtggtttctgtaccagacataaagcgctccatttttggtttcagtgcgtcggccatcactgcgatagcgtccgcgtaattaacgagttgatcagcgagaccagatttaatcgcgtccgcgccgataaatactgcagcttccgtggccagcaccctggattttttcagcccggtataatccgagactttttgcgcgaactgctcgcgcgtgctgttaatactcagctggaattcgtcgcgaacgtcgcctggcagctgggaatacgggttcccgtcaactttgtgcgcgccggcgtaaatcagcgtcacgtcgacgccagcaatctccagcgctttttcgacgcagcggtgagccatcaggacaccgattgaaccaactgtgcctgtctgcgttatcagccggcgcgaacacgctgacgccagcaaataggcagcgctgcaggccgtatcgctggccagcgcccacacaggtttttgctctcgcgcccgggcgattaaatcagcagtatcaaacgcgccggcgacttcaccgccaggggagtcaatatcaagcaggacccctcttacatcgggatcagaaatcgcctgctgcaggcgttttgcgattccgttgtaaccgctcatcccgctaactgggttgatataacccaatttatgaaccagcgtcccggtaaccggcagaacggctataccacgctccacgcgatacgatttctggcgtgttcgctcgctgctgtcccaccccatcgcgagcgcgttcatttcgtcgctgttcattacctctccagacgctgtatcaatcagtcgcccggtaccgaaccggtcgctcagcgccgaaaaaaatacccgcgcgtaggtgggctccagcaaaagcggttgattgaacgccctggcggcgaggtgcggaaaattgttccacggcatcagttacctccccagccgttatcgttggtatttgaggggtgagatacagaccatgacggttcgctcagccccatttcacgccggcgctgaatttcgtattcctgctgctccataacctcctcgtaatcctgtccctgcagcgccagttcgttctgatacgtgctgaggccagttgtgatgcgcatggcgctctcctgaacctctttcagcccatcaatcgccatgcgaccagcgccgatccagagcgcgttcgtccaggagttccgcgcctcataaaacgaccggacagctgagcgtggcagggtgataattccgcgtgccagcgcctcttcaaaccagcagcagaacatcagcgacgcctgtcgcgcggcgataaaacgtcgacgccccatgaaaaatcgccagctgacgttagcactggcccgggcgctggagtaactgacctggctgtagtcccggctaagttcttcgtatgacgcgccaacgccagctgcaacgtaacgcaggagtgatttttccagtgaactaaaaccagcatcagcattctgagcggtctgtaaactgagtttgtcgccgggatgcaggtggggaactttcacaccaccgagcttgatatttgcgccgttgtagtacgtcacgtaactctgaataaacgaattcagcgggttcgagtcgatatcagtacccgcgccggcgatatattcaaacgcctgctggctgtcgagctctgattcgattgtcgcggcatacatcgccttcacaatggcgctctgcagttgtgtttgctgcagtgtgtcgagcattttcaggcgctccatgacgctgtaaaaaatattgtccccgcgggtctgtccgtcctcgagcggctcgaatatgtggatgaatgcgtgtctgccgctgctgagctgtgctgggatacgccggcatttaccgactccgccgagagggtaagtatcctcggctatccagtacccgaccgcggccccgtttttatcgatatcaacaccggcgcggcgaaattgagtgtcagctgcgtaacccggatttcgaatccgtttcggtgagaccattttgaatcgcgtccgaaaaacgctgccggcactactctcccagactggttgtacgcaggtttcaccgttgaaagcgtgagtagccacgccctcgcggatcatcattgtgaacgtccgtttccgctcgatatcaatcgtgcagtgagggtcctcggcgtattcagtccacgctacctcgacgtcacgcgccagcgccctggcgtcctcgcgagatatgccgaggtaacgccagttagggcgataactcaacttaaacagattgccgacgatatgatcctggtgcagctgcacggcgttcgacgcgacgccgttattgcgaacgagatcgtctgctcgcgcgttaccgcgataaaaattcggcaacagtgctgcgtcggcgctctgctgtggtgcgttccagtcaatcagctggccaccgaaaccgggaccgccgccgttataccccgcgtaacggcgtagtggcgttttaccgtcaggcccgagcagctgctgcggagcagtcataatcgaactcccacgggccgacggcgtcccgtactcagaccgagctgtgatttcagctcgataatgtacgttcggagctgctcaatactcgcctgcgagtattgatagctccggttatttcctgctgaatcgccgtgagacagcgacacggtcgatttacccgttaataatttgtgcagagcgatttccgcttccagcagtcgttctgttaaaacctcgcgcgacgtcattagctccctccgagcattctggccatctcctcgagagacagtttgttttgtgattttttccgttgctcagcgagcgtctcgagattgagctggaaacgaattttactgatacgcagcgctgccagtccgtacacccagcagtcgagcgcctcatttcgccggccctggttgtcccaacgataaacgacgcgaccgttaatgagtttcgggatcagaacctcggagacgagctgttttgcctctgtcgtgctgaatatttcgtcgtcgttgggaaaatgaatcgcacctggtgtcgccgatttggagtcgggttccagctgcaggcgcattgcgagcaggtctttcgctgtatctgtcccaatcaacgacaaatacactttattcgcgttacgggtacgaggcatatttacgaccggctggccgtatgagctcgcgcccttgattggaatgacccacagcggaccgagtttcagcgagcggttataaacgacctgcgcatctataccaccggtatcccacgcccagcgactgacgccgattgtggtcccgtcgctgcgccggtattgtttgcgaatcactccgtcgacgcgctgcagcgtgtcctcctcgtcgtagcgaccgaggacgattgttttatcaatcagccagcactcctcctcggctccccagccccacacataacactcgtagcgcccggacgtctgagagtcgatgccgccggttatataaacaacaccgtcaggaacctggctggcgtatttttcccggcgattaaccagaatatcgtgctccagctgctcgctggccacgtcgctccagagttcgccgagcgtcgtgttatgaaacgtcttttccttgagcgggtcgcctttcgctttcagccactcgctgacaatctcgccccatccatcgaggtttagtgaatacagggcgtttatcacgatcgcagcgtgtttcggcgcgcgaacgacgccaccgtcatgatcaaaaaaatgaatgccgtctcgggtccaggtgcagtcctcggctatccagcgccccccgaactccattttttcgagatcgcgatagtaaaaatgttccgggcagtggcagcactggtaatacgcggacgacgatttcgcctcattggtctgcaggctgttatcccatttgaggccgtattcgataccgtcgaaaccaaataccaggacctgctcctcgccacaatgcggacatgtcaggtaaaaacgaaacgtcagatcggcagcgtcttcgagcatttcgatatgacttttgccggtaacggtcggcgtcgaaccaaaaatcgccttcgggtacgctgcccctttaattcgaacgagcgccagttcgatgggcgagccctcgcctttgccttttttggcaacttccagcggccagccgtcaacttcgtcaccctcgacaacctgtttcgttagtcgccggaaatttcctggtgcactcgcgccgcgaaaatcaagaatcgcgccgctcatttctttacgctggagattgttacgctcgttgcttttatcccagtcggggaaaattttctgaatcactggcatttcggcgatagcggggtcaacctcgtcggcgacaaatccatccgattcgtcatcgataggctgatagaccacggcactacgttttttgtgctcagcgaaatagagcagcgccgcgacgagtatttttgtataaccgagacgagctgatttgcgaacagacacgatttttatcgcgtcgttcgtcatcatattgagcatcactacctggaccggctgagtcgtccagtgcccagcgatgtggctggagccctcagggagataaaaatatttatcagcccactcaacacccgtcatcggtatcgtcacgcgcaagggattcaaccctgtcgagatcgcgttcgatatcgctgacatcgtaatcgctgaagtcgattcgtatgtctgacaactcatttaaggctatcgccagctcctctcgtaatacactgctcgcctcttgcggcatctccggccagacctttttcaggcgtggtggccacgactcgacgcgggtacgtaattcaacggcgacgcgagatacagcgacgctaattaattcaattggcgcgtagcgtttagcgagaatgcggcgttttactcgtgccatcaggattcgctcctgtcgttcctcatttttcagccactgctcgcgatttttttcgggagaattgtcccccgcttccggttcgtcattactagttgtgtcgcgtctattgctccggagataacgaatataaaaatggcgccatgcgtctagctcccaatcaccacgtccttgtgggactggtgctcccggaagttttgcgagatcgcgcagacgacgatcagatagcagtaaatgtgctgcaacctcagcctgacttgccattaaaaatcctcaccggaaccggaaatacccaaaatgaaaaaatactaaaaacgagcgagtttttgcgcgtctaccgaccctcggtgttttggatttcggaaaggacccgcgaccagggggctatctccctttcaggacaggaattaggggctcgcatcaggttcatcaaactcaactgtagcctcgcggcgtacttgtttgcatacatcagctttgtgtatctgattatcagcgtaagctttcactttggcagcgagccatccttttgtgctgtatcgatagactaccgaacaagttttccatatatgtatatatcttgaacttattgatatcattacagaaaaacaacaaccatgagaacgcaaaaaatgaacagagaaaaaattgaagccttaatgagcgtaatattaaaagaagctgacccaaaaaagtttcctgagaagttatcagaggacgccatcgtatctttttatcgtgcgaactcctcttatctaaattgttcaactttaagagaatggctaagcggcgaagcagagccttcagatgagatacttcagaagattatggttcagaacaactggcgcgcaacaaaatggtcaaatcagttaccttaatctgttgaatgtgtgccgccttaaaacaaaaaggcggcctctcgttaaagtaaattaccctattttccaccaaggcatgaagcctctgccccggttgaatcaatacaaccaccgttcgcagtagtaatcttcgtacccggcgcgatcatcacgtgtttaatactgctgccgtcagcgcgagtcgcttcggtgtatgtcgaatgtgtgcagcctgccagcgtgaagaacgcgacagctaagagaagttttttcacgttttacctacgcttctgagatagatttctcagcattcaccaatggaagtgattgtggaacatccgtttgacctgctggccagcggtagcctgttacgcgggaacgaggaaaagctttgatatttacttcatctgcctggttgccccccaaaatcattaaatccccggcggcatttttcccgacaacaaagccgacgtgaccaccgccagaccgagacagaatagcaatgcatccataggcgggttccttcagttcattaccccaatcgagataagaattcgcagactcaaaacgcgttgagcgaatacccgcacgttccagcattgccccggtgaaagcagcgcaccacggtgtttcgtcatccttaatgccgccgcgtttgatatctttccagtactgcacaatttctgcagcatgttgcttccctttcatttcccgaatgccgaggttttttcgagcctcgattagccatttaggatccgtcattacctgtctcctgttatttttgaaatattccctcgtgcccgccagacagcaatacagatggtgaggttgagaaagaactctgcgggatcaacctgcgtatatttcccgtaaaaaattcgtagggctatccacgcagcagacagaatcgcgaggtaagcagttagtgagatgaaaaaacgatagcggcctgttttttgaaaaaacatcaaccgtagtgccaacatcagacaaataattgcattggcattgatgaggacagagtgcagggtcattatttcatccccttatctgatttccataaacgagcttgtgacatgatccgcaacagtgatgtaacgctgattactgatgcaaccagtgcaccaatggcgggagagacttttactgttacgggagggttcaggtgactcagcaccgcgttcagaataccggtaatgatggctgacgctgtctcagcgcaatacacaccgccaacaaacgaaatgagtgcgaaaataacctgcttccagaatcgatgctgcccggacgacaacacatagagagctgcgcctgccagcgcacagatcatcactgcaggtgttgcctcaggaaataacgcagcaaacgtcacccccgccgagccagcagcaacgccagcggttagaggttcagacatgatttttcctgattcagaaacgacaaaaccccgccgtggcgaggttcttagaaatttggcaacataccaaattagcgtcaaatatggcctattttgttcgtttttgcaagcattatacgcaaacgtcgtctaaatggttctgataatgatgcaaagttgcaagaagtgccacttcatcaagtgctctcaccgcctctttcattcctacccagtgcagggagtatgtttcgcaccaggtggaacgagaaaccgacagcattcctgctagggtagatccggccatctccttaaaatcctcgcgtttatttgctgctgcgaccgcctgcacggacaaccacaccaacgaaagcaggcgcttaactacttttctctgagttgacgcggggatgtttcctttatagcgactccagacagcctcacaaatagctgtttggtgtttgaatgccaggttaccaccgtagcaataacgcaaccacgcttgctgacttgcatccagcgagttcacagaacgccgccaggatgaatcagcaaattcaaaatcactcatcggcggcgcggggcgacgtctgctgaaactttccagagcgtaaactggcgtaaccagagcatcaacggtccggtcctccagctgcacacggtgaatatgtttgcgtgggtatgcgtttttgtctgccggtgggtgctcacacaatgcttccagttgccccttagaaccaccagacaaatcccgcaatgcagcgcgcagttttatgcgggtatattccaataaatactcgttactcatcagataaactcctcatcgcgccagatagccagtgttctgaaaactccctcagcgtgcatcaacaacaatgagtcgtgctcgtaatccgtttttgtacgtccgtcgatggcatcgtgacacgcactgcaggcgatagcgccctgcatatcgtgaggtttgatggctgtgccgcacgttccggcgagacgataatgagccagtacgctggtttcaggattgtgattgcacacaccggggatacgcacagtacaagcgcgactgcgtgcagcttttctcaggtcgatttttttcatgatgtgtaatccagcagctgcgcggcggcattttcagcggcctcctgagaggcaaatgtgcggaacaaaatataattccacagcacatctaaaacggctgaatacagttgaccgaactcaagatcgtccatcgctgcaaacgagattgaacgaggtacgcgcatcaaactgccgtctggcatctgatgaacgtcataaaacccagcttccataatcgcccatgcgcgaaacgcctcgaacgacttcaccgcactgatactccccgcgcgcttttccgactcatcgagaagatactgatcagccagctctgccatgacatcaccgtgtccaccgtaatacgccagtaacttaacgtagccgtgaaccagttcgcgctccgcaggagaaatagcccctccggacggatgccagtaatcaaagcccaggttgagaagtgagaaaaatttacgatgaaacgcgggattacgcgcctgtttgaaatctgcatacacaacagcacctggtttgactcgttcagtcaaccagcgttgcgtgtctggtgtcgcgggtattagcgaaccgccggataccctgataaatgaatgctgcgccatggttaactccaggtggcgcggcagtgttcagaaaatgatgccgggtgttcaatccagcaccgatattataccgtgttttacgcgtcagggggtgtaatagtatatccagctatttctgccaactcgagaaacgccatcaggctggcaatccgctcatcatcggtcagaacacgcacaccaacgatgtgtccgtccttacgcttgataacagcccggcatgtttccggtagttcgcctatagattcttgaatattcat

SEQ ID NO: 18 - DNA, 3202 bp, Artificial Sequence
  ST131 E. coli_NA114 CP002797
tcacagcagcacctcctgaccttccggcttcacaaattcaaaaccttcgggtagttcgccatcaggtgctccgaaaataatatcccggtaaacctcacgcatctctgtgacaccaaaaactgacaggcgaatattttctgacataaagcctggtttaagcattttttccagtttctctgcggccagtatgcaaccagcatcagccccaaactcattcacccacggcatttcaatatggtaaataatattactcaccactttttcgtgggagagttctattcgttcgccatcttcaatataaacaggttcattatgaacacgcaccattaaggaaatgaatgcatcagcaatgtagtagcgtaagactgctgttcgtaaagcatgcgcctttaaattttgattattattcatcgcgcatcactcccttaattgtgctggtttccttaacaacgccatccagttgttcggcaatacaggccactaaagccgcagcgccttccgctgaaacagttctcgctccattcactgtgggcacggacaagacctctgcaagaaactcactggccagtgccgcccgtaacagacgactctccccctgttcactcaatatgcagccttgcttaaaattcatggatttcatgcaaccacctccccggctcttttcacagggatacgggcagcaaagctcaggataaattcagtggcaagtttaaggcgtgcagcatgctcgcattctgcgattacacgcacagggcgtggacgggcatcacggtctgtacggcggacagccagaaagacaaaggtaaattcagggtgagataaagcagggactgtagccataatggcaacctccttcagatagcgggtaacgctaccaccggagttcctacgctcatgggtggtagcccagacgggggtaggaataccggctctgaaggataccggccagcccgaaagctgccccgcctggactaccataattctgatgatgtggcgtaaaaaataaaaaaatacaatcagccaccacaccataaattttaggtgagtcaaagctgcgacacaaaaaaacacgcctagcgcgtgtagtatcgccttcagataacacgggttcctacgcccggctgccgattttgcggcaacctttaaactatatcccgcgcattcctggtgaggcaagaaatttataaccgaaaactgatcattgcctggattaattaacgctggcatgcttaagcctctgcttaaaaatagttcttcatttttcctgatgctgtaaacgagacccgcctctgagcgggtctttctgtcgctaagtcaatccgcaaacgtcagggtgcactcatcaacagccgcgtacagttcaggttcaccgagtgcataaccttcattgcgcaggtaggaatgcacgtattcactgctttcatcatcaaactcagcacgaaaccagcgtgttatcgcctgagtgtaaatatgcagattatccagcgtcgttactgccagacgcttacccggcataaacgggggaataaacgcaaagcggccagcaacagaacttgtagccatctgtgccgcattcacatcagacgggcggtctgcggcattaaataacttcaggcgttgatgtgctgccagctctgccccaaccagtacaaccagacgaggatcttcacgaaactgctcagcaataagctctgtaatcagatggttggccagagcatcaatatttttccaggtgccagtctctcccagagtgacaggctcagagatgatttgctttccgttcccgtaggctttcgccagtgcatgccagccaatattgacatcctcgccttttttattgatttccgggtttgtagtatttgcaatcgacacaccgttgaaaccgacccgcagcatgtccagcgcatatgcctgggcaaaaaagctctcaaccgttttctcaaactcatccggcacgccggtatggtaaatatgagacatatcggaatagctgatccgggcgcaggtatccgtctctgtcagaaagaactccgtaccattgattgccatttttttgtggaaacggccaccatccacacgtccggtatacagttcactggcaccaatattgacggcattacctgacacggcgttaacatcacgtagggtaatatttttaataagccacccggattccagaattgaggtacgcagtatattttcagtaggttcgcttaatgcaaagcgtctgtttgattcttcctgatagttagtgccagtgacgaatttattatgataacgacgtgctgcgtccggcctttcagttttgaaggcatacatagtttttcacctttttaagtatttaagaaaagattttatttttgtactcagtaaccagattgtatttcattacatgctcaacataatcccggtcacttacgactgaacgtaatacataattatctgctcgatagtactccctgtaagccatagctcttgccattccaccgttatttaatacataacgatcagttggaacgctacaacgactaataatttcactcacggaatcagataaagacttgagtgcatcagtaataaatttaatatcacggacaacgtcactgtcattaaatacagaaataaaatcagcagggatctggccggattgaggaaatatattcagtaaacgcttatcgattcccttcccaatgtagtttccttcatcggccagtactcccggcaaaagcttgatcgtgctcaatcgactatgttgttcattcaaccgggaaacagccagatggtaataccgttcagcatccgctcgttcagcatgccccattttattcgttttggcttcagcaatgctactgacgccgttttttgagaatgtgcttttccatgcaacttccgcatttgtcacttcacgctgccggaaagataactcttcctcttcctgacgacacaactgggcataacggctcaggagcaggttgatctcattcgccacagtttccgtatcaggtactgttaaccccgtaagtgaatacagcttttcacctttagctcgcagcttattcagtgaatatatttcaataatacgcacaatacaggccgtgtcgctatgagtctctttaaatgatcttactttgtaactatctaacaccgacacatgttcgttcttaattgatgccaatttcttcttaaaaacattgtcaaccat

SEQ ID NO: 19 - DNA, 40 bp, Artificial Sequence
  ST131 E. coli_NA114 CP002797
agataacagagggaagtttaacctccctcttgcaaatagt

SEQ ID NO: 20 - DNA, 1975 bp, Artificial Sequence
  ST131 E. coli_NA114 CP002797
gtgataagtgaatcagtgatttccgcagctgtacgagcacaacagtcagaagatggtaaagtgggaaattgtggccggatctggcatgtcgctttcttttttgatggagtgggacgaaatatcgaccgggatgcgacagactgccgtttgagtaatattgcccgcctgttccgggcatatcctgatgaacaaaaaaatacaggtgatacttgttataataaattttatttctccggaatgggcactgcctaccatgacgattctgtcgacaatattcacgctcttatggatattggtttagataactttctggaggatatgaaaagtctgccagaggatactatatcggatgcaggggagtctgttattaaaggagaaaagaaatggacggatgtgcttggtaatctccttgaggatctcaataatccggtagagtgggttaaagggattggcaagatggcggctaagtcggtaggcaaagcaggaattgagtccacaccttggttgcgtgatagcgaagccatgagcgcctattttatgacgggagaacccgtacgccttgaagctgcaaagcgattgtttaagaagttttatacggaaaatatgaagggagatgttcccatcaaaaaaatttctgtttcattatacggttttgaccttggagctacactggcacgtaaatttttggataattttcttaaggaagtttgcctgaaaaataaagaaaaaaaacaccaatataaaaacgcgttggtagatattgcttttattggtctttttgactgttctcggcattcaccgactagtagtaataatgggttggactatttttttatgtggttgggtttaccagggaaaatgattggttcagtattaggtgaaaaagctattgatcaggactcaccactgccggacgtagtaaataacgcacttcaccttgtagcagctcatgaacggcgtccatggcgtggtctgtatctgttggggaaaaaaacggatacagcgaataaaaaatacagggaagaactgcttcccggttgtagtgaagatattggtggtggcttgaaaccggatgaacagaaacccagtgcagaactgtgcagggttgcactatacaatatgtatcatgcagcatgtcaagctggtgtgccatttcctgattttgacactttggatcaatatgatacgaaagttgccagctattttattatgaatgatgctgttcagggaggatctgtaaattactgggttagtcgttatcaaagagaggaagtgaaggataaagtatttagtgaggcaatgcaggagaagtatctagataactattttctctggttgggtgagcaatattatatgtatcagtatgaacgggaacgtctggataaggagttgacatcggctcatcgtgggaaaataagtgactatgggccacttgcaggtacgggaattaatccaaatactaacgctgatgatataaaatcgcagatttcagaacttgactcactatggggatggttgaatgatgtaaaaagtgtagctattggattacgaaatgactttatagtcaacccgaggaaaaatgatagccgaataaaacttaaacctcaggtttataatgctgctgtcaatcgggctgtgaaattccttgagttttctcgcgcagtttataatgataaaaatctgccatcatcatcatctttgacggcaaatacactatattccttttttgtgcatgatatacaaaaggttgatcacacaccttctatatctgaagatttcttcttacgtcgctcatctgaaattccagatgcggatgaacctccaggaaataaagatgatggaaagaaacatactcgcagggatgattagaaattataagtagcttactttaattagttatattttttgcatttttccctttttaaagagaatcatacttggatttatttttcataaatataaaggagtacagaga

SEQ ID NO: 21 - DNA, 1320 bp, Artificial Sequence
  ST131 E. coli_NA114 CP002797
ctatctctttccaaagctgatctgaataacattatctggagtaccattatcagactcaatgatctcatgctcaataaaatccccccatttcagaaacagattccgtaatttttccgcttgctgcccgacctggtaggttctgattaacctgtctttgggcacatgatttaacagaagttctataagccgttctggtgcgtccagatattcagcgcagcctgtagcaatgctgcggcgtaaatcatgtggtgagaaaggagcgacttctgccatatattttgttttcaggcgttcaagagcacgagttattgcgtcaccggtgacaggaaaacttggcttactgagcgactcgaatacccagccgccttctgaaataatgcgctgagtcttcaatagagaaagcgccagaggatgtagcgtcacgatatgacttttaccgtttttggtttcaggtattgtccagcggttgccattaatctgctcccaacgcatattcaccgcttcggaacgtcgcacccccgtcaggatgatgaggcgtaatgcgttagcaatcgcttgtgaaagaataacgctggacgcgatgcctcgccctccggttgcgacagatccgccgccgaaccccgcttcatccagtgccttccataacatttgcaactcgtcttgtggcaaccaacgctcacgcggttttcccattgaggcaccaaacattgcgggtttaagcaagcgagcagggttgtgatcgatcaatccctgcaaagcggcatggtcaagcgtcatattgagaagaataagtcccttgcggacaccttcggcgctggtctttctgatccgtatgagatgttcataaatgtaggctctggacaggtcgcagacgcgtactgaacctaaacctttctccagatggcggcgatatgttccctcataatcagccagagtacgcgccccaatgggtttattggtttcacggaatgccagccagttttttcataattgtgaaaaaatcggcatttgttcattttgctgtttttccgtttttcgcgcataacgcggatcaataccttgtttaaccagcgccagcgcgtctgcatgtgcttcacgcgcctgagacaatgtcattgcagggtaggagcctaacgttattttaatctgttttccgttgaggtaagggcggtaaatccagctcttcgtttgactcccggaacgggattcgacagacaggccggaacctacttttgatttgacgctgacggttatccgactttttccttcagggggagtcaggtttgcgacttgcctgtcctgggttatcaatattgccat

SEQ ID NO: 22 - DNA, 1281 bp, Artificial Sequence
  ST131 E. coli_NA114 CP002797
ctatttcatcagactttcgattaagccgctcattgccatatagcccatgattagcgtaaatacgcttatggcaataaacatgacgttttcccagcgtgagcaacgatatttgcccattaacgaaggcttgttcgccagccagaacatacataaaacagtcagcggcagaataatggcgttaaaggcctggctggcaatcatcaccaataccgggcgtgcgtggaacagcggcacaaccagacaaagcagagacataaccgtcatcaggatacggtaacctgggctaacttcgttgctggtatcacgccagtcagccagcagccagggcgttaacagcaaatttgggaactggctggaaagcccggctgcaacaataccaataacaaagagccccacggcagctgagccagcaataggttcgagaataatgaccatttgcgaggcattatttaaacctgtgccagtttggtacaacgtacccgcagccgcagccatcaccgccgcactgatgatgaacatcattcctgcactgacaatggcatcacgattttgatgaggcaaattgtccacaccccaaccattactttttaccagggtcgtgcgcataagaaacattgctggtgctacggtggtacccaccatgccagaaattatcagaaatggcgtactgttatcgccagcgacaggatcgggaacagacggaataaatccggccagcagatctgcgaccggcggcatcataatgaaaaaattaatcacaaacgccagccccataatgccaaccagcgcggagaggattttgtcgaacccggcgttgctgttggtccagagcatcacgttaatcataatgaccatcgcgatagcccaggcgagagctgagataccgccactaaaccaggtcgtggaccatgttgccattacatccgccagaatacccattacccccataatggaaccgctcacggcaacggttaacccgatcagaaacaacatcgcaacgccgggatggatatggcgacgaaatgcagtcaacgcgcgttcaccagtgacggaagcataacgaccataaatacggattaaaaaccaggtaacaatgcaagaaagcgccacggcccacatgagcgagacaccatagttagcacctgctttagccatagtggtgatgctgcctgttccgatgctgtagccaataagaaagatgccaggtgcaaaggccgcgagttttgtcattattcgccgaaataaagaaggcgtttggggaacagaggttgtcatattcat

SEQ ID NO: 23 - DNA, 10786 bp, Artificial Sequence
  ST131 E. coli_NA114 CP002797
aaaccaccttcgggtggtttttttgtatcttctttctcatttccgaagatgcgtccgaagatgatttttctctggacgcatcatcattcattttctctggccaccaactacgggcactactttaactttccggtcatatgctgctgtctgtcgcgggttcttatgcccggaaattgcttgcttctcttccaggctgccttccagatcagagataccttttgctttcagatcatgaaatgtgaagtctatttgcagatgaggatatttttcctgtgctgccgctttgacatcacgccaacgagagttaaaaccatcacgggtatacttgccaccggtagtttgatgaatcacaaacaggctactgattcccgtttttaatggcagagaacgagccagagctatcgctttctgcagacgaggcgaccaggctttgatttgcttaacgccggtttttccctgacgaatgtagatcccggaatcgaacagctggtctttctgcaatgaaagtacatcactctgtctggccacgcataagtaggcaatctccattgctacgcgaacaacatcaggagacacttcataaacggcctgatattcttcatccgtaatataacgttcgcgagctttctctttaaactgtttaactccctgacaaggattcctctgaacataacctcgttcataaccccaacggaatacccgggacataaagcttttttccctgtttgcctgagttcggcttgataaaccgcgttgatccatatagcgccgaatatgttcaggtttgattttatccgggtcgatcttcccgaagacaggcagaacttttccggaatattttgtgtagtctttccgggtttcgactgccaaatccataaaatcaggggaggtcatgaattgttccgtgagcgcctgaaaagtatttctttctttttcgtcaccgaccgctttttcatatgccagccatacggctgatcgtggtgcatccaatgcgcacaggcgtatcgctttgttatctttattcctgaattcgtaggccgctttgccctggtaaacacgaggcggcatccagttatctgctgggttctttctctttccggccattaaatattgctccaaaatcaggttcatcatcttttcgcgtcgtcacctcctgtaaattgcgaaattttattgggttcatgaaatggccccaggttgtttttgggtgtccgtcagccctttccatgaagaaaattccagcccggcgaagagcctcacattgctttgatttaagtgggcttcccgtcagctcgaccatttcttctctggtgattatgtcgtgatcgcttttcatggtctttcctcattatgtagcttattgcgtcatctgccttctggcaggcacgggcaatatcagactcggtcaaagtctgtttcctgacgctggccgacagtcgaccaattttgatatcaaatgcggagagcagagttgctcccggttgccatcgcagcattgtgatcctccggtgtctggtgaatcgcaatgctagcgatggtgagtttttatttctgattaggcgtaatcagttcttgtggggtgaatgccgatttctcccgagtgaccttaatgttttctggcaggtgtagcccaagttcgcagcggctccgtgcttcaataatgccattggtaccatcggcaagtaccaggtgaaccgcgtcaccacgctttaatgtgagtttaagcattagcgtatcctcagtgaccgttcgccgcgttccagatgtgcgcctggtaccgggtttaataactcggctggtggggtttctccacgcgccagtatttcggcggcaaccgcctccgcagattcaatcacttctttaatggctcgcttgtttggtgtaataaccgtttctacatcaacaagtgacacgccctcatactcatccgggatcttatctttgctatcgataattacccggatagccccctgtgcatcagtaaatgtgttttttgcggttttcagtttatccaggccagcagcttgcaggcaggtgagcatatgctttcgaattgctttttccttgccttcaaaggacgtcttacgggttgccaggcgagacatttcttcagcgcaggtattggcattgccctgtaaattacggcagatgaccatcatggcgtccagtttgtctcccagttctccttccatgccttcaagcgtgtcggcgatcatctctggcgtcagttcatcggcggtttctaacaggtcaatcagactggcatattcttttgcaatagcgatagcagtgctcatgctgtttgctcctgtgcagcggtaagggcggcgaggcgttcagttttaatgtcggtgatacggcgcagacgtgatcccagatacgatgaatattctttatcgcctttggtttccgctgctttcctgtgtacatccacttcgcgggcgatgaggccaaataccttgtttacttcgttggcggttaccgcggcggccagcgtattggcaacgttgataagcttctcatccagttccttacgcagacgggtagcatcttccgcattctcgctggcattcttgatatcaaactcagctttattcctctggcggtattccggattgtcgtacaggcccatgaaaatatcggcgctgaatccgagaggggatagcgctttcttaacagcatccgtccaggatttcttcggtgcttcgccgtcgcttgttggtccgtatttagtgtcgtagatataaggtgtgcatccgtaagcctcggactcaccgcgtaccccattcaatatgtaccagactctcactttcatggtgtggtgcatttcacataggtacccgccaacaccgtcttgaatcagatcccattctattttttcaccaacctgttttttgcgaactattggtgcgcctttatcaaagcgctcctccagcacctctaccccccagccaataccaaatggcccaaattcgcgagtcgctttcatcgctatataagtgccgttgatggatgtgccgccgccattctgagagaaagcactggtgaagcgctcatcggttttgaatacgttcttccacaatgccaggttatccgtgtcatctgagtcctttaactccgtttcgatagctgagattgcattctgccgcttgtgctcttcggcatggatccgctcaaccagcgcgtcaacgttctgcaccagatcctttacctgctcgctaagttgttcttgtggaggagtctcttctgccagggtaatagctgcaggttccctctgtgatacggcgttattttcaggtacgtcatacacatggcgaggcgtgacaaactgctcacgcaactgctgcaaactacgttctccagcatttgcagcttcgcttttttcgccctgatttgagggtgtttctgacatcatcccatcaattgagaatcgtccgccgccgtggttggtgacttttacatcatccttgggtgctgcttcctttggcttctcttcaacagcatttgcatcacgctgcccgttagcttgcagccagttgtcgatgtgactgcgcagggattgtgggaagtggtacgtgtcttttgcgggggcgctctgaataacaccgaatactgatgcgcggtcataggcaaggatctgatccgttgtgcgcaaggccattgaccagcgtttaaaatcttccctgtcctgactgattatttcgtctgccgctttcaggatgctgggggtgattttttccggggcaacaggtaacagggcgcaggcaatttcacgatccagagtggcgtgagtctgtttgtaaggacgtgcggatgctgtttctggttttacctccggcagcatttcatctcgtttaccgggattctctacccatttttttataaatttggagatagtccaactggtcgggttttgttcctgggtatcgcaacttgccaaaattgcatgtaccagattattcaatccagcaatatgcatgtactcgatgggcttacacgccaccatggcatcaagaacgatgcggttaaacgcgtcaacctcatcctcactgtcagtatcactgttatcgagcatctccaggtattcacgcgtctgcacaagcaattcactgtcgacttcgttggcgtcgtggctgaacagcagaacggcggcgaatcgttcacgagccggcagtttcatcaggtcgctaacctgtgagatatctgctgaactgcaatctgaatcaacaggagtgttaactacccatttttcgccatcaaaactgtgttctgtggcaaacgtctcatcaaactcaccgacgcctgggcgcggttggcccggggcatcttcccagattttaggtttgaaatagttgtcgccgtggtcagggtaggcttcccacaatttgccgatgatgatgttctcggcaacttttttgttaggggcggcgatggcgatggccagcgccgggatcgcgccgtttttgagcgcgccttttttcgcttctaacaagccgttaaaaatggtcattggtctttcctcattacaggtcagtggtttttagtaggggagctcatcggtatctggcgtgtattcgatgcaaagtaactgctggattttgtcatcgatagcagcaatacgctgttgggcgtcggcgactgttttctgtttttgttcacgcagttgctcgacctgtaaaccgatgatgtcgattggctcgggctggttaacggtaatttcaatttcgcggctttccagaaggatgtacctgtctggaaaattttttgacatatcaacagtcgcaacaagcagcttttcgtggctgaaagttgaagttgcataatgaataaagagtgttactgggatggtgcgcgcttccatagcgactccttggtaatgtatactcagagccgatcagcgaatattgatcagcttccatagcgtcagtgtcgttggtctttcctcatcacaagtttggtcacttgtgataaatccgaatggtttggtcgccgttcggggtaactggcccgccttgtgcgggtcttttgctatctaaagggtgccggttacgtttccggcgtcgttatgtttccattacgaccgtacagggctggtcaggcccgggctggtctttcctgttttgagctggtcaggctcatggtcaacgctggtcaggcgtggtacttcctccggtctttccctggtgtcacgctgcgggtttggcctctcctgttggggactcaggcacagcgataaaacctggctgtgtgaaaaaatggcccatcgtgcggactgggcaaagactaaacacagcaaattgatgatgggcgcccggaatcgaaccgggtaacgggcagggagttcccgttaaacacctgttcaccacaaccgggagcgcactccgccatttcaaaatttaacgacaaagctcaaagttgagtcgatgaagtgcgctctcgtgttgtagccaggactcttccctggtggtcacaccgtatcgccttgatggtgaatcaatcgctcatacctggctgtggcttgcacattccggctacctgctatggagacaacgcgttaaggctccgcttccatccagaccgcttcgacacatgtgccatatgccggtgaagtctttccctctgtcaccgtgttggcgccgacgcgcaaaatttggtgccgtctttccggctgtcagaacgtgtttctgaacaactgccgctgggtaagcgttgttgatgaaagtgactttacaatttgtatttgtaatggtcaatgtgtttttaaaaaataatttgtagtgaggggcaaaaaaatattcactgtgtaaaggtgtttttgtgttaggggaaagttgcatgccagcagcggctggcctggaaatttgaactgaactattttgtctggtttttggcttcaagtagttcttttagcagtagatcgaagtgctctcttttttcttttagctcattgattaatttatttttttcgctctcaggcagtcctctgaatagttgcagtagagcctgttcttgcggggttagttcttgattgtaattacccgggtgtagtgtggattcatccaaatattccccttcattcatgaagaaaaaatgaacagggtatccggtcacttttgctagcttgtctaatttatcttttcttggagttaccccattacaccatgcctgaactgattgagccgtaaccccaatatggcgagctaactcagattgagaccagcctaagtcttttagaatccgttgtagtcggctagcaaaagccatgtttttcgtttttgtgttcataccgtaaggatacaaggtttaattgtagggagcattgcaaatataatttgtaattacagttattgtttgtaatgatgacgcggacacaaggagtccatatggataaacaactgcaagacaaaattctttccgtaatgagtcaatccgagttagggcggcgattagggaaaaaaccacaaacggtaagcctctggttcaaaggacgggttcccggtgaagaggttttgcgcacatctgaagctcttgaatggcgcgtgacccctcatgatcttagaccggatctttacccaaacccagatgacagcttgcctaaaacggaagcagcttaactgttggtgagccccaaatctgattaagcgtaatcaatttttagcgacaggagacgctatggaaaaccaagaggaattacaaaaagagattttgacctgggcggcaagggcagggcaggaactcgtcacgattgaaatctgccgggcctggttcagtcagggacgcaatgatgagttgagactacatgaatttgaggacgcggacggcaacgtggactggagagccatcaacaacaatcggcagaaaatctttcgctggttacgtggcgagacaacggcggcgcgccgaaaaactcaggtgctggccagtgtgatgaaagccgtgctacccgcagaacggcgggcacgtctggagtcgccgggcgatcccgttttgctggcaacgctggcggcaaaagaaggggtggaagcgattaacgctgtacatctccacttcgcgccagagttaactattcaggaaattgacgaagcaatagcggcgctggtagcgacgcgaggagcagtgatacgcactgcgcaagaccaccatgcatgagcgcctgaccagcgtttaacttataccgaggaaagaccgatgttaagacatattgaccgcattacctggcgtaacggctggcacctgaatggacgcccggcacatgttgcagagatccgccccatattcgatggccgcgtcgcagctgcacgttctgtatgggaaaagtacgaagaagaaaaagcgaaactgcgtgagcaaaatctctctggcgctgcctatgaggctggctgtcgcgttctctcagaggctttgggcatatgaacatcctcccgttacttgatagacccatagccttccagagaagttttatccgtcttgaaatgggcgtgactgccgcattgtttctgtcgcagttgacgtactggacaaacagaacaactgacgacggctgggtatataaaacccaggatgagtgggaagaggaaacagggctttcacgatacgagcaggagggcgcgcgcaaaaaactacgtggtctcggcgtcttgctggaacgtaaacagggcttgcctgccaggctgtactacaaaatcgataatgatgtgttgtgccagctacttacgtccgcatacaaggatgcggaaaaaccacatacaggtgaggggaaaaccaccaggcctgtacgtggaaaaccaacaaacattcttacagagaatactacagagattattactgatggtgaatcggctgacgccgacacccaacaacctgccgaccagagaataaattatcaggtgatactcgatacctatcacgagattttgccagagatgccaccagtgaaaatcctcactgacggcagaaaaaaacaccttcgcactttctggaaaaaattcaatttcaatgagtcccgttggcgggcctatctggaatttatcggtggcaattgtcgctggatgctggaggacaggccaaatggcaggggaggattctggcggcgtaaaaatcttgattatctgattaccgaacgctgctatgtttcggtcaaggaagaacgcgcaaatgataaataacgcgattgcactgtacagcgttgacgcagaacaggctgttctgggctgtctgatgctgaataccgaccacgacagaaccaatgcggtatttgcgttactgaaaccggaaacgttttatatcaatgcgcatcaggttatctaccgggagatcaaaggattatttcaagctgataaaccaactgacctgataacactgaccgaactgatggaatcgaaggggctgtatgagcgcgtggggggattcgcatacctggctgaaatgagtaaggcggcgattccggcctcaatggtcaactacgcgaaaattgtacgtgaaaaagccattcttcgttatgccgtggaaaagttgaattcttgtctggagattatgatccagcctgcagatatgactgctactgacaggatcaccgccgttcagcaagtgattgggcaggtggccgaacattcccgtaccgggcataagggcggtttgcgtccggcaagtgaagtcgtcgatgactggatagatgaccttgagcgccgtttcaatgatccagcacatgcagccggactaacgctcggaatcgaaagccttgatcgcttaatggcaccaaagcaggcgttaagaggttcattggtcgttattggtgcccggccaaaaatgggcaagacagccgcgtttaataaaattgcggtgcattttgctctgaatcatcgtttgccgacgctggtatttagcctggaaatgactgacagaagtctcattgaacgcatggttgcgcaggaagcaaaggtcaattcggagattttttatcttggccctaacgatgaatctgaattgagtctcgcaatagcaaaggcgggggagattgccgaatccaacatgatgattgacagcacggcaggtgtaacccttgcgcatattgtcgctgaatgtcgaaaagttaaacggatgcgtggctcggttggcctggtggcaattgactatttgacgctgatgaagaccgaatcggcagagcgtagggatattgcgtatggcgatatcacaaccggtttgaagaacctggcaaaagagctggattgtgttgtggtcctgctgacgcaactcaaccgaaagcttgaagaacgtgccgataagcggcctaacccgagcgacagtaaagataccgggcagatcgaacaagattgcgatgtctggattggcctctatcgtgatgctgtttataacgaaaatgccgacccaaatctcatggaaatgttgctacgtctgaaccgcgaaggaccatcgggaacagcctatgccctgatgaaaaatggctcagttattgatatcagcgatgaggaggtttatcgccttaccaacgaccgctcagaaaaaaataaacgttattcccgtagcgataattctcgcacaaatgaattttaaccacgcctgaccagcgtgaaataaccgaggaaagaccaatgaccaccactccaggcaaattagagtacccgtcggacaaaggacacatagacgacggtaaaaactatctcgacgtcattttgtggaatatgaacgtcgggccacgtgcgcgcactcgcgcggtttttgtgccaagacctaaagcaggcaatttttcaacccctgctaagcctgcccgtcaggcatccgtagccgcagcgccagcagtgaaacgtaagggcaaaacgcacaccgggatcgcgattcgcagaaatggtgagcggcaagtgaaactccacgaaacagcgacaacctggtgtgcctcaccgcatgaaacttacgacaagataaccgggcagcgcattggcgcgcctggtcgctgccgcctgctgctgagttccatcactccgatagcaaagaaaggggcgtgatatggccgggcaatcagattacctgccgcccggcttaccacacaaccgcgccaaatggccccaggaataccagcttaaagagcactacgacatgcgagcggcggcgctgatccgccaactctttgagaaacgtattccgaggggaagcgtgattgagcaaatcggaatgacgccagatacgtaccgggaatttttcagagagcgtctgaactactggagaggggtgatggaacaatgaaatacaaacggtgggtgcgtgcggaagtcgtaatcatcaaacaatgtgcgggcagcatgacagttgaacgtatagggcagcttattggcagaaccggtgcagcggtacgcacaaaagcgcgcgagctgaaaatctgtatgtatctgcggggtaattatcaccagtcagtgaaatacctccaggaagatatcgagctggcaagggaattacatcagtcgggtattaatcgccaggatatcgcagaaaaactcgaaatgccgatcggagcggtgaatcagtttgtttattttgagcggagaatttcatgaaagaactttttcttgcatttgtgccccggtttattaatgaccagatcgcactgactgataatggtgaacaatatgaaattgcctgcagcatggtggatgtgaatcctggcgaacggtatgacgcgatgtgtgacctgaaaatatttacctggctgggttgggctattccgtgtggagaaccaaccaatattcgtccgtttgagagcagggaggctgtatga

SEQ ID NO: 24 - DNA, 2839 bp, Artificial Sequence
  ST127 E. coli_536 CP000247
atgtcacgtaactgtgaatctccaaaaggaacattagtaatagatctatcaacaaagaacaaggagataatatcgcaagctgctgcaatggaacaaatcgacttaaacgattatatagtcaaccagctattacttaattcgctaagagttatatttggaagtggtaatgttacaagtgatgacataaggaatattgataacattatcaaaaattctccctcaccagacgaagatatgcttaatgcgatttcgatgtataatgaaacctttaaagaattaattaaacctgaaagattagataaaagagataaagcttcaaagagaaaaccatcctatggtaaggcagcccgagataatttagctaaaggagtacctatttattacacaacaggaggaataccgaaagggctaataataaaagaatacccctctgggaagaaagaattagtagatttctcctctggtaaagaggtatatatcaaggattacaaaggatgatagccattgagtagccccattgtctatttcataacgagcttaagatatggatgtttccagtcacctttcttgagcttgcggcgagaaacgaatccatcatgctctaaagcacaaataccagagtgtgcggcaacaaaatcatatacagccctactctcttcgccttgtagcatatgagcgagaagcttgcgtcgcatgacatcagagcgccaccttatcttcgccgcaggatctttgtaagtacagttgaagatctgtccgactgcatttctcacacataggccatagcggtttgttttacccgtagacaaataataatcgattaattcattaagatcatatttaagcggttccaacaatgttcgccagcgaagaagtattcgccgggttttgatttcaccataggacttatacagaagctgtacagtcttgcttttacggcttaaatttacatgacctgcactaaacacagcagaatatctaaattgcttaactaaaccctcgcttatttttaataccttggcgatatatttagtttcaaggatatggaggttctcatctgatacaccaatctttgttaactcatcgcgcagaatttcaagctgacaacttttaatgtcatagttatatccttcttcaaggcatccccacttcattccttttggtaatccctgaaagcctgtaccattttcaaaagaacgccctcctaccttagctgttttgtagggttgcctgtatgagataactaggggattatcatctaccttacggcaatcagtctctgcaagatgagaaataaagttgaaataataactcttgtttttaggcgaaggattatcgttacaataggttaaaagcttatctatgtttattttaatctcatcgagatttgcataaacatttttcaccaactctctgaattgtttatcaggaatatctcttttggatgtcaggtgctttaattttttattattgaagagtagctcatttaaggaatactcttttttattatatatatcgcctaatgatacataacgtatatccttgttaagtttgctctcttagaacagttcagaaagaaactttttggacaaggcaaattctctgcatttatgcctgttaaaatcatgtcgaaatatcttaatgagaccagcattctcaatctcttccagggcacgagaataaccaaggttccgctcttgatcgaaaacagaagaggctatttttaaacctatactctttggaattgggacagagtaagtccatttcttatcatctttctcctgatactttagtttctttcttttgatagttgtacagcaggcaattaaatgctgtagaaatttggcaatttcttgataataaatacgagatcgtcgtctatcactttcattatcaaatgggattgaaaaaccatatttctaaacttaataaaagtcatcctatctgtgaatatttttgcttctgcaacagttgttcttttcataatattttgctttttttatatatttaaaataaattggaggtaatttttaaatgttctacatgtataaatccatcagggaaacctcgataaaccttttaaaagctagtaccatctgaaaaaaatcatcatgatcttaatatttcatcctgtagtcatagatacttatctttattcaatgagacaagataaaaatagagatataacctgttttgagctaacaccacttttttggggggtatttttgatcttgaagtttcatctacaacatatttatcttaatctaaataaggttgcaatgcatgaatcttagcaaatatttcaaggatcgcgctatcacttttttgatttccccctactatagcccatatatctactatatatatacatctcatcatatatatacatgtcatagcctccataaaaaaaatctatatctatgttatcagcgtgcgtgagcacgtagtatgttgaagatgttaagcttcaacatacatatcaagtaaggtccattattctgactggattttttatcagaataaacgctgatctaatgggagtgttctctgaacaaaggcaatatctccatttataaatttcttgtactgattaagatctttgtaagtgtcttcgtggattaagaacacaggcctgctacctctaatctgagacatgtttattgatagaaaaaattcgaacatttctccctgaactaaaggtgtagcctctgctttatttggtccttcccacacaatagccagccctatgcagttatgtttattgtatttaaccttacatacggtttcatatatacccgtgatcattgaagatacttgatttttcagcaacagaagatca

SEQ ID NO: 25 - DNA, 1371 bp, Artificial Sequence
  ST127 E. coli_536 CP000247
ctattctaagtcttcctcatcaacatcactatcataaggaaagagtgtaacttcttcctctggtataatcggctcgcaggcctgaagtttatcgttatcgacaaaactggtagccataatactgttcttgaaattattgtcacttatccattcagaattccaaacatgctcatgaggcctatcgtgaacgccagaaacgatagagcaaagcgtctctttatattcaatatctctgttaatatgataggagatacgttgcagccagatatctaaaatcccagtacctaagataggtttaaacttatctttgatctgtttgagcaactctaatttctctacgtcattatctacaaactttaatacattactcaacactaacgcacaagatgaaaatgttcttggattattaatcattagatcagaaataatactgactatcgctcgaacctcctcctttatgctactcatgcgttctaaacgcttatttatcttagcaagataggcttcaagctgacctgaatttttatacttacgagaaaagataacaagcttcaaaagagtcttttgaatagttgtggcattaacacttcttccaaataccatcaacgcttcttgtttatctggtttcattgaaccaaggactaaatcttcagtaagtgaagttttagaagcattaagctgtagccctaacccttgcagaataacagttagatgtctcgctatcgcctctgcatcctctttactatttgtgaagattctataatcgtctcgatatcgaatgatatgatagtcattgatattctggctttcgagtttctgcccaagcaactcatcagcataaccaagaacgatttccgcaataaaatccataagggcagaaccttgaggaatgccgttagtctgcccccatcgcatctgtcgtaaggcataatcaatttggtctccaagatttctacctgctcctcttgctgctttagcatcttcttttgtgtaaattgcccaaggtatggaatgggtataaattgatggataaaaatttgcgatatcagtactgaacaggtatttaaactgaagagatttattgatagattcttgttcaacatcacgccaccatcctgatacagcttttgccttatcagaaactccatcttcttcagattctcttggaaggcttgcacacacaatttttgtattgctttggaactcaccaaacctttcaagcaagagttgccatgattcatcttccgttattttatggacaagatgaacgtaaattgcaggatgaattaattcgaatggtcgccaacaatagtggccatctttattcgtttgtaagatatagttgtcaacgacggatgaaaagtgatccac

SEQ ID NO: 26 - DNA, 5544 bp, Artificial Sequence
  ST127 E. coli_536 CP000247
gtgtggatcactattgcaccgttcgtgacaatagttaacattatcgaaatcgcatgcttttttgtaaccgatatcattaataccgttttgcttttgttcgagagcatttttcactgcgagcagaagaggcgcaaagtcaaaataagcaggaagtgcaaaactacaatagctcttgtgcttcaagaaaaagcttcttgcgctctctgctgtcattgcggtaatcaatgtcatgaatactaaccctaattattacttttgagtctctattaaacactcctttgaacttaatgtcttatgttgagccactaaaacgcatcaatggtcacaattctgctcatcagttttcactcaatagtatcagagatatactattacccttcttacaacgcttatttattggccaaccatcatcccattgataaatgatctatcaatgggatgatagaaccgcttttattcgaagcgttgttcgcgcatctcatcctcctgctgctcttgcaacttttcatgttctatccaatcgtttaatttctcggaaacaattctagctgccgcagcaatatctgtacgctcatgctctattaatctactaatagtatttgctctgggttgcattacgttaactcgacttccccaccaagatgtcggtgttacgcgataagcgaattcttccagtacggcctttggcacaggagctgcttcaaggaagcgaatcgctgaaacctgcattgtgaccttatccaaatcatcccccacagaccaaaggcggataccctttgcgagaggtgcccatacattaatatcgtttcgagtaagacaccattcgatcaatacattcacgtcgatattcgcgataggggactctgtcacatcatcataatggataaaatgaatacgtcgttgttgctgttcttctgtcccttcaaaaatacgagagaggaacgcttctggcattgatgccgcagtcatctttatagtgtcttcaaacgaatgaatacagccatactgttcatcaacaacagcaaagatcgtatttaaccactctaacttccccgcctcgttgccttcgagacgcaatatggcaccaacaacacattccatgccgtaatccattcccccagcggaatcattgtgctctcttttgaacctctggattgcagctttcaatcctatttgaagcagatcaaagccaagagtactcgaaccgtctttctggtcacttaacttcatgcttaaagcttccaaaataacatcatcgccatttggcatacttaaaagcctgtgagcaagatcaacaatgcgcggctctggcaaatgagcgtattggtctccccaaagaatttgtccatacattgacggcatgatgtcggggtcatctaaaagagccaaacagcgatccaagtcagtttcagtgaattctcgccaaggatgtaaataaacaattccgtatcgcaactcaggatgacgcacacattgatcgagaaattcttgggctaatattgaatcaatagtatctgtctcgtcgataaagccacggataactgcaaaatccttttgagaaccggtttgtatatccaaataagcaacgagttgttgccaacactcccgtaggtcaagtgcccctcttgctaaaccttgaccaaatttaacacggtagggcatcccccccaccaagaacagattaggccccaactcatcaagcgtatgctgagatgacgcgaaatcctgccccaattgataagctttagagtcaaggtatgtcctaagattagcgaacttattaggttcatcaggagaagtatcggcactaaatgccaaataatcgcgtccgttacttaatacataagccattatttccggaaccaagtcatgtggttctaactcactctccagagcgacaagatcgcttggcaacggctctaaatcttcgatatctctgcgcttggtgtaatcaaaatatatcgttgaacgaacggccttccatccttcaacccacggatagtaagaatgtaatttgtgagcagcatcaatgagcattgtcctggcagcctcttgacgccatattgatcgaaaagccctcgctaaaagctgccgcgcagtttcctcaaccgcaggaattccgcaagttcctgcattaaccgcaacttcaatgaatgcactacgccattcaacaagttcttcataggttggttgaaaaccataatctcttggtcgtgcaccaaactcattagccccgaaccccgtccaaggtgggccgctgagggctgttgaaagcatcataaggcccaattctttgcgtccaacgacgtctgatgaaaggcattgtttcaaaattgcaaggcgctggtctaaagaggcctgtgtaccagacaggtatggctgataaagcctcttgatcttaccccgtacagagtcatggttgttgttttcaacctcataatctaccgcatgaattagcagcctgacgcaacgttcaaaagtttccggttcatatgccaacgattgcaggagattgaacatagttgtccgtcgtagactgtaacgccgttccatacctttaaagtcagatgaagtaagctcttcttcaatcctgttgagaagtgtttcaggcgcaaccggaccgatgtatgccaacatttgtattgatgcatcatccaactcagttcctcgtccaagcaaatctcctggttgaagccacgcttcaacgatttctttcgctacagcgtgttcatggagcaatcctagccggtgtgcgaatgatattaataaacgttggcgaccgggagcttcaaaggtgtctctgagttcatgaacggggatgctatcaagtgccgatgcagctagcttgtttgcaatagcatgaggtagaacagcccgccaattagctcttttctggataacgtgacggttcagtaaattagtaactgttttgaataactgcctgtaggagtacccgccgagtgagcctaacgcctccaattcgttaatcccgccttcactggttgagatggagaaggaatacactaaggacaatatttcagcttgttcccgcagatcatcatcgggatcattacgctgctcaaaaaggcggttaaacaactgtgcatcagacaattgagctagactttcaccctcttctaccctctctgctattgccaaggatacacgtgcattcccatcagcaaacgctgctattctacgggcattgtgatggcctatacgggggaagcgacgaatcaaaagctgttcagctacctccggtccaaacgcctctatgcggattacctcggttgtttgcggtttatcgtccctaatatcgtattcgatggtaataagtcttaccttgttacctacggctgatatcttacctgccaataaagagtgtagtgctgatgggcaattatcgaggatcataatagctcgtcgatcttcgacaataagccgctccagcatagccgttgccgagggggctgggtcagcacctgtatcaacatagattacagaggttctatctagagggtcagatccaaccgattcatcaaaaagtgcctgaacgatccgagtcttacctacaccagacaatcctgttatgcgaatagcttttttagtagaacggattagttctcgcatcggattgatagcatcttcaatgctaagcttctgcccttgccctgatggaagagtgaccgtgactccaggagctaagattagagtgtctacgacgccattgggaggattactccacgcaccgtatggctgccagcctgaatatccctgtccgagtatcccttttacccacaacataactgacggatgctggcgtaaccattgagtcagtttcgagcgatcaaagaagtcgagatggatattgccattatttggctcatccttaactgcttcctgcattgcttgcaggcgtttcatcttagaaataggagaacaatcatcactgaggcagacaattacgtatcctcctcttttagaaacttgctcagagattaacggtagtaatccttgttcagtttccatttcattcttgatcgcggacgcactcatcgaaggcttcttggcctgaaagatcgtgtcgggcctctcgataaaccctgtacttagtttgtcgataggaacctgaacatgaacgtcaattcctccgtctggtgcattgatggaaccagaccaattaacccatgcgggactgtggccatgagctgctacctctgcctcagcaagacgagcagtcaactcttcaagttgactatcggtgagtcggagtaattcatctttctcaatgtcaaagatcgccacatccaaaccttagtaaaattattacggccacagtcagatttagcctcctagtctaactaaagttgaaagagttgtccgctgaactgcccataaccatagaaaaacgttgaagttattgggaagcgtcggcttaggcaatatcgttaatcatgcccgcaactggcttaacgccagcatgcagatgggatttagctctaatcttcaatgaatgacggtctaaactgactcaggtaaagggggaggcatctacctcccctgtgatttgctcttactgtgatagttccttttcactcagaaaacgagcacgacggttacaggtcacttttgttctggcttgccgaatgcttgatttagccaacattgcaggattaacaaagtgttcatctaagcattgtcgggcaatcgcgaagtcgtaggctaaactcaactctcgcattacattacccgcctgaactcttgctcctcgttcaacaatactgttaaccaactccaccacatccttacgcgtaacagccatagcttccatatgccccagcactctgatggcatcgccttccagagttctccggcactctttctggcctttaactttgcgtgcgccacggatcagcttaccatcagagtctttacggtcctcaatcctctccgtcagatataactcgactacatctttgactgttacaggcttctgcttaactttctgcttttcgagtttaagctgcttcttttcctcatccagctcagttctgggacatcgcccctgctttctgattatcttcagttcgtgtaacctaacgcgggcatcagccagagtggtcacaggataatggcctatcaagatctgtttatctttacctgttattggactggtatagcggtagaaaaatgtgcagattcccatttttccacgcgtaacacgaaggcctctattttcgcccgtatctgccagagtcatgccgagcttcatggtatctataccgcgagcggttagaggtttattaggttgtttttcagacacagaaactccatgtaactggttgatattaatgatgtaaatcttataagtgtaggttgttttcgaattcaggttatcaccagactcttacattgaaaacctacactttgacctacacttttttttgagatttggtgatttttcatgagacaacgtgacaccacaaaagattgtaaatctcttaaaaagtattgtttttcaatgaaatggaaatcataagtcca

SEQ ID NO: 27 - DNA, 1540 bp, Artificial Sequence
  ST127 E. coli_536 CP000247
tcagtttacgcttacccattcataaatacgcggtaatgttaatactacactctcctccgagtaaacagttgcgaacttctgctgtgcgctcatagcagaccttgcaggtcttccgacggtccgcttcgtgccaggggcagacatagataaagcatgctctgcatgaatcattgaggagatagtcactcttgaaatgtaaatcccctggaattctcaccattccataactggtatttgtaagctttagtatgatataaatattattttatggattttaagaaaaaccaataaaagttctattttttttataaatggagaacaaagatgaataatggcgaagataaaaaaacagctttcttgataaaggattagaaatatccaaacctatcgcaggattattttcttctgcaatcggtggcggtatagcggggtcttcaggaggtatgcctggagtcgttataggggcggttgcatattaccttgcagatgttagtaatcgagggttatctcggattcaagaaaatagagcattaactgcgctaaatatcgcatctcgagaaatcgaaaaaaattaacttacagcagaacagtagagatatggctagtgacctctgcgtatagacattttttttaacataaccacacacatatattatatcaatatattattgttaatcaaagcactgctgagtgcttgcattgtaatactttcaggtgaatatctgtgccagtaaagcttcaccttttgctcaaactctggactcagatttataattttaccggaagcaagttcaaagcttatttgttgtttaggtatcatacaacatgccaggtgttgtaataacattgtgacatatgcttctgaggaagatacaatatgactttgtagcgttcctggcaccaacccaaaatattgctgcaaaaatttatgatgcatatctgtatcatgattaaaggatacaatcggggcctttaataaattttcctttgatacgccctggctgaaatatctggaataaaactctggtgttgcacaaaaaatataatcgagagacccaacataatcacatagcccgccaatgattgcctgcggctgagaactgatacaacctacaacctctccttttaacagacgcttaaggtttttattttcatcatccacttttatatccagtcttatagatggattttttaaaaaaacacacaaggcaggaattagccatgttgccagcgtgtcagaattcactgcgatcggaatggtcacaatgcttttatttgcatcaaggttcaatacatcatgctcgaatatattaacatgatatagcagtcctgctaatttctgaccataatcagttaaatgtggaggtgaagtacgagtaattaactgaacaccgcactgattttcaagtttttttattttctgagatattgccgattgtgaaataccaagaattcgggccgctctgtaaaaccctttttgcttgataacgagatcaagattttttaacaatgaataatcaataaagtttctcatttgcat

SEQ ID NO: 28 - DNA, 48 bp, Artificial Sequence
  ST127 E. coli_536 CP000247
tagatatttgaccccggactgatgatcaccatcagtccgggttaatat

SEQ ID NO: 29 - DNA, 1209 bp, Artificial Sequence
  ST127 E. coli_536 CP000247
tatttcttcccctccggttattttgttttcatactgattattaccacacatctgttacaaagcaagggggacattataagcatgatcttataatcagattattattattaatttttaattataacatatgtattctgtgaactagttgtttcaatcaattcatctgaaatcgataaagaactgcatggaaagttgaatcaaccaagaaagatatcacacgaaatcacctcgttcagggaggtgacatttgctataaaaatgaacaggaaatgctgtgaggagggaacataaatactattcatgtaccgccatacaggagattttattgtgtagatattctttgatataatctctggggccaatactaaactctttcttaaaagaataattaaaagaggatgatgaactgtagcctaactctattgcaattttttttatcttatcattattttgtattttgcttatagctttcaataattttatccagttataaaattgatataatcctgtcccggtgtacctcctgaaaatccgcttaagatagtcctcctgtaaataaacacttgcggccagttcacgaatcgttgtagtattattattcagcatattaagaacagtcagtctgactctgacatcagtattatcatctgcgataaaatcaatcaacttcatctccagtagtgataatataagtgtaaccgatactttcccattaatagatggagtaatatttccaattgttattttttctcctgttattttaaatattctgtctattgttttttttattgtttttgaatatgcaaaatagtgcgtattttcagaagtattctgaaaagatgtaatttcttttcttgtcagtgtaacactataataaatgttattgtcatagccaaagtggtttaaaactattaaacccgcatcattaaatcccagttcttgtggagaatacaataaccgaatatgaggggaaaggacttcttttataaaagaaaatactgtcatatacaacctcaatatatcttatactaactgtatttgctatgtaactttaacatgcattcagttatcagagatattctatcaaaatggtattattttacacacgcagatatgcctgttaacaattatttatgttttacacattaagctcgacaagcttattgcgagaataacttcctcacaacaaacttgtcgaatattacagat

SEQ ID NO: 30 - DNA, 388 bp, Artificial Sequence
  ST127 E. coli_536 CP000247
taattttatcctctttagccaaagatttattaataattaacttcctcaatgacatatttagtatttacttatagtaattcgatgatgtgatattgatcatgaaaattttattaagcacgaacctttagttttgcattttagatatagttatatattctggataaagttcagttttttgttttgtgatgtatttactcaagaatttccttatgcataatttcttcgctatgaagcatggagcgaagaatacttattctgtaatctcttgttttattccgaagatatgtaagtgtgaaacctgccagcactgcttgttataccaatttcttgcaagacaactgagaacctttcaaaatcagatggagttctaatgaattcaaacttcgta

SEQ ID NO: 31 - DNA, 26596 bp, Artificial Sequence
  ST69 E. coli_UMN026 CU928163
ttagagaaccttagactgttctgggaaccagttattaattatatgtcttcgacagactttagatatttcaaatacctgtcgtgacactaatgcaccatcaggatggcgctggtcaggtatgtcattttctatgtcataaaaattatgcgttatttctgggggaaagaaaacgtttttgtctaaaggcacgcttgcatagggattataatacacatgcaaaccatcaaaatgagtttccttgtagtcccttgttcggcacattacaatatccgcgccaacaacctcattgttgtagaattgtcgcttcgtagtaacataatggtcaggaccaagttttctcgtatgagaaccttcgttcttcataccttcattggcaataaaaacattcacatcaagagctcggaatctcgtatatcggacatatcgatcgacccgactctgaacgatagccttaccaaaagtacctgtcgttgaaaacacgatagcactgatttctttgtatgagtcatttgtaaaaatacctaaaggtatttcagtgtcttcattttttttaatcgataaaacccttctttgcctaccatttactaaatctctcatcgtaggttcttcaatgccaaacaatacccggttaatgattgtattattctgggttaaagatagatctgaatcaaatggggccaatgctaacacaaaaggcttaccgattacatgttccattgttgagtatgggaacttcttaccattaattccggtaaagagatcgcgcttatctttaattttcccaagaagttttagcgtagattgatgcttaaacttagcttcactgaaaatttcactgactggcggtttatggggcttgtcagagaccatagcctctacgttaaatttataacctaatggcgtgctaacacaaaaatcaggactgtcttttgtatagtcaaccgaataccccaaacgaataaacatctcatttagatataattcccacatagaagaattaaaggtagtctgaaattcgttaataaatttagtttgttcaccactgcgttccagtaatccgcgtccccatgattggatcaccgccctaacaggagcaagtttaggatctgtgtagattgttttaaaattgtgatttagtttatttctagctatttttgttgagaagagatccattatttacctctataaacactttgtaaaactaaacatgtgtattttactgcgctgattgtcgaacatatgcttcaaattgtaatgcggtattctacatcagaaaaacctcttttatattttcctcattaaatttatttacaccgaaaaatagccgcatgaatcatgtctgaacagatgaacttctctaatatagcctaaatacctacgactgcgctataaagagggaacctcaagcactaatagcacaaagaaaatatgcgaacacaaatttgacgcgaaactatgacttagataggatgaatctaggtggtgatggtcacctggcgcctgtctcgcaaagcaatagcccatcgttcagaagtataccaaatacttggccgccatccattatgacgctgcccggttagcaggctatcggtatccaaagcgttactttattggaggtacagccatgtctgactattcatacctaagcattcttaaaacgtgcgctaaaaagctggctcgcattcagaaaattaagctttccaaagcactcgaaatcgttgcccgtgacgcgaaattcaccagctaccatgagctgtccgaagtcgcaaaacgtgccccgctagaacaacggctggttttcgctgcaataggcgaagatactcaagaggatgtgatttatcgcgatgagattttttatgccatagaccgcgaggtggaagatatgctctccggcgacatagcggaaacaaacgcctatggttttagcatagagaatctggaggcaacaagcataaattacgacccaaagcgaggcattctggtcgtccaagtcatatttgattatcagggtgaacaagattctgaacgccctttctctggttcgtcctttgaaattgttgcgaaaatggagctacttcttcgcgacaatgtatggagcctcgtagaaaatagtctggaattgacctctgttaattcaaatgtcgataatgactggtacgattaagacatataacagtaggaggcattccaaatttttggcggagtgcctttttagtaactatatccagatgaaatcatcgagttatcacatgagcatccatcttacgtgtactcaaaacacttatgggcttctaaattcaggaagtccctactccgcttgctggcgcttctttgagcctcgtggcgtccatatacaccaattaggccagaccatgctcaaacaacttgtcgagaaatgctatgtgcaaaaaccactggacatgttctcgatgacagcaggcaaactaactgggcttgatcaatctgggcctaaactggcttccatcatatgcagagggatagagcaggccaaagatgtacaactcggagagttactttttgcctgcggtatctatggtgtggaagaagaagaagcctggttactagctaaaagattcagtaatcttgaggccttgtatggtgccagcatagattctctgatgagttataaccttcttaatgaggcagtagctgtcaatacgtataacttcttccgtcatccgctcaatgtgtcggctcttaacgagttacaaacagaaggtggattgaaggtgcgccatggatagaattaacaccaaggctgtgcccggccttttatctatgcaacgtagccgatgacagttaacggtgagacagcaacacctgttgtaacatgtttgtactcgtgcattgccatccgtaggcctaaacatatctgactcccacaggatggggataaaatacaacatttgcattttcaatttgttatcgaaaaatcattagtcctgatgggaaataatatcttcccggactaatgattttctatccggactccacttttttatccgggcctatacctcccctcatcgttccgcgctataaccgctccgacactctgttcccgtcccgtgaggtgcaatcatgaaaaccagtgcagacagtaacgatgcttttcccgaaagcggcaacgtgcgtatgcggcaggtagtccagtttctggcgatgagtgaatcgtcggtttaccgcctgattaaaaacaccgactttccccgccccgtccacctctcttcccgactggtggtcttcgatgctgctgaaatccgtcagtggcaacagcgccgcgccgccatccgttaatccacgctcagggatgagccgactgataccccgtcatgaaacgcgccccttttctctgtaagcagtctccggaccgcacgctcgaagtggtgatccttgccggaagcctggcatgggagacctcacacgtgtggcgaaaagatcccgaccgggaagatgatgttcccccgatggtactcggcccggatgaactggccgatctgagcaatctgaccattatcaggcccgacacgctctacgtccgggtacttcgcaccggcgatatcagtgaagaagagctgctgaaaattgccgtaaaactggcgcacgcgggcgtgcagatggcccggctgatgacccctgacggtgagttactggagaactggaccggacagcttgaacgcctgcgacaggaaagaccttctgacatcctgccggatcacttccgcctcgatgaagaagcactgtggtttgataagctcaccgaacggcgcgacggtgaaagcgatgtccagccccagcgcatctgttcccctctgcgcgtcactgccatcacctgcgacagccatgacggcagctatggccgcctgctggaatggcacaccaccaccgggcaattgcggcgctgggccatgccgatggcgatgctcagcggcaacggcgaggagctgaggcgcattctgctggagaacggcctgacctatatctccacccgccctgccctgcgcagtctgctgtgcgaatatatctcgcggtcactccccggacgccgggtcacctgcgcggagaaaaccggctggcacaacggcgtgtacgtgctgccggatgaggttatcggtcccgatggcgacaacgtcattctccagggcagccactacctgactggcggctttgcacaatccggtacgctggcagagtggcaggagcaggtcgccgcgctctgtgcaggcaattcacggctggtctttgccgtctgctgtgcgctggccgccccgctgctgcgcctgacaggaacgggcggcggaggctttcatctgcgcggtgagtccactgacggtaagaccacggtgatgaaagtggccgcttccgtctgtggcgggacggattactggcactcctggcgggccaccgggaatgcgctggaaggaattgccagccgccacaacgatgccctgctgccacttgatgaactgcgcgaagtggacccgcgtgaggccgggatgattgcctatatgctggcgaacggacagggcaaggggcgcgcccgcaccgatggcgaagtgcgtaaccgcaggcactggacgctgctgctgttctccaccggggagctgtcgctggcggaacacaccgaatgcgccggggagcgcctctacgccgggatggacgtgcgcatggtacaaatccccagcgacaccgggcagcatggttcgtttgagcagttgcacgggtttgccagcggccagcagttcgccgatactctctgcgaccgggtggcccgcttccacggcaccgcctttcgtgcctggctggccttcctcaccagcgacctggacgccagcaccacgctggcgagagagttacttcgccgctaccagaccgccctgatgccggacaacgccggaaaccaggtgcagcgcatcgtggcccgttttgccctgctggcggtggccggggagatagcgacactgaacggtatcacgggctggcaggaaggctcggcgtatggggcggtgcagatctgccttcacgcctggctgaacgagcgcggccatattgccaaccaggaagatgaaggcgtgctggcgcagatcaagcggttcattaccgcgcaccagtacagccgctttgcctcgtgggatggcccggaccgcccgctgaatatggcaggctttcgccgggtggaaaaagatccgctgacgggcgaggaacacacgctgttcttcatcctgcctgagggctggcgggaaatatgccggggattcagtcctgccagagcggccagattatgccaggaagcggaatgcctgctgcccggcagcgacgggaaataccagtcgcaggttcgcctgccggagatcggcaaagccagagtctaccgcctcacctcccgcatcctcagtatctgagtgccatctcgcgtgtcttaaaaaaaggtggtacagggtgggacaagtggtacacgcagagctggcgcgggtttcagcgtcccactttagataatttggtttaaaaaaagtggtacagagtgggacagccgttgctgaatctgtcccacttttctgactggtatttttcaggtggtacagcgcaaagccacggctggcgcggttgtaccacttgtcccacctgtaccaccttaaaaacagaagaaatgaaaatggcgaaaagggactgcaaaagcaaaggctgaaaggaagagagaataaggggaacggacttactcttaaatggataacctatatttttatattcgtaccatttacatgatggagtgcaaagttaccattcaacatgatttggattagatagcaaaattctctatagaatgttttctagatttcagagggatttaacgatagcttatctcacaaatcacaaatcggtcaaaaaatatacagaaaaccaagttttgcccagaaatccttttaaaatttttgagttaaaaatcattgtgttgtattaatttcaggacgccacatgtcaaatatcgacacccaatatagtgtataaaaacacaggaggactcattgtgatttatataataaaataaccttatacatctttctggaaaccaagtaatggttaaaattcacattttagatgccggacatggtgattgcttgttagtagactgcgatggtgtcaaattgttaattgacgctggcccaagtacatttaggtatagaaaaaaaatttcagccaagttagctgaacttctgaatggcgagtctgttgatatcgcttttgtaactcacaatgatgatgaccatatcggtggtttcaaatacctaattgaaaataaaataaacatcaagagatttgtattcaatagtttatctaacattaaacatgtaataaagaattcaagcaataaaatatcgaccaaacaagatataaatcttgatagaatagtgaaagatggaagttttgtcttttcgacactaacttcagatgactctcctattttaatcagaaatataaaaataacacctattactccttcaaaaaatatccttttaaaatatttagaacaacaagagcgtaaaaacacagaaattaaaatatcatcttcatctgaaaaatatagtattaaagaagcactgcagttgctaagcaatggaaatgatatgtttgtaaaggatccatcggcaactaataaaaccagtttatcatttatgattgagtatgggaatttttgtggattattccttggtgatgcacacgaagaagacatctactcatatatggctaatcattgtgccaatagaaaaatatcatttaacgttgttaagttagcacatcatggtagtgaaaaaaacacgagttcacggttattgaagttaattggtacaacagaatatatcatatgtgcgaataaaaccaagcataatcatccaaataatttaactcttgcaagaattttaatacatgaacaagaacctaaaattcatatgagtagcagcaatcaggagataataaatttactcaatcaaataaatttattaggcttcggcattactgcaacccattctgaaaatggagttaatactttaatctatgagtagaaaaaatgctgtaaaaataaaagtcaatgattcggaatcaagtggctattttttcaaagcaagctccaaggatgattcttttgtaatttcatccaagcacggcctttgttctagacagtcagattgtgaagaatttcttgataatgttcagaattgttgccgtttatgcactcaagatttaaatattgacaatatatcttttgaaattgaaggaaataagaagctaaagccaataagttatttcagtcttgaaaacaaagatattgtcatcaccaaagtggatggggtatctaattatcctttaaggataggaaagatagaaaaagaaaagtattatacttatggttacaagagtaaatgtgataagcctggcagaattttattaaatgagccagatttattagatgggatttgttatttcaatatatgttccgatgcaacacctgagttgattgagaaaagtgaagaatattatggaatttcaggttcattagtttttgattctccagaaaaagatgttttaaccgcacatgcagtaataacaactaatgaaaccaataatgacttaggttcagaaatattacatgatattgatttcaaagaaattaataactttttcggctgtgaagtattcttgggtaaaaaactccaatttcaactagatgatacttttagtaaaaatttttcaaagattggagaagtgcttgttagcgacaaactccaaattgaaatatttatttcatcttataaaggtataccatattttaatttgacaccaattgctgatgcattaattcaaagtaagttttattatgtttttggaaatgttagtagaagtgatcatataaatataatcgctgcatcaaacattatactcaacaaatctcatttagaaccagctaataaattattaactagcaaattaactgaatcgatattatcagcacctcatatttattccactagcattgatgataataattatcatcatattcattttaaacaaatgacttctggtgatattgagctcataatatcatgttatggtggaggagatgatcttcctaaagatataaatggcactttaactgagttgttacacaatataaatacttataacttaaataaaaattcaataattgagaggtcttttcttaatcaaaaatttacagatgcacaatgcgaaattttatatgagatttttttgagcgattaactgattcaatcgacagaatttcattattgcatgtaattcctttggaccattatttaataaaagataaatcagaaattaaaaatgaaattattaccattgttgagaaagcgtgtcaagaattagacagcaacattttagattatcttgagcatggaatatctataaatctattaatatttccaactaataataaagatgaattatcagggttaatgcaaaggttgataaataattatgaatgatgctattaattatatctgcaatagaataaaaaatagtgacggatttagttactgttatgaaaaactattgatatctatttcaaagaatctattcaatccaactagcagcaaccccattactcagcgtgaatatcaacaattgctaaaattcagcgattttttatctaattccgaaaaagaagaagatcgaaacctagcgttaaaaataatatctgcaatatatgatctttataaagaagatcatagttgccaattactgacaaaatccatccttagcaaacttggactttttgccgctgaggaagtatttactgatagtgatataaaactaccattatcctatgaaatttctagcaaatacagaaaaataaaaaatcgtataaatggttctgagtacatattcactaatcgacaatgtgatgtgtacagtgagatcatgcagaatgattattttagtttttctggcccgacatccctcggcaaatcattcttgattaaacatgctgccgttgatcttattgaaaataataaactaattattttcatccttccaacaaaagctttattagaagaatatctaattgacttaaaatctatactcaatgaaaaaggcgtgaaagatataaatgtaagtaaatccgtatcacaagttgacaaagaaagtaaaaatatattggtgtttacacaagaaagatataactcatttttatatgaaaagtcttatgataatatggatgttgactttttatttattgatgaagctcataaagtacttgacaaaaagaataatagagctataaccctatataaagtaataaataactccatagaaaaatatggtaaattaaaagtcattttttcctgcccagtaatttccaatcctgatgtgtttttcaaaacatttaatataccgaatagcaagaaaacaagaagtttatgtattaaagaatctccggtaaatcaagatttgtactttgcaaattatcaagataataattttgtctattttgattctattttaaataaaaaaattaatttcaatgtaaataatgcttatcaaaatgactttgaaataatcagaggattagcacaacaatccaagtcaaaccttatctacgtatcaagtaaaacagaatgtataaggaaatgtaacgaatttctagagtatttaattcgggaaaaaaagtatcgataacaacagacgatgaacttatttccgagtcaaaaataatcatggagtatattcataaagattttaacttagctcatgtattaaaatatggtatagcatttcaccatggttcactacctctatttataagaaaaagaatagaggacttatattccaaaaagaaaataaattttatcttttgcacatcaactttacttgaaggggtaaacttacccacaaagaatgtttttatttatccattccctaaaaagactgttaacgatgaaaaaaaatgccgattagatttttggaatctggcggggcgagctggaagatacagaagtgaattgactggtaacatagtatgcctgaataccgctgataataactggagcaatgcagatgctaaaattagcctaggtaataatgttattatagaggatgaaataaactcaacattactacgccatcaaaaaatattaaattattttgatggtaaagttaaatctccaactaatgacattattcaactcagctcacttattctatcagaaatattaacttatttagataaaggttatatagggcgaattttaaatacctttaatgaaaagattcggtttatgcttattgattcaggaaaaaaacatcttgatcgtaatagaattattgaaatagataaagtgaccttttcagaaaatcatatattctcaacatccatacacgctaaagcccaactcaatgctaaaaataaagaaaaattattgcgttcttatagcagagaggatgttttcaattatttggaaacaataaatgaaatatatggcctgcgtaacacccctgattcattaaaccaactgtgcattgtgacttattcgtggctcatgggtaatacgctgaatttattaatatcaaacgctattaaatatagcaattcagttagagatcctattagctatagatgggttaaatttgataaaaccaatcctgatcatataaacgcaaaaataatggaggcaatacaatgcattgaatctgaagttacttttaaacttgaaacatgtattgctcatttttatcagctatgtcaatcaattcacggcgatgaaaatgcaggcattaatctttcaccttatttagaatatggaacattagatactaacatcatagaacttcaagagtttggtttctctcgtttagccgcaattgaaattatagcaaagcacaaagagtgtgttacattcaaaacaaatgagacatcattgcaaataaacactcagaaattaagagctaagattgaaaagcacagtgtaattgatagagaactatcatggttgaatttataattcagccatgataaataataatttgaaagataaataggttgtaatgatataacctatttatcttctcgttacatgaatttcataaggatttttatacggggtataaaatatcattttatatttaacgcggtatactggtaacctaaccgaatgattaatcatcatctaaattataatcatggtaaatagccagttgcttgttgattggtaatttattgatttttaaggccaactcgatagaggatgccgataacatctttctttcttcatctgaagttcgttcaacaccttcaacttggtagagcaaagcatttagcatcttttttataaatatacctcgctgttcatcagacaaatgttcagccaaagtcattagtagacctaattccctttttactcgttcgctatcttgagattgacctgtttcctcacctagtaaccaagccggactaactccagtagcatgagcgatcttctgaagttgctcaattccattcggaagcgaatctccggcaacatatccacggatcgtagagtacgggatatcacatttttgtgccagatcacgcatggaatcgcctgtcatagcgagaagtttctctaatctggcactaaattcactcttaatcatcatactccgattttgtaaccatatgaatgataagaaaaacagatgaactatcgaaaattagatatttatcactttaatttgtctaattttcgatctaatctatctgtacacattgcagacaatcaccctctgcaaacccaactgctgagcatcgcatactcagggcagaattgccacgcatccgctcgtcctgaaaaccgctcagttcacaaaaacacgccacaagcggggataacaaagaaagcagtatgagccatagctgtttatcgccattaccggagccccagacgcttacgcctgtcatgtcttaacctctgtccgataagtctgtgcgctccgggccttttacccggaggactccttatgtggacagtcagcctcgctgaggcgtttattcatgtattccaggtcgcaggcaaaatcttcctcgacatgatgaccgggcttatcccgatgctcatctgcctgctgctggccatcaacttcctgatgaagctggttggtaccgtgcggatggagaaagttgccgccctgctcggccgctcgcgcattctcacctacggcgtgctgccggtgctcggctggttctttatgagcagccccggcgcgctcactttaggcaaactcctgcccgaaaagagcaagcccggctatgaggatgcgctcggcacccccgcccatcccctcaccagcctgtttccccacgtagtgccatcagagctgtttgtctggctcggcgtggctgccggggttaaggcgctcggcctgcccgtcacctcgctggcgctgcgctatatcgccgccgccatgctgattggtttaatccgcgggattatcaccgagtacctgttcctgcggctcagcaagcgggggaatgcgccatgatgatttactgagcacgtatcgtcgccatcgggctgtttgtcgccgatggtctcaccgacaaaatgcttatcaccttcgacagcaacgggccgaaggactgtctcgattactcgctatcgctggagccgtcgttccgggaagagagcctgatgatactccccggcgaccgcctgctgctggcgggccacgattttctggtgactgccgtcggcaaaggggcacagcaggcgctgtttgaactcggccacctgacgctggtgttcaatggtgacctgaatccctgccatgtcggcgcggttcatctctccggcccggtgccgaacctgcgcgacctccacggcaatctggtgattgaggaggggcggccatgaacacggtgctcattccccccggtcccggcggctatggtaaggggctttggctacccacagggagcggcaaaaaggtactgtcgctgaccgggcgcgagattcatccggtggcgatagaaatcggcgcacttaccgaatcggaagtggtgaatggctttgccgatatcccgccggataacgacatcctgtgcgtggtgattaactgtgccgggtccctgcgctgcggcctgtatccgcagaagggtatcccgaccatcaacgtgctgcctacctggcgggccggaccgctggcgcactttattagcgaagagaactacgtctccggtgtgacgatagagcaactggttctggtggataccgccgaagcgccggactgtcagcctgagtcagttccagctccggagccagtcattaccaccgctcctccacctccgcgcggtgcggagaaactggtgcgcatggtcgagcagaccggcaccgccgtcggccacgttatcgccctgctgttcgccgccagccgtgaggcggtggacgtgtcgctgcgcaacgttatcccgtttatggcctttgtctcgctgctgattgccctggtgcaggagaccgcgctcggcagcctgattgcccacgccctgacgccgctggcgaactcactgtggggactggtgctgctgtcgctcatccgcggcattccgtttctctccccggtgctagggccgggtgcggctatctcgcaggtgattggcgtgatgattggcacacagattggcgcaggggcgatatctccggccttcgccctgcccgccctgtttgcgatcaatgtgcaggtcggctgcgacttcgtgcctgttggcctttcgatgcaggaggcaaagccggagaccatcgcaaaaggtgtacccgcctttctgctgtcgagacaactgaccggaccgcttgcggtaatagtcggctggctgttttccctgggcttattctgaggacataacgatgaacaaaatcagagccgccgtggtcggcgcaggtatctacggcaaacaccatatgaatgcgtacctccataacccggacaccgtgctggtcgccatctgcgacaccgatacagaacgctgcgacgacctcgccatgacctgtggcgtgatgggttatacccggctggatgtcctgttacaggaggaagccatcgacgtggtctcggtggcgacacctgacccgtaccacactgaatccatcctcaccgccctgcgccacggcaaacatgtgctggcggagaaaccgctcgccacctcagtacgcgagtgcgagcttatcgtggagatggcgcaacagcgcggcctgctggtcggcgtcgatttccacaaacgctgggacccggcggtgatgcgcatcaaagccgaactggagaaaccggagaccgggcggatcctgcgcggccatatcagtatggacgacgtgattaccgtgccgacggactggctggactgggcgggcgcaagctcccctgtgtggtttctcggctcacactgctttgacctggtgcgccacctttccggtcaggaagtggtgtcggtgtatgccgccgggcagaaacggctgatgaccgaatgcggcctcgacaccttcgacagcgtgcagagccttctgacgatggccgacggcagttcgtgggtggtggaaaactcgtgggtgctgccggagggcttcccgaaggacaacgacgggcgcattgatattctctgtgaggcaacgtatatccgcagcacctcgcagcatcgcgggctggaaatcaccacgccaggcatgacgctcacgcccaacagctacttcatcaactaccgcaacggtgtcgccagcggtttcggtatcgatccgataaacgactttgttcgcgcggtcaggaaccgttcgccgtacccggtgacggcagaggacgggctggcggtcagccggatttgcgaggcggtgcaccgcagtctggagagcgggaaacctgaagatgtttaaacaaacggcgcagggtagctccctgtgccagttccatatgattttgctaattctactcactggtatattcactcgttaggctttggcccgcagatatattaatgttttccatcctgccgcaacaaaaccccacacacgattgtatgttttttagtcatcaaggtgaatagaggtattttatggctatgacctttttcacctccctgccaatagtatcatgctaaacctctcatctatgtttattgcgccataatagtcaacaagtatttcaaaaaagtcccaagcaaattataatgctggagtttcataatgtccctttaaaaaccattttaagaagagctattatgtccctccctacaaattttaatgatattcttcgtctttttgaaaaagattacgatacagccaaagaagataatgccttaagtgcacgcggacaatttctgcaactttacccccttaatcgcctaaaaaaaatgacgcttgatgactatgtcatcggcaaaggcacggcctcattttgtgcttgtgttgaagtaaaaaccagaacatgggcaaatatgcaaggtgcgacggcgctcaaatttggtatttattatggaaaatcaaaatcagatccagccgtccgctatcgttttactcagaaatttggcgatgatgatagtactaataaagaagttttcgctaatgttaaagacgctttactagacctaatacagtcagggaaagaattagattttagagcgattgacgagaaccccctatcccaaatgtttaaggctaaaatattgagcctttactttccagaacactttataaacatttgcagcaaagatcatcttaaagaaattgctatgaaaatgggtataaaagagcaacagtttattagtaaatatcaacatttgttattcaagaaaaaactagagcataaaatcacccgaaactggagcaatccaaaatatatgtccttcctttatgcccagttcatacgtaaggatcttagcagcgctcctgctgtgatagttaaaaaaccacaaaaaagaaaccatcccgaagtcaatttcgaagaaataacggacaatcgtgatttaataggcaaaaaaagcgaagaatatgcattaaactgggaaaaaaaccgcctaatcggtctcggctattcaaaactagctgaggaaatagatgatcgccgtaatcgtccaacttatggttacgactttctttcttttaatgccccaggtgatgagcgatacatcgaagtaaaatcaattggccgagatggaaaagagggagcattccgttttttcctctcaggaaatgaactcaccgtttctaatttaagtaaccaccgtaaaaactattatttttatcttgtacagtatgggaaagatggagagccatgcaatctatatgtaaaacatgctcaagatctttacactaatagtgaaatgaccccttgtgcttacgttgttaggttcgatctggaagaacctgcttaatagtttcgacaacctccgactcataactttaaaagtgtgaaatcaggcgcagagtcacctgagccttcatctcagtgatactgcaacaacacctgcgaaatcttcctcgcttcatcagcaggatccgctgcatgcgtgatcgctgatcccacaatcaccacatccgggcctaacagcgcatagtctttcaccgtctggctactgatgccgcctgctacggcaatccgtgctttccggcgcgccttcagcattgttatcaaatcatcaatcggcttgcgccctgccgcctgctggtcggtgcctgtgtgtaccgccagcatatccgcgcccgcctcttccaggaggcggacccgcgcagggagatcgtcgacgcagatcatatccaccaccacctgctttccagcctccttcgccgcacagatgcatgactggatcgtcagcaggtcggtcacacctaacaccgtgacatagtctgctccggcgtcgaaaagcagctgcgattcaaaatggccgccatccataattttcgcatccaccagtacttccttatgcgggtatttctctttaatgactttaatcgcgttcacgccttcccgaataagaaaaggtgtacccacttcaataatatcaacgtcgtcaaccaccttatcgataaataccagcgcttcaggcagggttaactcgtccagggcaagctgtaatttcatgttggtttccttgtgtcgaatgactattcgaggttggcgtgtagcgcaaaacctttggttaagcgatatcccgtcgactgcgccagattcacaatcatggcatcgcccagcaccatgaccgcctcttcaaacaggctgccgccgggcagaatgcctttcacgttatcgagtccatccggcagtttgtcggtataggccgggatcctgaccactacctccgccagattccccagcgtggactcggggaaaatggtcagcaaggccacagtgccacccagttgcttcgctttcgttgccacattcaccagcgaagccgtttcacccgatgcgctggccagtaacagcaggtcgccatttttcagcgcaggcgtgaccacatcgccgaccacatgcaccttcaggccgatatgcatcaggcgcatcgcaaaagctttcagcattaatagcgaacgccctgcgccaaaaacaaacaccgtgttcgcttcagcgatggcctgctccagacgtgccagcgcagcgccatcaatccgcgacatcgcctgatgcaggtcagtacaggcgacacctgcaacagattgcgtttccatcatgcgtcctcctgacagagcgggaataaatcatcctcatactggagcatccccatgatttcccggacatagttatcgacatgccccagaaagtgaaaacgttccggtgcggcgcgatagcgttgataaagcggcgtatcgaaggtgatgccaagatgcgggttccctttgacggtggcgaatttgatgcgcggaatatgcgggtagtaatgattcttgcgcacgcagagctgcccgttctcttccacgatgtagcaggccataccgtgataaaactgcaccgtgtcgtagtgaaccgcgcagggtttataggccagattgctgaacaccagcggcccgtcgccaatattgatggagcagtggccgtaattcggcggcacgataatgctttccccctccttcaccaccgccacgataagatcgtccaccagcagatcttttggcgcgactgcaaaatccggcgacttttgcaggatatatgccgcagtccccttaatcacttcatataattccggatgggtgttgcgccgcgtgtcgttatagccgtgataatgaccgctggttttcttgcgctccctaccaatcgttcccgacatgatgatggtgatgtcgtactgatactggtcggcagcaagccgttcctgatcctccgcaaaactgagcccgcgataaacatcgtaagccggttcatcacgctgcgcttcggtgaactccggtaagaccaccgccatctgcccggcgcttttgcggccaaagccgagatagttcagcgggggtttcagcaccatcacgccgtcgtcatcgagatacagcggtaagccgctgtggtgtagctgtttcattttgcctcctgtgtatacgcgttagctgcgcggatcgcctctttcagccgatcggatttcatgccaaagacgctttgaatgttgttcccgacctcgacgacacccgccgcaccaagctgtttcagcagcgctttgtccaccagcgacttatccgtcacgtcaatgcgcagacgcgacatgcaggcttcgatgcgggtcatattccccagcccgccataggccagaatgatgccgctcaccaggtcgttctgttcgttttcctgcgccacagtgctttcttcgacttcacaccccggcgtcagcaaattccagcggcggatggcaaaggtaaacagggcgtagtacaccgctcccatcaccagcccgaccgggaacaccatgtaccagtgaggcgcgcgcggcaggacgccaaagaacaaatagtcgataagcccgccggagaaagagagcccgatgtgcacgctgaaccactccatcagcaggaacaccagccccgccaggacggcgtgaatgccatatagcacgggcgcaacaaacaggaaggagaactcaatcggctcggtgatgccgcagacgatggaggtgatcgcgccagagagcataatgcccttcacccgcgctttgttttccgggctggcgcagcggtagatcgccaccgcaatcgccggaatgcagaacatcttgatcggcgtcagcccggccataaaggttccggcggtaatcggcacctgatctttcaactgggcaaagaagataagctggtcgccatgcaccacctgcccggctttgttcacgtactcgccaaactgcagccagaacgtcggccaccagacatggttaagaccaaacggaatcagcagccgctcgacaaaaccgaacaggaaggcggacacgcccggcccctgcaccgtcatagtattcgacaggccattaatcagatgctgaaccggcggccagaccaccgtcatcaccagcccgacaaacagcgcggcgaaggaggtcactatcggcacaaatcgcttgccggagaagaactccagccatgaaggaagctggatgcggtaatagcgtttatacagccaggcggcgataatcccgataatgatgccgccaaacacgccggtctggagcgaagggatgcccagcaccatcgtgtagtcacgtacctgcgccaccgactcgggcgtgatccccagaaactggccgatggtgacgttcataatcagaaaaccggcgatcgccgacaacccggcaatcccctgatcgtcactcagtcctaccgccacgccgaccgcaaacagcagcggcaggttagcgaaaatcgcactgcccgcttcagccatcagcttcagcacgtgaaccagccagtcagcgccgagaaacggcaggctggcgacaatattgggatcctgaaagctgacgccgaaggccagcaaaataccggcggcaggcagcatggcaatggggatcattaacgatcgcccgatgctttgtaacatttgcagaaggttgaatcttttcatctcgatctcctgagcgcgtctttatgacagggattcgcacaacagcaggttctgctccgaacaccttcagtatccgaaccggagatgattaaaaaaacagcaaccgacaacgaaagttatttccggttttcgcttaaaaccggaaaatttatgaggacaatcacgccggaaaccgtgagaaagggataaaagaggaaatcgcgttcggtgggaggccatcatatgagtatcttcgaggcgcattttcgcaggctgcacgcccgctacggcgcaggtcagacgcacgaattacagatgcaggagatcgccgccatcttcggctgttcagtgcgtaattgccggattgctttgaaaaagatgcatcaggaaaaatggctcgactggcagccccagcgcgggcgtggcaagcgctcacggctccacctgttaacctcgccggaaaagctgttcagccagaacgtcaataagctactggagaagcaggattacggcaacgtgctgcggtttatcggcaacgacaagtatctgctggatcgcctgagcctgtggcgctttggggtacaggataaaagcagtgaaacgcgggtgcgcatcccctactatcgcaatctggatccgcttaacccgctcgtccccctgcggcggaccgaacgacatctcctgcgacagtgcctcagcgggctgacgcgctatgacgccgttcagggcaggatcatccccgatatcgcccactactggacccacaacgacgactttacccgctgggagttctggctgaaatccaccgcccgctttgccgatggcagcgaactgaatgccagcgccgttcagcgctgcctgctcgccgccagccagagcccgcagttcgcgccgcttttcagcccaatcaaaaccatcaccgctgacgccccctggcatctggtgattgaaacgtaccatccggtcagacggctcgactgcctgctcgccactcagccgacgatgctgttcgattaccagcacggacacatccgctgtaccggcgctttccacctgcaggaacacagtgacaactttatggttttgcggcgcaatcaacactggcaccaggcgaggcccggactggatgagatcaccattttcacctgggctccggagcatatcagcatgagctttattcccctgctgcggggcgaagaggcgcaggatgaccgcccgctcaacgagcgtagtctggagcagggctgctgttttgtgctgctcgacggtgaaggtgcctttgcggatgaggccggaagacggtttatcaattacctgctgcaacctgtcgaactcctcagccagacgcagcttcctgacgaatacgcacgcatcctctccgttgctcagggcatgctgccgcagtggaatcaccgcccggtggatttcggcgggatcaccgcgccgtttaacctgcgtcagccggttatcatcagcacctttcagcagccggaactggtggagcttgccggagcgattcgcatcctgcttgaacgctggcatattcgcgccgagatacggatcgacgcatttgacagcttcaacagccagccgcgccctcctgcggatatctggctcagcaactttatgctcgataccctttcggttccggcttttctggaatggctggcttccaccacgctgtttacgcgactgcctgaaacccagcgacaaaacctgaacgccctgctgccgaccattctgaacagcgataatgaacaggcgttcgcaaccattgccgcctttttccatgagatgacccaccagcgatatgtcattcctttgctacatcactggatggaatttgcgaccgagaagtcatttacctggcgcgatttaaatacgctgggatggccggattttagccaactttggctcgaataatgctacgcccgcaaaagctcgctaaagggcctttaaaggcctttaagggtaagggagggaagtgcgggatttggggcggggaacggtagacaaattcagtcctcacgacggagactgttcaaatgtctctgtactgacgccccaatatggctttaccaccgtctccgattgttgaacacgtcaatgtcatcgaagatatctgcacgatccagatcctgggttttgtaggcccgttttctgatgcactgctttttcagcgaactgaagcaaggccatagaagtaaaacagtaccgctggcaactgggcattttctcgctttagcggagaccaacagtatgcttagattcagtttatgccgtcagccgatagtgcgactgcaccagacccgaagggaagctgcggctggataatagcgtcaaatcgatatcatggggtaatactccgaatagcggttttcccgaaccgagcagaacaggaacagttgtgataacgatatcggcaaccagaccttcgcgcaggaacgactgtattaactgcccgccatcgatataaacccggtgcacgttcttctccataaggtcagcaagagcctcccttggcgtgcggcatgaaaactgtactttacccttcagcgccttgggcacgggggtatcggttagctgccgggaaagcaccagcacgggccgatcataaggccattcgtcaaaagtcagcaccttctcataggtgcctcgtcccataatgatccaatctttgtccgcgatgaatgcggcatagccatgatcttctgtcgggtcatcgcgctttaacagccagtcgatatcgccatcctgtcgggcgatataaccatcaagactgaccgcgatgaaaacatgtgtggtgaccatcaagtgctccgttttcaggttgcatatatctcgttgagtttagcaatgttcgctccaggcacgaagcgagcaagttacctaagttgaaggtccgcagggagcgaggagcagaagttcgaagcacatcatgaagtaagttttaaaatgggagataacttgcttttacatattttccaatgcaatatcatacccttttaaaaataaatcagatatatgaggtgaaaatgtctagtcttaatttattcgatgatgtcgtacctttagaaaaacagcatccaatatatataatgatgaaaaaggaacgctatgaaccggagagagaagtaattaaccaatgggcaaaaggatttctagacagggataataagtttacaaaagaatttcaaacaagttttgaaccttgcctatgggagttatatctttttgcttatcttaaagaattgggtttaaggaacgacttttcatacgatgcacctgacttcatcgtcaatgaacctgggttttgcattgaggcaaccatagcccttccggcacaaggagcccctggagcgcatggtttcagtacggaagatatgccaagagattttaataagtttaattctgaggcatctattcgacttagcaatagttttatatcaaaggtaaaaaaattaagatcgagatattctcaacttcctcaatgtaaagaaaaaccttttgttatcgccatagcttccttcgaccgaccttttgcgcactttgccgcagccagacccatacttgcaacattatatggtttatatcatgatgaagaagcaaccattgaatcaggatcaaagagcataatttcttataacgttgaggctgcggtaaaaaaagaaaatgttaatatagccatggggttattttgcacgcctgattattcggatgtaagtgctgtgatttatagtagtttagctacttggggaaaagtaagagctttggcggacaatccttcagctctaacaatttatacaacctttaccccaagagaaaattcattatatcctaaagttcatcaagccaaaaaaaatgattatatagagcatctggcagatggattgtatattttacataacccatttgccaagtatccgttgcctaaagagacgctaagccaccctagagtagcacaaggttatgttgaatctgatggatacgtcaattttgtagcgccagaggattttcttttattgcgttttttacaatcattcaatcttaaagattagtaaatatggtcatatttatttactttacatagataaagtctaaagatggcacttcctttaaactcaactacctatgatttagagtggttgtccggcctgcttccgtaaccgttagttcgactgacgagattcctgtttccctctaaaagacttggttgattaaaacaggtcaccgaatagccatttagtccactttgaccacgaagcgaccgaactaactgagcagaaggtccgttatgagcgaggaacggaagttcacagttgatctcacacccaaatagtcgcattactaacattgctgcgtgttaatcaacaggcagcaggtcactcttattttcttgataggctttgtatagtatgaataaaccttacaaagttatgaagaagctcgtgattttcctctgatataccatctgggtcaaagtgcatcacgtcgttacgaattattctgaccttgtcgagctctttagtaaatattttcttatctatttgaagttcggtttttttccacaattctggattttcaaaaagtcgtatatattccccaaaggttaaatctgcgaccgactctataacccgttcgctgtcaaatggattaacgatgctaacaagctcatctttcgtaaattttccatctatcagcttacgaatatgattttcgatttcagatagtaaaagaaaaggttctgacaactgtctgaactgagaactaagatcggtagtagttataatcccactaattctttgatcagaagatcgcactaatacgtaagcatactcaataattctaggtagtgcagaaaaaatagagtcactggcgtttatttccctgtgtggcttcatataatcacgcacaaccgcagattctgattgggtcaacgctagtattggagcaatgctttcccaactaataacacctttaacatctcgctcgctggtcataacgggtagttgagagtagtcatgccgcaacatcaatgtgatggcttctgttaatgtagcgtcaggtttcacggtcactagtttaacatccgatgcttcgagtcgactaacacggaaggctggctcggaaactgcaccggaaattagttcatcatgcggctgaaatactgtttcaggtgtagccaattcggcgttacttgattcggtttcttcatgtaaacattcacctgcttcatggggcgttgcgtcattccccggaggtgcatagatagtaatttttgcatccagcggcacactatcaaaatcaggttcggttttaactcctaagctttctaatgctttgcgaattttgtacgcaaccatgtagcctcgacgctctgcgccaaaccagcttatgaattctcgcactgatactgggtttgaggacataccgtcttttagttcacgaaggatttcgttaagtctttctcgattttctactgccataaaattttctccatgtaggttgtcggctactgttaggtttcaatcttgattcaggcttctcacgctttttttaaaatctggacaacgaggctgcactgactttttcagctcacctcagattgcaccatcccatacacatagctcagattcctaatgttcagtataaggaggccagaatacgaattgcccttaccgctagtgccagcgcgttgagtttagtagatgccttattcatttagaatttgataaaaatacaagactcggacaacaagctgcaatattaatcgttatttttccactcgaggcccaccgtccgtttctggcacagggctgctgttgcagccctgaaaaccttagacttcaatcgattcaaagatctctaatgcatcatctacttcaatacccagataacggactgtgctttccagtttcttatggcccaacagaagttggatcacccgaagattcttagttttcttgtagataaagtagggttttgttcttctcatggaacgtgtgctgtaaagcgaatcttcgagaccaagctttcctgcccacccatgaaagattcggttgtattgtagggttgatatgtgctgattagttccgatacgagaccggaacaagaagtctttattgtgcaaataatcaagctttatcaatgcagcaatagcttctcttgttcctttggttatctcaaattgcacagggctaccggttttctgttgcaacaccgttgctctgcttgaaaccaagcgaccatatgccacatcagatactttgagtttgaccagatcacagcctcgaagcttactgtccagggccatattgaacagagctaaatcgcgcgttttgccttccagttcaagccggattcgggtcccccagatatgagatatctgaagtggtcttttttggccgatgatacggtctttgttccacggtgacgtgttcatactcaaatctcctgcaatgtgggagatttgagtatggttgtcccttatgagcgcaggctgtgtgaaaacactcaccaa

SEQ ID NO: 32 - DNA, 791 bp, Artificial Sequence
  ST69 E. coli_UMN026 CU928163
ttacaaatctgcttgatgaggacgatatactggataccaaatattactgcaacaaagaacttcactgtttttgtcagaaataaaaatcgtaatttccatatcatcccaacctttaacatctctgactaccatccataatggaatatcaaggccgtatgatttcataccatcaggaagaaatttaatagcagtgattttgtatgccagcgagccattaagattatactttttacaatatttttctgctatcgaattcactttgttttcagtaagaataggagcgattattttattattcatgagactccacattgctgtaaataaagaataatttcaacttgcatttccactaactcaccaaaattttcaaagcataactcacaagggaaatcaccgacatcatcggaaaatataccaaaatcatgaatgagaatataaatttttaggctgttgcggtcaagaaaagctacactcccatcgccaaaactgccaacaggaaaaggatcaatgttgttcaataatggatattcttttttataatcttcccattgttgcaaaaatagagggtaatatctgtccagatccttcaaagtataaatttgaggatcgacaaaatgacatgcgccaaaatgagacaataactgccgatagtcttttggaatatgtgttgcgtatttatcctcaaataaaagaaggttatcttcagtttccggagcggttacgcaacctgtaagtcctgttgtttttacggcaggccaggcggtttctaaaatttgaaacattttttccat

SEQ ID NO: 33 - DNA, 15 bp, Artificial Sequence
  Primer 1f
ggcaacaagcataaa

SEQ ID NO: 34 - DNA, 15 bp, Artificial Sequence
  Primer 1r
agggcgttcagaatc

SEQ ID NO: 35 - DNA, 15 bp, Artificial Sequence
  Primer 2f
ttccatttcccatga

SEQ ID NO: 36 - DNA, 15 bp, Artificial Sequence
  primer 2r
tgcataccatttaag

SEQ ID NO: 37 - DNA, 15 bp, Artificial Sequence
  Primer 3f
gctgcgttgcctttc

SEQ ID NO: 38 - DNA, 15 bp, Artificial Sequence
  primer 3r
atagcggtcgattac

SEQ ID NO: 39 - DNA, 15 bp, Artificial Sequence
  Primer 4f
ttctcaatctcttcc

SEQ ID NO: 40 - DNA, 15 bp, Artificial Sequence
  Primer 4r
ctctgtcccaattcc

SEQ ID NO: 41 - DNA, 15 bp, Artificial Sequence
  Primer 5f
attccatcgcaagac

SEQ ID NO: 42 - DNA, 15 bp, Artificial Sequence
  Primer 5r
aatgtccgggattat

SEQ ID NO: 43 - DNA, 21 bp, Artificial Sequence
  First probe
ttacgacccaaagcgaggcat

SEQ ID NO: 44 - DNA, 24 bp, Artificial Sequence
  Second probe
tccgatgtaacctgcaactacgcg

SEQ ID NO: 45 - DNA, 24 bp, Artificial Sequence
  Third probe
tcagtgcaagctggcatagcacta

SEQ ID NO: 46 - DNA, 24 bp, Artificial Sequence
  Fourth probe
accaaggttccgctcttgatcgaa

SEQ ID NO: 47 - DNA, 24 bp, Artificial Sequence
  Fifth probe
aactgttgtagtgggcctgttcca

SEQ ID NO: 48 - DNA, 21 bp, Artificial Sequence
  Primer 1f
tctggaggcaacaagcataaa

SEQ ID NO: 49 - DNA, 21 bp, Artificial Sequence
  Primer 1r
agagaaagggcgttcagaatc

SEQ ID NO: 50 - DNA, 20 bp, Artificial Sequence
  Primer 2f
tcgcattccatttcccatga

SEQ ID NO: 51 - DNA, 21 bp, Artificial Sequence
  Primer 1r
cggcgttgcataccatttaag

SEQ ID NO: 52 - DNA, 21 bp, Artificial Sequence
  Primer 3f
gatattgctgcgttgcctttc

SEQ ID NO: 53 - DNA, 23 bp, Artificial Sequence
  Primer 3r
gtagcttcatagcggtcgattac

SEQ ID NO: 54 - DNA, 22 bp, Artificial Sequence
  Primer 4f
accagcattctcaatctcttcc

SEQ ID NO: 55 - DNA, 22 bp, Artificial Sequence
  Primer 4r
ggacttactctgtcccaattcc

SEQ ID NO: 56 - DNA, 19 bp, Artificial Sequence
  Primer 5f
acccattccatcgcaagac

SEQ ID NO: 57 - DNA, 20 bp, Artificial Sequence
  Primer 5r
gcgtcaatgtccgggattat

SEQ ID NO: 58 - DNA, 21 bp, Artificial Sequence
  Region 1 - ST73 E. coli _CFT073 AE014075
tggttttaccattttgtcgga

SEQ ID NO: 59 - DNA, 20 bp, Artificial Sequence
  Region 1 - ST73 E. coli _CFT073 AE014075
ggaaatcgttgatgttggct

SEQ ID NO: 60 - DNA, 20 bp, Artificial Sequence
  Region 3 - ST127 E. coli_536 CP000247
cgcataacaggattgtctgg

SEQ ID NO: 61 - DNA, 20 bp, Artificial Sequence
  Region 3 - ST127 E. coli_536 CP000247
gctattctacgggcattgtg

SEQ ID NO: 62 - DNA, 20 bp, Artificial Sequence
  Region 11 - ST131 E. coli_NA114 CP002797
gactgcatttcgtcgccata

SEQ ID NO: 63 - DNA, 20 bp, Artificial Sequence
  Region 11 - ST131 E. coli_NA114 CP002797
ccggcggcatcataatgaaa

SEQ ID NO: 64 - DNA, 20 bp, Artificial Sequence
  SEQ ID NO 64
actaatcaggatggcgagac

SEQ ID NO: 65 - DNA, 20 bp, Artificial Sequence
  SEQ ID NO 64
atcacgcccattaatccagt

SEQ ID NO: 66 - DNA, 20 bp, Artificial Sequence
  Region 1 - ST69 E. coli_UMN026 CU928163
atctggaggcaacaagcata

SEQ ID NO: 67 - DNA, 20 bp, Artificial Sequence
  Region 1 - ST69 E. coli_UMN026 CU928163
agagaaagggcgttcagaat

SEQ ID NO: 68 - DNA, 20 bp, Artificial Sequence
  Region-2, ST73
ccgcattcaaaagcataagt

SEQ ID NO: 69 - DNA, 20 bp, Artificial Sequence
  Region-2 ST73
aagattgggtaaaccagcaa

SEQ ID NO: 70 - DNA, 20 bp, Artificial Sequence
  Region-1 ST95
atatctacgacgctgtaacg

SEQ ID NO: 71 - DNA, 20 bp, Artificial Sequence
  Region-1 ST95
gtaatcactacgaggttccc

SEQ ID NO: 72 - DNA, 20 bp, Artificial Sequence
  Region-2 ST95
gagcatctcttgtcatcact

SEQ ID NO: 73 - DNA, 20 bp, Artificial Sequence
  Region-2 ST95
cgtatgggtagggagaaatg

SEQ ID NO: 74 - DNA, 19 bp, Artificial Sequence
  Region-3 ST95
cggaaggaaaagtgaaggt

SEQ ID NO: 75 - DNA, 20 bp, Artificial Sequence
  Region-3 ST95
ctcaacttgccatctcaaca

SEQ ID NO: 76 - DNA, 20 bp, Artificial Sequence
  Region-4 ST95
taagtgcgcagagcataaaa

SEQ ID NO: 77 - DNA, 20 bp, Artificial Sequence
  Region-4 ST95
cagaaaatcttgtcatggcg

SEQ ID NO: 78 - DNA, 20 bp, Artificial Sequence
  Region-5 ST95
aaaatcatctcctgggaacc

SEQ ID NO: 79 - DNA, 20 bp, Artificial Sequence
  Region-5 ST95
tcatgcgtatgaagatgagg

SEQ ID NO: 80 - DNA, 19 bp, Artificial Sequence
  Region-8 ST95
aatccgcaggttatcaagt

SEQ ID NO: 81 - DNA, 20 bp, Artificial Sequence
  Region-8 ST95
ggatcatgagcagtaatcgt

SEQ ID NO: 82 - DNA, 20 bp, Artificial Sequence
  Region-1 ST127
tctctgcatttatgcctgtt

SEQ ID NO: 83 - DNA, 20 bp, Artificial Sequence
  Region-1 ST127
gaggtttccctgatggattt

SEQ ID NO: 84 - DNA, 20 bp, Artificial Sequence
  Region-2 ST127
cttgctgctttagcatcttc

SEQ ID NO: 85 - DNA, 20 bp, Artificial Sequence
  Region-2 ST127
ctcttgcttgaaaggtttgg

SEQ ID NO: 86 - DNA, 20 bp, Artificial Sequence
  Region-4 ST127
attttcctttgatacgccct

SEQ ID NO: 87 - DNA, 20 bp, Artificial Sequence
  Region-4 ST127
cttggtatttcacaatcggc

SEQ ID NO: 88 - DNA, 20 bp, Artificial Sequence
  Region-6 ST127
attgtcatagccaaagtggt

SEQ ID NO: 89 - DNA, 20 bp, Artificial Sequence
  Region-6 ST127
gtgaggaagttattctcgca

SEQ ID NO: 90 - DNA, 19 bp, Artificial Sequence
  Region-7 ST127
agcatggagcgaagaatac

SEQ ID NO: 91 - DNA, 20 bp, Artificial Sequence
  Region-7 ST127
tgaaaggttctcagttgtct

SEQ ID NO: 92 - DNA, 20 bp, Artificial Sequence
  Region-2 ST69
acatctctgactaccatcca

SEQ ID NO: 93 - DNA, 20 bp, Artificial Sequence
  Region-2 ST69
cttttcctgttggcagtttt

SEQ ID NO: 94 - DNA, 20 bp, Artificial Sequence
  Region-1 ST131
gatatgcgtattggtcaggt

SEQ ID NO: 95 - DNA, 20 bp, Artificial Sequence
  Region-1 ST131
gtaaggagcatgtagggaag

SEQ ID NO: 96 - DNA, 20 bp, Artificial Sequence
  Region-2 ST131
ggtatatcctgggctgattg

SEQ ID NO: 97 - DNA, 20 bp, Artificial Sequence
  Region-2 ST131
cggaagctgagttaatggta

SEQ ID NO: 98 - DNA, 20 bp, Artificial Sequence
  Region-3 ST131
caggtgtagattccagtagc

SEQ ID NO: 99 - DNA, 20 bp, Artificial Sequence
  Region-3 ST131
aacacaaaatgtcacagcag

SEQ ID NO: 100 - DNA, 21 bp, Artificial Sequence
  Region-4 ST131
gcatttcgatggcatataagg

SEQ ID NO: 101 - DNA, 20 bp, Artificial Sequence
  Region-4 ST131
tcactttaccagttcggatg

SEQ ID NO: 102 - DNA, 20 bp, Artificial Sequence
  Region-5 ST131
gctcgtcattaaaaacaggg

SEQ ID NO: 103 - DNA, 20 bp, Artificial Sequence
  Region-5 ST131
gcatgttaaccacgcaataa

SEQ ID NO: 104 - DNA, 20 bp, Artificial Sequence
  Region-6 ST131
gctcgtcattaaaaacaggg

SEQ ID NO: 105 - DNA, 20 bp, Artificial Sequence
  Region-6 ST131
atataaacaaaccgggcact

SEQ ID NO: 106 - DNA, 20 bp, Artificial Sequence
  Region-7 ST131
tgccattccaccgttattta

SEQ ID NO: 107 - DNA, 20 bp, Artificial Sequence
  Region-7 ST131
ttcacttacggggttaacag

SEQ ID NO: 108 - DNA, 20 bp, Artificial Sequence
  Region-9 ST131
caataatccggtagagtggg

SEQ ID NO: 109 - DNA, 20 bp, Artificial Sequence
  Region-9 ST131
ttatttactacgtccggcag

SEQ ID NO: 110 - DNA, 20 bp, Artificial Sequence
  Region-10 ST131
tgtgaaagaataacgctgga

SEQ ID NO: 111 - DNA, 20 bp, Artificial Sequence
  Region-10 ST131
gccatctggagaaaggttta

SEQ ID NO: 112 - DNA, 20 bp, Artificial Sequence
  Region_12, ST131
gggcatcttcccagatttta

SEQ ID NO: 113 - DNA, 20 bp, Artificial Sequence
  Region_12, ST131
atcgaatacacgccagatac

SEQ ID NO: 114 - DNA, 20 bp, Artificial Sequence
  ST73-Region-2
cgtcactgccaccgtaataa

SEQ ID NO: 115 - DNA, 22 bp, Artificial Sequence
  ST73-Region-2
gaactcttgaagtcagggttga

SEQ ID NO: 116 - DNA, 24 bp, Artificial Sequence
  ST73-Region-2
ttcttgttggctgcgtcttctcgt

SEQ ID NO: 117 - DNA, 20 bp, Artificial Sequence
  ST95-Region-2
gctccgctttatccgttctt

SEQ ID NO: 118 - DNA, 23 bp, Artificial Sequence
  ST95-Region-2
tgtcattgggtatgggagtattg

SEQ ID NO: 119 - DNA, 24 bp, Artificial Sequence
  ST95-Region-2
ttaccggctgctgcacacatagat

SEQ ID NO: 120 - DNA, 22 bp, Artificial Sequence
  ST95-Region-3
gtcatctttgccaggtcaaatc

SEQ ID NO: 121 - DNA, 20 bp, Artificial Sequence
  ST95-Region-3
tgccatctcaacaggttcaa

SEQ ID NO: 122 - DNA, 24 bp, Artificial Sequence
  ST95-Region-3
caactgccaccacgcccaattaac

SEQ ID NO: 123 - DNA, 24 bp, Artificial Sequence
  ST95-Region-4
gtcattgatgaagctcctgtattg

SEQ ID NO: 124 - DNA, 21 bp, Artificial Sequence
  ST95-Region-4
tgtggctcggatttgtattct

SEQ ID NO: 125 - DNA, 21 bp, Artificial Sequence
  ST95-Region-5
gaaccagcgaccattgtagta

SEQ ID NO: 126 - DNA, 23 bp, Artificial Sequence
  ST95-Region-5
ctaacaggaagggctcgttatag

SEQ ID NO: 127 - DNA, 27 bp, Artificial Sequence
  ST95-Region-5
agcttctcacatcgaggactactgtca

SEQ ID NO: 128 - DNA, 22 bp, Artificial Sequence
  ST95-Region-6
tccactggatacgccttatttg

SEQ ID NO: 129 - DNA, 22 bp, Artificial Sequence
  ST95-Region-6
gggtgtatttcgctttcattcg

SEQ ID NO: 130 - DNA, 25 bp, Artificial Sequence
  ST95-Region-6
aggtggacttagtgggtgaacttgc

SEQ ID NO: 131 - DNA, 21 bp, Artificial Sequence
  ST95-Region-8
tcgagagtcaggcaagaaatg

SEQ ID NO: 132 - DNA, 23 bp, Artificial Sequence
  ST95-Region-8
gcagtaatcgtcattcccataga

SEQ ID NO: 133 - DNA, 24 bp, Artificial Sequence
  ST95-Region-8
acatcaggcttgtcgggtatgagc

SEQ ID NO: 134 - DNA, 24 bp, Artificial Sequence
  ST95-Region-9
catccactcgatcttcgtctactt

SEQ ID NO: 135 - DNA, 23 bp, Artificial Sequence
  ST127-Region-2
tgccttatcagaaactccatctt

SEQ ID NO: 136 - DNA, 22 bp, Artificial Sequence
  ST127-Region-2
cttgcttgaaaggtttggtgag

SEQ ID NO: 137 - DNA, 24 bp, Artificial Sequence
  ST127-Region-2
tctcttggaaggcttgcacacaca

SEQ ID NO: 138 - DNA, 21 bp, Artificial Sequence
  ST127-Region-3
ctcggttgtttgcggtttatc

SEQ ID NO: 139 - DNA, 21 bp, Artificial Sequence
  ST127-Region-3
cccatcagcactacactcttt

SEQ ID NO: 140 - DNA, 26 bp, Artificial Sequence
  ST127-Region-3
acctacggctgatatcttacctgcca

SEQ ID NO: 141 - DNA, 23 bp, Artificial Sequence
  ST127-Region-4
ccctggctgaaatatctggaata

SEQ ID NO: 142 - DNA, 24 bp, Artificial Sequence
  ST127-Region-4
aaaggagaggttgtaggttgtatc

SEQ ID NO: 143 - DNA, 26 bp, Artificial Sequence
  ST127-Region-4
agacccaacataatcacatagcccgc

SEQ ID NO: 144 - DNA, 26 bp, Artificial Sequence
  ST127-Region-6
ccctcatattcggttattgtattctc

SEQ ID NO: 145 - DNA, 28 bp, Artificial Sequence
  ST127-Region-6
cccgcatcattaaatcccagttcttgtg

SEQ ID NO: 146 - DNA, 24 bp, Artificial Sequence
  ST127-Region-7
ccgaagatatgtaagtgtgaaacc

SEQ ID NO: 147 - DNA, 22 bp, Artificial Sequence
  ST127-Region-7
gaaaggttctcagttgtcttgc

SEQ ID NO: 148 - DNA, 24 bp, Artificial Sequence
  ST127-Region-7
tgccagcactgcttgttataccaa

SEQ ID NO: 149 - DNA, 24 bp, Artificial Sequence
  ST69-Region-1
ttacgacccaaagcgaggcattct

SEQ ID NO: 150 - DNA, 22 bp, Artificial Sequence
  ST69-Region-2
gcatttccactaactcaccaaa

SEQ ID NO: 151 - DNA, 18 bp, Artificial Sequence
  ST69-Region-2
tgaccgcaacagcctaaa

SEQ ID NO: 152 - DNA, 26 bp, Artificial Sequence
  ST69-Region-2
caagggaaatcaccgacatcatcgga

SEQ ID NO: 153 - DNA, 20 bp, Artificial Sequence
  ST131-Region-3
ttcacgtggcgtctgaatag

SEQ ID NO: 154 - DNA, 22 bp, Artificial Sequence
  ST131-Region-3
gttcgttattgcgttggatgag

SEQ ID NO: 155 - DNA, 24 bp, Artificial Sequence
  ST131-Region-3
aacttcgccaccacgttaccgaaa

SEQ ID NO: 156 - DNA, 23 bp, Artificial Sequence
  ST131-Region-4
gcatttcgatggcatataaggag

SEQ ID NO: 157 - DNA, 21 bp, Artificial Sequence
  ST131-Region-4
tcactttaccagttcggatgt

SEQ ID NO: 158 - DNA, 29 bp, Artificial Sequence
  ST131-Region-4
acgcgctacatattacggaaaccaattct

SEQ ID NO: 159 - DNA, 21 bp, Artificial Sequence
  ST131-Region-5
tcagctggagtcagcaatatg

SEQ ID NO: 160 - DNA, 22 bp, Artificial Sequence
  ST131-Region-6
ccagagaacggtaatggtgtag

SEQ ID NO: 161 - DNA, 24 bp, Artificial Sequence
  ST131-Region-5
agggagctgtttgaactctggcaa

SEQ ID NO: 162 - DNA, 23 bp, Artificial Sequence
  ST131-Region-6
gggtccggtgaaagacttatatc

SEQ ID NO: 163 - DNA, 20 bp, Artificial Sequence
  ST131-Region-6
atgatggcctggtgttactg

SEQ ID NO: 164 - DNA, 23 bp, Artificial Sequence
  ST131-Region-6
tatttatacgtctgcccgccgca

SEQ ID NO: 165 - DNA, 22 bp, Artificial Sequence
  ST131-Region-7
cgattcccttcccaatgtagtt

SEQ ID NO: 166 - DNA, 20 bp, Artificial Sequence
  ST131-Region-7
gcggatgctgaacggtatta

SEQ ID NO: 167 - DNA, 24 bp, Artificial Sequence
  ST131-Region-7
tcattcaaccgggaaacagccaga

SEQ ID NO: 168 - DNA, 24 bp, Artificial Sequence
  ST131-Region-8
agataacagagggaagtttaacct

SEQ ID NO: 169 - DNA, 23 bp, Artificial Sequence
  ST131-Region-9
ggtagagtgggttaaagggattg

SEQ ID NO: 170 - DNA, 20 bp, Artificial Sequence
  ST131-Region-9
tacgggttctcccgtcataa

SEQ ID NO: 171 - DNA, 24 bp, Artificial Sequence
  ST131-Region-9
atagcgaagccatgagcgcctatt

SEQ ID NO: 172 - DNA, 21 bp, Artificial Sequence
  ST131-Region-10
gcatggtcaagcgtcatattg

SEQ ID NO: 173 - DNA, 22 bp, Artificial Sequence
  ST131-Region-10
cctgtccagagcctacatttat

SEQ ID NO: 174 - DNA, 24 bp, Artificial Sequence
  ST131-Region-10
atacggatcagaaagaccagcgcc

SEQ ID NO: 175 - DNA, 21 bp, Artificial Sequence
  ST131-Region-11
atccgccagaatacccattac

SEQ ID NO: 176 - DNA, 20 bp, Artificial Sequence
  ST131-Region-11
tttcgtcgccatatccatcc

SEQ ID NO: 177 - DNA, 24 bp, Artificial Sequence
  ST131-Region-11
atcagaaacaacatcgcaacgccg

SEQ ID NO: 178 - DNA, 19 bp, Artificial Sequence
  ST131-Region-12
tcgcttctaacaagccgtt

SEQ ID NO: 179 - DNA, 21 bp, Artificial Sequence
  ST131-Region-12
aatacacgccagataccgatg

SEQ ID NO: 180 - DNA, 27 bp, Artificial Sequence
  ST131-Region-12
accactgacctgtaatgaggaaagacc

SEQ ID NO: 181 - DNA, 24 bp, Artificial Sequence
  ST95-Region-4
aagtttgctgccattgacccaacc

SEQ ID NO: 182 - DNA, 23 bp, Artificial Sequence
  ST127-Region-5
tgatgatcaccatcagtccgggt

(19) United States
(12) Patent Application Publication
(10) Pub. No.: US 2016/0208317 A1
(43) Pub. Date: Jul. 21, 2016

(54) ASSAY FOR THE DETECTION OF INFECTION-CAUSING E. COLI STRAINS

(71) Applicant: The Secretary of State for Health, London (GB)

(72) Inventors: Michel DOUMITH, London (GB); Michaela DAY, London (UK); Neil WOODFORD, London (UK)

(73) Assignee: The Secretary of State for Health, London (GB)

(21) Appl. No.: 14/915,033

(22) PCT Filed: Aug. 29, 2014
(86) PCT No.: PCT/GB2014/052615

§ 371 Date: Feb. 26, 2016

(30) Foreign Application Priority Data
Aug. 30, 2013 (GB) 1315527.0

(51) Int. Cl. C12Q 1/68

(52) U.S. Cl. CPC C12Q 1/689

(57) ABSTRACT

The present invention provides nucleic acid products and corresponding methods for screening a biological sample for the presence of an E. coli strain causing an infection.

The present invention relates to nucleic acid products and to corresponding methods for screening a biological sample for the presence of an infection-causing E. coli.

Some strains of Escherichia coli deviate from their commensal status as intestinal flora of mammals and take on a more pathogenic course with the capability to cause disease both within and outside the gut. These pathogenic strains are broadly categorized as either diarrheogenic or extra-intestinal pathogenic E. coli (ExPEC). ExPEC strains have retained the ability to survive in the gut without consequence, but have the capacity to disseminate to and colonize other host sites including the urinary tract, blood and central nervous system, resulting in disease with variable spectrum of clinical severity ranging from asymptomatic bacteriuria, to cystitis and pyelonephritis, to septic shock with multi-organ system failure.

Urinary tract infections (UTIs) are common bacterial infections associated with considerable morbidity. However, uropathogenic E. coli remains the predominant cause of these infections and is responsible for 70 to 90% of acute community-acquired uncomplicated infections, 85% of asymptomatic bacteriuria and for more than 60% of recurrent cystitis. E. coli also represents the biggest cause of bacteraemia with more than 30000 cases p.a in the UK and has more than 20% of mortality, making it one of the most common and challenging bacterial diseases seen in clinical practice. In addition, successful treatment has been complicated by a rise in both the number of antibiotic-resistant strains and the prevalence of antibiotic-resistance mechanisms.

Antibiotic resistance in ExPEC strains (unlike those causing gastrointestinal disease) is a growing concern. E. coli was historically one of the most antibiotic susceptible members of the Enterobacteriaceae family, but has now become one of the most resistant. In the last 10 years, ExPEC strains in particular have developed an alarming penchant for acquiring antibiotic resistance, with >20% of bacteraemia isolates now resistant to fluoroquinolones (predominantly through mutations in DNA gyrase) and >10% resistant to third-generation cephalosporins (through production of various extended-spectrum β-lactamases, ESBLs, particularly CTX-M types), recapitulating rises seen across Europe. This forces increased reliance on carbapenems which, in turn, drives resistance to these, the most active anti-Gram-negative antibiotics.

Recent DNA-based genotyping methods such as multi-locus sequence typing (MLST) have further advanced our understanding of these ExPEC lineages. Screening of more than 2000 bacteraemia-causing E. coli strains isolated between 2000 and 2010 from different UK centres has shown the consistent dominance of just five ST types (e.g. ST69, ST73, ST95, ST127 and ST131) among those isolates. These were collectively responsible for 30 to 60% of bacteraemia cases consistently through the ten years included in the study. Interestingly, their antibiotic resistance profiles differed markedly. Whilst four of these ST types (ST69, ST73, ST95 and ST127) remained relatively susceptible to most antibiotics, the fifth, ST131, has shown increasing resistance to a wide range of classes of antibiotics over the 10 years of the study. Starting from year 2003-2004, isolates belonging to this particular ST-type were predominantly resistant to β-lactams, fluoroquinolones and aminoglycosides. Indeed, the latest is a globally emerging lineage of E. coli and is currently under intense investigation because of its extensive antimicrobial resistance profile, which often includes ESBL production, specifically of CTX-M-15, plus fluoroquinolone and aminoglycoside resistance. In a large number of surveys of human E. coli infections, E. coli-ST131 group was repeatedly reported to be responsible of a large fraction of urine tract infection cases overall, and up to 80-90% of those representing multi antimicrobial-resistant phenotypes, in particular those showing resistant to third-generation cephalosporins. Variants of the ST131 clone frequently host plasmids encoding CTX-M-15 ESBL, but have been identified with many other β-lactamases, suggesting not only that the clone is successful and widely disseminated, but also that it is adept at acquiring locally prevalent plasmids.

The E. coli ST95, a recognized avian pathogenic E. coli (APEC) clonal group, has been found to be predominant within APEC and human ExPEC isolates with diverse host species. E. coli ST69 was identified initially in an apparent outbreak of extraintestinal infections in Berkeley, Calif., during which it accounted for 11% of all UTIs and 52% of antimicrobial-resistant UTIs. It has been subsequently identified around the world, usually as a cause of sporadic human disease and isolates belonging to this group were often associated with resistance to amoxicillin, trimethoprim and sulfamethoxazole.

In a recent published study in the north west of England (Gibreel T M et al 2012), E. coli isolates belonging to ST73, ST131, ST69, ST95 and ST127 were responsible for 16.6, 13.3, 9, 6.3 and 3.6% (collectively c. 50%) of human urinary tract infections, respectively. These findings largely agreed with our results, which were from bloodstream infections, and based on a collection of isolates more representative at the UK national level. Other international molecular epidemiology studies in Canada, France and USA have also pointed out the prevalence of these five ST types among isolates causing urine tract infections or bacteraemia.

A limited number of E. coli STs are collectively responsible for the majority of E. coli urinary tract and bloodstream infections. These STs vary in their antibiotic susceptibility. Rapid identification of particular STs could be used to tailor empiric therapy given to patients to be potentially suited for the particular ST.

STs are defined on the basis of multi-locus sequence typing (MLST), which provides consistent results that are easily comparable across laboratories. However, MLST is time consuming (6-8 h), labour intensive and expensive to perform. Alternative molecular screening strategies are needed to address these limitations and make such testing suited to a larger number of bacteriology laboratories. A screening PCR test has been described to specifically identify isolates belonging to the internationally-disseminated ST131 clone. However, this assay is based on detecting two single nucleotide polymorphisms in the pabB gene and, like many allele-specific PCRs, can suffer from reliability and specificity issues. Otherwise, there are no commercial tests or comparable assays available for the rapid identification of these E. coli STs. There is therefore a need for a more efficient identification system.

The rapid identification of these STs, which are markedly different according to their antibiotic susceptibilities profiles, will allow the treatment options to be adjusted rapidly while waiting for the time-consuming antibiogram (18-24 h). This option potentially contributes to preventing the overuse of some of the most powerful antibiotics (such as carbapenems) by identifying those patients with E. coli infections that might respond to narrower-spectrum antibiotics. By preventing over-reliance on e.g. carbapenems, this strategy will reduce selection pressure for the spread of resistance to these last-resource antibiotics.

The present invention solves the above-identified problems by providing a rapid multiplex PCR assay for targeting and identifying specific E. coli strains in a single isolated sample.

### SUMMARY OF THE INVENTION

By providing a rapid and reliable identification of specific E. coli ST-lineage probes and validation thereof, the invention also provides a rapid multiplex PCR assay for targeting specific E. coli strains in a single isolated sample.

Sequence analyses of 300 publically-available genome sequences deposited in the Genbank database were used to identify sequence-specific DNA regions that are conserved within, but differ between the five major ST types and ST-lineage respective genomes (lineage differed by one or two locus types according to the MLST typing scheme). The number of specific regions identified for each ST varies from 2 to 12 as detailed in Table 1. Five of these ST-specific regions have been selected as targets for the development of a rapid multiplex PCR assay (in the first instance) and their sensitivity and specificity has been evaluated.

ST-specific primers targeting the five selected regions were designed to amplify fragments distinct in sizes in order to facilitate their detection in a single PCR reaction. The expected sizes of amplified products were 104, 200, 310, 404 and 490 bp for the ST69, ST95, ST131, ST127 and ST73-specific targets, respectively (Table 2). Amplification reaction mixtures containing each of the ten primers at a final concentration of 0.2 μM used purified genomic DNA as a template and were performed with the following cycling conditions; an initial denaturation step at 94° C. for 3 min; 30 cycles of 94° C. for 30 sec, 60° C. for 30 sec and 72° C. for 30 sec; and one final cycle of 72° C. for 5 min. The assay was evaluated using 532 isolates of known MLST types; these enclosed isolates of diverse ST types and were obtained from human, cattle or poultry from three different countries as detailed in Table 3. All isolates belonging to ST or ST-complex 69 (n=19), 73 (n=57), 95 (n=23), 127 (n=11) and 131 (n=48) amplified the expected product sizes and were all correctly assigned to the corresponding ST groups by the PCR assay (Table 3). The remaining isolates (n=374) which belonged to other diverse ST types had no PCR products except for two ST12, one ST1056 and one with a new identified ST type (combination of alleles not assigned to an ST type in the MLST database) which were in consequence misidentified as ST95 (n=2), ST73 (n=1) and ST127 (n=1), respectively. In summary, the assay allowed the identification of all five major ST types with an overall accuracy of 99.25%.

The present invention solves one or more of the above-identified problems by providing a simple, one-step assay for detecting the presence or absence of multiple infection-causing E. coli strains in a single isolated sample.

In more detail, a method for detecting the presence or absence of one or more infection-causing E. coli in a sample is provided. Said method comprises applying said sample to one or more wells, wherein said one or more wells comprises: a first probe that binds to a nucleic acid target sequence, wherein said nucleic acid target sequence is defined by E. coli ST 69 region 1 (SEQ ID NO: 31) plus region 2 (SEQ ID NO: 32) nucleic acid sequences, and wherein the first probe comprises at least 10 contiguous nucleotides having at least 80% complementarity to a corresponding 10 contiguous nucleotide sequence of said target sequence; a second probe that binds to a nucleic acid target sequence, wherein said nucleic acid target sequence is defined by E. coli ST 73 region 1 (SEQ ID NO: 1) plus region 2 (SEQ ID NO: 2) nucleic acid sequences, and wherein the second probe comprises at least 10 contiguous nucleotides having at least 80% complementarity to a corresponding 10 contiguous nucleotide sequence of said target sequence; a third probe that binds to a nucleic acid target sequence, wherein said nucleic acid target sequence is defined by E. coli ST 95 regions 1-9 (SEQ ID NOs: 3-11) nucleic acid sequences, and wherein the third probe comprises at least 10 contiguous nucleotides having at least 80% complementarity to a corresponding 10 contiguous nucleotide sequence of said target sequence; a fourth probe that binds to a nucleic acid target sequence, wherein said nucleic acid target sequence is defined by E. coli ST 127 regions 1-7 (SEQ ID NOs: 24-30) nucleic acid sequences, and wherein the fourth probe comprises at least 10 contiguous nucleotides having at least 80% complementarity to a corresponding 10 contiguous nucleotide sequence of said target sequence; a fifth probe that binds to a nucleic acid target sequence, wherein said nucleic acid target sequence is defined by E. coli ST 131 regions 1-12 (SEQ ID NOs: 12-23) nucleic acid sequences, and wherein the fifth probe comprises at least 10 contiguous nucleotides having at least 80% complementarity to a corresponding 10 contiguous nucleotide sequence of said target sequence; allowing nucleic acid present in the sample to contact with the probe within said well; detecting the presence or absence of sample nucleic acid that has bound to one or more of said probes; wherein the presence of sample nucleic acid bound to one or more of said probes confirms that nucleic acid from one or more of said infection-causing E. coli is present within the sample, and wherein the absence of sample nucleic acid bound to one or more of said probes confirms that nucleic acid from said infection-causing E. coli is absent from the sample.

In a further embodiment, the first well comprises the first probe and wherein the second to fifth probes are substantially absent from the first well; the second well comprises the second probe and wherein the first, third to fifth probes are substantially absent from the second well; the third well comprises the third probe and wherein the first to second and fourth to fifth probes are substantially absent from the third well; the fourth well comprises the fourth probe and wherein the first to third and fifth probes are substantially absent from the fourth well; and the fifth well comprises the fifth probe and wherein the first to fourth probes are substantially absent from the fifth well.

In one embodiment, the probes comprise a tag and/or a label. Said tag and/or label is incorporated during extension of the probe(s) such that the amplification product(s) become tagged/labelled. The probes can be labelled with different labels or tags. Each probe can be immobilised within its respective well, and said immobilisation can be permanent or transient.

In one embodiment, an array of target nucleic acid sequences is provided. The array is defined by: region 1 plus region 2 nucleic acid target sequence of E. coli ST 69 (SEQ ID NOs: 31 and 32), wherein said target nucleic acid sequence comprises at least 10 contiguous nucleotides thereof; and region 1 plus region 2 nucleic acid target sequence of E. coli ST 73 (SEQ ID NOs: 1 and 2), wherein said target nucleic acid sequence comprises at least 10 contiguous nucleotides thereof; and regions 1-9 nucleic acid target sequence of E. coli ST 95 (SEQ ID NOs: 3-11), wherein said target nucleic acid sequence comprises at least 10 contiguous nucleotides thereof; and regions 1-7 nucleic acid target sequence of E. coli ST 127 (SEQ ID NOs: 24-30), wherein said target nucleic acid sequence comprises at least 10 contiguous nucleotides thereof; and regions 1-12 nucleic acid target sequence of E. coli ST 131 (SEQ ID NOs: 12-23), wherein said target nucleic acid sequence comprises at least 10 contiguous nucleotides thereof.

In one embodiment, the array of target nucleic acid sequences is provided for use in detecting the presence or absence of an infection-causing E. coli.

In one embodiment, a set of nucleic acid probe sequences comprising at least 10 contiguous nucleotides having at least 80% complementarity to a corresponding 10 contiguous nucleotide sequence of a target sequence is provided. The nucleic acid probe sequences are defined region 1 plus region 2 nucleic acid target sequence of E. coli ST 69 (SEQ ID NOs: 31 and 32); and region 1 plus region 2 nucleic acid target sequence of E. coli ST 73 (SEQ ID NOs: 1 and 2); and regions 1-9 nucleic acid target sequence of E. coli ST 95 (SEQ ID NOs: 3-11); and regions 1-7 nucleic acid target sequence of E. coli ST 127 (SEQ ID NOs: 24-30); and regions 1-12 nucleic acid target sequence of E. coli ST 131 (SEQ ID NOs: 12-23).

In another embodiment, a probe nucleic acid sequence comprising at least 20 contiguous nucleotides having at least 80% complementarity to a corresponding 20 contiguous nucleotide sequence of a target sequence is provided. The probe nucleic acid sequence is defined by: region 1 plus region 2 nucleic acid target sequence of E. coli ST 69 (SEQ ID NOs: 31 and 32); or region 1 plus region 2 nucleic acid target sequence of E. coli ST 73 (SEQ ID NOs: 1 and 2); or regions 1-9 nucleic acid target sequence of E. coli ST 95 (SEQ ID NOs: 3-11); or regions 1-7 nucleic acid target sequence of E. coli ST 127 (SEQ ID NOs: 24-30); or regions 1-12 nucleic acid target sequence of E. coli ST 131 (SEQ ID NOs: 12-23).

In one embodiment, a set of nucleic acid probe sequences comprising at least 20 contiguous nucleotides having at least 80% complementarity to a corresponding 20 contiguous nucleotide sequence of a target sequence is provided. The set of nucleic acid probe sequences is defined by: region 1 plus region 2 nucleic acid target sequence of E. coli ST 69 (SEQ ID NOs: 31 and 32); and region 1 plus region 2 nucleic acid target sequence of E. coli ST 73 (SEQ ID NOs: 1 and 2); and regions 1-9 nucleic acid target sequence of E. coli ST 95 (SEQ ID NOs: 3-11); and regions 1-7 nucleic acid target sequence of E. coli ST 127 (SEQ ID NOs: 24-30); and regions 1-12 nucleic acid target sequence of E. coli ST 131 (SEQ ID NOs: 12-23).

In another embodiment, an array of polypeptide markers encoded by a target nucleic acid sequence is provided. This array is defined by: region 1 plus region 2 nucleic acid target sequence of E. coli ST 69 (SEQ ID NOs: 31 and 32); and region 1 plus region 2 nucleic acid target sequence of E. coli ST 73 (SEQ ID NOs: 1 and 2); and regions 1-9 nucleic acid target sequence of E. coli ST 95 (SEQ ID NOs: 3-11); and regions 1-7 nucleic acid target sequence of E. coli ST 127 (SEQ ID NOs: 24-30); and regions 1-12 nucleic acid target sequence of E. coli ST 131 in (SEQ ID NOs: 12-23). The polypeptide markers are provided for use in detecting the presence or absence of an infection-causing E. coli.

In one embodiment, a test card for use in a method of the invention is provided. Said test card comprises at least five wells, wherein the first well includes the first probe, the second well includes the second probe, the third well includes the third probe, the fourth well includes the fourth probe, and the fifth well includes the fifth probe. The test card is provided wherein: the first well comprises the first probe and wherein the second to fifth probes are substantially absent from the first well; the second well comprises the second probe and wherein the first, third to fifth probes are substantially absent from the second well; the third well comprises the third probe and wherein the first to second and fourth to fifth probes are substantially absent from the third well; the fourth well comprises the fourth probe and wherein the first to third and fifth probes are substantially absent from the fourth well; and the fifth well comprises the fifth probe and wherein the first to fourth probes are substantially absent from the fifth well. The probes are immobilized on the surface of the respective wells; preferably wherein the probes are present in lyophilized form adsorbed to the surface of the respective wells.

One key prior art problem that has been addressed by Applicant is the provision of a robust set of probes that are mutually compatible (i.e. retain accurate binding specificity) within a single set of assay conditions (i.e. a singleplex format). One particular advantage associated with the method of the present invention is speed. By way of example, the method of the invention is typically completed in about 30 minutes. This speed is owing to the fact that the invention allows PCR to be conducted on crude extract, thereby omitting the genomic DNA extraction step before amplification. In contrast, existing multiplex assays utilising traditional PCR amplification and ultra-fast high resolution agarose electrophoresis is done in about an hour at the very least.

Another advantage associated with the uniplex (aka singleplex) assay method of the present invention is an increased sensitivity, which enables quantitative detection of E. coli (for example, bacterial load) in the sample, in addition to simply determining the presence or absence of a particular E. coli in the sample.

E. coli strains in the sample can be subjected to load calibration for each target. This enables the quantification of specific load of each E. coli strain in the sample. Advantageously, this feature of the present invention allows the determination of the predominant strains in samples where multiple strains are present. For example, the uniplex assay method of the invention permits one to ascertain the predominant E. coli strain in samples where multiple strains are present. In addition, the method of the invention allows for the quantitative detection of E. coli strains in samples over time, which is particularly useful when there is fluctuation in bacterial load of specific strains.

Moreover, while existing systems employ hybridisation performed on a membrane, the assay method of the present invention is carried out in a closed (e.g. sterile) system, thus reducing the likelihood of contamination, which provides another advantage.

Probes 1-5 respectively permit sensitive detection of E. coli of the following ST lineages:
1) ST69
2) ST73
3) ST95
4) ST127
5) ST131

Thus, the above-defined method provides a rapid assay for the detection of any one or more of said infection-causing E. coli strains in a uniplex (aka singleplex) assay format. Similarly, said method provides a rapid assay for the confirmation that all of said infection-causing E. coli strains are absent from a sample in a single (uniplex) assay. A uniplex assay means that each of the multiple individual detection well assays is performed under the same assay conditions and/or substantially at the same time. In use, a single sample is applied to the test card, which sample is then populated into each test well.

In one embodiment, the test card may include one or both of said sixth or seventh wells (plus corresponding probes). Alternative ‘control’ probe/probe targets may be employed. Said ‘control’ probes may be used in combination with any of the hereinbefore described embodiments.

Control probes 6-7 respectively permit sensitive detection of:
6) Escherichia coli Bacteriophage MS2 (MS2 IC); and
7) Human Ribonuclease P gene (RNAse P).

The presence of one or more ‘control’ probes allows (substantially simultaneous) confirmation that the assay is otherwise performing normally. For example, the sample is spiked with E. coli bacteriophage MS2 (MS2 IC) prior to nucleic acid extraction. Detection of bacteriophage MS2 nucleic acid in the sample using bacteriophage MS2 probe allows confirmation of the various stages involved in the uniplex assay being completed successfully. Bacteriophage MS2 simply provides one example of an internal control, although any suitable alternative may be utilised with the method of the present invention.

In one embodiment, the test card includes a probe which permits detection of human ribonuclease P gene (RNAse P). The presence of human RNAse P nucleic acid in the sample indicates that human biological material has been collected. Alternatively, other human genome markers may be used as probe targets and their corresponding probes may be included on the test card.

The assay method of the present invention may include a nucleic acid amplification step, in which case each probe of the present invention is employed in combination with a pair of (forward and reverse) primers—said primer pair cooperate to amplify a stretch of target nucleic acid, which is then recognised by the probe (by binding thereto) during the detection step. By way of example, primers 1f (forward) & 1r (reverse) coordinate with the first probe, and in use all three nucleic acid sequences are included in the first well. The same applies to primers 2f & 2r in combination with the second probe (within the second well) through to primers 7f & 7r in combination with the seventh probe (within the seventh well).

Example primer sequences of the invention are exemplified in Tables 2, 4 and 5 and are also set out below.

Primer 1f comprises a nucleic acid sequence that has at least 80% sequence identity to GGCAACAAGCATAAA (SEQ ID NO: 33), and primer 1r comprises a nucleic acid sequence that has at least 80% sequence identity to AGGGCGTTCAGAATC (SEQ ID NO: 34).

Primer 2f comprises a nucleic acid sequence that has at least 80% sequence identity to TTCCATTTCCCATGA (SEQ ID NO: 35), and primer 2r comprises a nucleic acid sequence that has at least 80% sequence identity to TGCATACCATTTAAG (SEQ ID NO: 36).

Primer 3f comprises a nucleic acid sequence that has at least 80% sequence identity to GCTGCGTTGCCTTTC (SEQ ID NO: 37), and primer 3r comprises a nucleic acid sequence that has at least 80% sequence identity to ATAGCGGTCGATTAC (SEQ ID NO: 38).

Primer 4f comprises a nucleic acid sequence that has at least 80% sequence identity to TTCTCAATCTCTTCC (SEQ ID NO: 39), and primer 4r comprises a nucleic acid sequence that has at least 80% sequence identity to CTCTGTCCCAATTCC (SEQ ID NO: 40).

Primer 5f comprises a nucleic acid sequence that has at least 80% sequence identity to ATTCCATCGCAAGAC (SEQ ID NO: 41), and primer 5r comprises a nucleic acid sequence that has at least 80% sequence identity to AATGTCCGGGATTAT (SEQ ID NO: 42).

The biological sample is typically a sample that has been taken from a patient (i.e. an ex vivo and/or isolated sample). In one embodiment, a nucleic acid extraction step may be performed on the sample—conventional nucleic acid extraction protocols are well known in the art. The extracted nucleic acid sample is then applied so that is contacts each of the wells (and thus each of the probes within said wells). In another embodiment, the sample taken from the patient is directly applied to a well.

The nucleic acid ‘hybridization reaction’ (comprising probe and primers working together) step of the present invention is typically performed at a temperature of 50-70° C. (for example, 55-65° C. or 56-64° C. or 57-63° C. or 58-62° C. or 59-61° C. or approximately 60° C.). Said temperature is typically held for a time period of 10-30 seconds (for example, 15-25 seconds or 17-23 seconds or 19-21 seconds or approximately 20 seconds). If a nucleic acid amplification step is included in the method of the invention, said ‘hybridization reaction’ (comprising probe and primers added in excess at the beginning) step is preferably included in each cycle of the amplification step.

If a nucleic acid amplification step is employed, this step is typically performed at a temperature of 90-100° C. (for example, 92-98° C. or 94-96° C. or approximately 95° C. degrees) for a typical period of 0.1-10 seconds (for example, 0.5-5 seconds or 0.7-2 seconds or approximately 1 second) followed by a reduced temperature of 50-70° C. (for example, 55-65° C. or 57-63° C. or 59-61° C. or approximately 60° C.) for a period 10-30 seconds (for example, 15-25 seconds or 17-23 seconds or 19-21 seconds or approximately 20 seconds). If a nucleic acid amplification step is employed, said step typically includes 35-55 cycles (for example, 40-50 cycles or 44-46 cycles or approximately 45 cycles). A reverse transcription step is typically employed at the very start at a temperature of 40-60° C. (for example, 45-55° C. or 48-52° C. or approximately 50° C.) for a time period of 3-7 minutes (for example, 4-6 minutes or approximately 5 minutes).

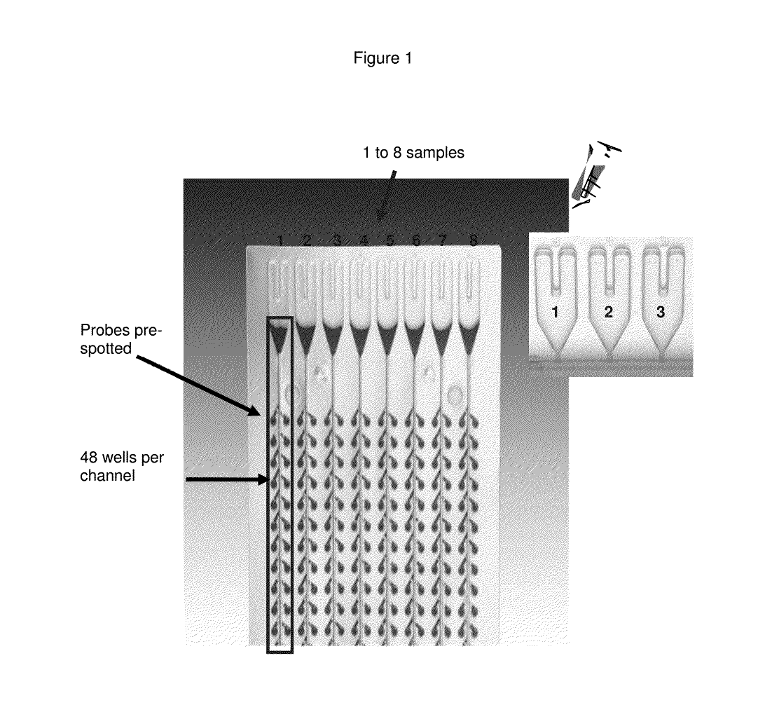

In one embodiment, the method may be performed in an Applied Biosystems 7900HT (high throughput) instrument. By way of example, said instrument may employ a 384 well test card (aka plate) RT-PCR platform that allows, for example, 8 different samples to be analysed in parallel via 8 distinct columns present on a single test card—see FIG. 1. Each column may comprise 48 individual target wells, thereby permitting each sample to be (substantially simultaneously) screened for 7 different E. coli strains (effectively 5 E. coli strains, as two ‘control’ wells are employed). Alternative apparatuses and systems (including corresponding test cards) are available commercially and have equal application in the context of the present invention.

In one embodiment, the method employs PCR such as RT-PCR.

In use, a sample (typically extracted nucleic acid samples) is mixed with 2-times to 5-times concentrated buffer (e.g. PCR buffer; also referred to as reaction mix). For example, Xμl of sample is mixed with the same volume (Xμl) of 2-times concentrated buffer. The sample (including buffer) is then applied to each well—typically a volume in the range of 0.1-50 μl, or 0.5-30 μl, or 0.5-20 μl, or 0.5-10 μl, or 1-5 μl is delivered to each well. Preferably approximately 0.5 μl, 1 μl, 4 μl, 3 μl, 4 μl or 5 μl of sample (including buffer) is delivered to each well.

In one embodiment a test card is provided. In another embodiment there is no test card and the sample is applied to one or more wells.

A well of the invention is herein intended to embrace any structure providing a volume for retaining a sample.

In the case of a test card comprising a columnar arrangement of wells (see, for example, FIG. 1), the sample (including buffer) may simply be applied to a reservoir at the top of each column, and the test card then spun in a centrifuge to deliver sample plus reagent mix (in the volume range as identified above) to each of the wells forming in each column. In the case of the AB7900HT system, each well typically comprises 48 wells so sample is applied by centrifugal delivery to each of said 48 wells. In more detail, up to 8 samples may be added respectively to the 8 reservoirs at the top of each column (e.g. with a fin pipette). Referring to FIG. 1, the little pods indicate the discrete assay wells, which in turn include the corresponding probes (and optionally the corresponding primers). The illustrated test card shows a set up in which 48 wells (also referred to as pods) are present per channel—in use, each well typically receives a final 1 μl reaction volume by centrifugal delivery down the columnar channel.

Each well includes one specific probe type of the present invention (and optionally the corresponding primer pair). In one embodiment, said probe is present in its well in a lyophilized form. Thus, once the liquid sample has been applied to the well surface, the lyophilized probe (optionally including the corresponding primer pair) becomes re-hydrated, thereby allowing the detection step to proceed within a liquid medium.

A well of the present invention is designed to hold slightly more than the relevant liquid volume (sample plus buffer/reaction mix) of the assay that is to be performed in said well. Each well is discrete to allow location of a single probe type within a single well, thereby permitting the method to detect the presence or absence of specific target E. coli strains. Following application of sample to the test card, all of the wells containing probe(s) may be sealed shut by use of one or more films/sheets, thereby preventing accidental migration of liquid (and potentially probes) between wells. A well of the present invention may be positioned in the same horizontal plane as the test card, though equally may be positioned above or below said plane.

Compared to a standard battery of multiplex reaction set-ups, the present invention offers time and resource savings in both reaction set up manipulations and permits collation of data from multiple instruments.

The present invention also provides a test card for use in the hereinbefore described methods. In one embodiment, the test card is made from a plastics material. For the purpose of assisting the user, the test card should have sufficient rigidity to support the weight of the card (including applied sample), for example when in a substantially horizontal position as typically held by the user during normal use.

The test card comprises a plurality of wells (optionally arranged in a columnar format to permit sample application by centrifugal delivery), wherein at least seven wells are provided, and wherein the first well includes the first probe, the second well includes the second probe, the third well includes the third probe, the fourth well includes the fourth probe, the fifth well includes the fifth probe, and the sixth well includes the seventh probe of the present invention as herein defined. Each well typically only includes (a plurality of) one specific probe of the present invention. By way of example, in one embodiment, the first probe is present in the first well (though typically absent from any of the other wells), and the second probe is present in the second well (though typically absent from any of the other wells), and so on.

Each probe may optionally be associated with its corresponding primer pair. Thus, in addition to the first probe, the first well may include the first pair of corresponding forward and reverse primers. Each well typically only includes (a plurality of) one specific primer pair of the present invention. By way of example, in one embodiment, the first primer pair (and the first probe) is present in the first well but typically absent from any of the other wells, and the second primer pair (and the second probe) is present in the second well but typically absent from any of the other wells, and so on. Alternatively, more than one probe (and optionally its corresponding primer pair) may be present in a single well.

Each probe may be immobilised within its respective well—said immobilisation may be permanent (e.g. via a covalent link, optionally introduced by any commercially available chemical cross-linking reagents) or transient (e.g. via a non-covalent bond such as a hydrogen bond, or via an ionic bond). For example, the first probe may be immobilised within the first well, and the second probe may be immobilised within the second well, and so on. Immobilisation of the respective probes makes the test cards easier to handle, improves storage stability, and minimises the risk of probe migration between wells. The probes are preferably immobilised within the wells by simple adsorption on to a surface present in the wells, such as on to a wall of a well. Thus, in one embodiment, a probe-containing solution is prepared, applied to the surface of a well, and then allowed to dry on the surface of the well. Conventional stabilising compounds (e.g. sugars) may be added to the probe-containing solution prior to application to a well surface.

The test card may include one or more additional wells. Each of the above-described test card embodiments may further include one or more wells for detecting atypical E. coli strains. Each of the above-described test card embodiments may further include one or more ‘control’ wells.

In one embodiment there is provided a polypeptide marker encoded by a target nucleic acid sequence, and a method for the detection thereof. Such a polypeptide marker can be detected by conventional protein detection methods including the use of antibodies, HPLC, mass spectroscopy. In one embodiment, the polypeptide markers of the invention are at least 10 amino acids in length. In one embodiment, the polypeptide markers of the invention are at least 20, 30, 40, 50, 60, 70, 80, 90, or 100 amino acids in length.

### DEFINITIONS SECTION

Reference to at least 80% sequence identity includes at least 82%, at least 84%, at least 86%, at least 88%, at least 90%, at least 91%, at least 92%, at least 93%, at least 94%, at least 95%, at least 96%, at least 97%, at least 98%, at least 99%, and 100% sequence identity (to each and every nucleic acid sequence presented herein and/or to each and every SEQ ID NO presented herein).

The one-letter reference code for nucleotides employed throughout this specification means:
A adenine
C cytosine
G guanine
T thymine
U uracil
I inosine
X inosine
R guanine or adenine
Y thymine or cytosine
K guanine or thymine
M adenine or cytosine
S guanine or cytosine
W adenine or thymine
B not adenine
D not cytosine
H not guanine
V not thymine
N any nucleic acid base

All nucleic acid sequences presented herein are presented in a 5′-to-3′ (left-to-right) orientation.

The probes of the invention are designed to hybridise to their target nucleic acid sequence present on the target E. coli strain in question. It is preferred that the binding conditions are such that a high level of specificity is provided—i.e. hybridisation of the probe occurs under “stringent conditions”. In general, stringent conditions are selected to be about 5° C. lower than the thermal melting point (Tₘ) for the specific sequence at a defined ionic strength and pH. The Tₘ is the temperature (under defined ionic strength and pH) at which 50% of the target (or complement) sequence hybridises to a perfectly matched probe. In this regard, the Tₘ of probes of the present invention, at a salt concentration of about 0.02M or less at pH 7, is for example above 60° C., such as about 70° C.

Premixed buffer solutions are commercially available (eg. EXPRESSHYB Hybridisation Solution from CLONTECH Laboratories, Inc.), and hybridisation can be performed according to the manufacturer's instructions.

Probes of the present invention are screened to minimise self-complementarity and dimer formation (probe-probe binding), and are selected so as to have minimal homology with human DNA. The selection process typically involves comparing a candidate probe sequence with human DNA and rejecting the probe if the homology is greater than 50%. The aim of this selection process is to reduce annealing of probe to contaminating human DNA sequences and hence allow improved specificity of the assay.

Any of the probes described herein may comprise a tag and/or label. The tag and/or label may, for example, be located (independently of one another) towards the middle or towards or at the 5′ or 3′ end of the herein described probes, for example at the 5′ end.

Hence, following hybridisation of tagged/labelled probe to target nucleic acid, the tag/label is associated with the target nucleic acid. Alternatively, if an amplification step is employed, the probes may act as primers during the method of the invention and the tag/label may therefore become incorporated into the amplification product as the primer is extended.

Examples of suitable labels include detectable labels such as radiolabels or fluorescent or coloured molecules, enzymatic markers or chromogenic markers—e.g. dyes that produce a visible colour change upon hybridisation of the probe. By way of example, the label may be digoxygenin, fluorescein-isothiocyanate (FITC), R-phycoerythrin, Alexa 532 or Cy3. The probes preferably contain a Fam label (e.g. a 5′ Fam label), and/or a minor groove binder (MGB). The label may be a reporter molecule, which is detected directly, such as by exposure to photographic or X-ray film. Alternatively, the label is not directly detectable, but may be detected indirectly, for example, in a two-phase system. An example of indirect label detection is binding of an antibody to the label.

Examples of suitable tags include “complement/anti-complement pairs”. The term “complement/anti-complement pair” denotes non-identical moieties that form a non-covalently associated, stable pair under appropriate conditions. Examples of suitable tags include biotin and streptavidin (or avidin). By way of example, a biotin tag may be captured using streptavidin, which may be coated onto a substrate or support such as a bead (for example a magnetic bead) or membrane. Likewise, a streptavidin tag may be captured using biotin, which may be coated onto a substrate or support such as a bead (for example a magnetic bead) or membrane. Other exemplary complement/anti-complement pairs include receptor/ligand pairs, antibody/antigen (or hapten or epitope) pairs, and the like. Another example is a nucleic acid sequence tag that binds to a complementary sequence. The latter may itself be pre-labelled, or may be attached to a surface (eg. a bead) which is separately labelled. An example of the latter embodiment is the well-known Luminex^{R} bead system. Other exemplary pairs of tags and capture molecules include receptor/ligand pairs and antibody/antigen (or hapten or epitope) pairs. Where subsequent dissociation of the complement/anti-complement pair is desirable, the complement/anti-complement pair has a binding affinity of, for example, less than 10⁹ M⁻¹.

The probes of the invention may be labelled with different labels or tags, thereby allowing separate identification of each probe when used in the method of the present invention.

Any conventional method may be employed to attach nucleic acid tags to a probe of the present invention (e.g. to the 5′ end of the defined binding region of the probe). Alternatively, nucleic acid probes of the invention (with pre-attached nucleic acid tags) may be constructed by commercial providers.

The sample is for example a clinical sample (or is derived from a clinical sample) such as: blood, sputum, nose and throat swabs, bronchoalveolar lavage, tracheal aspirate, nasopharyngeal aspirates, lung tissue samples, cerebrospinal fluid, archaeological, faecal samples. The sample is preferably a human tissue/sample or is a sample derived therefrom (e.g. a nucleic acid extracted sample).

If an amplification step is employed, this step may be carried out using methods and platforms known in the art, for example PCR (for example, with the use of “Fast DNA Polymerase”, Life Technologies), such as real-time PCR, block-based PCR, ligase chain reaction, glass capillaries, isothermal amplification methods including loop-mediated isothermal amplification, rolling circle amplification transcription mediated amplification, nucleic acid sequence-based amplification, signal mediated amplification of RNA technology, strand displacement amplification, isothermal multiple displacement amplification, helicase-dependent amplification, single primer isothermal amplification, and circular helicase-dependent amplification.

If employed, amplification may be carried using any amplification platform—as such, an advantage of this embodiment of the assay is that it is platform independent and not tied to any particular instrument.

In one embodiment, a general amplification step (eg. pre-detection) may be employed to increase the amount of target nucleic acid present in the sample. In this embodiment, PCR amplification primers are typically employed to amplify approximately 100-400 base pair regions of the target/complementary nucleic acid that contain the nucleotide targets of the present invention. In the presence of a suitable polymerase and DNA precursors (dATP, dCTP, dGTP and dTTP), forward and reverse primers are extended in a 5′ to 3′ direction, thereby initiating the synthesis of new nucleic acid strands that are complementary to the individual strands of the target nucleic acid. The primers thereby drive amplification of target nucleic acid sequences, thereby generating amplification products comprising said target nucleic acid sequences.

In one embodiment, an amplification step may be employed in which the probes of the present invention act as primers. In this embodiment, the probes (acting as primers) are extended from their 3′ ends (i.e. in a 5′-to-′3′) direction. The resulting amplification products typically comprise 100-400 base pair regions of the target/complementary nucleic acid. This embodiment may be employed in conjunction with a general amplification step, such as the one described above.

The detection step may be carried out by any known means. In this regard, the probe or amplification product may be tagged and/or labelled, and the detection method may therefore comprise detecting said tag and/or label.

In one embodiment, the probe(s) may comprise a tag and/or label. Thus, in one embodiment, following hybridisation of tagged/labelled probe to target nucleic acid, the tag/label becomes associated with the target nucleic acid. Thus, in one embodiment, the assay may comprise detecting the tag/label and correlating presence of tag/label with presence of E. coli nucleic acid.

In one embodiment, tag and/or label may be incorporated during extension of the probe(s). In doing so, the amplification product(s) become tagged/labelled, and the assay may therefore comprise detecting the tag/label and correlating presence of tag/label with presence of amplification product, and hence the presence of E. coli nucleic acid.

By way of example, in one embodiment, the amplification product may incorporate a tag/label (eg. via a tagged/labelled dNTP such as biotin-dNTP) as part of the amplification process, and the assay may further comprise the use of a binding partner complementary to said tag (eg. streptavidin) that includes a detectable tag/label (eg. a fluorescent label, such as R-phycoerythrin). In this way, the amplified product incorporates a detectable tag/label (e.g. a fluorescent label, such as R-phycoerythrin).

In one embodiment, the probe(s) and/or the amplification product(s) may include a further tag/label (as the complement component) to allow capture of the amplification product(s).

By way of example, a “complement/anti-complement” pairing may be employed in which an anti-complement capture component binds to said further tag/label (complement component) and thereby permits capture of the probe(s) and/or amplification product(s). Examples of suitable “complement/anti-complement” partners have been described earlier in this specification, such as a complementary pair of nucleic acid sequences, a complementary antibody-antigen pair, etc. The anti-complement capture component may be attached (eg. coated) on to a substrate or solid support—examples of suitable substrates/supports include membranes and/or beads (eg. a magnetic or fluorescent bead). Capture methods are well known in the art. For example, Luminex^{R} beads may be employed. Alternatively, the use of magnetic beads may be advantageous because the beads (plus captured, tagged/labelled amplification product) can easily be concentrated and separated from the sample, using conventional techniques known in the art.

Immobilisation provides a physical location for the anti-complement capture component (or probes), and may serve to fix the capture component/probe at a desired location and/or facilitate recovery or separation of probe. The support may be a rigid solid support made from, for example, glass or plastic, such as a bead (for example a fluorescent or magnetic bead). Alternatively, the support may be a membrane, such as nylon or nitrocellulose membrane. 3D matrices are also suitable supports for use with the present invention—eg. polyacrylamide or PEG gels. Immobilisation to a support/platform may be achieved by a variety of conventional means. By way of example, immobilisation onto a support such as a nylon membrane may be achieved by UV cross-linking. Alternatively, biotin-labelled molecules may be bound to streptavidin-coated substrates (and vice-versa), and molecules prepared with amino linkers may be immobilised on to silanised surfaces. Another means of immobilisation is via a poly-T tail or a poly-C tail, for example at the 3′ or 5′ end. Said immobilisation techniques apply equally to the probe component (and primer pair component, if present) of the present invention.

In one embodiment, the probes of the invention comprise a nucleic acid sequence tag/label (e.g. attached to each probe at the 5′ end of the defined sequence of the probe that binds to target/complement nucleic acid). In more detail, each of the probes is provided with a different nucleic acid sequence tag/label, wherein each of said tags/labels (specifically) binds to a complementary nucleic acid sequence present on the surface of a bead. Each of the different tags/labels binds to its complementary sequence counterpart (and not to any of the complementary sequence counterparts of the other tags), which is located on a uniquely identifiable bead. In this regard, the beads are uniquely identifiable, for example by means of fluorescence at a specific wavelength. Thus, in use, probes of the invention bind to target nucleic acid (if present in the sample). Thereafter, (only) the bound probes may be extended (in the 3′ direction) in the presence of one or more labelled dNTP (eg. biotin labelled dNTPs, such as biotin-dCTPs).

The extended primers may be contacted with a binding partner counterpart to the labelled dNTPs (eg. a streptavidin labelled flurophore, such as streptavidin labelled R-phycoerythrin), which binds to those labelled dNTPs that have become incorporated into the extended primers. Thereafter, the labelled extended primers may be identified by allowing them to bind to their nucleic acid counterparts present on the uniquely identifiable beads. The latter may then be “called” (eg. to determine the type of bead present by wavelength emission) and the nature of the primer extension (and thus the type of target/complement nucleic acid present) may be determined.

### PREFERRED EMBODIMENTS OF THE PRESENT INVENTION

The first probe comprises a nucleic acid sequence that has at least 80% sequence identity to TTACGACCCAAAGCGAGGCAT (SEQ ID NO: 43).

The second probe comprises a nucleic acid sequence that has at least 80% sequence identity to TCCGATGTAACCTGCAACTACGCG (SEQ ID NO: 44).

The third probe comprises a nucleic acid sequence that has at least 80% sequence identity to TCAGTGCAAGCTGGCATAGCACTA (SEQ ID NO: 45).

The fourth probe comprises a nucleic acid sequence that has at least 80% sequence identity to ACCAAGGTTCCGCTCTTGATCGAA (SEQ ID NO: 46).

The fifth probe comprises a nucleic acid sequence that has at least 80% sequence identity to AACTGTTGTAGTGGGCCTGTTCCA (SEQ ID NO: 47).

Primer 1f comprises a nucleic acid sequence that has at least 80% sequence identity to TCTGGAGGCAACAAGCATAAA (SEQ ID NO: 48), and primer 1r comprises a nucleic acid sequence that has at least 80% sequence identity to AGAGAAAGGGCGTTCAGAATC (SEQ ID NO: 49).

Primer 2f comprises a nucleic acid sequence that has at least 80% sequence identity to TCGCATTCCATTTCCCATGA (SEQ ID NO: 50), and primer 1r comprises a nucleic acid sequence that has at least 80% sequence identity to CGGCGTTGCATACCATTTAAG (SEQ ID NO: 51).

Primer 3f comprises a nucleic acid sequence that has at least 80% sequence identity to GATATTGCTGCGTTGCCTTTC (SEQ ID NO: 52), and primer 3r comprises a nucleic acid sequence that has at least 80% sequence identity to GTAGCTTCATAGCGGTCGATTAC (SEQ ID NO: 53).

Primer 4f comprises a nucleic acid sequence that has at least 80% sequence identity to ACCAGCATTCTCAATCTCTTCC (SEQ ID NO: 54), and primer 4r comprises a nucleic acid sequence that has at least 80% sequence identity to GGACTTACTCTGTCCCAATTCC (SEQ ID NO: 55).

Primer 5f comprises a nucleic acid sequence that has at least 80% sequence identity to ACCCATTCCATCGCAAGAC (SEQ ID NO: 56), and primer 5r comprises a nucleic acid sequence that has at least 80% sequence identity to GCGTCAATGTCCGGGATTAT (SEQ ID NO: 57).

### Sequence Homology/Identity

Any of a variety of sequence alignment methods can be used to determine percentage identity, including, without limitation, global methods, local methods and hybrid methods, such as, e.g., segment approach methods. Protocols to determine percentage identity are routine procedures within the scope of one skilled in the. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position—Specific Gap Penalties and Weight Matrix Choice, 22 (22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262 (5131) Science 208-214 (1993); Align-M, see, e.g., Ivo Van Walle et al., Align-M—A New Algorithm for Multiple Alignment of Highly Divergent Sequences, 20 (9) Bioinformatics: 1428-1435 (2004). Thus, percentage sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992.

The same methods can be utilised for determining percentage complementarity between sequences. In this case, the percentage identity is determined between complementary base pairs, for example, between the alignment of one nucleotide with its complementary nucleotide.

Variants of the specific sequences provided above may alternatively be defined by reciting the number of nucleotides that differ between the variant sequences and the specific reference sequences provided above. Thus, in one embodiment, the sequence may comprise (or consist of) a nucleotide sequence that differs from the specific sequences provided above at no more than 2 nucleotide positions, for example at no more than 1 nucleotide position. Conservative substitutions are preferred.

By way of example, variant probe sequences may comprise nucleic acid sequences selected from: (SEQ ID NO: 1); (SEQ ID NO: 2); (SEQ ID NO: 3); (SEQ ID NO: 4) or (SEQ ID NO: 5).

Fragments of the above-mentioned sequences (and sequence variants thereof as defined above) may also be employed, for example, fragments comprising 10, 9, 8, 7, 6, 5, 4, 3, 2 or 1 base pair of the defined sequences described herein.

TABLE 1
Sequence positions of specific ST lineage targets

position

| | | | | |
|---|---|---|---|---|
| SEQ ID NO: | Region | start | end | Size (bp) |

ST73 E. coli_CFT073 AE014075

| | | | | |
|---|---|---|---|---|
| 1 | 1 | 2001254 | 2001324 | 70 |
| | | 2001324 | 2002124 | 800 |
| | | 2002124 | 2002243 | 119 |
| 2 | 2 | 5116943 | 5120014 | 3071 |
| | | 5120028 | 5122040 | 2012 |
| | | 5122071 | 5122415 | 344 |
| | | 5122185 | 5122343 | 158 |
| | | 5122193 | 5122630 | 437 |
| | | 5122627 | 5123328 | 701 |
| | | 5123325 | 5126603 | 3278 |
| | | 5126603 | 5127508 | 905 |
| | | 5127508 | 5127677 | 544 |

ST95 E. coli_UTI89 CP000243

| | | | | |
|---|---|---|---|---|
| 3 | 1 | 1397376 | 1398467 | 1091 |
| | | 1398483 | 1399484 | 1001 |
| | | 1399484 | 1399596 | 112 |
| 4 | 2 | 2598930 | 2599265 | 335 |
| 5 | 3 | 2900299 | 2900429 | 130 |
| | | 2900429 | 2900626 | 197 |
| | | 2900623 | 2901411 | 788 |
| | | 2901411 | 2901499 | 88 |
| | | 2901499 | 2901792 | 293 |
| | | 2901792 | 2902003 | 211 |
| 6 | 4 | 2925552 | 2926201 | 649 |
| | | 2926201 | 2926449 | 248 |
| | | 2926449 | 2926523 | 74 |
| | | 2927725 | 2927877 | 152 |
| | | 2927877 | 2928212 | 335 |
| 7 | 5 | 2941113 | 2941712 | 599 |
| | | 2941741 | 2942067 | 326 |
| | | 2942072 | 2942983 | 911 |
| 8 | 6 | 3114339 | 3116108 | 1769 |
| | | 3116083 | 3116916 | 833 |
| | | 3117178 | 3117642 | 464 |
| 9 | 7 | 3124006 | 3125577 | 1571 |
| | | 3125577 | 3126218 | 641 |
| | | 3126218 | 3126499 | 281 |
| 10 | 8 | 3861904 | 3862236 | 332 |
| | | 3862243 | 3862728 | 485 |
| | | 3862745 | 3863491 | 746 |
| | | 3863491 | 3863628 | 137 |
| | | 3863628 | 3864338 | 710 |
| | | 3864340 | 3865260 | 920 |
| | | 3865260 | 3866144 | 884 |
| | | 3866144 | 3866248 | 104 |
| 11 | 9 | 4523060 | 4523100 | 40 |

ST131 E. coli_NA114 CP002797

| | | | | |
|---|---|---|---|---|
| 12 | 1 | 290463 | 291668 | 1205 |
| 13 | 2 | 565237 | 566022 | 785 |
| | | 566055 | 566852 | 797 |
| | | 566852 | 566906 | 54 |
| | | 566906 | 567973 | 1067 |
| 14 | 3 | 569537 | 570157 | 620 |
| | | 570157 | 570204 | 47 |
| | | 570204 | 571145 | 941 |
| | | 571145 | 571204 | 59 |
| | | 571204 | 572724 | 1520 |
| | | 572724 | 573075 | 351 |
| | | 573075 | 574025 | 950 |
| | | 574025 | 574388 | 363 |
| | | 574388 | 575218 | 830 |
| | | 575211 | 576146 | 935 |
| 15 | 4 | 933939 | 934139 | 200 |
| 16 | 5 | 1354460 | 1354759 | 299 |
| | | 1359910 | 1360133 | 223 |
| | | 1360330 | 1360887 | 557 |
| | | 1363715 | 1364611 | 896 |
| | | 1364611 | 1364727 | 116 |
| | | 1364712 | 1367369 | 2657 |
| | | 1367366 | 1367578 | 212 |
| | | 1367592 | 1368761 | 1169 |
| | | 1368761 | 1368948 | 187 |
| | | 1368948 | 1369085 | 137 |
| | | 1370029 | 1370199 | 170 |
| | | 1370199 | 1370517 | 318 |
| | | 1371008 | 1371368 | 360 |
| | | 1371368 | 1371652 | 284 |
| | | 1371652 | 1371763 | 111 |
| 17 | 6 | 1971120 | 1971539 | 419 |
| | | 1971539 | 1971977 | 438 |
| | | 1971977 | 1972750 | 773 |
| | | 1972750 | 1973565 | 815 |
| | | 1973565 | 1975205 | 1640 |
| | | 1975205 | 1975314 | 109 |
| | | 1975314 | 1975595 | 281 |
| | | 1975608 | 1976120 | 512 |
| | | 1976138 | 1977640 | 1502 |
| | | 1977640 | 1978026 | 386 |
| | | 1978026 | 1979210 | 1184 |
| | | 1979203 | 1979829 | 626 |
| | | 1979832 | 1980752 | 920 |
| | | 1980749 | 1981081 | 332 |
| | | 1981093 | 1981995 | 902 |
| | | 1981976 | 1982512 | 536 |
| | | 1982509 | 1983189 | 680 |
| | | 1983221 | 1983562 | 341 |
| | | 1983598 | 1984017 | 419 |
| | | 1985086 | 1985145 | 59 |
| | | 1985145 | 1985474 | 329 |
| | | 1988352 | 1988585 | 233 |
| | | 1990351 | 1990434 | 83 |
| | | 1990434 | 1990739 | 305 |
| | | 1990739 | 1991674 | 935 |
| | | 1991674 | 1991868 | 194 |
| | | 1992355 | 1992627 | 272 |
| | | 1992627 | 1993010 | 383 |
| | | 1993010 | 1993123 | 113 |
| | | 1993123 | 1993794 | 671 |
| | | 1994680 | 1994758 | 78 |
| | | 1994758 | 1994937 | 179 |
| 18 | 7 | 2480863 | 2481273 | 410 |
| | | 2481266 | 2481502 | 236 |
| | | 2481894 | 2482177 | 283 |
| | | 2482177 | 2483181 | 1004 |
| | | 2483204 | 2484064 | 860 |
| 19 | 8 | 2795123 | 2795162 | 39 |
| 20 | 9 | 2928397 | 2930265 | 1868 |
| | | 2930265 | 2930371 | 106 |
| 21 | 10 | 298525 | 299301 | 776 |
| | | 299509 | 299844 | 335 |
| 22 | 11 | 4343837 | 4345117 | 1280 |
| 23 | 12 | 4859427 | 4859514 | 87 |
| | | 4859514 | 4860560 | 1046 |
| | | 4865298 | 4865489 | 191 |
| | | 4860560 | 4860972 | 412 |
| | | 4861720 | 4863936 | 2216 |
| | | 4863936 | 4863967 | 31 |
| | | 4863967 | 4864287 | 320 |
| | | 4864287 | 4865298 | 1011 |
| | | 4865298 | 4865489 | 191 |
| | | 4865489 | 4866099 | 610 |
| | | 4866615 | 4866839 | 224 |
| | | 4866836 | 4867591 | 755 |
| | | 4867581 | 4868996 | 1415 |
| | | 4869035 | 4869445 | 410 |
| | | 4869447 | 4869683 | 236 |
| | | 4869988 | 4870212 | 224 |

ST127 E. coli_536 CP000247

| | | | | |
|---|---|---|---|---|
| 24 | 1 | 2835907 | 2836404 | 497 |
| | | 2836428 | 2837312 | 884 |
| | | 2837312 | 2837717 | 405 |
| | | 2837717 | 2837917 | 200 |
| | | 2837917 | 2838745 | 828 |
| 25 | 2 | 2839884 | 2841254 | 1370 |
| 26 | 3 | 2844403 | 2844642 | 239 |
| | | 2844642 | 2844856 | 214 |
| | | 2844856 | 2848707 | 3851 |
| | | 2848707 | 2848976 | 269 |
| | | 2848976 | 2849725 | 749 |
| | | 2849725 | 2849946 | 221 |
| 27 | 4 | 342066 | 342251 | 185 |
| | | 342251 | 342718 | 467 |
| | | 342718 | 343605 | 887 |
| 28 | 5 | 343605 | 343652 | 47 |
| 29 | 6 | 345148 | 345442 | 294 |
| | | 345442 | 346164 | 722 |
| | | 346164 | 346356 | 192 |
| 30 | 7 | 349823 | 350210 | 387 |

ST69 E. coli_UMN026 CU928163

| | | | | |
|---|---|---|---|---|
| 31 | 1 | 3051269 | 3052441 | 1172 |
| | | 3052441 | 3052815 | 374 |
| | | 3052815 | 3053432 | 617 |
| | | 3053407 | 3054033 | 626 |
| | | 3054033 | 3054619 | 586 |
| | | 3054619 | 3056688 | 2069 |
| | | 3056688 | 3057361 | 673 |
| | | 3057361 | 3058347 | 986 |
| | | 3058340 | 3059563 | 1223 |
| | | 3059590 | 3059805 | 215 |
| | | 3059798 | 3061000 | 1202 |
| | | 3061000 | 3061039 | 39 |
| | | 3061039 | 3062322 | 1283 |
| | | 3062322 | 3062477 | 155 |
| | | 3062477 | 3062956 | 479 |
| | | 3062956 | 3063317 | 361 |
| | | 3063317 | 3063838 | 521 |
| | | 3063838 | 3063988 | 150 |
| | | 3063988 | 3064209 | 221 |
| | | 3064206 | 3065186 | 980 |
| | | 3065197 | 3066210 | 1013 |
| | | 3066210 | 3066453 | 243 |
| | | 3066453 | 3067706 | 1253 |
| | | 3067706 | 3067778 | 72 |
| | | 3067778 | 3068413 | 635 |
| | | 3068413 | 3068437 | 24 |
| | | 3068437 | 3069003 | 566 |
| | | 3069000 | 3069842 | 842 |
| | | 3069839 | 3071365 | 1526 |
| | | 3071365 | 3071573 | 208 |
| | | 3071573 | 3073267 | 1694 |
| | | 3073267 | 3073619 | 352 |
| | | 3073619 | 3074158 | 539 |
| | | 3074158 | 3074332 | 174 |
| | | 3074332 | 3075402 | 1070 |
| | | 3075402 | 3075474 | 72 |
| | | 3075474 | 3075698 | 224 |
| | | 3075698 | 3075733 | 35 |
| | | 3075733 | 3076830 | 1097 |
| | | 3076830 | 3077184 | 354 |
| | | 3077184 | 3077864 | 680 |
| 32 | 2 | 1679641 | 1679937 | 296 |
| | | 167993 | 1680431 | 494 |

TABLE 2
Example primer sequences used for identification of ExPEC major ST-lineages

| Primers | | Size(bp) | Positions | Comments |
|---|---|---|---|---|
| ST73_for | TGGTTTTACCATTTTGTCGGA | 490 | 2001936 . . . 2001916 | Region 1 - ST73 E. coli _CFT073 |
| | (SEQ ID NO: 58) | | | AE014075 |
| ST73_rev | GGAAATCGTTGATGTTGGCT | | 2001447 . . . 2001466 | |
| | (SEQ ID NO: 59) | | | |
| ST127_for | CGCATAACAGGATTGTCTGG | 404 | 2847996 . . . 2847977 | Region 3 - ST127 E. coli_536 |
| | (SEQ ID NO: 60) | | | CP000247 |
| ST127_rev | GCTATTCTACGGGCATTGTG | | 2847593 . . . 2847612 | |
| | (SEQ ID NO: 61) | | | |
| ST131_for | GACTGCATTTCGTCGCCATA | 310 | 4344866 . . . 4344847 | Region 11 - ST131 E. coli_NA114 |
| | (SEQ ID NO: 62) | | | CP002797 |
| ST131_rev | CCGGCGGCATCATAATGAAA | | 4344565 . . . 4344584 | |
| | (SEQ ID NO: 63) | | | |
| ST95_for | ACTAATCAGGATGGCGAGAC | 200 | 3124468 . . . 3124487 | Region 7 - ST95 E. coli_UTI89 |
| | (SEQ ID NO: 64) | | | CP000243 |
| ST95_rev | ATCACGCCCATTAATCCAGT | | 3124668 . . . 3124649 | |
| | (SEQ ID NO: 65) | | | |
| ST69_for | ATCTGGAGGCAACAAGCATA | 104 | 3053203 . . . 3053222 | Region 1 - ST69 E. coli_UMN026 |
| | (SEQ ID NO: 66) | | | CU928163 |
| ST69_rev | AGAGAAAGGGCGTTCAGAAT | | 3053306 . . . 3053287 | |
| | (SEQ ID NO: 67) | | | |

TABLE 3
Strains used to validate PCR assay

| Isolate | Country | Origin | Detailed source | ST by MLST | PCR product size (bp) | ST by PCR | Comments |
|---|---|---|---|---|---|---|---|
| E1 | UK | Human | Blood | 10 | — | — | |
| E2 | UK | Human | Blood | 10 | — | — | |
| E3 | Netherlands | Human | Urine | 10 | — | — | |
| E4 | Germany | Pig | Organ | 10 | — | — | |
| E5 | Germany | Pig | Organ | 10 | — | — | |
| E6 | Netherlands | Human | Urine | 10 | — | — | |
| E7 | Germany | Pig | Urine | 10 | — | — | |
| E8 | Netherlands | Poultry | Caecum | 10 | — | — | |
| E9 | Netherlands | Poultry | Meat | 10 | — | — | |
| E10 | Netherlands | Human | Urine | 10 | — | — | |
| E11 | Netherlands | Poultry | Meat | 10 | — | — | |
| E12 | Netherlands | Poultry | Meat | 10 | — | — | |
| E13 | UK | Cattle | Faeces | 10 | — | — | |
| E14 | UK | Cattle | Faeces | 10 | — | — | |
| E15 | UK | Cattle | Faeces | 10 | — | — | |
| E16 | UK | Cattle | Intestine | 10 | — | — | |
| E17 | Netherlands | Human | Urine | 10 | — | — | |
| E18 | UK | Cattle | Faeces | 10 | — | — | |
| E19 | UK | Poultry | Caeca | 10 | — | — | |
| E20 | Netherlands | Human | Urine | 10 | — | — | |
| E21 | UK | Human | Clinical isolate | 10 | — | — | |
| E22 | UK | Human | Blood | 12 | — | — | |
| E23 | UK | Human | Blood | 12 | — | — | |
| E24 | UK | Human | Blood | 12 | — | — | |
| E25 | UK | Human | Blood | 12 | — | — | |
| E26 | UK | Human | Blood | 12 | — | — | |
| E27 | UK | Human | Blood | 12 | — | — | |
| E28 | UK | Human | Blood | 12 | — | — | |
| E29 | UK | Human | Blood | 12 | — | — | |
| E30 | UK | Human | Blood | 12 | — | — | |
| E31 | UK | Human | Blood | 12 | — | — | |
| E32 | UK | Human | Blood | 12 | — | — | |
| E33 | UK | Human | Blood | 12 | — | — | |
| E34 | UK | Pig | Large intestine | 12 | — | — | |
| E35 | Netherlands | Human | Urine | 12 | — | — | |
| E36 | UK | Human | Clinical isolate | 12 | 200 | 95 | Disagreement between methods |
| E37 | UK | Human | Clinical isolate | 12 | 200 | 95 | Disagreement between methods |
| E38 | UK | Human | Blood | 14 | — | — | |
| E39 | UK | Human | Blood | 14 | — | — | |
| E40 | UK | Human | Blood | 14 | — | — | |
| E41 | UK | Human | Blood | 14 | — | — | |
| E42 | UK | Human | Blood | 14 | — | — | |
| E43 | UK | Human | Blood | 14 | — | — | |
| E44 | UK | Human | Blood | 14 | — | — | |
| E45 | UK | Human | Blood | 14 | — | — | |
| E46 | UK | Human | Blood | 23 | — | — | |
| E47 | Netherlands | Human | Urine | 23 | — | — | |
| E48 | Netherlands | Human | Urine | 23 | — | — | |
| E49 | Netherlands | Poultry | Meat | 23 | — | — | |
| E50 | Germany | Human | Clinical Isolate | 28 | — | — | |
| E51 | Germany | Human | Clinical Isolate | 34 | — | — | |
| E52 | UK | Human | Blood | 38 | — | — | |
| E53 | UK | Human | Blood | 38 | — | — | |
| E54 | Netherlands | Human | Urine | 38 | — | — | |
| E55 | UK | Human | Clinical isolate | 38 | — | — | |
| E56 | UK | Human | Clinical isolate | 38 | — | — | |
| E57 | UK | Human | Clinical isolate | 38 | — | — | |
| E58 | UK | Human | Clinical isolate | 38 | — | — | |
| E59 | Netherlands | Human | Urine | 38 | — | — | |
| E60 | Netherlands | Poultry | Caecum | 44 | — | — | |
| E61 | Netherlands | Human | Urine | 46 | — | — | |
| E62 | Germany | Poultry | Faeces | 48 | — | — | |
| E63 | Netherlands | Poultry | Meat | 48 | — | — | |
| E64 | Germany | Cattle | Organ | 58 | — | — | |
| E65 | Netherlands | Human | Urine | 58 | — | — | |
| E66 | Netherlands | Human | Urine | 58 | — | — | |
| E67 | Netherlands | Human | Urine | 58 | — | — | |
| E68 | Netherlands | Human | Urine | 58 | — | — | |
| E69 | Netherlands | Poultry | Meat | 58 | — | — | |
| E70 | UK | Cattle | Faeces | 58 | — | — | |
| E71 | UK | Cattle | Faeces | 58 | — | — | |
| E72 | UK | Cattle | Faeces | 58 | — | — | |
| E73 | Netherlands | Human | Urine | 58 | — | — | |
| E74 | UK | Human | Blood | 59 | — | — | |
| E75 | UK | Human | Blood | 59 | — | — | |
| E76 | UK | Human | Blood | 62 | — | — | |
| E77 | UK | Human | Blood | 68 | — | — | |
| E78 | UK | Human | Blood | 69 | 104 | 69 | |
| E79 | UK | Human | Blood | 69 | 104 | 69 | |
| E80 | UK | Human | Blood | 69 | 104 | 69 | |
| E81 | UK | Human | Blood | 69 | 104 | 69 | |
| E82 | UK | Human | Blood | 69 | 104 | 69 | |
| E83 | UK | Human | Blood | 69 | 104 | 69 | |
| E84 | UK | Human | Blood | 69 | 104 | 69 | |
| E85 | UK | Human | Blood | 69 | 104 | 69 | |
| E86 | UK | Human | Blood | 69 | 104 | 69 | |
| E87 | UK | Human | Blood | 69 | 104 | 69 | |
| E88 | UK | Cattle | Faeces | 69 | 104 | 69 | |
| E89 | Netherlands | Human | Urine | 69 | 104 | 69 | |
| E90 | Germany | Human | Clinical Isolate | 69 | 104 | 69 | |
| E91 | Germany | Cattle | Organ | 69 | 104 | 69 | |
| E92 | UK | Cattle | Faeces | 69 | 104 | 69 | |
| E93 | UK | Human | Blood | 73 | 490 | 73 | |
| E94 | UK | Human | Blood | 73 | 490 | 73 | |
| E95 | UK | Human | Blood | 73 | 490 | 73 | |
| E96 | UK | Human | Blood | 73 | 490 | 73 | |
| E97 | UK | Human | Blood | 73 | 490 | 73 | |
| E98 | UK | Human | Blood | 73 | 490 | 73 | |
| E99 | UK | Human | Blood | 73 | 490 | 73 | |
| E100 | UK | Human | Blood | 73 | 490 | 73 | |
| E101 | UK | Human | Blood | 73 | 490 | 73 | |
| E102 | UK | Human | Blood | 73 | 490 | 73 | |
| E103 | UK | Human | Blood | 73 | 490 | 73 | |
| E104 | UK | Human | Blood | 73 | 490 | 73 | |
| E105 | UK | Human | Blood | 73 | 490 | 73 | |
| E106 | UK | Human | Blood | 73 | 490 | 73 | |
| E107 | UK | Human | Blood | 73 | 490 | 73 | |
| E108 | UK | Human | Blood | 73 | 490 | 73 | |
| E109 | UK | Human | Blood | 73 | 490 | 73 | |
| E110 | UK | Human | Blood | 73 | 490 | 73 | |
| E111 | UK | Human | Blood | 73 | 490 | 73 | |
| E112 | UK | Human | Blood | 73 | 490 | 73 | |
| E113 | UK | Human | Blood | 73 | 490 | 73 | |
| E114 | UK | Human | Blood | 73 | 490 | 73 | |
| E115 | UK | Human | Blood | 73 | 490 | 73 | |
| E116 | UK | Human | Blood | 73 | 490 | 73 | |
| E117 | UK | Human | Blood | 73 | 490 | 73 | |
| E118 | UK | Human | Blood | 73 | 490 | 73 | |
| E119 | UK | Human | Blood | 73 | 490 | 73 | |
| E120 | UK | Human | Blood | 73 | 490 | 73 | |
| E121 | UK | Human | Blood | 73 | 490 | 73 | |
| E122 | UK | Human | Blood | 73 | 490 | 73 | |
| E123 | UK | Human | Blood | 73 | 490 | 73 | |
| E124 | UK | Human | Blood | 73 | 490 | 73 | |
| E125 | UK | Human | Blood | 73 | 490 | 73 | |
| E126 | UK | Human | Blood | 73 | 490 | 73 | |
| E127 | UK | Human | Blood | 73 | 490 | 73 | |
| E128 | UK | Human | Blood | 73 | 490 | 73 | |
| E129 | UK | Human | Blood | 73 | 490 | 73 | |
| E130 | UK | Human | Blood | 73 | 490 | 73 | |
| E131 | UK | Human | Blood | 73 | 490 | 73 | |
| E132 | UK | Human | Blood | 73 | 490 | 73 | |
| E133 | UK | Human | Blood | 73 | 490 | 73 | |
| E134 | UK | Human | Blood | 73 | 490 | 73 | |
| E135 | UK | Human | Blood | 73 | 490 | 73 | |
| E136 | UK | Human | Blood | 73 | 490 | 73 | |
| E137 | UK | Human | Blood | 73 | 490 | 73 | |
| E138 | UK | Human | Clinical isolate | 73 | 490 | 73 | |
| E139 | UK | Human | Clinical isolate | 73 | 490 | 73 | |
| E140 | UK | Human | Clinical isolate | 73 | 490 | 73 | |
| E141 | UK | Cattle | Faeces | 75 | — | — | |
| E142 | UK | Human | Blood | 80 | — | — | |
| E143 | UK | Human | Blood | 80 | — | — | |
| E144 | UK | Human | Blood | 80 | — | — | |
| E145 | Germany | Cattle | Organ | 85 | — | — | |
| E146 | UK | Human | Blood | 88 | — | — | |
| E147 | UK | Human | Blood | 88 | — | — | |
| E148 | UK | Human | Clinical isolate | 88 | — | — | |
| E149 | Germany | Cattle | Milk | 88 | — | — | |
| E150 | Germany | Pig | Organ | 88 | — | — | |
| E151 | Germany | Pig | Organ | 88 | — | — | |
| E152 | Germany | Cattle | Organ | 88 | — | — | |
| E153 | Germany | Pig | Urine | 88 | — | — | |
| E154 | Netherlands | Poultry | Caecum | 88 | — | — | |
| E155 | Netherlands | Poultry | Meat | 88 | — | — | |
| E156 | Netherlands | Poultry | Meat | 88 | — | — | |
| E157 | Netherlands | Human | Urine | 88 | — | — | |
| E158 | UK | Cattle | Intestine | 88 | — | — | |
| E159 | UK | Cattle | Faeces | 88 | — | — | |
| E160 | UK | Cattle | Carcass | 88 | — | — | |
| E161 | UK | Poultry | Caeca | 88 | — | — | |
| E162 | UK | Poultry | Caeca | 88 | — | — | |
| E163 | UK | Poultry | Caeca | 88 | — | — | |
| E164 | UK | Poultry | Caeca | 88 | — | — | |
| E165 | Netherlands | Human | Urine | 88 | — | — | |
| E166 | UK | Human | Clinical isolate | 90 | — | — | |
| E167 | Netherlands | Human | Urine | 90 | — | — | |
| E168 | Netherlands | Human | Urine | 93 | — | — | |
| E169 | Netherlands | Poultry | Caecum | 93 | — | — | |
| E170 | Netherlands | Poultry | Meat | 93 | — | — | |
| E171 | Netherlands | Poultry | Meat | 93 | — | — | |
| E172 | UK | Human | Blood | 95 | 200 | 95 | |
| E173 | UK | Human | Blood | 95 | 200 | 95 | |
| E174 | UK | Human | Blood | 95 | 200 | 95 | |
| E175 | UK | Human | Blood | 95 | 200 | 95 | |
| E176 | UK | Human | Blood | 95 | 200 | 95 | |
| E177 | UK | Human | Blood | 95 | 200 | 95 | |
| E178 | UK | Human | Blood | 95 | 200 | 95 | |
| E179 | UK | Human | Blood | 95 | 200 | 95 | |
| E180 | UK | Human | Blood | 95 | 200 | 95 | |
| E181 | UK | Human | Blood | 95 | 200 | 95 | |
| E182 | UK | Human | Blood | 95 | 200 | 95 | |
| E183 | UK | Human | Blood | 95 | 200 | 95 | |
| E184 | UK | Human | Blood | 95 | 200 | 95 | |
| E185 | UK | Human | Blood | 95 | 200 | 95 | |
| E186 | UK | Human | Blood | 95 | 200 | 95 | |
| E187 | UK | Human | Blood | 95 | 200 | 95 | |
| E188 | UK | Human | Blood | 95 | 200 | 95 | |
| E189 | UK | Human | Blood | 95 | 200 | 95 | |
| E190 | UK | Human | Blood | 95 | 200 | 95 | |
| E191 | Netherlands | Human | Urine | 95 | 200 | 95 | |
| E192 | UK | Human | Clinical isolate | 95 | 200 | 95 | |
| E193 | Germany | Pig | Organ | 101 | — | — | |
| E194 | Germany | Turkey | Meat | 101 | — | — | |
| E195 | Germany | Cattle | Organ | 101 | — | — | |
| E196 | UK | Human | Clinical isolate | 104 | 490 | 73 | 73-SLV |
| E197 | UK | Human | Blood | 106 | — | — | |
| E198 | UK | Human | Blood | 106 | — | — | |
| E199 | UK | Cattle | Faeces | 106 | — | — | |
| E200 | UK | Cattle | Faeces | 106 | — | — | |
| E201 | UK | Cattle | Faeces | 106 | — | — | |
| E202 | UK | Human | Blood | 117 | — | — | |
| E203 | UK | Human | Blood | 117 | — | — | |
| E204 | Germany | Poultry | Unknown | 117 | — | — | |
| E205 | Germany | Pig | Organ | 117 | — | — | |
| E206 | Germany | Pig | Organ | 117 | — | — | |
| E207 | Netherlands | Poultry | Meat | 117 | — | — | |
| E208 | Netherlands | Poultry | Meat | 117 | — | — | |
| E209 | UK | Cattle | Faeces | 117 | — | — | |
| E210 | UK | Cattle | Faeces | 117 | — | — | |
| E211 | Netherlands | Human | Urine | 117 | — | — | |
| E212 | UK | Human | Clinical isolate | 117 | — | — | |
| E213 | UK | Human | Blood | 126 | — | — | |
| E214 | UK | Human | Blood | 127 | 404 | 127 | |
| E215 | UK | Human | Blood | 127 | 404 | 127 | |
| E216 | UK | Human | Blood | 127 | 404 | 127 | |
| E217 | UK | Human | Blood | 127 | 404 | 127 | |
| E218 | UK | Human | Blood | 127 | 404 | 127 | |
| E219 | UK | Human | Blood | 127 | 404 | 127 | |
| E220 | UK | Human | Blood | 127 | 404 | 127 | |
| E221 | UK | Human | Blood | 127 | 404 | 127 | |
| E222 | UK | Human | Blood | 127 | 404 | 127 | |
| E223 | UK | Human | Blood | 131 | 310 | 131 | |
| E224 | UK | Human | Blood | 131 | 310 | 131 | |
| E225 | UK | Human | Blood | 131 | 310 | 131 | |
| E226 | UK | Human | Blood | 131 | 310 | 131 | |
| E227 | UK | Human | Blood | 131 | 310 | 131 | |
| E228 | UK | Human | Blood | 131 | 310 | 131 | |
| E229 | UK | Human | Blood | 131 | 310 | 131 | |
| E230 | UK | Human | Blood | 131 | 310 | 131 | |
| E231 | UK | Human | Blood | 131 | 310 | 131 | |
| E232 | UK | Human | Blood | 131 | 310 | 131 | |
| E233 | UK | Human | Blood | 131 | 310 | 131 | |
| E234 | UK | Human | Blood | 131 | 310 | 131 | |
| E235 | UK | Human | Blood | 131 | 310 | 131 | |
| E236 | UK | Human | Blood | 131 | 310 | 131 | |
| E237 | UK | Human | Blood | 131 | 310 | 131 | |
| E238 | UK | Human | Blood | 131 | 310 | 131 | |
| E239 | UK | Human | Blood | 131 | 310 | 131 | |
| E240 | UK | Human | Blood | 131 | 310 | 131 | |
| E241 | UK | Human | Blood | 131 | 310 | 131 | |
| E242 | UK | Human | Blood | 131 | 310 | 131 | |
| E243 | UK | Human | Blood | 131 | 310 | 131 | |
| E244 | UK | Human | Blood | 131 | 310 | 131 | |
| E245 | UK | Human | Blood | 131 | 310 | 131 | |
| E246 | Netherlands | Human | Urine | 131 | 310 | 131 | |
| E247 | Netherlands | Human | Urine | 131 | 310 | 131 | |
| E248 | Netherlands | Human | Urine | 131 | 310 | 131 | |
| E249 | Netherlands | Human | Urine | 131 | 310 | 131 | |
| E250 | Netherlands | Human | Urine | 131 | 310 | 131 | |
| E251 | Netherlands | Human | Urine | 131 | 310 | 131 | |
| E252 | Netherlands | Human | Urine | 131 | 310 | 131 | |
| E253 | Germany | Human | Faeces | 131 | 310 | 131 | |
| E254 | Netherlands | Human | Urine | 131 | 310 | 131 | |
| E255 | Netherlands | Human | Urine | 131 | 310 | 131 | |
| E256 | Netherlands | Human | Urine | 131 | 310 | 131 | |
| E257 | Germany | Poultry | faeces | 131 | 310 | 131 | |
| E258 | Netherlands | Human | Urine | 131 | 310 | 131 | |
| E259 | Netherlands | Human | Urine | 131 | 310 | 131 | |
| E260 | Netherlands | Poultry | Meat | 131 | 310 | 131 | |
| E261 | Netherlands | Human | Urine | 131 | 310 | 131 | |
| E262 | Netherlands | Human | Urine | 131 | 310 | 131 | |
| E263 | UK | Human | Clinical isolate | 131 | 310 | 131 | |
| E264 | Netherlands | Human | Urine | 131 | 310 | 131 | |
| E265 | Netherlands | Human | Urine | 131 | 310 | 131 | |
| E266 | UK | Human | Blood | 141 | — | — | |
| E267 | UK | Human | Blood | 141 | — | — | |
| E268 | UK | Human | Blood | 141 | — | — | |
| E269 | UK | Human | Blood | 141 | — | — | |
| E270 | UK | Human | Blood | 141 | — | — | |
| E271 | UK | Human | Blood | 141 | — | — | |
| E272 | UK | Human | Blood | 141 | — | — | |
| E273 | UK | Pig | Small intestine | 141 | — | — | |
| E274 | UK | Human | Blood | 144 | — | — | |
| E275 | UK | Human | Blood | 144 | — | — | |
| E276 | UK | Human | Blood | 144 | — | — | |
| E277 | UK | Human | Blood | 144 | — | — | |
| E278 | Netherlands | Poultry | Caecum | 154 | — | — | |
| E279 | UK | Human | Blood | 155 | — | — | |
| E280 | UK | Human | Blood | 155 | — | — | |
| E281 | Netherlands | Human | Urine | 156 | — | — | |
| E282 | Netherlands | Human | Urine | 156 | — | — | |
| E283 | UK | Poultry | Caeca | 156 | — | — | |
| E284 | UK | Human | Blood | 162 | — | — | |
| E285 | Germany | Food | Meat | 162 | — | — | |
| E286 | Netherlands | Human | Urine | 162 | — | — | |
| E287 | Germany | Cattle | Organ | 164 | — | — | |
| E288 | Netherlands | Poultry | Caecum | 165 | — | — | |
| E289 | Germany | Turkey | Meat | 167 | — | — | |
| E290 | Germany | Cattle | Organ | 167 | — | — | |
| E291 | Netherlands | Poultry | Meat | 167 | — | — | |
| E292 | UK | Cattle | Lung | 167 | — | — | |
| E293 | Netherlands | Human | Urine | 167 | — | — | |
| E294 | UK | Cattle | Faeces | 167 | — | — | |
| E295 | UK | Human | Clinical isolate | 167 | — | — | |
| E296 | Netherlands | Human | Faeces | 189 | — | — | |
| E297 | Netherlands | Poultry | Caecum | 189 | — | — | |
| E298 | Germany | Poultry | faeces | 191 | — | — | |
| E299 | Germany | Cattle | Meat | 195 | — | — | |
| E300 | Netherlands | Poultry | Caecum | 206 | — | — | |
| E301 | Netherlands | Poultry | Faeces | 212 | — | — | |
| E302 | Netherlands | Cattle | Faeces | 218 | — | — | |
| E303 | Netherlands | Pig | Faeces | 218 | — | — | |
| E304 | Netherlands | Poultry | Caecum | 224 | — | — | |
| E305 | Netherlands | Poultry | Caecum | 224 | — | — | |
| E306 | Netherlands | Human | Urine | 224 | — | — | |
| E307 | Netherlands | Pig | Faeces | 278 | — | — | |
| E308 | Netherlands | Poultry | Faeces | 302 | — | — | |
| E309 | Germany | Poultry | Faeces | 345 | — | — | |
| E310 | UK | Human | Clinical isolate | 354 | — | — | |
| E311 | Netherlands | Human | Urine | 354 | — | — | |
| E312 | UK | Human | Blood | 355 | — | — | |
| E313 | UK | Human | Blood | 357 | — | — | |
| E314 | Netherlands | Poultry | Faeces | 366 | — | — | |
| E315 | Netherlands | Cattle | Faeces | 366 | — | — | |
| E316 | UK | Human | Blood | 372 | — | — | |
| E317 | UK | Human | Blood | 372 | — | — | |
| E318 | UK | Human | Clinical isolate | 372 | — | — | |
| E319 | UK | Human | Blood | 375 | 490 | 73 | 73-SLV |
| E320 | Netherlands | Poultry | Meat | 388 | — | — | |
| E321 | UK | Cattle | Faeces | 392 | — | — | |
| E322 | UK | Human | Blood | 393 | — | — | |
| E323 | UK | Cattle | Faeces | 394 | — | — | |
| E324 | UK | Human | Blood | 404 | — | — | |
| E325 | UK | Human | Blood | 405 | — | — | |
| E326 | UK | Human | Blood | 405 | — | — | |
| E327 | UK | Human | Blood | 405 | — | — | |
| E328 | Netherlands | Human | Urine | 405 | — | — | |
| E329 | UK | Human | Clinical isolate | 405 | — | — | |
| E330 | UK | Human | Clinical isolate | 405 | — | — | |
| E331 | UK | Human | Clinical isolate | 405 | — | — | |
| E332 | UK | Human | Clinical isolate | 405 | — | — | |
| E333 | UK | Human | Clinical isolate | 405 | — | — | |
| E334 | UK | Human | Clinical isolate | 405 | — | — | |
| E335 | UK | Human | Clinical isolate | 405 | — | — | |
| E336 | UK | Human | Clinical isolate | 405 | — | — | |
| E337 | UK | Human | Clinical isolate | 405 | — | — | |
| E338 | UK | Human | Blood | 410 | — | — | |
| E339 | UK | Human | Blood | 410 | — | — | |
| E340 | Germany | Cattle | Meat | 410 | — | — | |
| E341 | Netherlands | Poultry | Caecum | 410 | — | — | |
| E342 | UK | Human | Clinical isolate | 410 | — | — | |
| E343 | UK | Human | Blood | 415 | — | — | |
| E344 | UK | Human | Blood | 415 | — | — | |
| E345 | UK | Human | Blood | 420 | — | — | |
| E346 | Netherlands | Cattle | Faeces | 420 | — | — | |
| E347 | UK | Human | Blood | 421 | — | — | |
| E348 | Netherlands | Human | Urine | 424 | — | — | |
| E349 | UK | Human | Blood | 428 | — | — | |
| E350 | Germany | Cattle | Organ | 428 | — | — | |
| E351 | Netherlands | Poultry | Caecum | 428 | — | — | |
| E352 | UK | Cattle | Large intestine | 446 | — | — | |
| E353 | Netherlands | Human | Urine | 453 | — | — | |
| E354 | UK | Human | Blood | 491 | — | — | |
| E355 | Netherlands | Human | Urine | 533 | — | — | |
| E356 | Netherlands | Poultry | Meat | 540 | — | — | |
| E357 | UK | Human | Blood | 544 | — | — | |
| E358 | Netherlands | Human | Urine | 545 | — | — | |
| E359 | UK | Human | Blood | 550 | — | — | |
| E360 | UK | Human | Blood | 550 | — | — | |
| E361 | UK | Human | Blood | 567 | — | — | |
| E362 | UK | Human | Clinical isolate | 617 | — | — | |
| E363 | Netherlands | Human | Urine | 617 | — | — | |
| E364 | UK | Human | Blood | 618 | — | — | |
| E365 | Netherlands | Human | Urine | 636 | — | — | |
| E366 | Netherlands | Pig | Faeces | 641 | — | — | |
| E367 | Germany | Human | Clinical Isolate | 648 | — | — | |
| E368 | Netherlands | Poultry | Caecum | 648 | — | — | |
| E369 | Netherlands | Human | Urine | 648 | — | — | |
| E370 | UK | Human | Clinical isolate | 648 | — | — | |
| E371 | Netherlands | Human | Urine | 648 | — | — | |
| E372 | Netherlands | Poultry | Caecum | 665 | — | — | |
| E373 | Netherlands | Poultry | Meat | 665 | — | — | |
| E374 | Netherlands | Poultry | Meat | 665 | — | — | |
| E375 | Netherlands | Poultry | Meat | 665 | — | — | |
| E376 | Netherlands | Poultry | Meat | 665 | — | — | |
| E377 | Netherlands | Poultry | Meat | 665 | — | — | |
| E378 | UK | Human | Blood | 681 | — | — | |
| E379 | Netherlands | Human | Urine | 694 | — | — | |
| E380 | UK | Human | Blood | 724 | — | — | |
| E381 | UK | Cattle | Lymph node | 744 | — | — | |
| E382 | Netherlands | Poultry | Meat | 746 | — | — | |
| E383 | Netherlands | Poultry | Caecum | 752 | — | — | |
| E384 | Netherlands | Poultry | Caecum | 752 | — | — | |
| E385 | Netherlands | Human | Urine | 767 | — | — | |
| E386 | Netherlands | Human | Urine | 767 | — | — | |
| E387 | Netherlands | Poultry | Meat | 770 | — | — | |
| E388 | Germany | Food | Meat | 794 | — | — | |
| E389 | UK | Cattle | Faeces | 925 | — | — | |
| E390 | UK | Human | Blood | 973 | — | — | |
| E391 | Netherlands | Human | Urine | 973 | — | — | |
| E392 | UK | Human | Clinical isolate | 973 | — | — | |
| E393 | UK | Human | Blood | 998 | — | — | |
| E394 | Netherlands | Poultry | Meat | 1011 | — | — | |
| E395 | Netherlands | Poultry | Meat | 1049 | — | — | |
| E396 | Germany | Poultry | Faeces | 1056 | 404 | 127 | Disagreement between methods |
| E397 | UK | Cattle | Gut | 1089 | — | — | |
| E398 | Netherlands | Human | Urine | 1266 | — | — | |
| E399 | Netherlands | Poultry | Meat | 1276 | — | — | |
| E400 | Germany | Human | Clinical Isolate | 1283 | — | — | |
| E401 | UK | Human | Blood | 1323 | — | — | |
| E402 | Germany | Poultry | Faeces | 1324 | — | — | |
| E403 | UK | Human | Blood | 1386 | — | — | |
| E404 | UK | Human | Blood | 1394 | — | — | |
| E405 | UK | Cattle | Faeces | 1421 | — | — | |
| E406 | Netherlands | Human | Urine | 1431 | — | — | |
| E407 | Netherlands | Cattle | Faeces | 1433 | — | — | |
| E408 | Germany | Turkey | Meat | 1480 | — | — | |
| E409 | UK | Cattle | Faeces | 1494 | — | — | |
| E410 | Netherlands | Turkey | Meat | 1564 | — | — | |
| E411 | UK | Human | Blood | 1611 | — | — | |
| E412 | UK | Human | Blood | 1611 | — | — | |
| E413 | UK | Human | Blood | 1618 | 490 | 73 | 73-SLV |
| E414 | Netherlands | Poultry | Caecum | 1683 | — | — | |
| E415 | Germany | Turkey | Meat | 1850 | — | — | |
| E416 | Germany | Poultry | faeces | 1968 | — | — | |
| E417 | UK | Human | Clinical isolate | 2178 | — | — | |
| E418 | UK | Cattle | Small intestine | 2217 | — | — | |
| E419 | UK | Human | Blood | 2531 | — | — | |
| E420 | UK | Human | Blood | NEW | — | — | |
| E421 | UK | Human | Blood | NEW | — | — | |
| E422 | UK | Human | Blood | NEW | — | — | |
| E423 | UK | Human | Blood | NEW | — | — | 59-SLV |
| E424 | UK | Human | Blood | NEW | — | — | 538-SLV |
| E425 | UK | Human | Blood | NEW | — | — | 1193-SLV |
| E426 | UK | Human | Blood | NEW | — | — | |
| E427 | UK | Human | Blood | NEW | 200 | 95 | 95SLV |
| E428 | UK | Human | Blood | NEW | — | — | |
| E429 | UK | Human | Blood | NEW | 490 | 73 | 73-SLV |
| E430 | UK | Human | Blood | NEW | 104 | 69 | 69-SLV |
| E431 | UK | Human | Blood | NEW | 490 | 73 | 73-SLV |
| E432 | UK | Human | Blood | NEW | 310 | 131 | 131-SLV |
| E433 | UK | Human | Blood | NEW | 404 | 127 | 127-SLV |
| E434 | UK | Human | Blood | NEW | 490 | 73 | 73-SLV |
| E435 | UK | Human | Blood | NEW | — | — | 538-SLV |
| E436 | UK | Human | Blood | NEW | 490 | 73 | 73-SLV |
| E437 | UK | Human | Blood | NEW | 310 | 131 | 131-SLV |
| E438 | UK | Human | Blood | NEW | — | — | 830-SLV |
| E439 | UK | Human | Blood | NEW | — | — | |
| E440 | UK | Human | Blood | NEW | — | — | 86-DLV |
| E441 | UK | Human | Blood | NEW | — | — | 2604-SLV |
| E442 | UK | Human | Blood | NEW | — | — | 1394-SLV |
| E443 | UK | Human | Blood | NEW | — | — | 144-SLV |
| E444 | UK | Human | Blood | NEW | — | — | 58-SLV |
| E445 | UK | Human | Blood | NEW | 490 | 73 | 73-SLV |
| E446 | UK | Human | Blood | NEW | — | — | |
| E447 | UK | Human | Blood | NEW | — | — | 2626-SLV |
| E448 | UK | Human | Blood | NEW | — | — | 80-SLV |
| E449 | UK | Human | Blood | NEW | — | — | 18-SLV |
| E450 | UK | Human | Blood | NEW | 310 | 131 | 131-SLV |
| E451 | UK | Human | Blood | NEW | 200 | 95 | 95-SLV |
| E452 | UK | Human | Blood | NEW | 104 | 69 | 69-SLV |
| E453 | UK | Human | Blood | NEW | 404 | 127 | 127-SLV |
| E454 | UK | Human | Blood | NEW | 490 | 73 | 73-SLV |
| E455 | UK | Human | Blood | NEW | 310 | 131 | 131-SLV |
| E456 | UK | Human | Clinical isolate | NEW | 310 | 131 | 131-SLV |
| E457 | Germany | Human | Clinical Isolate | NEW | 104 | 69 | 69-SLV |
| E458 | Germany | Poultry | Meat | NEW | 104 | 69 | 69-SLV |
| E459 | UK | Human | Blood | NEW | — | — | |
| E460 | UK | Human | Blood | NEW | — | — | |
| E461 | UK | Human | Blood | NEW | — | — | |
| E462 | UK | Human | Blood | NEW | — | — | |
| E463 | UK | Human | Blood | NEW | — | — | 88-SLV |
| E464 | UK | Human | Blood | NEW | — | — | |
| E465 | UK | Human | Blood | NEW | — | — | |
| E466 | UK | Human | Blood | NEW | — | — | |
| E467 | UK | Human | Blood | NEW | — | — | |
| E468 | UK | Human | Blood | NEW | — | — | |
| E469 | UK | Human | Blood | NEW | — | — | 638-DLV |
| E470 | Germany | Poultry | Faeces | NEW | — | — | |
| E471 | Netherlands | Cattle | Faeces | NEW | — | — | |
| E472 | Netherlands | Poultry | Faeces | NEW | — | — | |
| E473 | Germany | Poultry | Meat | NEW | — | — | |
| E474 | UK | Human | Clinical isolate | NEW | — | — | |
| E475 | Netherlands | Poultry | Caecum | NEW | — | — | |
| E476 | Netherlands | Poultry | Caecum | NEW | — | — | |
| E477 | Netherlands | Human | Faeces | NEW | — | — | |
| E478 | Netherlands | Poultry | Meat | NEW | — | — | |
| E479 | Netherlands | Poultry | Meat | NEW | — | — | |
| E480 | Netherlands | Poultry | Faeces | NEW | — | — | |
| E481 | Netherlands | Poultry | Faeces | NEW | — | — | |
| E482 | UK | Cattle | Small intestine | NEW | — | — | |
| E483 | Netherlands | Human | Urine | NEW | — | — | |
| E484 | Netherlands | Human | Urine | NEW | — | — | |
| E485 | Germany | Pig | Organ | NEW | — | — | |
| E486 | Germany | Cattle | Faeces | NEW | — | — | |
| E487 | Germany | Cattle | Organ | NEW | — | — | |
| E488 | Germany | Cattle | Faeces | NEW | — | — | |
| E489 | Germany | Poultry | Meat | NEW | — | — | |
| E490 | Germany | Cattle | Faeces | NEW | — | — | |
| E491 | Germany | Human | Clinical Isolate | NEW | — | — | |
| E492 | Germany | Human | Clinical Isolate | NEW | — | — | |
| E493 | Germany | Human | Clinical Isolate | NEW | — | — | |
| E494 | Germany | Poultry | Meat | NEW | — | — | |
| E495 | Germany | Poultry | Meat | NEW | — | — | |
| E496 | Germany | Poultry | faeces | NEW | — | — | |
| E497 | Germany | Poultry | faeces | NEW | — | — | |
| E498 | Germany | Dog/Cat | Urine | NEW | — | — | |
| E499 | Germany | Dog/Cat | Urine | NEW | — | — | |
| E500 | Netherlands | Cattle | Faeces | NEW | — | — | |
| E501 | Netherlands | Poultry | Caecum | NEW | — | — | |
| E502 | Netherlands | Pig | Faeces | NEW | — | — | |
| E503 | Netherlands | Poultry | Caecum | NEW | — | — | |
| E504 | Netherlands | Poultry | Caecum | NEW | — | — | |
| E505 | Netherlands | Poultry | Caecum | NEW | — | — | |
| E506 | Netherlands | Poultry | Caecum | NEW | — | — | |
| E507 | Netherlands | Pig | Faeces | NEW | — | — | |
| E508 | Netherlands | Poultry | Caecum | NEW | — | — | |
| E509 | Netherlands | Poultry | Meat | NEW | — | — | |
| E510 | Netherlands | Poultry | Meat | NEW | — | — | |
| E511 | Netherlands | Poultry | Meat | NEW | — | — | |
| E512 | Netherlands | Poultry | Meat | NEW | — | — | |
| E513 | Netherlands | Poultry | Meat | NEW | — | — | |
| E514 | Netherlands | Poultry | Meat | NEW | — | — | |
| E515 | UK | Cattle | Faeces | NEW | — | — | |
| E516 | UK | Cattle | Faeces | NEW | — | — | |
| E517 | UK | Cattle | Faeces | NEW | — | — | |
| E518 | UK | Cattle | Faeces | NEW | — | — | |
| E519 | UK | Cattle | Udder and milk | NEW | — | — | |
| E520 | UK | Poultry | Caeca | NEW | — | — | |
| E521 | UK | Turkey | Faeces | NEW | — | — | |
| E522 | UK | Turkey | Faeces | NEW | — | — | |
| E523 | UK | Turkey | Faeces | NEW | — | — | |
| E524 | UK | Turkey | Faeces | NEW | — | — | |
| E525 | UK | Turkey | Faeces | NEW | — | — | |
| E526 | UK | Turkey | Faeces | NEW | — | — | |
| E527 | UK | Turkey | Faeces | NEW | — | — | |
| E528 | UK | Human | Clinical isolate | NEW | — | — | |
| E529 | UK | Human | Clinical isolate | NEW | — | — | |
| E530 | Netherlands | Human | Urine | NEW | — | — | |
| E531 | UK | Human | Clinical isolate | NEW | — | — | |
| E532 | UK | Human | Clinical isolate | NEW | — | — | |

TABLE 4
Example primers to identify the ST-lineage specific regions of more than 100 by in length

| Forward | Reverse | Product size (pb) | region | ST lineage |
|---|---|---|---|---|
| TGGTTTTACCATTTTGTCGGA | GGAAATCGTTGATGTTGGCT | 490 | Region-1 | ST73 |
| (SEQ ID NO: 58) | (SEQ ID NO: 59) | | | |
| CCGCATTCAAAAGCATAAGT | AAGATTGGGTAAACCAGCAA | 498 | Region-2 | ST73 |
| (SEQ ID NO: 68) | (SEQ ID NO: 69) | | | |
| ATATCTACGACGCTGTAACG | GTAATCACTACGAGGTTCCC | 508 | Region-1 | ST95 |
| (SEQ ID NO: 70) | (SEQ ID NO: 71) | | | |
| GAGCATCTCTTGTCATCACT | CGTATGGGTAGGGAGAAATG | 201 | Region-2 | ST95 |
| (SEQ ID NO: 72) | (SEQ ID NO: 73) | | | |
| CGGAAGGAAAAGTGAAGGT | CTCAACTTGCCATCTCAACA | 250 | Region-3 | ST95 |
| (SEQ ID NO: 74) | (SEQ ID NO: 75) | | | |
| TAAGTGCGCAGAGCATAAAA | CAGAAAATCTTGTCATGGCG | 450 | Region-4 | ST95 |
| (SEQ ID NO: 76) | (SEQ ID NO: 77) | | | |
| AAAATCATCTCCTGGGAACC | TCATGCGTATGAAGATGAGG | 431 | Region-5 | ST95 |
| (SEQ ID NO: 78) | (SEQ ID NO: 79) | | | |
| CTGAAACTCGGTACTTTGGA | TATTTTCTGATGGGGGTTGG | 495 | Region-6 | ST95 |
| ACTAATCAGGATGGCGAGAC | ATCACGCCCATTAATCCAGT | 200 | Region-7 | ST95 |
| (SEQ ID NO: 64) | (SEQ ID NO: 65) | | | |
| AATCCGCAGGTTATCAAGTT | GGATCATGAGCAGTAATCGT | 457 | Region-8 | ST95 |
| (SEQ ID NO: 80) | (SEQ ID NO: 81) | | | |
| TCTCTGCATTTATGCCTGTT | GAGGTTTCCCTGATGGATTT | 470 | Region-1 | ST127 |
| (SEQ ID NO: 82) | (SEQ ID NO: 83) | | | |
| CTTGCTGCTTTAGCATCTTC | CTCTTGCTTGAAAGGTTTGG | 263 | Region-2 | ST127 |
| (SEQ ID NO: 84) | (SEQ ID NO: 85) | | | |
| CGCATAACAGGATTGTCTGG | GCTATTCTACGGGCATTGTG | 404 | Region-3 | ST127 |
| (SEQ ID NO: 60) | (SEQ ID NO: 61) | | | |
| ATTTTCCTTTGATACGCCCT | CTTGGTATTTCACAATCGGC | 471 | Region-4 | ST127 |
| (SEQ ID NO: 86) | (SEQ ID NO: 87) | | | |
| ATTGTCATAGCCAAAGTGGT | GTGAGGAAGTTATTCTCGCA | 302 | Region-6 | ST127 |
| (SEQ ID NO: 88) | (SEQ ID NO: 89) | | | |
| AGCATGGAGCGAAGAATAC | TGAAAGGTTCTCAGTTGTCT | | Region-7 | ST127 |
| (SEQ ID NO: 90) | (SEQ ID NO: 91) | | | |
| ATCTGGAGGCAACAAGCATA | AGAGAAAGGGCGTTCAGAAT | 104 | Region-1 | ST69 |
| (SEQ ID NO: 66) | (SEQ ID NO: 67) | | | |
| ACATCTCTGACTACCATCCA | CTTTTCCTGTTGGCAGTTTT | 387 | Region-2 | ST69 |
| (SEQ ID NO: 92) | (SEQ ID NO: 93) | | | |
| GATATGCGTATTGGTCAGGT | GTAAGGAGCATGTAGGGAAG | 155 | Region-1 | ST131 |
| (SEQ ID NO: 94) | (SEQ ID NO: 95) | | | |
| GGTATATCCTGGGCTGATTG | CGGAAGCTGAGTTAATGGTA | 200 | Region-2 | ST131 |
| (SEQ ID NO: 96) | (SEQ ID NO: 97) | | | |
| CAGGTGTAGATTCCAGTAGC | AACACAAAATGTCACAGCAG | 554 | Region-3 | ST131 |
| (SEQ ID NO: 98) | (SEQ ID NO: 99) | | | |
| GCATTTCGATGGCATATAAGG | TCACTTTACCAGTTCGGATG | 102 | Region-4 | ST131 |
| (SEQ ID NO: 100) | (SEQ ID NO: 101) | | | |
| TAAATGAAGCGGGAAATCCA | GCATGTTAACCACGCAATAA | 482 | Region-5 | ST131 |
| (SEQ ID NO: 102) | (SEQ ID NO: 103) | | | |
| GCTCGTCATTAAAAACAGGG | ATATAAACAAACCGGGCACT | 593 | Region-6 | ST131 |
| (SEQ ID NO: 104) | (SEQ ID NO: 105) | | | |
| TGCCATTCCACCGTTATTTA | TTCACTTACGGGGTTAACAG | 550 | Region-7 | ST131 |
| (SEQ ID NO: 106) | (SEQ ID NO: 107) | | | |
| CAATAATCCGGTAGAGTGGG | TTATTTACTACGTCCGGCAG | 507 | Region-9 | ST131 |
| (SEQ ID NO: 108) | (SEQ ID NO: 109) | | | |
| TGTGAAAGAATAACGCTGGA | GCCATCTGGAGAAAGGTTTA | 349 | Region-10 | ST131 |
| (SEQ ID NO: 110) | (SEQ ID NO: 111) | | | |
| GACTGCATTTCGTCGCCATA | CCGGCGGCATCATAATGAAA | 310 | Region_11 | ST131 |
| (SEQ ID NO: 62) | (SEQ ID NO: 63) | | | |
| GGGCATCTTCCCAGATTTTA | ATCGAATACACGCCAGATAC | 259 | Region_12 | ST131 |
| (SEQ ID NO: 112) | (SEQ ID NO: 113) | | | |

TABLE 5
Example primers and probes for identifying ST-specific regions in real-time PCR or
hybridization assays

| Region | Primers | Probes | Product (bp) |
|---|---|---|---|
| ST73-Region-1 | Forward: TCG CATTCCATTTCCCATGA | TCCGATGTAACCTGCAACTACGCG | 84 |
| | (SEQ ID NO: 50) | (SEQ ID NO: 44) | |
| | Reverse: CGGCGTTGCATACCATTTAAG | | |
| | (SEQ ID NO: 51) | | |
| ST73-Region-2 | Forward: CGTCACTGCCACCGTAATAA | TTCTTGTTGGCTGCGTCTTCTCGT | 107 |
| | (SEQ ID NO: 114) | (SEQ ID NO: 116) | |
| | Reverse: GAACTCTTGAAGTCAGGGTTGA | | |
| | (SEQ ID NO: 115) | | |
| ST95-Region-1 | Forward: GATATTGCTGCGTTGCCTTTC | TCAGTGCAAGCTGGCATAGCACTA | 134 |
| | (SEQ ID NO: 52) | (SEQ ID NO: 45) | |
| | Reverse: GTAGCTTCATAGCGGTCGATTAC | | |
| | (SEQ ID NO: 53) | | |
| ST95-Region-2 | Forward: GCTCCGCTTTATCCGTTCTT | TTACCGGCTGCTGCACACATAGAT | 119 |
| | (SEQ ID NO: 117) | (SEQ ID NO: 119) | |
| | Reverse: TGTCATTGGGTATGGGAGTATTG | | |
| | (SEQ ID NO: 118) | | |
| ST95-Region-3 | Forward: GTCATCTTTGCCAGGTCAAATC | CAACTGCCACCACGCCCAATTAAC | 119 |
| | (SEQ ID NO: 120) | (SEQ ID NO: 122) | |
| | Reverse: TGCCATCTCAACAGGTTCAA | | |
| | (SEQ ID NO: 121) | | |
| ST95-Region-4 | Forward: GTCATTGATGAAGCTCCTGTATTG | AAGTTTGCTGCCATTGACCCAACC | 131 |
| | (SEQ ID NO: 123) | (SEQ ID NO: 125) | |
| | Reverse: TGTGGCTCGGATTTGTATTCT | | |
| | (SEQ ID NO: 124) | | |
| ST95-Region-5 | Forward: GAACCAGCGACCATTGTAGTA | AGCTTCTCACATCGAGGACTACTGTCA | 106 |
| | (SEQ ID NO: 125) | (SEQ ID NO: 127) | |
| | Reverse: CTAACAGGAAGGGCTCGTTATAG | | |
| | (SEQ ID NO: 126) | | |
| ST95-Region-6 | Forward: TCCACTGGATACGCCTTATTTG | AGGTGGACTTAGTGGGTGAACTTGC | 92 |
| | (SEQ ID NO: 128) | (SEQ ID NO: 130) | |
| | Reverse: GGGTGTATTTCGCTTTCATTCG | | |
| | (SEQ ID NO: 129) | | |
| ST95-Region-8 | Forward: TCGAGAGTCAGGCAAGAAATG | ACATCAGGCTTGTCGGGTATGAGC | 142 |
| | (SEQ ID NO: 131) | (SEQ ID NO: 133) | |
| | Reverse: GCAGTAATCGTCATTCCCATAGA | | |
| | (SEQ ID NO: 132) | | |
| ST95-Region-9 | # Short region 40 bp | CATCCACTCGATCTTCGTCTACTT | ND |
| | | (SEQ ID NO: 134) | |
| ST127-Region-1 | Forward: ACCAGCATTCTCAATCTCTTCC | ACCAAGGTTCCGCTCTTGATCGAA | |
| | (SEQ ID NO: 54) | (SEQ ID NO: 46) | 116 |
| | Reverse: GGACTTACTCTGTCCCAATTCC | | |
| | (SEQ ID NO: 55) | | |
| ST127-Region-2 | Forward: TG CCTTATCAGAAACTCCATCTT | TCTCTTGGAAGGCTTGCACACACA | 97 |
| | (SEQ ID NO: 135) | (SEQ ID NO: 137) | |
| | Reverse: CTTGCTTGAAAGGTTTGGTGAG | | |
| | (SEQ ID NO: 136) | | |
| ST127-Region-3 | Forward: CTCGGTTGTTTGCGGTTTATC | ACCTACGGCTGATATCTTACCTGCCA | 112 |
| | (SEQ ID NO: 138) | (SEQ ID NO: 140) | |
| | Reverse: CCCATCAGCACTACACTCTTT | | |
| | (SEQ ID NO: 139) | | |
| ST127-Region-4 | Forward: CCCTGGCTGAAATATCTGGAATA | AGACCCAACATAATCACATAGCCCGC | 131 |
| | (SEQ ID NO: 141) | (SEQ ID NO: 143) | |
| | Reverse: AAAGGAGAGGTTGTAGGTTGTATC | | |
| | (SEQ ID NO: 142) | | |
| ST127-Region-5 | # Short region 47 bp | TGATGATCACCATCAGTCCGGGT | ND |
| | | (SEQ ID NO: 143) | |
| ST127-Region-6 | Forward: ATTGTCATAGCCAAAGTGGTTT | CCCGCATCATTAAATCCCAGTTCTTGTG | 88 |
| | (SEQ ID NO: 88) | (SEQ ID NO: 145) | |
| | Reverse: CCCTCATATTCGGTTATTGTATTCTC | | |
| | (SEQ ID NO: 144) | | |
| ST127-Region-7 | Forward: CCGAAGATATGTAAGTGTGAAACC | TGCCAGCACTGCTTGTTATACCAA | 76 |
| | (SEQ ID NO: 146) | (SEQ ID NO: 148) | |
| | Reverse: GAAAGGTTCTCAGTTGTCTTGC (SEQ | | |
| | ID NO: 147) | | |
| ST69-Region-1 | Forward: TCTGGAGGCAACAAGCATAAA | TTACGACCCAAAGCGAGGCATTCT | 103 |
| | (SEQ ID NO: 48) | (SEQ ID NO: 149) | |
| | Reverse: AGAGAAAGGGCGTTCAGAATC | | |
| | (SEQ ID NO: 49) | | |
| ST69-Region-2 | Forward: GCATTTCCACTAACTCACCAAA | CAAGGGAAATCACCGACATCATCGGA | 119 |
| | (SEQ ID NO: 150) | (SEQ ID NO: 152) | |
| | Reverse: TGACCGCAACAGCCTAAA | | |
| | (SEQ ID NO: 151) | | |
| ST131-Region-2 | Forward: ACCCATTCCATCGCAAGAC | AACTGTTGTAGTGGGCCTGTTCCA | 107 |
| | (SEQ ID NO: 56) | (SEQ ID NO: 47) | |
| | Reverse: GCGTCAATGTCCGGGATTAT | | |
| | (SEQ ID NO: 57) | | |
| ST131-Region-3 | Forward: TTCACGTGGCGTCTGAATAG | AACTTCGCCACCACGTTACCGAAA | 115 |
| | (SEQ ID NO: 153) | (SEQ ID NO: 155) | |
| | Reverse: GTTCGTTATTGCGTTGGATGAG | | |
| | (SEQ ID NO: 154) | | |
| ST131-Region-4 | Forward: GCATTTCGATGGCATATAAGGAG | ACGCGCTACATATTACGGAAACCAATTCT | 102 |
| | (SEQ ID NO: 156) | (SEQ ID NO: 158) | |
| | Reverse: TCACTTTACCAGTTCGGATGT | | |
| | (SEQ ID NO: 157) | | |
| ST131-Region-5 | Forward: TCAGCTGGAGTCAGCAATATG (SEQ | AGGGAGCTGTTTGAACTCTGGCAA | 140 |
| | ID NO: 159) | (SEQ ID NO: 161) | |
| | Reverse: CCAGAGAACGGTAATGGTGTAG | | |
| | (SEQ ID NO: 160) | | |
| ST131-Region-6 | Forward: GGGTCCGGTGAAAGACTTATATC | TATTTATACGTCTGCCCGCCGCA | 136 |
| | (SEQ ID NO: 162) | (SEQ ID NO: 164) | |
| | Reverse: ATGATGGCCTGGTGTTACTG | | |
| | (SEQ ID NO: 163) | | |
| ST131-Region-7 | Forward: CGATTCCCTTCCCAATGTAGTT | TCATTCAACCGGGAAACAGCCAGA | 127 |
| | (SEQ ID NO: 165) | (SEQ ID NO: 167) | |
| | Reverse: GCGGATGCTGAACGGTATTA | | |
| | (SEQ ID NO: 166) | | |
| ST131-Region-8 | # Short region 39 bp | AGATAACAGAGGGAAGTTTAACCT | ND |
| | | (SEQ ID NO: 168) | |
| ST131-Region-9 | Forward: GGTAGAGTGGGTTAAAGGGATTG | ATAGCGAAGCCATGAGCGCCTATT | 127 |
| | (SEQ ID NO: 169) | (SEQ ID NO: 171) | |
| | Reverse: TACGGGTTCTCCCGTCATAA | | |
| | (SEQ ID NO: 170) | | |
| ST131-Region- | Forward: GCATGGTCAAGCGTCATATTG | ATACGGATCAGAAAGACCAGCGCC | 105 |
| 10 | (SEQ ID NO: 172) | (SEQ ID NO: 174) | |
| | Reverse: CCTGTCCAGAGCCTACATTTAT | | |
| | (SEQ ID NO: 173) | | |
| ST131-Region- | Forward: ATCCGCCAGAATACCCATTAC | ATCAGAAACAACATCGCAACGCCG | 102 |
| 11 | (SEQ ID NO: 175) | (SEQ ID NO: 177) | |
| | Reverse: TTTCGTCGCCATATCCATCC | | |
| | (SEQ ID NO: 176) | | |
| ST131-Region- | Forward: TCG CTTCTAACAAGCCGTT | ACCACTGACCTGTAATGAGGAAAGACC | 96 |
| 12 | (SEQ ID NO: 178) | (SEQ ID NO: 180) | |
| | Reverse: AATACACGCCAGATACCGATG | | |
| | (SEQ ID NO: 179) | | |

Sequences for the specific ST lineage targets identified in Table 1

ST73 Region-1 (SEQ ID NO: 1)
TCGTAATTCTTTTTAATATCCTTATAAAATAATTGTTTTAAATGGTGGTATTTAAA
CCACCATTTTGTTCTTAATTTTTGTATAAAGACCAGGTACGTTCTAGTATTTTTAA
TGCAGCAGGTAACTGCTCAATCATTTTACCAGAAACTTCATAATCGCTTTCTTTGT
TGTTTAATGGATTAACAGCAACGTAGGAAATCGTTGATGTTGGCTTAATTATTTT
AAAAACTTCCTTTGCAGATTCACCATAGATGAAAGGTAACTTTGAATTAATAGAG
ATTTCTTGTTTATCGACCATAAACCTGGTAATAGCAGAGAAATGAGTCTCCCCAT
GACTTATAACATAGCCTTTCGGAACGTTAATTTCTTTCAGCTCAGGAATATACTG
AACATAAAACTGTAAATCTTTCACATAGCAAAATTTTGCACTACTATCATTATTCT
TCGATTTACGGTTTTCGCATTCCATTTCCCATGAAAATAGTCCGTCTCCGATGTAA
CCTGCAACTACGCGTCCATGACTTAAATGGTATGCAACGCCGTCAACAGTGCCTT
GCTCTTCTGAAGAAAAACTGAATTCTTTATTTTCTTCATTATACGGACTGATAGAA
CAAACTATCTTTCCTTGAGGAAAAATTGGTCTTTTGGTGAATAACATATCCTCCG
ACAAAATGGTAAAACCATCAATGGTGGTAATTGTTTTTTTAATTAAATACTCACT
GCAATTGACTTCCTTTAAGGCTTCTTGTTTTTGAGCCCATGATAATTCTGCAGTTG
TTAATTTACGATAAAATTTTTCAGTGTAACTTTCAGCCGCCGCAAAGTGTGACAG
TGACGATAAAACAATAATTCCCATGAATGTTTTTTTCATGTATATATCCTTATAGG
TATGCTTATCAAATAATTCAATGGAAGTATATCGATAAAAATAAAGCCGAACAT
GTTAATAAACATACTCGGCTTTATTTTCGGAATTACACCCCTATTGTT
ST73 Region-2 (SEQ ID NO: 2)
TTACTTCACCACTTCCATCAGCTTACTGAAGCTATCCACTACGTCATACTTCACGT
TTTCTGGAGCAAACGTCTTGTTAACCGCATCGAAGAACTTACGGGCACATTTAAT
CTTGGTGGCTTCAATAGGCCGTAAATCCAGTGACGACATCGAGCCCTTGGTCTCA
GCAACGAAATACACATGCTTGACCGAGCCTTCTTTGAAGGAAATAGCCCAGTCG
GGGTTGTAATCGCCGACAGGGGTTGGGATCAGAAAACCACGTGGCAACTTGGCA
TATACCACCACATCCTTGCTGGTATCCAACTCTTCGACGAACTTGCGCTCAATTG
CAGAGTCGGTAAGCACGTAATCATAGATGTGATTTTGCAATTTATTCCCAGCCTT
ACTGAGATCCTGTTTGCTCTGACCCGTGGTAAAGATGTCCAAATCAAACTTGTCT
TCTAGCGTGTCATAGGCCAGATGCTCGATGATGACATTGGCCTTTTGTTCGTTGA
CCAAGCGAATGACTTCGGCAATGAAACTTTCCGGGTTGCTCTTGAATTGCGCGAA
GACGTCAGCATGGAGACCTTTTAGAATTTCGGCAATGGTTCGTCTTGTAAGCAAA
GTCCCCTCTGATAACTTACCAATCAAGTCGTACTGCACAGCGGAGTGGATAGAG
GCGGTGTGCGATTCTGTTTCGGTAACTTTCAGCTCGAAGGCGATACCCTTTTTGA
GGTCATCGTAAGTTGCCGTATCAGCCTGCTTGCCACGGTGAATGGTGTATTGTAA
CGGCGTTACTCGTAACCCAGCATTCTTATCATTGAGCGCCTTGATCGCATTGTTCA
CTAGCTCAACATCATCAAACAGTACGCTGTAGGTAGCTTTGCGATTGATTCGCCC
CCACAGCGCTTTGAACTCCTGCTTTTCAAAGTTGGCGTTGGTCTGCAGTCTTTTGG
GCTTGCGTCCATCTTCTACCTGCATGAGCTGGCTTTCGCTGAATACGCTGTTAATC
AGCTGGAATACCTGCTCTGCATAGGGCTGCAGCTCGTCCGGCAATGCCTCTAATG
TGCCGTCTTTCTTAGCTTGGTGGTATTTCTCTGTGATATTGCGCTTCTTATCGACGT
AGCCATTCTGGATCAGATAAAACTCGATGTCCGTGGCTTGGTCGTCCGAGACCGT
AATCGGGCCTTCCTTCGTGTTCAGCATCTTGCCCGTGAAGTAGGCCTTGTCCGCC
ACACGCGGGCGAGCAGACAAGGAGTCGCTAATGTCCTTCTGCAAGGCGGTCACG
AAGTCCTTGTAACTTTCGCTGGCCACAACTGTCAGTACGTTAATGTCATGAACAA
TGGATGGGTGATCCATGCGTTCACCATTTTGATTGACGGACAAACGCAGGCCGCG
CCCGACCTCTTGGCGGCGTGAAATGGTGTTATCACTATGCTTGAGTGTGCAGATA
ACAAACACGTTGGGGTTATCCCAGCCCTCACGTAAGGCCGAGTGTGAGAAGATA
AAACGCACCGGCTCAGCTAGTGATAATAACCGTTCCTTGTCTTTGAGGATTAGAT
CATAAGCATCTACGTCATCCGACAAGCCCGCATTTTCACCGCGCGCAGCGGCTTT
GGGATCTGTGAGGCGCTTGCTCTTCTTGTCGATGGAAAAGTAGCCGCTATGCGTC
TTATCGGCCGCAATGCGGCGTAGATATTTAGCATAAGGGGCATCCCCCTCCATAA
ACCCATTATCAAACACTTCTTGAATGTGCTTAGCGTACTCCTCTTCAAATATTTTT
GCGTACTCTCCCTTTTCATCGACAGCGGAGTAGTCCCGGTACCTGGCCACTTCGT
CAATAAAGAACAACGAGAGAACCTTCACCCCTTTATCGAAGAGCTCCATCTCCTT
ATCAAAGTGCGCCTTGATCGTCTCACGGATCTGAATGCGGCGCAGCGCTATCTCG
GAAATATCACCAATCACATCACCCACCGTCAGTTCTACGCCATTGGTAAAGCTCA
AGGTGTCCGTATTGGCATTGATGTCCGATACCACAAACCCATCGCGGTATTGATC
CAGCTCGTTCGATAGCACAAACAGATTGTCACCCTTACCCAGTTTGCGAGTGACG
CGCTTGATGTTTCCACCGCTGAGTTTCTGTTCGAACTCTACAACCGCTACTGGCG
GTTTTTTGTTTGAGATTTCGATGGATTGTAAATAGAGATAGGCATTGGTTCCCGCC
AGTCCCTTCACCGAGATACCACGCACCTCAATCTTCTTCACCAGCTTCTGGTTGTA
GGCATCGAGCGCATCTAGGCGGTGAATTTTGTTGTGTGTGGTCTTGTGTGTAGCG
GAATAGCGCAACACCATCAACGGGTTGAAATTAGCCAAGGAATCTAGGGTCTTG
CCCCCCTCCATCCGCTGCGGCTCATCGAGGATCAAGATGGGTCGGTTTGCGCTGA
TCACATCGATCGGGCGACGTGACTGAAAATCATCGAGCGCTTCATAGATACGGC
GGTTATCTTTGCCAGTGGCGTTAAACGCTTGTACGTTGATCACCATGACATTGAT
ACCGGCATCTGAGGAGAAACTCTCCAGATGGTGCAGCTGCTTAGAGTTGTAAAT
AAAGAAGCGTGCCTTCTTGTGATAGGTCTCCTGAAAGTGCTCGGCCGTGATTTCC
AACGACTTGGCTACGCCCTCGCGGATGGCAATGCTCGGCACCACTATGACGAAC
TTGCTCCAGCCGTACTGTTTGTTCAGTTCGAAGACTGTCTTGATGTAGCAATAGGT
CTTGCCAGTACCCGTTTCCATCTCGATATCAAGATTTAGCTTGGCGACCTTGGTCT
TGACCAAGCTCTCCGACAACGGCAGATTTTGCTGGCGCTGCACCTGCTGGATGTT
ATCAAGCAATGCAACCTCACTAAGGGTTAGCTCAGCATTTTTAAAACCAGTATCT
ACCGCAAACAGATCACCCATACCGTTTTTGGTATTACCAGGGTCAAGGCGATAGC
TCATGGCCGCATCAGAGGCCGGTGGCTGCCCCTGAAAAATATCGACGACCGCCT
GCACTGCGGCAGCCTGATAGGGTTGATGTTTGAATTTAAGCTTCATTGCACCGTC
CCCCTCAGATACATTTTACTTCGGTGACAGGCGACATGAGCTTGAAAATCTGCTC
GACATTGATCTTGACCGCACTGTTCTTAAAGCCCGCGTCGCGGAACACAACGCGC
AGCGGCTGCAGTTTGGCCAGTTCCTTCACGAAGGTTTCGTCAATGCTGCCACTGG
CGTCGAAGCAGGCGGTAAGCACATTGCCATCGACAAAGAACACGTCTTTACCTT
GGATGGACTGCTTGGCGATAGGCAGGGCCAGATCGACGCCCCAATCCAGCATTA
CTTGGAACAGCAGATCCTCAGGCGTGCGATCAGGCTTGATGTTGTCAACAAACA
AGTCGAGATTGGCTTTTTCCAGCACATCGGGGCTGTAGTAGACGTCAGCCATGTT
CGATGTATCGACTTTAAGCAGGCGAAATCCTAAGTCAAGTTCCGTATTTCTTGTA
GCCAATATTTCTGAAAATTCTTCTCTAATTTTTTTTGCAGCTCTCCGAATGCGATT
TTTTCCAATCTCCGAAACTACTGAGTATCCAAGTTTTTTGGCTTCAGACTTCTCGT
CACATTCCTCGGGCAATTGAACCATTATGAATCGACGATTTGCCCCATCTTCCGC
ATTCAAAAGCATAAGTGCATGCGCCGCCGTGCACGAGCCAGCGAAGAAGTCGAT
CAATATATCGTCATTCTTGCAAATTGATTGAATTAGTCTCTTTGCTACCTGAACCG
GTTTCGGTGTATCAAAAGGAATTTTAAGGTCAGCAAGCAAAGCGTCGTCACTGCC
ACCGTAATAAAGTAGAGAAGGGATATTTTCGAATAAATTGTCATTCAGCCGATAT
TTTCTTGTTGGCTGCGTCTTCTCGTCTGGACCGAAAATAATCAACCCTGACTTCAA
GAGTTCTTGCATTGTTGCAGGAGGGTTTCGCCACCCACGCTCAGGAACAGGGCAC
TCCTTCCCTGTTACAGGATGAATTAGCGGAATGAAATAATCTTCCGGTGCTTTTTT
CTTATTCGGCCACGCCATAGAAACCGGACGGAAGACGTCGCCATTGTCATCGATT
TGATTATATGCCTTTTCCCCCCCAGTTAGATCTTGCTGGTTTACCCAATCTTTGTA
TTCTTTCCTTGCTTTTTCAGTTACCCCGCCCTCCTTGCTAAGAATCTGCTTGGCTTT
TGCAAGCATTTTTCCTGCATTCTCTTTTGGTCGCTGGAACTCGCAGGTTGTCTTGA
AGAATTCTCGATCTTTGCAGTAAATACAAATTAGCTCATGCTGCTGCGCGACACC
TGTGGCATCACCTTTTGGATTTCTCTTATCCCACACAATAGTTCCGAGATTATTCT
TCTCACCATATATTTCTGCCAGAAGCTTCTCCAGGTTGGGATATTCATTCTCATCA
ATATGAATTACGATTAATCCATCATCTCGTAGCAAGTTGCGCGATAACTTTAGTC
TCGAATACATCATGGACAGCCAGTCTGAGTGAAAGCGCCCGCTTGTTTCGGGGTT
GGCTGTTAAGCGGTTTCCTTCTCTATCCACCTGATTTGAACGAAGAAAAAACTCA
GAGACCTCGTCTACAAAATCGTCGGCGTAAACAAAATCGTTTCCCGTGTTGTACG
GCGGGTCGATATAGACCATCTTGATTTTGCCAAGATAATTTTCCTGAAGCAGCTT
CAGCGCGTCGAGATTATCTCCCTCGATAAAGATATTCTTGGTAGTGTCGAAGTTG
ACGCTCTCTTCAATGTGTTCGCCTTTTGAATTTTTTGTCGTACGTACTGGACGGAG
CGTCTTTGCAATCGGTGCATTTGCAGTGAGCAGTGCCTCGCGCTTACCAGGCCAA
TTCAGCTGGTAGCGTTCCTGCGGCCCTTCTACAATCGAATCAGACAGTTCCTGGC
GCAATTGGTCAAAATCCACTGCCAGCTTCACGCTACCATCCTCGCCCTTAGCTTC
GGTGACGCAGCCTGGAAACAGATCGCGAATGCGAGCGATGTTGTCCTGCGTGAG
ATTGGGTGAATGCATTTTTAGCTTTTCCATATTCTTTTCCTCTGGCCGTTATGCTGC
TTATTGAAAAGGCATAGCGGCTCACAATTCAAGTTCGTATTGGTATGGTTAGTTG
CCTTGCTGCCAGTCCTGGCAAAAGTGCAGGCGCAGGCTCTCGAGTGCAGATGGC
AAGCTGCCCTAGCAGGTCACGTCCAGGTAGGTTGCCGCACGGGTGATGCCGACG
TACAGGTATTTATCGAACAGCGCCGGGTGCAGAGTGGCGAGCTGGTCAATGCTG
ACAAAGAACACCGCCTCGAACTCCATGCCTTTGATGTGCTGGATGTCGAACACGC
GCACGTTACTTTCTCTTCTCCCTCTTCATCGTCTACCACAACACGAGCAATGGCAG
GCTCCGCCACCAGTACCTGATGGTTTTCGCGGTTCGCCTGACTGATCTTACCCAG
CCTTTGGATGGCTGAACTGGACGTCATTGCGTTGATGCTTGCCAGCGCATCGGAA
CCAAGTGTCCGCCCATCGTACAGCTTGCCCCTTTCGGTTTCAGCGATGCTTTCGA
GTTGCGCCAATGCTTCACCAGCGATCTCCTCGATATGTTTCCTTGACTCGTCATTT
AGGCTCTGCGTCATGCATTCTCTCTCCCTGTCAATTCTTCAAGTTCGTTTTTAAGC
TGTCGCAATTGCGCGTTGATCTCTACTTTGCGGTTAAATTGTTTTTCTTTGGCAAG
TCTACTGGCAGCTTTCTCAACCTCACGCTGCTTGGCTGCAACCATTTCGACACGT
GCGACCAAGTCCGCGAGACTTTCTTGTGGGCGCGCGGGCATGGGGATCAGGCGG
TGAAGCACCTGCTCATACAAGCCGCCTAGATCGAGTGCTAAGGGTATTGCGGCA
CGCTCACTGTCACTCGGCAACCAGGCTGTCGCAAAGTAGCTACTCAAGACCCAA
CGACTGGCGTCTGACTCATTGGGGCGTTTGTATGCAGCGATCACCTTTGTTCGGC
CATCAAAGCTCAGCTCGAATATGATGGGAAACTGCACTGCGCCATCGATACAAT
GTAAAACGTCCAGATTCAGCTCCGATGTCTTGAGCTGGATCGAGAAAATCTGAA
GCTCTGGTACTCCCGGCCTAGCGGGCAGGTTAATCGTTTCTGGGGCCAACTTGTA
TCGCCAGACGATCAGCTCCACCTGCTCGATGAACAAGTCCTTCAGCCTTGTATTG
GCGCCGCTATGTTCGTAGATCTTATTCTTTGGCAGGGTACGACCAAAGGCGGCCT
GCTTCGGGTAGTTTATCAACGCGATATTCGACTCAAATTGGATAGGCTGACTCAT
CCGGCCTCCTGAATCACTAGGAAGGTGATCAATTCGAAGTCGTCTAGCCCGACG
ATAGTATTGACCAGCGCGGTAGTCTTACCGGCGAAGAAGAGGCTGTCCAGATCC
TTCTCTTCCTTCACTTCGATCATTGAGCGGATAGCTTTGCCAAGCAGATCTGAATA
CACTTGCATCTTGCTGCCATCGGCCGTTTCCTTGTTGAACAAGAGACAGGCGTCT
GTAATAGGCTGGGTTTGCCCCTTGCAGCAGCTACGTACCAGATCCAGCAAGCGTT
TGACCTCCGTGTGATCGTGAATGACTTCACCCTCGCGATTAATGTAGACGAGGTA
ATAAGGGTGTAGCCGGTTGTGCTGACTGACGTTGACGCTTGGATTCCGGTTGCGC
AGCGTGAAGATCACACCCGGGCGCAGGCCCATCTCCGGCTTGGCAGACACTACG
GCGTGCATTCCGTTTGGCACGTTGCTCAATTCGCCATTAGCCTTCACATAGTTGA
GCAAATCCATTCGGAAGTCATTGAGCCCGAGATCAGTGATCGACACTCCGGTTTT
CAGGTCTTCCAGCTCAATCACTTCCTCTTGCAAACGTCGCAATTGCTCTTTGCGAT
AAGACACGTCGTTGGCTTGGGCGCTCAGCACATTGTCGTCACCGGTGGCTGTGAC
ATCAGCAATCATCATCCGGCTCTCAACCCGTTCCTTGAGGTTGATGTATTCGTCG
AGAGAGATGTCTGGCCAGTAGTTAACCAGTTGGATGCTGCTGTTGGGTGAACCG
ATGCGATCCACACGGCCGAAGCGCTGGATGATGCGCACGGGATTCCAGTGGATG
TCGTAGTTGATGAGGTAGTCGCAGTCCTGCAGGTTCTGACCTTCAGAGATACAAT
CGGTGCCGATCAACAAGTCTATCTCTGCCGCCTCATTTGGCAGCACGATGGCTTT
CTCCTTCGAGTGTGGTGAGAAAAGGGTGAGCAGTTCTTGGAAGTCATAGCTCTTC
TTAAGCGTGCTCTTCGGCACCCCTTTGCCAGTGACTTTTGCACTGTGCAAGGTTTG
TGTGGCCAGCAATTCCGGAGCCAAATTGGCATATAGGTAGTCAGCGGTGTCGGC
GAAGGCTGTGAAGATCAGCACCTTCTTGTTTCCAGGATTCAGCGGTGCTGCGACT
TTTTCCTGAACCAGTGCTTTGAGATGCTGTAGTTTGGCATCATCGGCAGCCGTGA
TCTTGTTCATCGACGTCAGTAAGGCATCAATGATTTCCAGATCAATCTTCAGGTC
GTGCTCCCAAGAAGGCAAGTCCATATCTGCGAGGCTGATTTTGACCTTGCCACCG
ATCTCGCTGTCACCGATGCAGGGCAGGTCGTCGTCCGCATCTAGGTTTTCTAACT
GGTCGGTGAGGTCATTGATTCTACTGTCAATGCCACTGAGGTCGCTAGTCTCATT
GAAAGTGCTGATCTTGGCCAGCGTATTCATGTGGTTCGCGCGCAGGGACTTGAGC
GTCAAGCGGAAGGACTCAATCGAACTTTCCAGACGCTTGAGCAGGTTGGTCGTC
ATCAATGCCTGCAGGCTCTTTTCGCGGTCGACTTGTTTAAGCTTGCCTTTCCCTGC
AACCTGAGTGTCATACATCTCTTCGTACTTCTTGAGTCGACTGGGCAGGATGTAG
CTGATTGGGGCGTAAACAGCGAGCTTGAGTAAAGAGAGCCGCTCGAAAATTTCG
TTAAAACTCATCACATCCGTTCGCTGCGTGAGCGAGCAGTGGAACGACAAGGGT
TTGCGGCGCTCAGGGAATTGGCCGATTTCCTTGGTATCGTAGAAGGTCTGAATAT
GCTTGCGCGAACGTGCGATGGTGACGCTGTCGAGCAATTCGAAGAAGTCGAAGT
CCAACGAATCTAGGATCGCCCGCGCTGTGCGATCTTCCGATGGAAGCTTTGCCCA
TGCATTGAAGCTAGCCTGTGCGCCACGGAAAATGTCCTCTACCGTCTTGCCAGTA
CGCAGCTTCTTGCTGAGGTTTTCGGAATCGCCTTCGTAGGCCAACGCCAGTTGGT
TTCGCAGGTCGTTGAAGCGGTTGTTCACCGGGGTGGCCGAAAGCATCAGCACCTT
GGTTTTCACGCCCTCCTTGATAACCTTGTTCATCAGCTTTTGGTAGCGGGTTTCCT
TGTCCTTATAAGCATCATTGTTGCGGAAGTTGTGCGACTCGTCGATGATAACAAG
GTCATAGTTACCCCAGTTGATGCGATTCAGCGGTGTGCCGAACGACTCACCGCTC
GTACGGCTGAGGTCGGTATGACAAAGCACGTCGTAGTTGAACCGGTCGCGAGCA
AATATGTTGGTCTTGAGGTTACGGCTGTAGTTCAGCCAGTTGTCCGCCAGCTTCTT
GGGGCATAACACCAGAACTGACTTGTTGCGCAGTTCGTAATACTTTATCACGGCC
AGCGCGGTGAAAGTCTTACCCAAGCCCACGCTGTCGGCCAAGATGCAACCGTTG
TAGCTCTCAAGTTTGTTGATGATCCCGGTGGCAGCATCTTTCTGGTAATTGAAGA
GTTTGTTCCAGATGAGTGTGTCTTGATAGCCCGTGCGGTCGTTCGGCAGAGCATC
CTCGTTAATGTCGTCCAGAAACTCATTGAAGATATTGTAGAGCATCAGGAAGTAG
ATGCTCTCGGGCGAGTTTTCTTGGTAGACGGACGTAATGTGGTCGCAGATCTGTG
CCGTTACATCCTCCAGCTTCTCGGGGTCTTGCCAGATCTGGTTAAACAGACTGAG
GTAGGTCGCCGTAAACGTCGACTCGTCCATTTTGTTGACGAGGTTTGAGACAGCA
TTGCCCTGCTGGTAGCCAAGATCGACGGCGGTAAATCCGTGCAGTGGCATGTAG
GCAGTCTCTGTGGCGGCGGCCTGAACACAAGCAAACTGCTGCATCGGAGCCTTG
CTGCGATTGGACTTGAATGTTGCCTTGCGCCGCATCCAGTCCGCGCACTCTTTTGC
AATTGCGCGCTGGGTCAGTTTGTTACGCAGCTGAATTTCGAACTCACTCCCGTAC
AAGCTCCGTTCGCGGTCAGCCTTCAGTATGTGGAATTCTTTGCGTTCCTTGCGAAT
CTTGTCGGTAACCTCGTTAGCGGTAAACGTCGGCGAGGTAAAGATGAATTGCAG
CTCGTCGATTTTTTCCAGCTCTGCTTTGAGCGCTTCGAACGCGTATATCGAAAAG
CAGGAGGCAGCGATCTTCAGGCGAGCACTAGGTTTGAGCGTCTGCTTTAGGTCGT
CTCCGAGCAGGCGGTTGATATTGTCGATCAGTTCCATTAATAGTCAGCCTTCTTA
GTGGCTCTATAGCCTGGTGCCAGTAAGGCATTTTTTACGCCGTAAATTGCGAGAT
GATCTTTGAGCCATAACCGGTACTCCGGTCCACGAAGGCAATGATCAGGTGAGC
AGTCTACCCGCCAGCGCTGCAACATATAGCCCACCACCGCAGCGCGAACGCGCA
TTCTGATCGAACCATCTGTCATCCCGTAGTCCATCTTGATAATATCGGGTCGCTCA
ACGCTCGGGTGCGGCACGAAATCCAGCTCGACGATGCGCGTCCACTGGATATCA
TTATGCGGTCGTTCGTTGGCCTGTGGCTCCTCGTCGAGCAATGTTGGTTCTTCGAT
GCGGGTGACGACGAAATCCCGAAACTCTCTACTCTTGCGATCAAAGGCCCGTAC
GTGCCAGCGCAGGCCGGTATCAACCAAGGCAAAGGGCACAATTACCCGCTTGGA
CTCGCCGCTGCTCATCGAGTGGTAGCGGATGGCGACGGGTCGCTTAGCGTGGATT
CCTCGGCAAATCGTGGCTAACACATCCATTTTGGGAATGCTTAGGGCTGCGGGGG
ATTCGCATGGTAGCAGTGCCTGCATTGAGCCATTCACTCCATCGCCAAAACCGAG
AGACAGCGCCGACAGAATGCGCAGCGATGCGTGCTCGAATAACGGGGAGAACTC
CTGACCGATGCGATAGATCTTGTTGCTACCATCGAAGGTGATGTTATGCGGCACA
ATTTCCCGGTATAAAGCCAAATCGCGAGTCGCCCCTGCCGGAGCCACACCGAAG
CGCTCAATCAGATCTGGGCGACCGATCTCACCGAAGAAGTAGAGCCGAAAATCG
ATGTAGGCCAGCCGATCGCGCTGGACCAGACTCAAGTCTTCAAGCCGCTTAGGA
GGGGAACTAGACATGAATAATCACTATATAGGAAAATGAAAATTGAATACGGTA
ATCCATAAAGAGCCATGGTTGTAATCAAATTGATGACAATCATGCATTAATACTT
ATGTAACAGCAAGAAATGATGTCTTAAACGTAGACAATAATCGTATTGGCCGTGT
TTCTATGATTGTAAAAGC
ST95 Region-1 (SEQ ID NO: 3)
GTGGTTGTCCGCTTTGTGCCAATAGCGGACATTGCTGGCTCATGGTATGTTAGTTT
AAGGAGGCAGGTTATGCTCTGTATTTCGCTGTGGTATAATAGTTGCGCAATTGAT
ACAAGGGAGCGTTACATCGTGAAAATTGTTTTCGAGGTTAATGGAAAGAAAGTT
CCACTAAAATCTTTAAAATACATAAGCAAAAAAGATCAACTCGAAGTAATGAAA
AACTGGTTCTTTGAAAACTTTGAAGATCCGGCAAATGCTTGTCCGTATGAAAGTA
GAGAAGGTGGCTATGCTTACATATATGGCGGGCCGTATGATGCGAGCGAAGAAC
TACAATCCATCTTCGGTCAATATGTTAAATCTGAGTATATTGAGGAATTAGTAGA
TGAATTACAAACACAATGTTTTGACTGGTCTGGAAACTCAAATAATATAGATGAT
TGGTACGATGATGATATCTACGACGCTGTAACGTCTTCCGGTAATCCGTATTTAA
AATTCATTGATAATATCGATAAAATAAAAAAACTTGCAAAAGATAAAACTGAAC
AACAGCAAAAAAACCACTTGCTTAGTTTGCTCTACACGAATGTTATTACAGCTTT
GGAAACGCTTTATGTTGAGCTATTCATTAATTCTATAGAGAAAGATGATGTCTAC
ATAGCCAATTGTATAGAAAAAGGTAAGACTGAATTTAAAGTAAGCAAAGATATT
GCTGCGTTGCCTTTCAAGGGTGAACCCATCGAGAAAATTAGGGGAGAGCTAATC
AGGTCAATCAAGGAACACTTGATCAGTGCAAGCTGGCATAGCACTAAAAAGGTA
ATCGACCGCTATGAAGCTACATTCGATATAAAAGTACAAAAAGACTGTCCGATA
GAAGCTATTGAATTGGCAACCCTCAATCGAAATCACTTAGTGCATCGTGGTGGAA
AGGATAAAGAAGGGAACCTCGTAGTGATTACAGACCAGGATCTTGAAACGCTGA
TTGAAAATGCATCGAACCTGGCAATTATGCTCTACAACAGTTTAAATGTAGCAAC
GAATAAAACCACCATTTTGCAACCAGATGACAAACCGTTCATTCATGAATTTTAA
TAGCGCAGGTCATACATGGATATTCGCACACGAAAGACAAAATTTTTAGAGTCG
TTAGATTCAACTGAAGTGATACGCAAAGCAGTAAGTCTCGCTATAGATTGCATTA
TTGATAATCATAATAGTAATGAAGATACCCCCTTAGTTATTACCAGCTATGATGA
TCTCTGTAGAATCCAGGTGCTAAATTATGTACAGGAGTTTTGCGAAGCTGCATTC
CCTGATATGGATGAATATTACTTTAGTCCTAACATACTTCGCATCAACGGCAAGA
CTAGTGAAGAAGCTTGTATTAACTTAATTAAACTCTTAAGAAGTACAAAAGGCAT
GCTATTCTGGTCCGATGCCCCATCGTGGTTCGCTAGCCTCCCCGACGGATTATTCC
ATGTTGTTAACATTGATCAAAAAATCGTTACTCGCGGCCTTAATAAGAAAAACTC
TAAGCCGACAATAATCAATAAAGATTACAGTGTAGATACACTACTATCAGAGCT
ATTTTTGAATGGCGCACACATGGAGCAACCCAACGTACACAACGTTTCTGAGGG
AAACATGAAGTTCTATGATGAATGCCATGCTGGATTAATCAGACCAATACCCGCT
CCAATAGGCGCGTCATACGATGAAGAGATCACAATTAATTCTCCAGATTGGCAA
AAGCTCGCGTGTGTAGCTTTACGCCGCTATCAGTCGAAGGAATGTCATGACGGA
ATGCAATGGGATACAACCGATCATGGTTGGACTGATGTAATCGCGTATCCATTCG
TCGAAGAAATACAATCAATGGATAATAGTGGTTATCGACAATGTTTAGTTGGGCT
GGTCACAATAAACAATTCAAATGCAAATTCTCCGTACCTATCTACCGTTTGGATA
CATCCATTTTATCGCAGGAGAGGGTTATTAAGTAAGTTATGGCCAAAATTACAAG
AGTTATATGGAAGTAACTTCGAAATTGAGCGACCTAATGAAAATATGAAAGCAT
TTCTGAAAAGTGCGAAACACGCAGATTACTAATTTATGAATCAGTCTGTGGAGTT
AGGGTTGGTGTCAACAAGTAGGGTAAGAGCCTCAACGTGATGCTATGCCGACCT
GCTCCCTGAGAATTCACACAGAAGGCTGCTAGCAA
ST95 Region-2 (SEQ ID NO: 4)
TTATTGCGTGAGCTTCGCATGATAGCTGGAGCATCTCTTGTCATCACTAATTAGCT
CCGCTTTATCCGTTCTTGCCCATATTATATTTCTTTTTATTATTTTTGCTGGATTAC
CGGCTGCTGCACACATAGATGGCACATCTTTAGTTACAATACTCCCATACCCAAT
GACAGATCCGCTTCCAACAGATACTCCTTTCATTATAGAGACATTTCTCCCTACC
CATACGTAACTAGATATAATGATATCTTTTGCCCAATTAATCCTTTTTTTGCTATG
AATATCAAATATAGGATGCCCATCTGATGCACGCAAAATAACATCTCTTGCTATC
AT
ST95 Region-3 (SEQ ID NO: 5)
TAACAGATGTGAGCGTTTGCTATGAGCGATATATAGTCATTCGTACCCTCCGATA
CTAATCATCACAGAAATTAATATATTCATTCCTTACTGAAAAGCTATGAAGTAGA
TATATAGGATTGGGGCGCGTATGGCACTGGACAGTTATGCATCTCCGAATATTAT
TCCGGAGCTAAGACAACCGAATGCCGCAGCAGGTAAGATCACGCTAGGGGGGCT
GGACAGAATGGCTGTTACGCCCAGTGTGAAGAAAAGAAGGCTAAGTATGGTAGA
ATTTCCATACGCACAGACCTTGTTATCCGACTATACAGCCTGGAAGGCACTATGA
CAAATCTACAACTGGAAGAAAGATTTAGGGCGCTTGAGAAAGATTACGATGCGC
TCATTTCCACAAAATATACTGCGCAGAACGTTCTCACTCACACTATGGAAACCTA
TGTTGATTCTGGTAAATATAAAAACTGGATCGCCAGAGTGAAAAAGTTAATAGA
GGACAGCTACGGAAAAGAGTCTGATTATTACAATGACTTCAATACTGTAAACAG
CAGGTGGTCTTCTAATTACAATACACTGATCAAAAGTTATAAACCGTTGTTTGAT
GCGGCGAGAGATGATCTTGCGCACTCTGCTGTCAGTACTAATACTCCTCAAGAAG
GTTCGCCGCTGAGCCTCGTACTTAATATTCTTAACAGGTTTCCGACCTTTGTTCGC
CAGTTAAAGAGGCGGTACAACGGCCGTGCGCCGCTTGAAGTTAATGATGAGTAT
GACGTACAGGATTTGATTTATGCACTGCTGACGCTTCATTTTAATGATATTAGGG
CGGAAGAGTATACCCCCAGCTTTGCGGGCGCGGCCTCAAGACAGGACTTTCTTTT
GAAAAAAGAGAAAATTGTGATAGAGGTGAAGAAAACCCGTGAATCTCTGGGAG
CGGGTAAAGTTGGTGGAGAGTTGCTTATTGATATGGCGCGCTACCGCGCTCACCA
AGACTGCGATACGTTGATCTTGTTTGTATATGACCCGGATTGCTACATAAATAAT
CCTTTGGGCGTGAAAACCGATCTGGAATCTAAAGACGCGGAAGGAAAAGTGAAG
GTTGTTATTGCGCAGTTCTGACACGCTTAAGTGTCACCTTACAGGATTAGGTTTGC
GGCCGTGCCAGCAGGACGAGCATGAAAAAGGCCGCTGGCACGGCCGGATTTGTC
ATCTTTGCCAGGTCAAATCAAAATCTACGTGAAAATCCGCAGGCTTAAGCTCGCA
ACTGCCACCACGCCCAATTAACGACGTTATGTTTACAACGTTTGAACCTGTTGAG
ATGGCAAGTTGAGTGTCATCTGTAAACGTGATGATTAGCATTTCAGTCTGATGAA
CCTGTACTCCAGTTTTCTGAAAACCAATCTGGACAGATTTCACCGTTTTCCCAACC
ATTTCACGGGCTTGATTGATAGAGTAATTAGCTTCAACTTGTAGGTCATTCCTGA
ACTTGGGTAGTGTCATTTTACATCCTCTTTAGTGAAGTTTTCGCTATTTAAATCAG
CAGCATTCCTTTTAAATCACTGGCATGTCAGCAGTGGATAGTCTGAGTGTAACGT
CCAGTGAGGGGAATAAATATTGGTCAGACTTTGCACAAAAGAGCCAACTATCGC
GGGCTCTCTCTCATCATGTGCGAAAATTGACCACAATATGAAATCCAGTTCCATT
CCGAAAT
ST95 Region-4 (SEQ ID NO: 6)
TTTAAAGTGGGATCTAACTCTAATAATTTAAAAAATTTAATTGGGTCTTGATATT
AATCACATAGACTCAATGCTCTTTAGTTCTTACATAGGTGGTTGTATTATGAGTAT
CCCCAGTTTAGATGTTCATAAAGTTGTTAAGTGCGCAGAGCATAAAAAGGCTTTA
GATGCAATATCTCTCAAACTTAAGAATAACATTATTGACTCAAGCTCTGAATACG
ATTTTCTTGGCGACACAATAATTTTCAATAAAGATAAAAAAATAGGTTAACCCTC
AAACTTTCAACATATGAACTAGAACTTATTTTCAAAGAAACACTGCTAGTCATTG
ATGAAGCTCCTGTATTGAAGTTTGCTGCCATTGACCCAACCGATGAAAATAACAA
CGAGCTGATTGATTTATTTTTATCCAAAGGTGGATTTATTTATATTAGAGAATACA
AATCCGAGCCACATTATGAGTATGGCGACACTTCTCTATTTTTACATATACTTGA
CGCTACTCTTAAAGAGCTTAAATCTGCAAACAAAATATCCTATTAAATAAATTTT
TGCTAACATAGCCCCGCGCCATGACAAGATTTTCTGTTCTAGATGCAAGGGCTAT
GTGTGAATATCTATAAACAAAAACCCCATCCGGTGATTTTTGTCATTTTTTAGCCA
TCTCAATTGCGGCATCAAAAAGTCGTTCAAGAATAAAATCAGGGTACTGTAATTT
TTCTTGCTCGATATGAAGCTCATAAATTTTCCCTTCCCACTCCCATTTAAAATTAA
ATCCAGAAGCAGAAGGTGCGAAATGTGTTTTAGCTTTAGGGTTAGCGTAACCAG
CAACAGTCAACACATCAAACTTAAAATCTTGAGGTTTAATACGCATAATCATATC
CTTTGCCAAGTTCATCATTATTCTTCGCGATTATAAAACAATGAATAAAATCATT
AAAGCATAGACGTTAAATATCCTCAAAATGTGATTAGTTGGGACATTGCGTCCTG
ATATACTCCTGCAAGTAATTGACCTGTGAGGTTATCTTGTCGATTCCACTTCGGA
GACGGTAATAATGGAGTTCAGCATCTGCTGTAAGTCTAGGGCTTTCTCCATCGCC
CATGCCGCTGGCTCCGGTCGTTGACTTTGTACAGGTGGCGGCGACTTGCAGGCGC
TTATGCCCAGCAGAAACATCAGCACGAAGACTTTCGATAGTCGCATTAGCATCA
GCAAGCTCCTTTGTATATCTGGCATCGAGTTCAGCTACATTACGTTGACGCTTCTG
CATATCAGCGATGATGGATGTGGCTTTATCGCGCTGTACTTTGTAGACGATGGCG
TCATCACGGTAATTATCAACAGCCCATGACAAGCAGACGATGATGCAGATAACC
AGAGCGGATATAATCACCGTTACTCTGCTCATACCTGAATCTCTTTGACCGTTCC
GCCGGCTTCTTTGAATTTTGCAATCAGGCTGTCAGCCTTATGCTCAAACTGACCA
TAACCAGCTCCCGGCAGTGAAGCCCAGATATTGCTGCAACGGTCGATAGCCTGA
CGAATATCACCGCGATCAATCATCGGTAAAGCACCACGCTCTTTAATCTGCTGCA
GTGCAACAGCATCCTGGCTTTTTGGAGAGAAGTCTTTCAGTCCAAGCTGTTTACG
GTATGCATCCCACCAGCGGGAAAGAAGCTGGTAGCGCCCGGCAGCCGTTGATTT
GAGTTTTGGGTTTAGCGTGACAAGTCTGCGAGGGTGATCGGAGTAATCAGTGAAT
ATTTCTCCGCCCACAATGACGTCATAACCATGATTACGTGTCGGTTGTAGCCCAT
TATCCGTTCCTTCTGACCAGGCCACCATATCGAGGAAAGCTTTACGCTGAGGATT
AAGATTTTGCATTTTTCACCCCTGTCAGTCGTTCCCAGAAGTACGTCAGTGCAAC
CGAACCCATCGCACCACTAATCCCCGCAGTTGCGAGAATCATGTAAATACTGAAT
CCACTTTCGATACTGATTAGGCCACCAATAACACCGGTGAATCCTGATACAACTA
TCTGAGCGAGGGCATTTATCCAGCTCCACGTTGCTTTACTCTGCTTCACATCAATC
AGGTAACGGACAAGCCCCCCCCAACCTGCGATGATCAGCAAAACAAGCCAGAAC
GCTCCGGCAAGATTCTCTTTTTCGTGCATATGAATAGCCGCAGTTTCGCCTCCGA
CAGAAAATCGGAGCGTGAAATGATATGTTGATTTATTATTAATTCGAAAATTACA
TTCTAATTCATCAGGCATTGATGATTTTATAATAATAAAGGCCGTTCGTTACGGC
CTTTATTATTGATATTTATTTTACATCCCAAGGTAGTTCCTCAAGAGAGAAGACTA
ATTCATTATCAATAATTTCGTATTCTGCATAACCTTCAACCTCTCCTGAGAAGTTA
TTGTCAACGCATCCTTCATAATAGCTATATGGTGCTATAAAGTGAAACCCATATT
CATCTATCTCTGTGATTTCAAAATTGTAGTAATCGTAAGAATATCGAATATCATC
ATCAGAAATAGAACTAAGTACATAGTTATTAAATTTATCGGAATGCTTAACGATA
AGTTCTGACAACGCGTCAAGATTCTCTGGAGAGATATTTCCTAACGGTAATCTGT
ACTCAACGGCATTAATCAT
ST95 Region-5 (SEQ ID NO: 7)
TTATTTTAAGTCATTATGCCAATCGTCTACTGTTTGAAACCAAATTTTTAACCTTT
GCATAAAGGTACGATCCTTTTTGGGAGGGTGGTACCTTTGAAAAGAAGTATGTAA
CGCTTCGATAACTCCCATACAAGCATCAATTACTTCTTGTGCTTTAACCGGGCGA
TCGGCATCTGAAAGCTTGTGAGCGACCTCACAATCCAGAGTTGCTTTCTCTAGTA
CAGATGAGAACACCTCGTCAGGTATATACAGTCCTCTGCTTTCCAAAATGCAAAT
CAGTTTGGAAACTTGTTGAGCTTTAGCCAAGATAGTTAACAAGTATGAAACAGA
AGCCTTTGTTCTATCCTTTTGCAACCTTAACAATCGATGCTTTGATGCTCTAATTT
TAGGTTGAGTTGTCTGACTGAGCCAATAGTTAGAAGAAATATCAGAAATTTCAAT
AACTAACTTTGAAATAGCATCAATAATTGATTTCGATTCAGAACGTGTCGCCAGT
TTTACAGAATTATTGTACGCAACTCGCCATCCAATGAAAACTAAGCAGACGCTAA
CTACTGTGATACACCACGAATACTGTCCTATTTGTGCTACTAATGACATTGGTTCC
GTACTGATCAACTATCGCCTCTTATACTCTTTCTGGTTGTGCTAAATCAATAAATT
TATCAATCTGTTCTTTGTAGAACGGAACATCACTTTTTATAATCAAACGTGCTTTA
ACGTTGGCTTTAGGTATCCCTTCCTTACGGATGAGACCTCCAAACGCCTCTTCTA
AGAAAGAGGAACCAACACCCAGTGCTATACCGCGAAAATCAACCACAATCTCCT
GTTCAGTACCCTTCAGCGCAGGCACCAAAAACTCTCTACGAAATCTTTCCGCACT
GTGTGGGCTATCCGTTGTGTAACGTCCGAAAGGTGTTCTGGAGAACTCCTTCGCT
ATGACGATATTTCTCATAAAATCATCTCCTGGGAACCAGCGACCATTGTAGTAAA
GTACCCGGTATGTACTCAGCTAGCTTCTCACATCGAGGACTACTGTCATTAGAAT
TATAGCTATAACGAGCCCTTCCTGTTAGTATCAGAAGTGTCTCATTATCCTCACA
ACCTGCCCCAATAGGTCTCTTGATATCTTCAGAGCCATTTCCCCGTCCGGAACCA
ACAAAGCGGGATTCCCCTACTAACATAGCTCTTTCCACAGAAGATACTTCGTTTT
GAATGCTATGACCATCACCATTAGGCACGAAACTTCTATAAATCCCCAATCCAAG
GTCACAAACGATAAAAACGACTTTATTTTCATCACGATTGAACCAAGCGCACTGC
CACCATCGTTTACCTTTGAGAAGCTGAATATCTGACTCGAACCCCTCATCTTCAT
ACGCATGATGAGAAACATTGAGCAAAGCTTCACTAATCGCTGTCAGCAGTAAGC
CTAACTGTTCATTATTAAGCAGAGCACTTTTCTGCAGCATGAGTACTGTTTCAAC
AATATGCTCGTAAGGTTCGACAGCTGACTGAAAGTATCGTTCCTCCCTCGTAAGT
GCATTCAGTTTCTCTTCTGTGCCAGCAAGCAAAGCACGAGCAAGTCCAGTACCAA
CGATCCATCTATGCCCAGATGGGTTATCATCCTTTTTAGGCCATTTAAAACGTAT
AAAATTAGGGTCCCGGGTTAAAAATTGAGCTCTATTTACTATGGCAAAGAACAGT
ACCGATGCCGCAGCTGAGGCAAATTTTACCTTGGACAAATCAACTATAACTCGAC
CTTTATTTTTTACTCCAATCGATTCAATCGAGTTGATGAAGTTCAATGTCCCAGCC
CTAGAATCATCTGAGTAAATGCAAAATTTGCTTGGGGGTACCAGTATGGTCAT
ST95 Region-6 (SEQ ID NO: 8)
ATGACATCGACGATGCATATGGACAGGGCCGATAAGAATAAAGTTGGCACCGTT
GAAATTGAGGATCTACGGGTTATTCCGGTTATTTTTCTGCCCGGAGTCATGGGTT
CAAACCTTATGGATAAGAAAGGAAAATCCATCTGGCGATATGATGACTCTATGA
GTCTGATGGGCTGGTCACTACCTACATCAGGACCAAAAGAGAGGAAAAGATTAT
TGCATCCTGACAGGGTGGAGGTCGATAATCGTGGGAGAATTCCCGCACCTCCGG
ATGCCCAAGAAAAACTTATTCAGTTAGGTCAACAATATCCGGAGGACCCATCCG
ATAAAGAAGCAATGGATAATTACACTCAGGCAGTACGTGACATTTTGGATAATA
TAGAGCCAGAAGCGAAATTATTTGGTTCAAGAAAAGATCGCGGCTGGGGAGAGG
TTGCAAATGCTAGCTACGGTTCCTTTCTCGATGTATTACAAACAGCATTGTACCG
GGACAAACCTACAAAGAAAGGTGAGACGTTAAGTGCGACCTACCAGCAACTACT
CGATGTACCTTTGGGTTTGGAGTATGGTCCTGACTCACTTGATGAAGAGTATCTT
GAAGTCATAAGGTTGTATCAATTTCCGGTCCATGTCGTTGGCTATAACTGGCTTG
GTTCTAATATGCTCTCAGCAATCAGACTACAAGAACAGATCAAGAAAATTGTTG
GAGGCTATCAAAAACGAGGAATGAAATGCCATAAAGTTATTCTGGTTACGCATT
CAATGGGAGGACTAGTGGCAAGGTACTTTTCAGAGTGTCTTTCGGGGAATACGG
ATGTCTACGGTATTGTTCATGGCGTATTACCATCAATTGGTGCTGCGGCGACATA
TACCCGAATGAAACGTGGAACTGAAAACCCAGAGTCAAATCCGGAAGGGTATGT
TATCAGTCATATCTTAGGTCGTAATGCAGCTGAAATGGTAGCTGTTTTTTCCCAGT
CGCCAGGACCAATGGAATTACTACCCATGAATGATTATGGTGAAGAGTGGCTTA
ATATTGTCGATCGTGATGGTAGTACATTAACGCTCCCTAAAAATCTTCCCATTAA
AGAAGGTATTGCCCCCGAAGAACGCACCTACGCTGAACTTTATCTAAATAGAGA
AAATTGGTGGAAATTGGTGGATGAGAATCTATTGAATCCTTTTAATACTTCATTA
AATCAAAAGCAAATTGATACTGATTGGAATATATATGAAAATTTAATTACTGAGA
GTGTCAATCCATTTCATAAGCAAATTGCTGGAAAATATCATATCAATACTTACTC
TTTCTATGGGAGAGCGAAATTGGGAGATATTCCTGAAGCGCATTTAACGCAGGA
AAATGTTCTTTGGAAAGGCTCATTATCAATGGGAAAAAAAAGTGATATATCTTTA
GAACCAAAATTCATTGATGGTCGTCTTGATTTAAATGAAGTTGGTAACATTAGAA
CTATTAAGGATGAATTTTCTCCTGAAGAACAGGCGTGGGAAATAAATACTGACG
ACGGTGATACATATGTTAAGATAGGACAGCGTTTTACATTACGAGACTCTTGTGA
AAACGGGGATGGTACAGTACCTCTTCGTGCAGGACAAATCGTACACAAGAACAT
CCTAGAGCGTTTGGCTGTACAGGTTTCTCACGAGGCTGCTTATCGAAATCCCGTT
AGTCAGGCATTTGCATTACGTTCTATTATAAAAATAGCTCAGGAGGTAAAAAAG
GATGGTAAAATGTCATACTCTGATTAACCGTAGAAATAAATGTCTGCTGATTGTT
TTTATAGTCCTTATTGGATGGATTATATTCAGACCTAAAGCATATACTTATTCACT
AAATGATAAAGAAAAAGAGATGCTCATAATGTTATCACAACATCCTGAAACTCG
GTACTTTGGATTTTATTCCATAGAACTTCCGGCTGATTACAAACCAACAGGAATG
GTTATGTTCATACAAGGATCGGCGATGATCCCTGTAGAAACAAAGCTACAATATT
ATCCTCCTTTTCTGCAATATATGACACGATATGAGGCAGAACTAAAAAACACCTC
AGCATTAGATCCACTGGATACGCCTTATTTGAAGCAAGTTCACCCACTAAGTCCA
CCTATGAATGGAGTCATTTTTGAACGAATGAAAGCGAAATACACCCCAGATTTTG
CACGAGTATTGGATGCATGGAAATGGGAAAATGGCGTTACGTTTTCAGTAAAAA
TAGAAGCTAAAGATGGTAGAGCAACCCGCTATGATGGAATTAGTAAGATTGCCG
AATACAGTTATGGATATAATATTCCAGAAAAAAAAGTACAGTTACTTACTATTCT
TTCAGGACTACAACCTCGTGCAGATAACCAACCCCCATCAGAAAATAAATTGGC
GATACAATATGCACAGGTTGACGCTTCACTACTTGGAGAGTATGAATTATCTGTA
GATTATAAAAATAGCAATAATATTAAAATAAGTTTGCAGACGGATAATAATAGT
TATATTGACTCATTATTAGATATAAGATATCCGAGTAATGGAAACAGAGCATGGT
ATAACTCTATATAAAGGTCGTAAGAAAATTAATGGTCTGGAGATATCTGAATGGT
GGGTTCGTAAGCAAATAATGTTGAACTCAGAACCATCACGAGATTATGAATTCA
ATCTGGCAATCCATGAGGATAAGAAAAACAACAAACAGTTATTAAAACTTGTTA
TGAATTACAGTGTTGACATCTTACACGCTGACAATGCATTAACTGAACATGAACT
GATGGCGCTGTGGGAAAGTATAACAGGGACAATTAAATATCATCCCACGCAGTG
GTAAAGCAGATAATCAGAACAGTCTGGAGATATCGGAATATACTCGTGGTACTT
CAAGTTGCATGGTACGCACAAGGCCCTTGTCGGAAAGGGCTTGGGCTACAGAAC
TCATTTACGTGCATCTTCAAGTTTATCGGGTATAGTAGATTAAACAGCAGATCAC
GCTAAGTACTGACCCGCTGCAAAATTACAGGTTTTATCTGACAGTCCATGATGAT
AAAAAGAATGGTGGTCAACGGTTAATACTTACGATGACTTATAGCATCAGACCTT
CTGAATTCAAAAGAAGTCTTAACCGAACATGAACTGGTTGCCGTGTTGCAGCAA
ATGACCAGTACGCTGAAATACCCCCTCTCACAAAACATAAAGATAATGGTATCTC
ATTTATATCAATGACCGGGAATAGGCAACTATACTTTCATTTTTTAAATTATTTTC
CTGTTGTAAATAAATCAGAGGTAAAAATTA
ST95 Region-7 (SEQ ID NO: 9)
ATGGCAATGGGAACAGGCTACTTTCTGGTCAGGGGGGACAAAACCACCTGCGGC
GGGAAGATTATTGAAGGCGCGGACGACCATACGATCATGGGTATCCCACAGGCA
CGGGATATGGATCGGGTCACCTGCGGCAGATATCCGGGAATGTTTATTATTGTGG
GCGGCGTGCCGGAAACGGATATTCACGGCAGGCTGATGGCGGGATCTCTGGACA
GCCAGAGCAGTTGCCCGTGCAAGGCGCGGTTTATTGCGTCAATGATGGATGATA
CCTATGAAACGGATGACGGGGGAAGCGAGCCGGAACAACATGCGCAGTCGGCG
AGGAAAAATCTGACTTCCGGTAATCCAGATAAAAAATACAGTCATCAGATAAAA
CTTCAACATGGCGAAAATAATGTTAGTGTACAGGACATACCTTATGTCTTTATCC
TAAATAATAACATGTCTCTCAGTGGCAAGACTAATCAGGATGGCGAGACTGAGA
GAATATATACAGATACTGCTCAAAAGGTAATTGCACTTACAGGAAAACTGGCTG
ATTCCTGGCTGAAAAGAGGGAAAAATTTTGGTTCATTAAAAGAAATTGACAATA
GAAAGATCGAATTAACAACTGAAGAGAATGAACCTGTAAAATATGTCAACTGGA
TTAATGGGCGTGATTATATTGTGATTGTGGCGGCCAGAACAGCCGTAACAAACTG
GATAGGAATGGAGGACTCAAAAGGAAATCAGTATCGATTTATAAATTGTGGATT
AGAACAATTACAACAGTTTCCTCCAGCCAGTAAACAGGATTCCAGTTCGCAGCG
AATTATGGTGGTTTTTTCGCTTGGGTATACGCAAAAAGATATTGATAGAATTAAT
GATTATACTAAAGCGCACGATGGAAGAATTATCTATGTCAAGAATAAGGATGAA
TTAGTTTCATTCCTGAATCAACGCAAAGAAAAAGGAAGAGTGATTAAGGAACTG
GTTATTCTGTGCCACGGTGTTATTAAAACAGCTTCATATCATTATCATCATGAAG
ACAAGGATATTGAAAAAAATGGAATGTTTAAACATGAAGATATTGCAGCAGTTC
ACGAGTCAGTTTTTGATTATGATGCCCATGTGACAACATATGCCTGTCGGGCCGG
TATCTCAGATGGTGATAAAGATTTTTCCGGGAAGGATGATGCAGGGCAAAAGGA
CAGCCCGGCACAGAAAATGGCTGATAACTGGGATGTCATGGTTAAAGCGTTTGA
GATGAGATCGGACTACAGTCTGGCTTATGGGACAGGCAAGGAAATTAAAGAAGC
TCAGGAATATGGTAGTGTGGTAGAAAAATATAAAAAAGATATAGATATGTATAA
TAAAGAAAAAGCAAAGGGGAATACTGAAGTTTCCCCTCCAGTGAAACCAGAGGG
ATATGATGAGAAATCAAAACGCCATGCAGATGTTACTACCAGAGACAAAAATGA
AAAGTCTGGCGGAGGACCAATAGCTCCTAATGGTGCATGGCATATGCCAAGAAC
AGGGGATTCGCCGAAAGGGCTGAAAAGCGGGTTACAGGATTATCAGCCTGAGGA
GTGGGTTCAATAATGTTCTTAAAAAGAAAATGGTATTACGCAGTGACGACATCTG
TCGTCATTACTTTGTGTGGTGGAGGATATTATATGTACAGGCAAGAATATCAGAT
GGTTGTCACTGTACCAACTGCTGACGCGAACGATCCCAACTGGCCAAATAAAAG
GATACAGTTTGATACCAGCGAATGGCTACAGCAACTTCAATATATTAAAATAGAT
GATCATTATATATTGAATACTCAATATACTCCAATTGCTAATTTGGATGACTTTGG
TATTACATTAAAATTACAGAACGCATTAAATGGGTCGGATAAAAGACTTCCTGCA
CTATATGGCCTTGCTGAGATGGATGCTCAGAAATTTAAAGACCTGATGCGCGGTA
AAATTAAATGTGAATATCTGAGGACGACATTTGATGCGGAAACATTAAAGCCTG
TCAATGATTATTTCCTTATTTCTTTTACTTATAAAGATAAGTGGTATGAATTTGAG
ACAGAAAGAAAAATATCTAAAACAAGTGATGATGGGTATTTTTTGTGGGCATTTG
ATAATACTGTCCACGAAGCAGGCTATTGGCATAACACAGATCCGGCTGCGTATTC
CTATAGAGATTACCAGAATGGTAAGGCTGTGAAATAAGTGTCAGGATACTCAGG
CGGCGGTACTGTGCGATACGCAGCTTAGTTTGGATTACTGATTGTATTGTGGCCT
ATATTGAAGCAGCTCAGCAGGAATTTGTTGAGAAAGCCAGAGAGACTGCAGATG
ATATCTGGGACTTGGGAACTATGTGGGCAAAAAATGTGCTAATTAAAGGAAGGG
AGGTACTGGAGCATGAGGTGAAGTAATATCTAAGAGAAAATCTTGATAATATAA
TTAAGTGGGGACTAAATTAGAGATGAATATTCAGGCAATAAAAGAAA
ST95 Region-8 (SEQ ID NO: 10)
ATGATTGTAGGTAAAACACCGCATACTACAGCTATTTCTCTGATGCCGGGAATGT
CACCTGAAGCACTTATGCAACAAGTAAAAACCATACTCAATCCTGATGTAGAAA
CAATCATATTTACGGATATTTATGGCGGCACACCTTCAAATATCGCCTACCTTCTT
TCACGAGAATTTCCCATTCGCTGCATCAGCGGTGTCAATCTGGCAATGTTAATTG
AAGCCAACATGTTACAGGATTCTGACGAAGAACAGTCCTTTGATGAATATATTCA
GCATATACATGATGCTGGTAAAGAGATGATTCAAACTTATTGTTTTAATAAAGGA
TGATAAACTATGAGTATAGTACATGCCCGTATTGATTACCGTTTAATTCATGGAC
AAGTCATAACAAAATGGCTAAAAAGGAGTGATGCAAATAAAATAATTGTTATTG
ATGATCCCTTGTCACGCGATCCTTTCCTGGCTGAAGTCTATAAGATGGCAGCGCC
AAGTGGTGTTGAAGTCATAATGACCTCCATTGAAGATACGTTACAACGCTGGAA
CAGCAATAGTTTCTATGAAGGTAAATTACTAATTCTTTTCAAATCGATCGATTCTG
CATTAAAAACTATACAAGGTGGTTTGATGTTAGAGGAATTACAAGTCGGTGGTGT
TGAAAATACTCCGGGAAGAAAAATTGTCTTTAACCAAATATCACTCAACCACGA
AGATGCCGACAAACTTCAAATCATCGAAGATAAGAATATCAAAGTTTATTTCCA
AACTATTCCTGAAGAAGATCCTGCCAGCCTACAAAAAATCAAAAATAAGTTACC
TTAATTAAGGAGAGGAGCTTATGTCTATTTTTACAGCAGCAATGATTGCCCTGGT
ATATTGGATATCCCAAGCAAAAGTATGGTACGGCTTTTCTATTATGCGTATGCCA
TTATCGATAGCACCTATTATGGGACTCATCTTTAATGATATGCCAACTGCTTTATC
TGTTGGGGCCACCTTACAGATGATCTATATCGGGAGTATTGCCCCGGGAGGAAAT
CCTCCTGCAGATGAAGGTCTAGCCTCGTGTATAGCAATTCCAATTGCACTTACCG
CAGGTATTAAACCAGAAATTGCTATCTCTTTGGCTATTCCACTGGGATTGCTCGG
CGTTGTTCTGGAAAACGTACGTAAAACACTGAATACCACATTTATCCACATGGCT
GATCGCTACGCAGAAAAAGGTGATATAAAAGGCATCCAACGCGCAGCCACAATA
TATCCTCTCCTGTTAGCCTTTCCGATGCGTTTTGTACCGGTATTCATCGCCTGCCT
GTATGGCCCGGATGCCATTGCCTCTTTTGTAAACCTGCTTCCCGCCTGGTCGACG
AATGGCCTGGCAATTGCCGGTAATATTCTGCCTGCACTCGGTTTTGCGATAACCA
TAATTGTTATCGGTAAGAAACAATACATCCCTCTGTTCATTATTGGCTTTTTCCTC
GTGACCTATTCAGGCTTGAATACCATTGGCATCTCTATTTTTGGACTCTGTGCCGT
CCTCTTATATATGCAGTCACAAAATACAAAAGGAACTAAAAATGGATAATATAA
TTGAGTCAGGATACTCAGAATCCTCCCCTATTACGAAAAGAGACGTTCAGATTGT
TTGGCTAAAATGGCACGCGTTCTCTGAAACATCATTGAACTTTGAACGTTTACAA
GCACTGGCATTTTGCAATGCAATGACAGGCGTATTGGCGAAGCTATACCCAGAT
GAAAAAGATCTCGCCAAAGCCTTACAGCGCCATTTAACCATGTTCAACACACAG
GCTAACTGGGGCTCGGTTATCGCTGGTATTAGTATTGCACTGGAGGAAAAAGCC
GCCCAGGAATCAGAAGAACAACGTGAATCAACAACACAACTTGTTACGGGTTTA
AAAACTGGTCTTATGGGGCCTGTATCGGGTATTGGTGACACGTTAGACTTTGGTA
CATTGCGTCCCATTGTTATTGGTGTCTGTATCCCGTTTGTTATTCAGGGATATGTT
ATTGCTGCACTTCTCCCCCTGATTTACCAGGTTAGTTATATGTTCTTCGCCAGTCG
ATTTGCTCTTAATATCGGCTATAAAAAAGGGAAAGAGTCAATTCTGGAAATCTTG
CATTCCGGGAGCATACATCGAATTATCGAAGGTGCTGGAATGTTCGGGCTGTTGA
TGATGGGGGCGCTTTCAGCAACGTATGTAAAAATTAAGACGCCATTGCATATTAC
AACCGGCGGTGGCAATACCATTGTCTTACAGGATATGCTAGATAAAATTGTCCCT
AACATGTTGCCACTTATTGCCGTGTTGGGTATTTATTTTTACATACAAAAATGTGG
CCCTCATTTCTTACGTATTCTGGTCACTATTCTTGTTTTGAGTCTTGCATTTTCTTT
CTTTGGCCTTTTATAAAATGAAAAAGATAATTATCACTCCCCGGCCTTTTGTGGGT
AAAGGCAAGGTCTATATTGATAAGTTAAAATCCGCAGGTTATCAAGTTGAATGTA
ATAACAGTGGAGGTCGATATAGCAAGGAAGAACTCATCGAGAAAATTAAAGAC
GCGAATGCCATTATCACCGGAAACGATCCTCTGAGCAGAGAAGTTATTGATCAG
GCAAAAAACCTGAAGGTTATTTCAAAATATGGCGTAGGGTTAGATAACATAGAT
GTCGATTACGCAAATAGTAAAGACATCGTGGTACATAAAGCTCTTAATGCTAACT
CTATTTCTGTTGCAGAAATGACCATTCTGATGATGCTCTCCAGTTCCCGAAAATA
TGTTGAAATCGAGAGTCAGGCAAGAAATGGTAAGGACATCAGGCTTGTCGGGTA
TGAGCTATATCAAAAAAATCTGGGATTAATTGGACTTGGTGCAATTGGTCAACAT
GTTGCTCATATTGCTCATTCTATGGGAATGACGATTACTGCTCATGATCCTCATAT
TGATAAAAGCAAGGTTCCATCCTATATAGAACTTAAATCACCTGATGAAATATAT
CAGTACAGCGATGTAATTTCTCTGCATTTACCTTTACTGGACAGTACTCGCAATA
TAATTAACGACTCTGTATTTGAGAAAATGAAATCTTCGGCTATTTTAATCAATAC
AGCCAGGGGTGGATTGGTCGATGAGAAAAGTTTATATACCGCACTTAGCAATCA
GAAAATTGCTTTTGCCAGTGAAGATATTGAACTGAGAGAACGTTCAAAAGAACT
CACCGAACTTAAAAATTATTCTATTACCCCTCATGCCGCTTCATTTACCGATGAG
GCCGACCATAACACAATGCAAATTTCTATTAAAAACGTATTACAGGAACTGGAG
AAAGAGTAATGAAAGAGCTCAAAGGGATTATTACCGCAATGGTAACGCCTTTCG
ATGAAACGGAAAAAATGGATATCACTGCGGCGAAAAAAATGGCGCGCTGGTTAG
TAGATAATGGTGTTCATGGTTTATTCATTTGCGGAACTAATGGTGAATTCCATTTG
CTTTCCGATGACGAAAAAGTTGAGTTAACAAAAGCCGTCGTTGAAGAGGTCGGT
AATGAAGTTACCATTTGTGCAGGTGCTGGGTGCTGCAGCACAAAACAAACCATC
GAATTGACTAAACGGTTAGTTGATGCGGGTGCCGATTATATTTCTGTTGTCACGC
CGTACTATCTGGTACCAAATCAGGAAGATTTATACCGTCACTATTACGATATTGC
CTTTAGCACAACAGCCCCCATCATTCTCTATAACCTGCCGGGCCAGACAGGATTA
AGCATCGAATACGAAACGGCAAATCGTTTAGCCGATATCAAAAATATCGTAGCG
ATAAAAGACAGTAGCGGTAAATTCGAAGTCCAGAAACAGTATCTGGAAATCGCT
AAACATAAAAACTTTAAGGTATTAAACGGCTCTGACTCGTTAATGCTGGATGCAT
TTAAAGAAGGTTCTGTGGCTGCTGTCGCTGCAACGTCGAATGTTCTGCCTACGAT
TGAAGTCGATTTATATAATTACTTCATGGCCGGTGAAATGGAGAAGGCGCAGGA
AGAGCGTAATAAAATGGATGCACTGCGCATGACGATCAAGAAAATGACCGCGCC
TGCAGTAATGAAGGAAGCATTAAACCTGATGGGTGTGAAGGCAGGTATTACACG
TAGACCAATTCATATGCCAAACGACGCGATCGTTGAAGACATCAAGGAAATGTT
GAAAGGCTATAATTTCCTGTAAAGGACAAATGCCGGATGTATCTCTTGTCCTACA
AGATAGCGTAAACAAAAACCCCCGGACTCAAATCCAGGGGTTATCTGTGGCAAC
TTAACGCGCTGCGCGAT
ST95 Region-9 (SEQ ID NO: 11)
ACTTTGAACATCCACTCGATCTTCGTCTACTTCCGGTTTAT
ST131 Region-1 (SEQ ID NO: 12)
TTATTTGACCGTGACCTGTCCGTTCATAAGTAGAGGGAGGAGCCAGTCGCGGAGT
TGGATTAAATGACTATTCTCTTGAGTGTTTGATGCCAGTTTATTATAAATTGGCAT
CATTTTATTCTCGAATAACGCCACAACATCAGGAGTGGGGCATATGATTTCACAA
TTAGTGATATGCGTATTGGTCAGGTTATTAATATTAGTTCCCAAGCACTGATTGTT
TATATAAGACTTAAACCATTTCGATTGCAGGAATGTATTTATAAAATAAAATGCA
TGTTTTTTTTCGAAATAATACACGCCATTCTTCCCTACATGCTCCTTACTACCACT
TGACATCACGATCAATATGTCAAATTTATTTAGCATTTGATTAACGTTTACTCTAC
TAGAAGGAATGAAGACTAAATCATCAATATCAACATTATTTCCCGTCACATTTGT
AGCGCGAAGAATGCCTATCGTATCTTTATCATCCTGAGTTCGAACATCCTCTTTG
CTATAAGTAACGCCACGATCTAACTCAATAAACTTACCAAGAATGGAGTCATTCC
AACCTGCCGGAATTTCCCGTTTCAGCGTGGCGTTATACTCCATCTTCCCGCCGGA
GGTTTTATACGGTTTTCCGTTGGCATCAGGGAAATCAAACTGTACAAACCAGTAG
TCATACAGTGTTTTCGCCATCGCTTCCAGTTCCGTGTTGATGCGGTTGTTGAGGGC
GATTTTTTTATCAATAACACTAAGAATAGTCACCAATTTTTCTTGACATTCAATTT
CTTCAGGTATCCTGACTGGATATTCTTTTAAAGAAACTAGATCAACTCCAGTTTGT
CCTCCGGTTCTGGCAGATAATTTATCAACATATTGGAAAACAGAGTCTTGCTTCA
AAAAATAGTATAAAAAATCACGACTTACACCTTCTTTAGGTATAACCTTCAGCAA
GGCAACGTTATATGCTCCTTTCAATCCGCGACATATTTGAAAAATTGGGGGACCA
TACCTACCAATCATAATATCGAATTCATCACAAAATTTCTTCGTCGACGATATTG
GTATATACGTGGGAAATGCATCCGTTTTGTAATCACGCGTCTGGATCAACCTGAC
ATATCCAGGTTTAGATACATAAATAAATTGAGATTTTGGAGGTTGACTTCCTCCG
ACAAAATCACATACATCTTTAATTTTCAGATAATTAAATTTCAT
ST131 Region-2 (SEQ ID NO: 13)
TTACCAGCAGACAAAGCCACCATCGACCACCAGATCCACACCCGTGCAGAACGA
CGCCGCATCGCTGGCAAGGAACAGCGCTGGACCCGCCATCTCTTCCACTTTCGCC
ATCCGTTGAATCGGCGTCTGGCTTTCAAACTCCCGAGTCTGATGTACCATTTCAG
GTCGTGTATTCATTGGTGTCGCGGTATATCCTGGGCTGATTGAGTTCACGCGGAT
CCCCTTACTCACCCATTCCATCGCAAGACTTTTAGATAAATGAATCACGCCGGCT
TTGGAACTGTTGTAGTGGGCCTGTTCCAGACCACGATTGACAATAATCCCGGACA
TTGACGCAATATTTATAATCGAACCGTCTCCGTTCTCTACCATTAACTCAGCTTCC
GCCTTACAGGAATTCCACACACCAGTCAGGTTGATATCGATAACGCGTTGCCATT
GCTCGCTTTCCATCTCCAGCGCCGGGTTTGCGTTAGCGATTCCAGCGGCGTTTAC
GGCAATATCCAGACGACCAAAATGTGTTTTTGCCAGAGCGACTGCTGCGCGAAG
ATCTCTCAATTCCCGAACATCGCCCGTGTAATACAAGGCTTGTCCGCCAAGTGCT
TCGATATGATTAACCGTCTCTTTGAGTCCGCCATCTTCCCGTAGATCAAAACACA
CCACTCGTGCGCCTGCGCTGGCTAAAGCCAGAGCGATCATTTGTCCGATACCGCT
ACCTGCGCCAGTGACAAAGGCGACGCGATCGTGGAGATCAAACAATTGTTGAGC
AAAATCTTTTGCGATCATTTTGATATCTCTCTTAGTAGCCCGCAGGGGCATTACG
ACATAATCAGGCCGCCGTCGATGTTGATGGTTTGACCGGTGAGATAACGCGCATC
GTCAGAGGCGAGGAAGGCAACCAGTCCGGCAACATCTTCAGGTTTACCGGCACG
CTTCATCGGAATACCTTCCACCCATTCCGCCATAAGCTCGCCTTTGCCATAACGCT
TGTCATCGGTACTGAGAATTTCTCCCCATACGCGGTCGTTGTAGTCCCACATTTCG
CTTTCGATAATACCCGGGCAGAAGGCGTTAACAGTGATATTCCACTGCGCTAACT
CGTGCACCAGGCTTTGCGTAATGCCGATCACACCCATTTTGCTGGCTGCATAGTG
TGGGGTATAGATAAACCCCTGACGTCCCTGGCCAGAAGAGGTGTTGATCAGGCA
GCCGTGATTCTGTTTCACCATATATTTGGCTGCTTCCCGGCAGCAGAGCCAGACT
GCCGTAGTGTTCACCGCCAGCACTTTTTCGAAATCAGCAGCCGGCATTCGATCAA
AATAATCGATGGTGATAATGCCTGCATTCTGAATGGAAACATCGATGCTGCCAA
AACGTGCAGCCGCTTGTTCATAGAGTGCCTGTACTTGCGCTTCATCTGTCACATC
GACTTCTAATGACAAAATGTCAGCCTGATAACATTGGCGCAGTTGCTCTGCAGTT
TCATGTACGCGTTCAGCGTTAGAAACCATCACCAGATTTGCGCCATCGCGGGCGA
AGCGTTCAGCGATCCCGGCCCCAATTCCCCGGCATGCCCCGGTAATAACTACAGT
CTTACCCTGAAAATTACGTTGCATATTTTTTCCCCTGTATAAGCCGGGCAATTTAA
TGCCCGGTGGAATGATGTTAATTAACTGGAAAGCGGTTTTACGGCAGGTGCGGC
GGCCATCTTTTCTTCATGGCGACGCATCAAAATGACTTTGTTTTGCAATTTTTGCT
GGAACTGATCGACCACCACGGCGGTGACGATAACGAGGCCTTTAATGACCATTT
GCCAGAAGTCACTGACACCCATCATCACCATGCCATCGGCGAGGAAGACAATGA
CAAAGGCACCGATAATCGAGCCAGAGACACGTCCCCGACCACCCGCCAGTGCAG
TTCCACCAAGAACCGTGGCGCCAATAGCATCCATTTCGAACATATTGCCGGTCAT
TGGGTGAGCAGTTTGCAGCTGCGAGGCAACAATCAAACCGACAAATGCGGCGCA
TAACCCGGAGAACGCATAGACGAACACTTTAACTTTTACGATGGGGACGCCTGC
CAGTCGCGCGGCAGATTCATTGCCACCAATGGCATAGATATAACGACCGAGCGG
CGTTTTAGTCGTGATCCAGTAGCCTAAAATCAAAAAGCCAATCATCAGCCAGATT
GGTAGATAAATGCCTAAAAACGTGCCAGAACCAAGGGTGGCGAAACCCGTGTTG
CCCAGCGTTTCCATGCCGTTGAGATTGGGATAAGTGCTGCCGTCATTAAACAGCA
GGGCAGAGCCACGAGCGACGTACATCATACCGAGGGTGCAGATAAAGGGCGCG
ACACCAAAGCGGGTGATTACCGCGCCATTCACCAGTCCCACTAAAATGCCAAAT
ACCGCCACGCAGAGGATCACTTCCGGCACGTTGAAGAAAATAATATTGCCGCTC
CACAATGGCAGGCCGTTTGTTAGAAGAGCCCCGGCAACCATTCCGCAGATCCCC
GCTACCGCGCCGACGGAAAGATCGATGCCGCCGGTCAGGATCACCAGCGTCATT
CCGATTGCCAGCAGCCCGGTAATCGCAACGTGTTGAGTCATGATCAACAGGTTA
GAGGCTGTCAGGAAGTTAGGCACCATCACGCTAAAGAAGGCGATCACTAATAGC
AGAGCGATAAACGTTCTGGCCTTCAACAGGTACATATAGATCATATATTTCTGAT
TCAT
ST131 Region-3 (SEQ ID NO: 14)
TTACTCGCCTCCCGCCCTGGCTATTTTCGCAGGCGTTATATCCGGGATAGTTTGCG
GAATATCCCATGCAGAAAATACGCCATAAACCTGAACCGTTTCCCCGGTTTTAAG
CTTTGCCGACTGAATCATATTGATGGCTTGCTCATTGATAGCACGACCATATTCA
CCAAATAAAACCTGATCGTTAAAATGCTCATAGCTTGCACCTTTATATGCATCTC
GCAGAAGCGTGCCGCGAAGAGTTGGCCCCGTCTGCACGACAATATTTGTGCCGC
TGATATCTGTGATGGTCATTTTTCCGCTACGGGATTTCGCATCAATATTGGTGATG
CTACCTTCGACCTTAACGTAGAAAATACACGGGTTTTCTTCCTGAGAACGATAGC
CCAGCGTTTTACAGGCAGCATCGACATCTTTTTCTGCCTGTAGCGCCGCCATTAA
CTCCGCAACCGGACGCGCATTTTCTACCACCTGCGGCACGATGTTTTTCTGCCAG
GTCTGATCGATATTTGCCATATGAGGATTAGGCGGGGATTTGAGATCGGCAAGTT
CCTGCTGCGAGACGATGCGACATCCACCCAGGCTCAGAATTGCCAGCGCCAGCC
AGACTTTTTTATCCATAATATTCTCCTCTGCGCCCTCATTGTGAGGGCGCAACGAC
CTGGGATCAGGATTTGATATTGAAATCCTGGACTTTATCTGCGTTATCTTTGGTTA
TCAGAATGCCGCGGAACATCACCCGCTGTTTGGCTGGTTTAACTCCTTTTTGCAG
GTAATTATCCAGATCGGTAACACCCTGCGCGGCAATGGCTTGTGCTTGTAACATC
ACGGTAGCCTGCAATGATTCGGCTTTCACGGCATCGCGCTCATCGTTACTGCCAT
CAATGCCGACAACAATAACGTCATTGCGATTGGCGGCTTTCAGTGCTGCGATAGC
TCCCAGTGCAACCGGACCATTACCACAAATCACCCCTTTAACATCGGGATGGGCT
TGCAGAATACTGTCCATTATTCGTTTGCCATCAATTAACGTGCCTTTGGCATCTTG
CTTCGCCACGCTGACCATTTCAGGGAACTGGTCAATAACCTGGTGGAAAGATTTC
GAGCGAGTTACGCAATTATTATCAGCAAGATTACAGGTGAGTTCGGCATATTTTC
CTTTCTCTCCCATTTTCTCGACAAATGCGTTGGCAACTTCCGAGCCAGCCTGGAA
GTTATTATGGGTAATTTGCACCAGTGCAACATCATCCACAGGTATTTCGCGATTA
ATTAATATAACGGGAATACCTGCTTTTTTTGCTTTTTCAATGGCTGCAACACTGGC
AGTGGAGTCGGCATTATCTAAAATAATGCCCTGAACTTTTTTACCAATTGCAGTA
TCGATCAGTTCATTCTGTTTTTTGACATCTTCACCATGAGAAAGAATGGTCGTTTT
ATATCCCAGTTGCTGGGCTTTTTCACTGGCACCTTTCGCTTCCGAGGCGTAATAA
GGATTATCCAGTGAATTGACCATAATCATAATGGTTCCTTTTTCTACCGCATGAG
CAGAAAAAGAAAGAGCAGTCAGGGTCGCAGCGGTTAACAGTGTTAAACGTAATT
TCAGAAACAACGTAAAGCCTGAATTATTTATGGGCCGGTATTCTCCTTACGTTGG
TTAGTCTTGTTCGTCAAATTGACGATGCCGGTTTCCATAATGTTCTGTTTACCGCA
TGACGCCACTCCTGCCATCTCTTTTGCAGTCGCTGATGACGCGCCATGTCCGGTTT
AAATTCACTGTGTTCAGTTAATAACGCTTTTAAATCTGCAACGGTACCGGGGATA
AGCGCTTTGCGGGCGAGCAGGCCCGCACCAATGGCTGAAAGCTCTGGCACATCA
CTGCACATCACCGGGCAACCCAGCAGATCCGCCTGATATTGCATCAACCAGTCAT
TTTTGGTCGGGCCGCCATCCACCATTAACGCCGATAAGTGAAAATCATCATGCTG
TTGCATTGCCTCCACTACATCTGCAATTTGATAAGTGATTGATTCCAGTGCGGCA
CGAATCAAATGCGCGCGCTTTACGCCACGGCTTAAGCCACACACCACGCCGCGA
GCGCTGTCATCCCACCATGGCGCGCCCAGGCCAGTGAGAGCAGGAACAAAATAA
ACACCCATAGTCGAATCCACTGAGGCTGGCAGGGTATTCAGTTCGAGGGCCAGC
TCCGCGTCTGTCAGTTCACTTAAACCGGTGCTGTCAGCCATCCATGCCACGGCAT
CGCCGGTGTGGGGGATATTTCCTTCCAGACCGTAAATAATATTTTCGCCATCATG
CCAGGCAATGGTTGTGGCAAGTGTGGCGATATCACACTGAGCAGAGTTGACGGG
GGCCATGACGGAAGAACCGGTCCCATAGGTCGCTTTCACACATCCCGCTTCTCCC
AACGCATGACCAAAAAGAGCGGCGTGAGAATCGCCCACCATCGCCATGACGGGA
ATACCATCCGGAATTGTTTTAAGCCCTCGGGTATAACCGAACAGGCCACTGGAA
GGTTTAATTTCTGGCAATGCAGCGCGTGGAATGTGAAACAACGCCAGCATTTCCT
CATCCCACTCGCCTGTTTTCAGGTTAAGTAATTGAGTTCGCGCGGCGTTAGAGTA
GTCGCAGCAAAATGAATCGCCAGCAGTCAAGTTCCATAGCAACCAGGCATCAAT
GGTGCCGAGACAAATTTCACCCTGGTCGGCAAGTTGCACACCCTCAGGTGTAGAT
TCCAGTAGCCAACGCATCTTGGACGCGGAAAATAGCGGGGCGATAGGAAGACCC
GTAGTCGTTTTAATTTGCTGCTCTTTGCCCTCGCGACGCAGTGTTTCACAAAACTC
GGCACTGCGCGTGCATTGCCAGGTAATCGCCGCATTGAGTGGTTTTCCACTCTGA
CGATACCAGCCGATAGCCGTTTCACGCTGATTACTGATAGCCAGAGCCGCTACAT
TTTCTGCCCCCACGTGGTTGATGGCCTGGGCAATAACATCAAGCGAGGCAGAGA
TTAACGCTTCCCCGGATTGCTCAACCCAGCCTTCACGTGGCGTCTGAATAGCCAG
TGGTTGAGAAAACTTCGCCACCACGTTACCGAAACCATCAAGTGCGATGGCTTTT
GCATTGGTGGTACCCTCATCCAACGCAATAACGAACTCTTTATTTCCTGCAAACA
TAGGTAGTCTCCTGAGTCACATTGCATCCTGGCGTGGAAGGAGATGAGGCGTTCC
TCATGGTCACGTTATTACTTCATCAGGCCCTGCTGTGACATTTTGTGTTAAGGCAT
GAATATATATTCGATACTGATTATTTATTTATACCTTAGCCAGATTATTCTTCCTG
AGCTAGAGAAAAAATCATCAAATGTGCTGGCTATCACACTTAGAAGCGTGGTGA
CAGGGTGAGAAAGCCTCGAGAAACAGGATTGTTGAATCCTTTCAGTTTATTGATA
TTAAAGGCTTTTTAAGCAATCACCTTTGCGAGAAGATTTTTTCGCAAAGGCGGGC
AGAAGAAAGGGAGGAGAGGATTAATCCGCAATCAGCATTCTGGCGGTGGGGAA
ATCAGTGATCAAAACATCAATATAACCACCCTGCATGGCGCCTTTGATGGCGGCA
ACTTTGTCTTTGCCGCCCGCCAGTGCCACCACATTCGGGCATTTTTTGATTTTATC
CAGTGCCATACCAATCACTGGATCTTCATCGTCCTGCAACACGGGCTGACCATGT
TTATCGTAGTAGTGCAGGCAGATATCGCCCACAGCACCGCGATCCGCTAGTACCT
GTAACATATCCTCGTGATAATAGTTGCCCGAGGTTTTTAGCAACTGCGAGGGTTC
AAGGATACCGATGCCGACAATGGCGATATCGACGTCGTCAAATCTGGCGATAAC
GTCAGCCACATCCTGGCTTGCCACCAGGCGTTTTTTCTCTTCTACCGAACCTTCAA
TGCTTTGAGAAGGAAGCAACCAGGCTTCGCAATTCAGATGCTGGGCCAGGGTCT
GTGTCAAAATAGTTGCCTGCACATTGCCGTTTGGCCCCACGCCGCCCAGCAGCTG
AATAACGCCGCTGGCTTTTAAGTTTTGGGCGTGAACTTCATCGACCATCGCGCGA
ATCGTAGAACTCCACGAAGAGACGCCAATGAAATCTTTTGGCCTTAAACGCGTTT
CCAGATAGTGCGCGGCGGCAGAGCCGATGGCGCGTTTAATGGTGTGATCACTGG
CATCATCTTCGGTATCGACAACAATCGCTTGTTTAATGCCGTAACGCTCTTCAAT
GCCTTTTTCCAGATTGAGAAAGATATTTGACGGCTGGACAACGCTGATTTTAACC
ACGCCTTCTTTCTGGCAACGGGTTAGCGCTCTGGATATAAATGACTGAGAAAGCG
AGAGAAGTTGGGCGATGTCAGACTGTTTTCGCCCCTCAAGATAGTAAAGAGTGG
CGATCTTCACCAGTAGCCGCTGTTCATCCTGCTTAGCCATATATTTCCCCGTAAAT
CATTAACGTACCGCCCTTATCTTAGAGGCTTTTATCATACGCGAAAAAAGAAGAC
AGTTAAGTGTGATGGAGTTCTAACTTTAACCAATCCTTAAAATCAGCGATGGACA
AAAATAAAAAACAGGCGGCATAATTGAATAAAGAATCATTATTGAATATATATT
CAAGATGATATCAGGTCAACTGATGATTACCCGGACATGTAACTGACGGTGTAA
GGAGGCAGAATGTTCCTGAGTATGATGCAGCAGCGTAAGTCTTGCCGCGCAACG
CCCTGGGATTTCTGGTTTTTTTAGTCCCCGGTGGAATAAATATTCCATGTTGAAAA
TAAATTCAGAGGTATTAAATGAATCCCTATAGCTATGATATCCAGACCCTTGAGC
GTAAAGCGCGGGCGGTACGCCGCCATATCGTGCGCTTAAATGCCAATAGCCCGG
CAGGTGGACACACCGGGGCCGATCTCTCTCAGGTTGAATTACTGACCGCACTCTA
TTTTCGTATTCTCAATGTCGCACCGGATCGCCTTCACGACGATGACCGGGATATA
TACATTCAGTCAAAAGGCCATGCTGTTGGATGTTACTACTGCGTGCTGGCGGAAG
CAGGATTCTTCCCACTGGAATGGCTTTCCACTTATCAGCAGCCAAATTCACACTT
ACCCGGCCATCCGGTTCGGCAAAAAACGCCAGGTATTGAACTGAATACCGGAGC
ACTGGGGCATGGTTTCCCTGTGGCTGTAGGACTTGCGCTGGCGGCGAAAAAAAG
TCAAAGCCGTCGTCGTATCTTCTTAATTACTGGCGATGGTGAGCTGGCTGAGGGC
AGTAACTGGGAAGCTGCACTGGCGGCGGCGCATTACGGGCTTGATAACCTGGTC
ATTATTAATGACAAAAATAATCTGCAATTAGCCGGGCCAACGCGCGAAATCATG
AACACCGATCCTCTGGCAGAGAAATGGCTGGCATTCGGTATGCAGGTTACTGAA
TGTCAGGGCAATGATATGGCTTCGGTTGTCGCGACGCTTGAAGAGCTTAAACAG
GAAGGCAAACCGAATGTGGTGATTGCCCACACCACTAAAGGGGCAGGCATTTCC
TTTATACAAGGGCGTGCGGAATGGCATCACCGGGTGCCGAAAGGCGATGAAATT
GAACAGGCACTGGAGGAACTCAAAGATGCGTAACAGTGAACATCTGGCAAATGT
GATGGTTCAGGCGTTTATCGATGCCGTAAATGAGGGGATCGATTTGGTGCCTGTG
GTGGCTGATTCAACGTCTACGGCAAAAATTGCTCCCTTTATTAAGCAATTCCCTG
ACCGACTGGTGAATGTGGGTATTGCCGAACAGAGCATGGTTGGTACAGCTGCCG
GGCTGGCGCTGGGCGGCAAGGTGGCTGTTACCTGCAATGCCGCGCCGTTTCTTAT
CGCTCGCGCTAACGAGCAAATCAAGGTTGATGTCTGCTACAACTTTACCAACGTA
AAATTGTTCGGTCTGAATGCCGGGGCGAGTTATGGACCGCTGGCGAGCACGCAT
CATGCGATTGATGACATTGCGGTTATGCGTGGTTTCGGCAATATTCAAATCTTTG
CCCCGTCTTCACCGCGGGAGTGCCGTCAGGTGATTGATTATGCTCTTCGCTATCA
GGGGCCTGTTTATATCCGTATGGACGGTAAAGCCCTGCCGGAAATCCACGATGA
GGACTACCGATTTGTACCAGGTGCCGTGGTGACTTTGCGCGAGGGCAGCGAACT
GGCGCTGGTAGCCACAGGCTCTACCGTTCATGAAGTGGTCGATGCCGCAGAACTT
TTAGCGCAGTCTGGTATCGCGGCTACAGTTGTCAGCGTACCGTCGATTCGCCCAT
GCGACACCAAAGCATTGCTGGCAGCGTTAAAAGGATGCAAAGCAGTGATGACGG
TTGAAGAGCATAACGTCAATGGCGGCCTTGGTAGCCTGGTCGCGGAAGTGTTGG
CAGAGGCCGGAACGGGGATCACATTAAAACGCGCAGGCATTATGGATGGAGAAT
ATGCCGCAGCGGCGGATCGTGGGTGGTTGCGTCAGCATCATGGATTTGATGCTGC
CGGAATTGCAGCTCAGGCACGGGAAATGCTTTGA
ST131 Region-4 (SEQ ID NO: 15)
AAGCATTTCGATGGCATATAAGGAGTATTACGCGCTACATATTACGGAAACCAA
TTCTTTCTTATAAAAACTATAGAAAAATTACATCCGAACTGGTAAAGTGATTAAA
TAAAGTCCTAATTTTACTACTGAAATGGTATCTTTATTTAAAGATCTTTGCTTTAT
ATACCAATTTTTTAACGCCCTGACAGGAGTAAACAA
ST131 Region-5 (SEQ ID NO: 16)
ATTGGTAGAGAATTTTGCAAATCTGGTTTTGTAAACGTTAAATCAGCAGCGCACT
GATAAACATCCAGATCTGATTCATGGCTAATTGTTCTGACATTTTCTACAGAAAT
ACGCACATCCTTATCCAGTTTCATCCCTTTCTCGATTGTCTTTCTTGCAATATCCC
CGACCAGATTTTTTGCATCATCACTACTACATTTTGGCGTTTTCTCGCCGCAGCCA
GCCAGCAAACCAGCAGCAATTAATACCAGAAGTATTGCTCTTTTCATCTGTAGTT
CCTTCGTAAATTAATGATACTTTTATTTCTTATTAAATAGACCATCCATTGCGCCT
GCCGCAGAAGCCCCGCAGTATCCCTCGCAACCACGTGTCATAATGCTGGCCTTAC
CGTTTGTCAGTTCACTAATTACTGCAACACACTCGCTGGCATTGTCATTCCAGAC
CCAGCGACGCTTCTGACTTTCTCCGGCAGCTATTTGCTGTGCTGTCCCCTCCATAT
TGCATACGCCCGAACCATCCCCCGTTGAGGCATTAATGTTGAATTCAGCGTTATT
CTGTCCCGGATGCAGGGTTAGAACCGCTCCGGGTTTTACGTACTGCGCTGCTGCG
ACTGGTAGCGTTATGGCTGCCATGAATAAGGCCAACAGTTTTCTGTTCATTAATT
AATTCCCTGTTAAGTGATACGGATGTTTTTGGTGATTTTCCCCAGTGCCAGCGGTC
TGCGTTTTTCCACAAAGAAATAAACGACGTTGAATGCAGACTTGCGATGATTCCC
TCGACCAAGAGAGGGCCAGTTGCGGAAGGATTCAATCTGTTCTTCTGAATAGGG
TGTGTCAGAGAAATCAAACAGCACAAGCAAGGCTTTGCCGTCATGTTTCATCAAT
TTCTTTACTTCTGTACTGTTGACGGTCGCAGAGACATTGGAACGTGCCGCAGTTG
GTTTACGTACTGCAAACTCAACGGCCACATCGTCAATGACAAAATCAATGTAACC
GTGCCCGGATACAGTTCCTGGCAGTTTGCTTTTTACTTCCGGTGAGACGTTATCTG
CAAACCAACCCAGCAAATAGCAACGTACCAGAGGCAGTAATTCCCGCTCGCTAC
ATTCGTTCAGACGCAGGGTTTTGACGAACTCCCTGCGATAAAGAGAACTGAAAA
AATACTCAAAGCATTCCGTTATTTCAGTGATGGTGGTTGCCATGGTTGCATCCTTT
ACCTGCTATTAGGTCCATTTTTAAGGGCATACCTTTCCGGCCATAATGTTTCCGGT
GTCGTATCCAGAGCCCCGGCAATAATGCGCTCAGCTCTGGGATAATGACGCTCTA
AGGCGTTATTTAATGTCCGTGGCGCAAGGCCGTGACTGCGTGACAGTAACGCCA
GCGTTAACCCTCGCTTGTGAAGTGCTGCCACAATATCGGCACGATGCCAGTTCCC
TGAACTTCCTTTGATTTGCTTTTTCTGCGATGCTGTTGAGTTACGCACAGTATTAA
CTCCATGGAATCAGTACATGGATATTAAATGCGAATAAAAGATACCTTTCAAGTT
CTTTTGGTATCTATTTATTGGTTTTCATATTAACGCATTGATATCTGGTGAAATTT
ATATGAGCAGTAAGAGAACAGTTTCGGCTATACCTACTCATCCTGAAGCTTCTGT
TGAAGAGGCTAAAGACTGGCTGACAGCAGAGGAATTCACTCATATTGAGGGGAT
GCCGGGCACTGCGCGTGGCGCCAGGATGCGCTTGGAAAAGCTGGCATCCCTGCA
CCCGGAAATACGTCGTAAAAGGACTGCCGGGAAAGGTGTGGTTTACCATATTAG
CGCAGCCACAATGTCGCTTGATGATGAGAGAAACAGGGCTAAGGGCGAAGCGTT
TACTCTCAACGACAGTGATCGCCAGCTAAGCCTCTGGGTGCAGCTGTATCGCAAT
ATGTCGCCAGCTTCGCGGGCGCAACTGATGGAGCAGGCCCTCAGGCTGGTAAAG
GACGATCTGATGTCTCAGGGGCCCAATGTTTCTGAACTGGCGGTACAAATCATGA
AGCTGCCCGAAGATGAGCGTAAGCGCCTCTTTGAAAAGCTGAACGGGAAGGTGT
GATAGCGTCAGTTTTCCGCCATGCCAGACAGAAGGAACCACCCCGGAATCCTGA
CCTGTCTGACATGGCAGAATGGAAAGTATGCCATCACTTAATAATGAATAAAAA
CCTATTGCCAGTTATTTCATTGCTGAACCACATATTAGTTTCTCTTTGTTACTATTT
GCATAAAAAGTATATTTTTGATATAATAATATTGCTGGTGAACTATATTTTACCC
AGTCAGTACGTTGTCATGCACTTACCATTTATTAGGTGATTAAACCCCACCTGTG
CTGGTGCAGACATAACGTCTTATCAGAATATATTGAACAGGAAAATCACTGATG
AATATTGTCCATCAGGAAGGGCTAGCTATGCCCGTCGATCAGGAATTAATAAAT
ATCATATTAAATGAAGCGGGAAATCCACCGCCGCATAAAGCCAAAATTACAGCC
GTCAGTCTGTTATTTAAAGACCTGAGTTACAGCGCTGAAAAGGGTGGTTACCATC
CTGTCGAAATACGGATTATTTCACGCAATGATGAATGGTATTTTGATTACATTAC
CGACTTCAGTTATATGGGGACGGTATACCCCGAACTTGAAAAGGAAATTGATAT
CAGCTGGAGTCAGCAATATGTCTTTCTTGCTTATGTTGGTGACCTGCCTGTTAACG
CCGGAAGGGAGCTGTTTGAACTCTGGCAATCCAACTTTATTCAGTATCACGGCAT
GAACATCTACACCATTACCGTTCTCTGGGAAAGCTAATCCCCCTGTACTAACCAT
CACTAATTTATGGGGAATTATCTGTGACGACTGACAGAGGGATACGTGCGTCTCA
ACAGGGCGAACTGGACTGTCTTTGCGGTGCTTATTGCGTGGTTAACATGCTGAGC
TGGCTGTATGACGGCAGGGTGAAACGACGCCCGTTAATGAACTGGCTTATTCAG
ACTTATCAGCAGCGCTGGCCTTTGCATGAATGGCTGACTGAAGGAATTGAAGAA
GACCGCTTAGACTGGTTAATAGCTCAGGTGTTGCAGAAAGGGCATTACCGCCGC
CAGTTCCCGGTTGAAATCACCAGACCCTTTGCAGGGAAGCGGGGGTTAACAGAC
GGGCGGCTGTTCAGGGAAATGCAGCGGTTTCTTGACGTCACTGACCACTCTCGCC
TGATTATGCTCAGCGACCAGTTTCACTGGTCCCTTCTGGTCAAAATGGACGAAGA
AATGCTCTGCTTCTTCGACAGCAACGGGCGAACCACCATGCCCCGTAAAGCTTTC
TCCCTGCGTACTGGCGTAACCCGCCGCCAGCTGTTTCCAGACGCCATTTACTTCA
TTGAACGGGAGTTCTGACATGACCATGTTTACCCAGCGTGAAACCCGTATCCTTG
ACCAGGCGCGTGACATTATTAGCCGGTACTACCAGCGTGGGGTTCAACTCTGTTC
CCCTGATGATGTTCGACGGTGCGTGATGGTTGAGCTGGCACCGCTTGAACATGAA
GAGTTTGGCATCATCCTACTCGATAATCATAATCAGCTGCTGCACCGCGAAATCC
TGTTCAGGGGTACGCTAAACTCGGTCAGCGTTCACCCCCGTGAAGTTATTAAGCG
TGTGCTGAAACATAACGCCGCCGCCGTCATCCTTGTCCATAATCACCCCAGCGGC
GAGCCTGAACCCAGCCGGTGTGACATCCAGTTGACCAAAAAGCTTCAGGAGCTG
CTGGAAATACTGGATGTGCGGTTACTTGATCACTTTATCGTGGCAGGAACTGAAA
CGGTGTCGATGGCTGAGCGAGGACTGGTGTGATGCTGAAAGTTACTTGGTTTAAT
GTACGTTATTGCGGGTTACGCGCATGAGCAGGACAGCGTTGCGTCAGGTCGTGCC
GGTGGTGCAAACCACCGTGTTCGATGCGTATATTGTCGGTCTGGCCCCCTCGGGA
CGGCGAGGTATCACCAGCCTGTTAAACCGCAGCGCCAGCATACTTAAACACGGT
GCGGATGCAGCAGACTATCCGTGGGAACAACTTAACTACGCCGCCGTTGCCAAA
GTGCGCGCGGCACTGCTGGATGACGGTTACGCCGTGTCATCGGTCAACATGGCG
CTGTCAGCACTCCGGGGCGTAGCGCAGACGGCCTTTAACCTCAATTACATGGATG
CCGAAACGCTGGCACGTATCCGGTCAGTTAAGCGCGTGAGCGGCGATGTCCAGC
GCAAGGGAAGGGCGCTGGGCAGGCAGGAAATCCGGGTACTGATTCAGGCTGCA
AAACAGCATCCGCAATCTGTACGGCGCTGTCGCGATGTCGCCATTGTGCTGATGC
TTTGCGGAACGGGCCTGCGGGCCGGGGAACTGGTTAAACTTGAGCGCCGGGACT
ATGACAACGGCATCCTGACGGTGAGGCAAGGGAAGGGGCGTAAATACCGTGAA
ATTCACGTGGCGGATGCGGTGGACAAGGCTATTCGTGCGTGGCTGAAAGTTGGC
GCAGACGAGGCTGACAGTGCGTTGTTTAGGCGAATTCAGCGCAGTGGTAAGGTC
GCAAGCCAACCGCTGACTACGACCGGGTTAACGGGTATCCTGGCTGAGTTACAG
CAAACGGCAGGTATAGCACGTTTTACCCCGCATGATATGCGGCGCACCTTCATCA
CCCGTTTGCTGGAGCAGGGCGTGGATATCAATACCGTCCGCCAGTTGGCAGGCC
ACAGCGACATATCCACGACGACGCGCTATGACTGTCGTGGTGATACTATGAAGA
TCAGTGCCAGCAAGCGCATCAGATGTTTTTAGCAGTTGAGGCGTTGCCAGACTCG
GTTTCAGTTTCATCTTCAGCACTCAACCGCGCGTACTTTTGATTCTGGATGTTAAT
TTGTGCATTGATATTTTCAGGCTGAAGCATCCACGCAACATCCTCACGCGCCCAT
TCTTTACCGTCAGAGAGTCGTTTTAACCGGGGTTTCCATATTCCATACTCATTAAG
TTTTTCTGCTACCGAAGCATATTTCTCACGATCTGACTCCCCCGTATGAAGCTTGT
CAAAACGACCTTTTACAAGCTGGTAAACAGTATAGATTTCCAGTGCCTTAGGGGA
CCTGGCCGGGACTATCATGGGGTGTTCCTCCGGCCTATAAATCCCGTTTGGCTCT
CCCTCATTGAGCAGGTCATTAAACATGTGGTTCACGCAGGCACTCAGCACATCTG
CCGCTTTCTCCGCAGCGAATTTATCCGGGCTAAAACCCGCTCCCGTCAGTTTCCG
GGCAATCAGTGCCAGTTTTTGCCGGGTATCCCGTCCCAGATAAAGCTTCTTCTCC
AGAAATTCAAAGACCTCGTTACTGCTTTTCCGTTCCCGGTGGCATCTGGCGTCAG
CCTTGTTGCTTTGGTTACCAATCATAGTCACCTCCATGACAGTGTTTTGTACAAAA
TATCACCATTGAGCCTCAGTGAAAATTGCTTCTTTATATGACCGTTCTGGAGACA
GAAATAGATGATCTCGAATGACATCCACATTAAAGATGCCCTTAACGTGCTCAAC
GAAGGCATCGTTGAGTGAATCCTTGCGTTTTTTGGTGGTTGTAAATAACCCGCTC
CTGAAACAGCTCAAAGGAGCACAAGCATGAACACCAACCGTATTTTGCGTAAAA
AAGAGGTACTTCATCTGACGGGGAACTCGTCAGCGACGTTGTACCGACTCATCTC
GAAAGGATTCTTTCCTTTGTCAAAGAGGCTAACGGGCGACAATGGCCGGGCGGT
AGGCTGGCTGGAGAGTGATATCAATAACTGGGTTAACAGCCGTATGCAGGCGGG
AGAATGAAATGAAAGAGATAAAATCGAAAACCAGTTCTGCGAAAAAAACGCTG
GTTCCTTGGTTCGAAACTATGGGGAAACAGGTTGGTTTATCACAAGAAACGATAT
CAGCAGTGTTAAAGGAGCAGCGTTTACGGATACGTAAGCTTAACTACGCATCTG
CATATACAGTGTCCAATATTCTCATACAGGATTGCGGCGGGGCCGATGACATGTG
GAGCAACTACGCTAAACTGGCGCTTATCCTTGAGCGTTGTAAATACATTCCCTGC
GGGAGCAGGGCCTGTGCATATTGTTCTACCGCTCAGCAATTACATTATCAGGGGG
TGGATGTAACCGGGGAATACGCTTTATATCAGTTGCCTGAGTTATTTTTAGATTTT
TTAGAAACAAACGACAAAAATTCCTCTTATCATTGGGATTACAATAAGTTAGTTG
AGCGGCATGAAGATGCTCAGGCGAGGTCATTCGGGTAATAGCCATTTCTGTATG
GTGAAAAACTGACATATTACCCTGCCACTGCATTTTGGTGGCAGGGTAATCAATA
CAGCTAGGAATATTAAATGAACGATTCTTATTACTTCAGAACATCTGACCCGGAT
TACATTCGTCACGCACCGTCAATGCTTGAGATTTTAAGTAAAGCATTCCCCACTA
TTATTACCCAGCAGGAAAGTGAGTATGAACTGCAGCGTCTTAACCATTTACTAAA
TAAACTGAAAGGCAGCAATCGCTGCTACAACATCATTGCTCAAAAACTAAGGCA
GTGCAGAAACGGATCTCCATGCAACAGTCTTATATGCCCGCATTGCCAGCGGGA
ACGCATTCTGGCGCAACTGGCTATGTTGCATGTCTTGCCGGGGAATTCAGCAGAA
TACGTGGGAGTGGTCCTGTTTTTCAACAAGGATACGCAAACTCCGCCGCCATGGA
AGAACATTGGGGCGCTCAGGGCGCAAATCGGTCGTTATAAGCAGAGGATAAGCC
GGGTACTTAACCGCCTGGGGTACGCCGGTCCGGCCACAGGCACCTTCAGTATGAT
GCGACATATGCCTGATGGCCCAGAAGAGCGTATTTTCTGGGTTCCACAGTTGTGT
TTGTTCCTGCCCAATGACAGTACGTTGATTAAGGGGCTAAAAGCGCATATGTCGC
GAAGTGGTGGGGCATTCATTGATGCATCGACGCTTAACACACCAGTGATCGTGCT
CAGGTATAAAGATCCCGCGAGACTGATTTCATGCGCCCTGAATCCAGTCTGGCAC
ACAGCAGATTACACACTGACTGATAAAGACGCTCTTGTTAAGTCAAGAATGGAG
CTACTGAAAGGCCGGACACTGTTAAAAAGTCTGCTTACCCTGGACTCACTCGGAA
CAGGTGTCGTTTCTTTTTCGTTTGGGCAACCCCCAGGGAAAGTGCACTGATTTAC
CGGTAATGCAGCAACTGCAATGCACTTTTGCTGTTGATTAAATTGCAGTGGAACG
ACGAATAACAGCGGCAACCACTGCATTTTCGTTGTTGTAAAACTGCAAAGCTTGC
CAGTGATTTTGATGGCGCTGATGCACTTTAACATTCCAACTGTCTCGATTTTAACG
CAACCGTCTGTTTTCTGCAGGGGCTCACGGATGCCTCTGCACACCCGATCCTTAC
CATCAACTGACGAGTTAGCGCATCTGTGAGGGCTTTCTGTGTTTCTGTCAGTGAA
TATTCTACACACAATGGGGGGAGCGGTCAGCGAGCACCATTGCCATTGAGCGCA
ACGGAGAGCTTGGGGACATGCAGCACTACGGAGGCTTCATCTCCTTCTCATGTCA
TTAAACGATACGGCGAACGAGCGCAGCGAGTGAGGGAAGGCGTCAGCCTGAAC
AGGGGGAAAGATTTGCGACCGGAGTGGAGCAAATCATGGGCGGAGCCCATCCTC
GCGACTGGGGTTTTGACTTTCGAATTGCCCAGCGTTGAGTTTGGTTAATGTAGTG
AGACGAGGATGCTGAGAGGGGACCGTGAGTACTGAAAAGGATTATCCAGTGAA
GATCCCTAAGTGGGTTTATGACCGTATTACTGAAATAACGGATTATGTCGTGGGG
GATACGCAATGGGCTGTTACCCGGCGGCAGACAGTACGGTGGTTCCTGGCTCAC
ATATGGCTGGAAACAGACGATGATGGCTGGACTATATGCACTGTCCGGGATATC
CGTAGCAGCTATGCCAGTCTCCTGGGACAGTGTGAAATTAGTTACCAGGGGGAG
TGCTTTATGTCCACGCTAACTGATTTCCTGCCGACACTTTCGGATATTGAATTTCG
TGCAGGAAAAGCGAGCAAAGACCCGGAAAAGCGTAGAGCCAGTGCATGGCGGT
TCAACCCCCTGCTGCCTGTGTGTGATGAGTCTGCCAGGGGTAAGTTAAATCTGGT
TGACTTGGATACTGGTGAGTCTGTTAGTTTTAGAGCTCTGCTTCAGGGGAACGGG
AAAGCTCCTGGGCATGCGATAGATGTAGAGAAACGCCAGACGGCCCTAAAACAA
ACGGAAAAGGACTTCTTGGCCAAAGTTGGGCGGGGGCGCATGAGTATACAGTTT
GTGAAGGAATTGCGTAGCCGTGAGCCGGATCACTATTACCGCGCAGGAATACGC
AGCCTTAACCATCTCTACAATGGCAGGATTGAGGGACAATATGTCACTTACGACC
ATCATTATCGTCTGACGTTTGGAGGGCGGTATTATGATCAAGCTTTCCAGAATCT
GCCCAATGAGTTCAAAGCTAAATTTCGGACCGGGTTACTCAACTATGACATCGAA
GCGTGCAATCTGGCCTGTCTGAACCATCTGTTCAGACAGTATGACGTCGATTATC
GTGTGAAGTCATCGATATATAAAACCATGATGGAACATACCGGGCTGACCCGTA
AACAGTGCAAACAGATGGTCCATACCACCACCTACAGAATTGGGCGTGTGACTA
TTGGTGTTAATGATGGCCTTGGGGCAAAAGTGTATGGATGGTGCGGTAACAGCA
AAAAAAAGGCGCTAAAGATTTTGCGTTGGTGGAACCGTTATATCAGTCCATTGAA
ATCTGCGCTTGAGTCATTGCTGGAACGTGTGCATGGCGCACACCGCAAGAGTTGT
TCATCACCGCGTAACTATCACAGGTACGCTAATGAAGTCGGATTAATACTGGATC
TTAATAGTGAGGTGTATCAGCGTGAAAAGACTCACCATCATCAATATGCGCGGA
ATAAGGCACTGTTGGCGTTTATGATCTGTGGTGTGGAGCAGGCGTATATCCGTGA
GGTTGTCAGTATGAACCCTGGTCGTGTCTGCATGCTGGATCATGATGGGGTAGTG
GCAACGGGCGCGCTTTCACTACCGGACTGGCGTGGTTTCACGATGAAAGTGAAG
GACTAGCTTACTTTCTTGAATTTAACTATCTGCGATCTCTGATAAGGGCGTATTAC
GTTGTCAATCATAATGGGATATGCGCATTGGAACGATGTGCATATCCAGTAAGTG
ATCCATGTCCGCTTTGAGCTGTGAGTTCAACGGGTCGATGCAACGCTGTCCCTAA
TCAAGGGGGACGTTGCCATGAAACGAAGAACTCGTATTAACTACACGCCAGAGC
AAAAGGTGATTATTTGGGACAGATATAAGCAAGGTGATTCCCTGCATGATATCCC
CCAGAATGTTCGACAGATTTCACTCTACTATCATGCCTACAATCCACCAGGCCGG
TGGCTACCGTCCTCCCGTGCGAAAGCGGCACCGGTTAGCGTTTTCGCTTGATGAA
AAAGAGGAGATATCCAGAGGACTGGTAGCAAAACGCATCATCAGGGACATCGCT
GCTAAATTATCAATAGCTCCCTCAACGATTAGCCGCGAGATCAGGAGGCACGGC
GGTGCAAAACAATACCGTGCAGCAAAAGCCGATCCTGCTGCGTGGGAAAATGCT
CTGAGACCAAAACCTTGCAAGCTAATTGAAAGCCCCACATTGTGTAAAATCATTG
CAGAGAAGATGCATCAGGACTGGTCGCCGGAACAAATTGGCGGTTGGCTAAAAC
GCTGTTATCCCGATAATCAGGAAATGCATGTGTCACATGAAATGATTTATAAAAC
GCTTTTTATACCAACCCGGGGAGCTTTAAAAAAAGAGCTGCAGCAATGCCTCAG
AAGCGGAAGAGTAGTGCGTAGATCCAAAACCTCATCACTTAAAGTAAACAGAGA
TACATTCTGGTCACGAATGCCCGGTTGTTCGTGCTTTGCGTTTTAATCGCATTTTA
CAGTTTCTGCCTGCGTGCAGAGGTTGATGGTATTTTCTGAAAAAGCGGCTCGCTT
TGCTAATGCATATGGCACAAATACAACAGTTTGTGTCATTCTGACCCATCTTTAT
ACCGCGACGGTGGAAACATGGATGAGCTGCTAACCCTTGGCTGCATTGCTTTATT
CTTCGCACTGGTGGTGGTTCCCATCCTCGCGATTATCGCGCTCAGCCGCAGCACG
GCTGTTCGTTCTGAGCTGGCGACCCTGCGTCGCCGGGTGGAGGTGCTTGAGCAGC
GCGGTGTGACTGAAGCACCCGCCACAGTGGTAACAACTGTGCCGCAGAGTGTTT
CGCAGATGGTTGTGCCTGTTGAAACGTCTTCGCCTGCGGTTAACATCGTGCTGTC
GGAGCCTGTGGCTGATGTCACGCCTGAGTCCGTTGACCCCTGGCGTCCACAACCG
AAGCCGCAAAACGAGGCGCTACCCACACCAGCGGTCGAACAATCTTCCGCTTTT
GGTGGGGTGATGTCGTCCCTGGTGCGCTGGTTTATGCAGGGCAACCCGCTGGCAA
AGCTGGGTATTCTGCTTCTCTTCCTTGGCTTCTCCTTCCTGCTGCGTTATACGGTT
GAACACTCCCTGTTCCCGCTTGAGCTACGTCTGGTGGCGACTTCGCTGTTTGCGA
TTGCCCTGCTGGTAGTGGGCTGGCGATTGCGGCATAAACAGCTGGTTTATGCGCT
GATCATACAGGGTGGAGCTACGGGGACGCTCTATCTCACCGTTTTTGGCGCATTC
TGGCTCTGGCAGATGCTGCCGATGACGCTGGCGTTTGCATTACTGGTGGCGATTT
GCGCGGCGAGCGTGGGACTGGCGATTATGCAAAAAGCGCTCAGCCTCGCGATGC
TGGCAAGTATCGGTGGCTATCTGGCACCGCTGTTGCTTTCCACCGGCGGCGGTAG
TCATGTGGCGCTCTTCTCTTTCTACCTTCTGCTTTCCGTCGGTATTCTCGCCATCAG
CATCTGGCAACACTGGCGCGAGCTTAATCTGCTTGGCCTGTTATTTACCTTTGGTA
TCGGCGGTCTTTGGGGACTCGACGGCTATCGATCAGAGTATTACCTGAGCAGTCA
GCTTTTCCTGATAGCCAATACGATCATTTTTGGTGTGCTGAGCGTGGCGCTATCG
CTACGAGCACAGGAGAAAGGTAAGCAGATTATTGATGGCGTGCTGTTGTTTGCAT
CGCCGCTGGTCGGTTTTGGCATGCAGTACGCCATAACCCGGCATATGGAATATGG
TCCGGCGCTTAGCGCGCTGGGTTACGGCGGTTTCTATCTGGCGTTAGCATGGCTT
GCATTGCGTCGCTATCCCTCTTTGGGGAGGCCGCTGGTGCTGGCGGCGCTGGCGC
TGGGTGGGGCATTTACCACGCTCGCTATCCCACTGGCGCTTTCGGCCCGCTGGAC
GGCGATGGCCTGGGCGCTGGAAGGTCTCGGCATCCTCTGGCTGGGCGTGCAACA
GCAGCAGCGGCGTATGAGCTATAGCGGCACTGCGCTGCTGGTGCTGGCAGTGTG
CAGCGCGCTGTGGGCGCAGATGAACGGGATGAGCGCGCTTTCTCTGGTGCTAAT
CTTTGCCGTACTCAGCCTCAGCTGGCTGGCTGCCGCCTGGCTGTGGCGCAATATT
CAGTTGCAGGGTAGTTGGGTTCTGTTGGCTGGCGGTTTGATCTTCTGGATAATCG
CGCTCATTGGGGCATCGCAACTGGTGCTGAAAAAGGACTCGCTGGTGCTGTCTGG
CGTGCTGGCGTTAATGGCGATATCGGTCTGGGGCTGGCGGATTGTCTCTGGTCGT
CTGGCCTGGTGGGAGCTGGATGTCAGCAAATGGTTGCTGTGGCCGACGATGCTG
GTGATGCTGCTGTCTCAAATTTCACAACATGAGATTTTTGCTGCGGGCTGGCAGA
ATCTGGCCTGGTGTCTGGCGTTACCCGCTGCGGGGGCATTGCTCTGGCGTGATGC
TGAGACGTTGCCGCCGCGCCTCTCCAGACTGGCGCACCTTTCACTCTTCTGGATG
ATTCTGCTCGCGCTGGCGGCGGAGCTATTCTGGTTTGCGCAAGATCTGCCATGGG
GCATGGCGGCGTGGGGCTCCGGCCTGATGATGGCCGCGGGGGGCCTGCTCATCT
TTCTCGTTCATGAGGCGGTTCATCGTCAGCTATGGCCATTCCGAAGCTGGCCCGC
ACTCTACGCTTCTCAGGCGATGATACCGGTAGCTGCGGTATTGGTTGTTCTGCTA
GTACTGACGAATTTGCAGGATGGCGTAGTTTATCGCCAGACCTGGCTGCCGCTGG
TGAATCCACTGGAGGAGGGGGCCGCTTTTGCGCTACTGGGACTCATTGTATTTTA
TCGCGTTTCGCAGCGCTTTTTCCCGGTGCAGATTTCGGTATGCCGTCCCTGGCCGC
TCATTGCACTGCTGGCGCTTAGCTTCTGGTGGCTGAATGGCATCCTGCTGCGCGC
ATTGGCCTGGTACGGCGAAGTGCCCTGGAGCGCGGAAACACTGTGGCATTCGCG
GCTGATTCAGACCTGTTTTGCCCTGTTCTGGATGCTGGCGGCATTAGTGGTGATG
CTACGTGCGACCCGGCGTTGCTCACGTCGCGAATGGCTTTGTGGATCCGTATTGT
TAGGGATTGTTATTGTGAAACTGATGTTGGTAGACAGTGCGCGTGGCGGCGGCCT
GGCACGCGCGGTCACGTTTATCGGCGTGGCAATTCTGGTGCTAATCGTCGGGTAT
TTTTCACCGTTACCGCCCAAAGCGGGAGAAGAAAAATGAAATGGATGAAAGCGG
TGTTATGCAGCGTGCTACTGAGTACTGCGGGCGCGGCGGTCAGCAGCGATGAAG
TGAAGGAAGCGCCAACGGACTACGCAACAGGCGTCGCGCTGGAAACCACGGGG
GCATTCGCCCTGGTATCGCATTTCGCTGCCGCAGGTGGTGTATCAGAGCACTGCA
TGGCCGGATTTGCGCGATGTGCGGGTTTTTAACCATCTGGGGGATATGGTGCCGT
TTGCGCTGGTGGCGCAGAAAACCCAGACCGTTACGCCAGAGACAGTGGCTCTGC
GTCTTTTCCCGCTTGATATGTCGCCGGTTCCGCCGCGTGATGAGGGTCAACGTAG
TGGGGATGCTGTTGTTTTACGTTCAAAAAACGGTATTGAAATTCATTTGCAAAGC
GATGATGTCACTGCGCTCGGGCAGAGCTATTTACTGACGTTGCCGGAAGAGAGG
AAAGACTCTCTTTCACTGACGCAATTGCGTCTGAACTGGGGGACGCCGACGGGC
AACTGGCAGGGGAAAGCCTCGGTTTATGTCAGCCGTGATTTACGCTACTGGCGAC
CCGTGCAGGAAGATGCGCCGTTAATGGATCTGACGCGGGATAGCGATCGTCTGA
AAATGGACGCCATCAGCACCAACCTGACATTGTCACTTGAAGGAAATCGCTACC
TGCTGGTGATCCTCAACTCGCAAAGCCCGGCACTGACGTTAAACAGCGTGAGCG
CTATTGCTGACAGTAATGAGCCGGAGTCCGAACGTATTGTGATTGGCGCTCGGGC
GGATAAGGTGTCGGATGATGAAGCCGTCTGGCGATGGACACAGCCGCAGCCCCT
GACATCGTTGCGGATCGATCTGGAAAATGAAGGCGTTTTACCGGTGGAACTGGT
CTGGCGCAGCGGCGAAAAAGAACCCTGGCAGTCACTGACAAAAACGGTGTTGTA
TCGCCTGGACGGGAAGCGTTCAGAAGATATTCGTCTTCCTGGCCAGTTAGTTGAG
GCTGTTCGAATACGTACAATTAATGCGCGATTGCCGGAGGCATTGCCAGCATTGA
GCGGTGCGCGCGACAGTTATCAACTGGTTTTTAACACGCAAGGCAAAGGTCCTTA
CATGCTGGCGTGGGGAAACCGGGCGGCGAAAAAGGCCGATGTTGGACTCGATAT
GTTGATCCCTGCGTCGCTGCGTAAAACGCAGGAGATAGATAATCTGCCGTGGGCT
ATACCGCAGGAGAGCGTAACTCTCGGCGGTGAATTGCGACTAACGGCGACGTCG
GCAGCCGAACAACAAAGTCAGTGGAAAACGCTGCTGGTGTGGGGGGCGTTAATA
CTTGGCGTTGCCGTTCTGGCGTTTATGGCATGGCGAATCTGGCGAGAAGTTAAAA
AGGACGGTGCGGCGTAAGCCATAAAAAAGCACAGTTGCATAGTTCACGGTGTTG
ATAAACTGTTACAACAGAGGCGTTTAAGCTGAGGCGGGGAAATAATCTATTTGT
GAATAATATGTTACTTCACTGGGGCGTGAGTCATGGGAATGATTTTGATACCTGC
GTTTTTTATTTTAGCCATCCTGATTATCGCTATCTTCATTTCAGTTAAGAATGGCC
GTCACGTATCCGTTGTATGCCTTGTTGCCGTGGGTGAGATTTTCGTATTCCCATTC
TATTTTACGGCCCTTAAGCGTTTCCGGAGCATCAAAACTTGGTAAAGATTTACCA
TGTTGACCCATTATGCGGCCCCTCTGGAGACCACTTTCAGGTATTTTCTCTACTGA
TATGTACACATGGAGAGATGTGATTGCAAATTCCCATCAGCATTACTACCAAAAG
AATCAGACAGGAAGCAGCATAGTATTAATAAGAATATTTTCAAACATAAAGGAT
GATTATGAGTAACCCAGGCCCACTTGCAGCGTTAGTGTATGGTTTTATTTTGATA
GTTTTGCTATCGGTATCTTTAATCCTTTATTCAATTGGACGTACCATAAAAAACGA
GATTGGAAAAGGTATAGCATACGCGCTTACTGGTATTTTATTAATTCTGTCATTAT
TAGTTGGCGCTTTCATTTTGACGCTTCTTACATAAATTCCTCGAAAAATACGCTGA
CAGCATCAGCTAATGGACCCGACTCTCCGTTGAACGCATGCTCGCCTGCGGCACC
TGTGCCATGGGCGTCCGCCGCTACGGGCAGGCATCAAGGTTCGCTTCACAACAG
CGGACCTGCTGCTACAGCTATCCACTTCATAGCGCCAGGGCCGTTACAAAACGAC
ACTCAATCGCGGTGTCATGGCCCCGAAGCTACTTATCATCGATGAAATAGGTTAT
CTGCCGTTCAGTCTGGAAGAAGCCAAGCTGTTCTTCCAGGTCATCGCTAAACGTT
ACGAGAGGAGCGCGATGATCCTGGACTCCAACCTGCCGTTCGGGCAGTGGGATC
AGACGTTCGCCAGGGATGCAGCCCTGACATCGGCGATGCTGGACCGTATCTTAC
ATCACTCACACGTAGTCCAGATAAAAGGAGAGAGCTATCGACTGAAGTAGAAAC
GAAAGGCCGGGGTTATACCTGAAGCTAATCCTGAGTAAACAAGGTGGATCAATA
TTAAACCGTTGGTGGTGACGGTTAGTGGATCACTTTATTACCCGTTGTTGACAGT
AAACGCAGGAAGTCAGATGTGATGCGCCGAAAGCTGCTTTCGCACATGTTACAG
GGACTGGAAAGCCGGGCTGTTTACGATTACGTTGCCAGTCATAAGGGCGTTTGCG
CACTGGAGCACGATGGGTTTTTTTCATATAAAGAGGTCACTGACTGGTCCCATCC
ATATCTGTTGATTGAGAAAAAGCATTAAGGGGAGCCACTCAGTCAATTACAGCTT
ATCTCGGTTGATAAACAAGACGGGACATTTAGCACTTTCATGATATGCATAGCCG
GCAGGTGATCTTAGCCTGCTTTCAGCGATGAACCACACAATATTCCCCTTTTGTT
ACTTTGATATGATTGCTCAAAATGGGTGGTAACACTGAGATCGTTGAAATGACTG
CATGTTATATTGTTACTTTTGTTACCGTTGAAAACATCACGTTGGAGAGCTAAAT
GGATTGGCCAAAGGAGTACAGCAAGACGACTCAAGTGGAGTGACCACATTCCGG
TAGACAGTTTCTGTCCTCACGGTGAGGCCTTTTCGAAGGCCTCCGGGCTGACGCC
ACCCAGATGACTGTGGCGCCGGGTCCGGTTGTAGAACACTTCAATGTAATCGAA
GATATCCGCCCGGGCCATGTCCCGGGTTTTATATATCCGCTTTCTGATGCGCTCTT
TTTTCAGTGAACTGAAGAACGATTCTGCCACCGCATTATCCCAGCAGTTGCCACG
ACGGCTCATACTCGGCGCCAGATTGTTGGCCCTGCAGAACCGCTGCCAGTCATCA
CTGCCATACTGACTGCCCTGATCGCTGTGCACTATGACGTTTTCTGCAGGCTTAC
GCCGCCAGACTGCCATCAGCAGCGCATCCAGTGCCAGCTCGCGCGACAGCGTTG
GTTTCATCGACCAGCCCACCACATTACGGGCAAAAAGATCGATAACCACCGCCA
GATACAGCCAACCCTGCCAGGTGCGGATGTAGGTAATATCAGTCACCCACACCT
GGTTGGGTTTTACCATCGTAAATTCCCTCTGTACGCGATTAGGAGCTATCAGTGA
CGGCCTTCCCCTGACGCCACGCGGGACCTTATAGCCATGTACGGCCTGTATCCTG
TTCCGCTTCATGATTCTGGCAACACGGTTTCTGCTGCATACTTCACCGATTTCACG
AAGATCGCCGTGAACCCTGCGATAACCGTAGACTCCGCCGCTCAGTGCATAAGC
ATCGCGGATGAGCTCCAGCAACCGCTGATTATCCTTTTCTCCGGCAGAAACAGGG
CGATGAAGCCAGACGTAAAATCCCGCACGGGCGACCTTCAGAACCCGACACATT
GTTACGATCGACCAGATTTCACGGTGATCGTTGATGAAGCGGTACTTCAGTCGGG
CTCCCTTGCAAAGTACCGCGCTGCCTTTTTCAGAATATCCCGTTCTTCCTCAGTAC
GTTTAAGCTGCGCTTTAAGCCTGAGAATCTCCGTCCGCGCATCCAGTAAATCCTG
CGCCTGATGCCCGCTGTTATCCGGTCTGACTGACCGGACCCACTTATAAAGGCTG
TGAGCCGATACGCCGAGGCGTTCAGAAACATCGGCAACGGAATAGCCCCGTTCA
GTGACCTGACGGACAGCTTCTTCCTTAAACTCTGGGGTAAATCGTGGTGTGCCCA
TAGACTCCTCCTATGCTCAAACTATAGGGCAGATTTGTCTACGGGCTTGGGGGCA
CTCCAGAACCTTGAGGAATAAGTTCTATCCTCGTGGTTCACTTTGTGCAGCAAGT
TC
ST131 Region-6 (SEQ ID NO: 17)
TTAAAACGCGGTTGGGTGAATTCCATATTCACCGTTTGATCCATACCCTATGCGA
TACATCAAAGTACGATCTGTTGTCACCTTGCCCGCTTGTTCGCTCATGCCACCACC
ACATACTCCTTTTGGCCACGCGCTGAAGACATGATCACCAATCGTTGGGTATACA
ACGACTTTCTGAGCTGTACCTAAATCTGCCACCTCTTTACCATCAACATAAACAC
GGGAAAAACACGCGCTTCCAAAGAAACCCGAGTCACGCTTGATGATCACCTCTC
CCGTACCGTCTCGTTTTTCGAGTAACGCGGTACTGATAATTTGTTTTTCAGGAACA
GGAGTGGCTTGTTCATTTGAGACAGGTTTTGTAGCACAACCGACAAGAGCAAGC
ATAATCCCACAGGCAAGACCATTTTTAACGACCATGTTTGAAAACTCCCAATATT
AAAAATATGTCAGCATCTAACGATAACAACGTTCAGCACCAGAGACTCTTCGTTC
TCCGGCTTACGCCTAAAAATATCGCAGGTGCCGTTTGCAGTCGACATCTGTTAAT
ACTAAATTCATACCGCCACCGGCTCGCCGAGCCCATTTACTCCAGAGACAGCCGA
ACCGAAAAACCGTTTTCAGCGAGCGTCAGACTCAGTGTGTTGATAGTCCAGTCCC
GGTCCTCCTCGGTCCCGAAACCTTGAGTTGTGACTTTGCATTCTGCTGTCAGCGA
CACTAAATCGAGCGTCGCTGGCGTCGTGTACGTCATAGAGAACTCTTTTTTAGCT
GCACCGCCCTTGAGCCCTGGTGCAGCTTCTTTCGCTGCGTCGAGCGACGGCTCAA
CTGCCGGCCATTCAATGACAGGATCGCCGCCACTACCTTGTCCGCAATTCTTTGA
TCTGCCCAGTTGCTGTGTCGTGATATTTAATCAGATACGTCGGTTCCGGATTTTCG
CCACCGCTACCACTATCGCCACTTTGTCCATTTCTCGAATAGCCCCAGCTGGAGT
TATCACGACGAGTTAGCGTGTAGTGCTGGAGCGGAGCACCGCTAACAGATTCGC
CCGCGCCGCGCGGCAGGAATATCCAGTACCCGCCCGCGGGTTTACTCACCGCATC
GAATCTCGTTGCGAGCCGCGACATCAGGTGCATATCTGACTCGCCGACCTGATCG
AGTTGCGTGATAACATTGTCTGCAAACCGCTTTGATACGCGTGGTTTTAGCCCAT
TATCGCTCGCTATTGTCGCAACGATATCGCTGACAGTGACGTCGCTCCAGGAACG
AGTCTTTTTGCTCTGAACTGTCGGAGATCCTTTTGCTGCGTTCATCGGAGCCGCTT
TTGCTGTGAAATCAACTACCCGGGGTTCACCGCCACTGCTAGCACCGTCAACGAT
GTATATCCCTTTATCAATTATCTGATTACCGAAACCGAGCCCAATACTGATTTTTA
CTCCGCGGGCCGGAATTTTCAGAGTTTCAGAAACTATTGCTACGCGGAGTTCGTC
TGTCTGTTTACTGGAGCCGCCGTAATCTGTCAGCGTGATATTGATCAAACCTTTTC
TTATTTTATCCGTGATGTTTTTATTATCAGCAGATATATAAAAATTAGGTTTCCAC
GCCTCCACGCCAGTATTTACGTAATCACTCATTCATCAATCCCATAATTGCGTTTC
TTTTTTAACCGGCTCTGGTTCGATATCTGGTAAAACAATTTTTTCTCCAGCACGAA
ACACGGCACACATATCCGTTACGCCGTAATTCGAAACCTGATATAAAACAGTCTC
TGTCGTTTTTGACGCTCTCCCGTAGACCGCAAGGCAGATCTGATCGAGACGGTCA
CCGTCCCGTGTAGTGTATGTAATCATTTGGGGCCCCATTAATTAAAAGGATTTCA
TAATGAGAATTAATTTTAGTAGTCGGAGAGGTATTAGTATTTCGGCGTCAAAAAA
AATACCATCAGGTTGTTTTACATTTATGTTCGTAGTTTTTTTTATTATTTTTATACT
TGTTAAGTGCTCTGGTAGCGCTTGATTTTGTATTAATCTAGCGAAGCTCCATAATA
TTCGAGCGTGAGTGAAAAGTCCTGCGATTTAGGTGTTCCGCCCATCAAAAAGCTG
TTTTGCGTTTCAGTTAGTTCTCGCAGCACCCAGTACCCTTTCACCTCCCCGGTCCC
CAGCACTAGCTGCTGGGGTTCTCCCGTGTTCCCGAGCGCTCGCAAATCTGTCAAC
AGGCCGACGCCGCACCCGTCAAGAAATAAAGCGTTTATCTTTCCATCGATAGTAA
TTGTGGGGTTCGGTCGTCCGGTTATCTGCAACGCGTCGTTTTTCCCGAATCGTGTT
TGCTGAGCCCAGCGCCAGGCGTCACTACGTTGCAGTTTGTTGTACGCTACTGTTG
ATATAGCAAAAATGAAATCACCCAGGCCGAGCATGATATCCTCAGCACCGTTTA
TTGCATCACTCATTGTCACCCCGCCGCAGCTGGTTTATCAAACATCGCGCCACGA
TTGTACGAACTCGCAGCCTGTCTCAGTCCATCGAGCACCCCCGACGCTGCAGATT
GTTGTATTGACTGCGGATCTGTCGCACCGACAATATTGATTTCGATTTTATTGTTT
TGCTCCGGCGCAGGTGTCGAGACTGCCTGCTTCAACGTATCAATATTTAACGTCG
ACGAATCCAGTGCTGCAGGCGCGGTATTTTTATTTACCATCCCGGATAATGGATT
AACAAACGCGAGCATTGATTCTGCCTGAGATTTTGCCTGTTCTTGCCGTTCTGGA
TGCCGCATATTTTCGACAGCCTCTTCGGCTTTTGCCTTCGCAATATTATCAGCAGT
CAATCCAGCGTTCGGTGAATATTTACCTCCCGAATCCATAAACTCTTGATATGCC
CGATTATATTCGTCATTATAAGTTTTCTGTTCTTCATCTGATTTTTCATCCGGAATA
AGCCACTCCAGTTTCTTTACGACAGCCCATATAATCTTCCCGAATTTGACGCAAC
CTTCTCCAAAATTGACGATGCCATCCCACATTTTTTTCATATTTTCTGGTTTTACC
CACTCAGTAATTGTGCTGACAAAACCACCCTGATTATCCTTAAACCAGGATGTAA
ATGTTTCACGTACTGAGTCAAACGTCGGCGCGAGTTCCCCCCCGATTTTACCGAG
TGTATCTGCAAGTCCCGACGTAATAGAGCCCCACAGATTTGTCACTGATACATGC
GCACGCGCCGCCCCTTCCGCACCCTCTTTTGTTAACAAATTCGACTTTTGCGCGTC
AGACATTGTCTGTTCCCAGCTTTTACCCGTCGCCTTCATATACGTCATGATCTTAT
TAGCCTCGCCCCCCATGAGCTGGTCAGCGAGGCTCGCCGCTTGCTTCTCATCTTTC
ATTTCAGAGATGCGACGCATAACCTCATTAAACTGTTTTTCCCGATCCCAGCCAG
CCATTCGTTTTTTCGTCAGGCCAATTTGAAATAACATGGGATTCAGGCTTTTTTCG
TTTCCGATTTCGCCGATTTTATTTGTAAGTTCTTCAGACAAATCCCCGATATTTTC
GCCGTTCAGGCCCGCGGCTTTACCAATATTTTCCCACGCAGCGTATTTTTCGACG
CCAACGCCGTAGGACTTCGCCAGACCGAGTTTTTCTGACGTCTCTGCATTCATTG
CCAGCGCACCAGCTGCAGCACCGCCAATGAGCCCTGCCGCGCCAAGTGTCCCCC
ATTTGATACCCCGCCCCGTAGCCCTCACCGCACCTGCGGCGGCTCCTCCGCCCCA
TTTTGCAGCTCCTTTTGCGCGACCCGTCCACTTCTCCAGCCGCTGTTTTTTAGCAA
GTTGAGTATTCAGCGCCTCCTGGTCCTGCGTTGTCGCTTTTATTCGACGGTCCAGC
TTTTGATACTGAGCAGATAAATCAGAAACGTCTTTACCTGCCAGTTTCATTGCTG
CGATTTTGTCTTTTAATTTATCCTGACTTTTCGTTAATTTCCCGACAGTAGACGAC
GTCTGCCGAATCGCATCATTGAGTTCTGTTGTACCGCGACGAAACGACGGGTCTA
TTCGCCCACCGAATACAACCTGCGTTTTAAAGTTTTGAGAAATAGCCATCGATTT
TCATGACCTCGTTTACATAGAAATTAAAAACCCGGTACGGCAGGTCCATTTGCGT
ATCGGGCGGGATGGCAAGTTTTTTCGCAATAAACGGAATCAGAATAACTATATCT
GATGTCGGTCTTTCGGAGGCTTTACCAGTTCGTTAAATGCGGCCTCCATTTGATC
GTAATCGCACGCAGGCAGTGCATATAAATCCTCACGCTCCAGATTTGCCAGGCGC
GCCAGCATTGTCGCTGTCCGCTCTTCAAGACCACTTTTATCGTTACTGAACATAAT
GCGGTCGCGTAATTTTGGTTCGCGAATAGTAATTGTATCGCGGCTCTCCCCCTTA
ATAATAAACGGGCGAGAGAGCGTAATAGTCATACTGTCTAATAATGATTCCATTT
TATTTGCCTTTTAATATACATGGAGAATACGAGCAACGCCCGCAAGAACGTCAAC
ACCATTAATTACTCGTTTCATTTTTGCAGGATGAATCTCGTAAATAATTTTTCCAT
TTTGAACTCGTTTATAATAGCTCGGCGCAATCGTTACTGATTGCCCAACTGCTGA
TTTTGAATCAGTCCCCTGCGCGTCATCAGTAATCGCCGTAATAAGCCCCTCATAT
GTATCAATACGTTCAAAATTGACACCGTAATTGTCAGTATATGCACTTCGAATTT
CGAACCTTGTTTTAGCACCTGGTATTAATCCCAAATACCCCATCACACCAGAATC
GGAGCCAGAGACTTTAAAACTGGCTGACATTTTCTCCATTCCCCCGTCCAGAGTG
ATAGCTGTATCCATAGCGCCCGTCTTAAAATCATCTTCGATTATTTTCATATCGAC
GGGAGTGTATTCAGTGCAATTTAAAACTCGTTCTCCCTGGACGAACAACGCAAAT
GCACGATAGGTGTTACTATCTGCCATATCAATTCTCCAGATTATTAGACTGTTTCT
TTAAAAAGAGAGGCCAGAGCTTCAACTGTGTAATCATTATTAATGCGGTACATTA
ACGTAATTGTTTGTGCCGGGGATTTCGGTCCAAAGTCAACATTAATATAAAGATT
ACCAGCGGCCAGACTTTCTTTTGTATTTAACTCTTCATCTAACCACGCCCGTCCTC
CGTTAATCGCGCCCTGCTCCTCTAATTTATGCAGATACGCGTTTATCGACTCTACA
ACGTCTGTTCCGAGGTGCAGGTCTATCGGGCGATCAATAAAATCGGTCATCATTG
CCCGGGCTATTGAATCCTCAATTACGTCAGCTGTCCGACGAACGCATTCAAAGGA
CCATTGGGGACTGAGGCTACACAGATAGTTACCCCAGTGTCTGAATCCATCATAG
CGAATAATTGTGCTGACCTGGCTGGCGTTCAGCAGATTTGCTGTGCAGTTCGTTT
CACCGATTATAAAATCATCGACTTGTTCGAGTCCTGTAATTCCGTAGATATTTTGA
TTCGATTTGCTCCACCAAAACCCTTTTTCGTTATCAATTCTGGCTCGTAAACCAGC
AGCGATAGCTGAATACGGACGAGATAAACCACTAACATCACTCGTAGAATAAAC
ACGAGGGCGCAGAATTTCCATTCTGCCGCCGTACCTCTGACGACGATTTACAACG
TCAGCAGGCGTAGCGCCTCGCGGTGAATCGATATATCCAACGCCTCGTAATTTAT
TTGTCACCACTTCAAGTTGTTCTGCAATATAATCGTTAGCACTATAATCTGGAGC
GATTAAAATTCTCGGCGTTACTTGATTTATTTGCGCAGACGTCAACAATGCGTTA
ATCCCGTTAACGACGTTCTGCCGCATTTTTGTTTCGTCGCTGTCCTCTTCGACGCG
GACAACAACAATAATGGCATCCTCCTGATTTAGTATTTCAGATACAGCGGGAGG
AAGGGTTCCAGACCCACCGAGCAATCCAGCTTTTTTTTGACTGCCTGCAATTAAC
GTCGGAACATTATATTTGAACGATTCGTCTTTACCACCGGATAAATTTGTTGGGG
GGAGCGCGAATACAGTCCCCGTTCCTGCGTGATCACCACCGAATGTGACGTTAAT
ATATTCTCCAATCTGGAGTCCTTCTTTATACTGCTGGTTCTGATCGTACATTTTTG
ACGGGGTCATGGCACCGTCCGTGATCATTGTTAATTTTCTCGAACCGTCGGGCAG
TGTGCTGTATCCTGGAGTTGCTACATTCCCGTTAATTCCCAAATCAACAATTTCAA
CAATCCAGTTGTTTCCGTCGGCCCCTGGCATGACAGTCGAAAACTCAACAAGATT
GTCTGCGAGCTCTGATCCCCATAATAGTGATGCACAAGTTGGGTAGCTGGAATCG
GGAGCAGTACCAACAATCCCAATCACGGAAATATCTACTGTGTTGATCTCTTTTG
TGCCGTCGTCGTATTCGAGCGTGCGAATACCGTGTAAAAAATTAGTTACGCTCAT
TTTATTCCTTCTCTATTTTCACCAATTCGCGTCGCTATTGCTTCTTTAAGCTCGGCC
AAATTTTCGATTTGCGCGTCAGATGCGGTTCCGGCATCTACTGCACACTGAATTG
AGAATGCCTCAATTGACAGATCGCCAACAGTCCGTAATGTAGTCTGGAGTTTTTG
CGTCTGCTCTTTTTTTACAAATTTAGAAATGCTCCCATAATCCCCGCTCAGCGCTC
TTTCATATAAAACCCTACCGTGTTCCTCGCAATCATTTTTCATTGCGGTGAATGGA
ATGTCGTTTCCAATCCCGTCAATATCGCAAATAATATTTATAGATAACCCATGCT
CGTCATTAAAAACAGGGTTTGTTATTTTTCTAAATGAATCAACGCTTATCATTACG
CAATCCTAATAGCTAAAAAACAGGCAGTCCCAATATCACCGACTCCACCGGGTG
AATACGCCATGGCTCGCCATGAGCCAATAAGTGGTGTGTCATAAAATGGTTTTGT
GGCAATATCCGTGTTATATGAACCGGTATTATTTATACTAGCGTAATATAAATCC
CCTCCAGCCCATATGGAGCCCGGGAGTATTTGTGGTGCATGATTAATAGCGACAA
GCGCATAAGTACCAACCGCGCCAACAACACCTATTTCCGGTGATGGGTCCGGTG
AAAGACTTATATCTATGTTGCCATTTTCATCCGGCGACTCCCCGTTAATCGTTTTA
ACCGGTTTAAGATTAGCCTCATTCCAGTATTTATACGTCTGCCCGCCGCACAGTA
ACACCAGGCCATCATAGTGAACTCCGCGTAATCTATAAAAAACCTTCTGATTGTT
TTCTCCACGGGTCGAAAAATCTAAATAATCGCCCGACACATCTGATAAACCAGC
AAATGAAAACAACGTCAAATCAGTGCCCGGTTTGTTTATATTTATTTGTTTTTTAG
AGGATAAAACGTCTTCAAAAATCACATCATCTTTAAAAGTATTTGGATGAGTAAA
TGCATTACCCTTATCTCTATATGCAACGTCCCCATCTATACCAATATTTTTCCGCG
CAGCCTGCTGAGCAGTTTCACCATTTGCGGGAATTTCAGATAAGTTTTTACTTATC
TGCAAATATTTTTTGTCAGCTTCCTCGGGAGTTACGTAGTTGTCATATACTACAAC
ATTAACCGTAGATGTGCTGGACACCGCGAGTGTGTATTTATACGTGATTGAAATC
AGCGCACCGTTGCTGTCCGTAGGTTTTAAAATGTCCGGCGAGCGCGCCACAGAGT
ACAGCTCGCCTTTATCTGTTAATATCCCCACCTCACGGATAGTATAGCCCCCAGT
GTCTGGCGGTACGTAAAGTGTAAAGATAACCTGGTTACCGCTATTTTCTGCGGAT
TTAATATCCCCACGATACGTCTCGTGAATTAATGCAGTTCGTGCCGGGTCCGGTG
TTACGTCGTCGTTTCCGTTTGCGTCGCCAATAACGAATTTATTCAGCACCACGGG
TACACCCGATGCCAGCGCAGCGGCTTCAAGTTGTGCGCCACGGTCTGTCAGTACT
GCGTAAATTTTATTTTCAGCCATTTGGCCCTATCTCCACATTTAAACTAACAATTG
GTGAGCCCGATATATATAGCCCGCCGTTAATTTTTACACCGCCGTCGCCGTCTGC
TGGGACGTTTACGCCTATTTTCGCGTAAGGTGTACCGGCTATAAAAAACTCACCA
TCGACACCCGTTTCGAAATTTATTTCTTTTAATAAGGAACGACAATTTTTTGCATC
AAATATTTGTGCAATAAATGTATTCAATAGTTCTAAATCTAACCCCATGCTTTTTA
GCAGATATATTTTAATTGCGAATGTATACGGGTCGTCCCTGGGCGTGGCTTCAAA
CCACTCTCTCATTTTGCTCTCAAAGCCAACAGTTAATAACGCTCGTTCAACAGCT
CCACGCGTTCCGCGGTGCTTATTCTGCCACGCAGCTGCTTTAATAATTTCGCGTTT
TTGCTGCTCACTCCAGTCGGGGTTCCAGTAGGTGACCGCGTACTCCCACGCCAGC
CACGGCAACAGATTTGCAGGGCACAAATCGGGATTTTTAATAATTCGAATATCGC
CAGGCAGATCGCCGACGTGAGATAAAACAACCTCAAGCGCCCTCTCAGGGGTTA
TTGCGTTCGGAGGCAGTACGCTTTGACTTGTCATGGCTACCCCATTTGCACGCTG
AGTTTGATTTCTGTGCAGTACGGAGCCTGCCCGACGCCAGCGATCACATCCTCTG
TTGGCGATATCAGAATCACACGTTCAGTGCCGTCACGATGTATCGCGTCGTAAAT
TCCTGACAGAGCTGCCGTCGCTCCGATTTTATGTTGCGTATCCACATATATCGCC
AAATCGCTCTGAGCACCGTTAAGCAAAACGTTCTGGTCGGGTCCGAGCCCGGCT
ACGATAACGGCCTCAACCCGGTAATTCAGGTTCGACGCAGGTTTTACAGTCACGA
AATCAGTCAGCGGTCGAGTATCGTCAGGAGACAGGGCCGCATTCACTGTATTCA
GTAGTGATTGAGGCGGAGTGCCGTCGCCGGTTCGCGACAAGACATACATATCGA
CGTATCCAGGCTGGGTCACCGGCGGGCCGTACGCCTGAGCTGACAGAACGTCGG
GATCACTCGACAGCGCGAAATAGTTATAGGCGTTCGTACTGCCGGCAGTATTCCG
CGCATACCATGATAGCTGTATTCGGTGTCGATACTCGTCGTCACTCTCGTAAACA
GCCTCAGTCGGGGGAATTACATCGGGATTAGCTGGCGTTATCAACAGCCGCAGG
CAGTCAAAATTAGCGCCAATCTGGTCGAGGTCACTCCCCTTTGCAAACGCGAGCA
ATACAGCGAGCATCCCCTGATTTACCCGAGCTGTAATAATGATTTCTCTGTATGC
AACTATTTCCAGCAGTTTAACGGCCGGGTCTGACTCCAGGAGTGCGTTAAATTGT
GGATATAATATCTGCAGCTCTGACAGTAGATTAGATTTAACATCTGAAAATAACG
GGGTTTTAACGAATGCTGGCGGCGGCAGCAGACTCATATCAATAGTTTTCACTAT
CGAATTCTGAACTGTTGATGTTATCAAAATATCAGCTCCATATTATTAAACTCTA
ATCGCTGTTGTGTTTCGATATCTGTTGCGATAATTAATATTGTTATTTTCCCGATA
TCCACTGCTAACACATTTATTCTGTCAATTCTGACGCGTGGTTCCCATCGTGCTAT
TGCGCCAGCGCTTTCCATGATTATTCGCATAGCAAGCGACGGATCTGTGGGATTA
TCAACAAGATCAAATAATTTGCTGCCATATTCCGGCAGCATTACACGCGTCCCCA
GCGGGGTCGTTAAAATATCAACGATAGACTGACGAATGTGATCAGTCCCCGATA
AAAGTTTACCGGTGTTACGGTCCATTCCAAGCATCTTTACGCCATTGGTTGGTTC
GGTTGTTGAGTTTCCCCGTCCGGACATATATGAGTGTGTCCGTTATACGTTGTGCG
GACTTCAGTTATTGTGCCTTGTCGGTCCTGCACCTCTTGCCCTGCTAACACACTAC
CGCGAGTTGTGATATTACCTGTTCCACCGTTATCTCCAGTGACAGAAATATCGTT
ACGGAATGTCGCTAATTTATCTACTACTAATGTCTCGGTAATATGCGTTGGACCA
GCTAGTGTTATACCACCCGGAGCCCACGCGCTGATTGTTTGAGCTGTCAGCGATA
TTCTGTTCGCAGCAGTAACACTAACAGAACTCGTTGCTATAACGTTCACTAAATC
AGCATGTGCGTTAATTGTATCTGTCGCTGTGACATTAATAATATCAGCGTCTGCG
TTAATTAATTTCCCACCATGGATATTAACTACATTTTCGCTATTAATAGTAACATC
TGCGCGTCCAAATATTTCTATTTCAGACTCACCGAGTATTTTTACCACGCCGTCCG
GTACGCCCTGCCATGTCAGAGTGTGATTTTTTGTGTTATACGTCATCGTAGCCCCG
TCACAGTATGCAGTGACATGATCATGGGGGTCATTGCTTGGCGGAGCTTTGTCGT
CAATGTGCAGCCCGAGCATAACTACACCGTTCCGCGTATCCCCACCCTCAGAAAC
AACAGTAACGGGATCGCCCACTGATGGTGCGCTCCAGTCGACGCGTTCTGAGGT
GGCGTGAGTAAACCACTGCAGCCAGCCTGACTGGTGTTCGCCACCAAATGAGAC
TCGGCAGCGTGGAGGATTCATTTTAACTGCAGCTATCGTGCCGCGTTTAACTGCG
TCACGCAGCCTGCGCGCGTTCTCCGCTGCAGCATATTCATCGTCCGATTGGGCCA
TAATAGTCGCCCTCATGTTCTGCGCCGATATCTGGTGCTATTCCGACGTATATATT
TTTCAGGGTCCCCTGTGAGGGATAATCGAAAATATCTGGACCAACGCCTACAACC
TGCGTGAACGACACGCACTGAACTGAATGCGAACCGACTGACTGCCCGTTTTTAA
TCCAGTCGCACGGTTCCGCGTCGCTGAATTTTGCCGGTTTAGTTCCGGGACCGAA
CATTCGCCCATTGATCCAGCTCGTCATATAGAGCGCTGCATTCTGCGATTTCAGT
CCGTACTGATCAGCTGCAAATTCACGCAGCAAATACAAATTACACGACAGCTCG
ACTGACTGAATTGAGCCTGGAACCGATTCGTCACTCTGTGACCAGTTGCTGATTT
CCAGGAACAGAGCTGGCGTTTCAAATCCAGCCGGAATTTCCGGATAAATTCCGA
ATGTTTTGATGAACGGTATTTGTAAAACCGCGCCTTTAACGCGATCAAGATATTC
GTCAAATGCATCCAGTCCGCTCATAATCGTTTACCTGTTTTTGGGTCTACGTGGAC
ATTACCTTTCACGCGACCGCGCAAATCTTTCTCAAAAAAGCCCATAAAAACAGG
GCCAATGTTTTCAAAAATATAATCATCGATAGCATCCTCCAGCGCGTCGTGCACA
GGTACGCGAGCCTCTTCGATTCCTCCGCGATCCAGTCGAATCCAGACACTTTTCG
CACCGTAGCGTTTCGCTACGAATGAATCGGGCCAACTCATCATCGCCAGCCCTGC
ACTTTTGGGTGTAAAAGTTGCCCCTCTAGCGCCTTTTTTGGTTTTCAAAAACAGGC
CGGTCTCAAGATCGCGAGGCTGTTTTTGCCTCCGTGGATTTTGTAATCGCCCCTTT
AGCTCAGATACCCGGAAATCATTCAGACCGTACCAGAGCTTGGCGCTGCTCAGG
TCTCCTGCCCCTTCTTTTGTAAAATTACGGCGCTTAATGAACGGCTTGATTCGTTT
ATCTACGGCCTTATGATTTTTAGCTGCCAGTGCAGTCAAAATCATTCCTGCTGAA
ATTCGGTGCATATGTTTTGCAGTTCGGTTCAGCGCCCTGTTGTATACCATTAACAT
TTGATGTTGCGTCGCGCTGATTTCGACTCTGAGCTGCTCCAGCGCCGAAACATCG
ATATCGAACATCTGCGCGTTACTACGTAAATCGGCCACGGTTCCCCCTTAAGTGG
GACCGAGGTCCCACTTAGTATTTTGAATACTCGCTGTTTTCGCTGGACGTGTGAG
GCTCTAAAAAAATATTCGTGAGCCCTGTGCCATCAGGTTGAGGCTCTTTAACCAC
GTAATCGGTCCAGCCAGACCAGGTAACAGAGCCGTCAGGTTCGATGCTGCGCTG
CTTTGGAACCTGGACGACGTCACGCGCAACAACGCCAGCGACGTCGTCCGAGAG
CGCCGTCAGGCTCGTTACTGTACCGGTAATAAAACCAGCGTGAGGAACGTCAAC
GCGCGCGTAAGGCTCGTTGAAAATAGCGATAATGGGGTCGGTTTTACCGCGTAA
TTTAACGGGCCGTCCCCACTCACGGATCATCTCCTCGTCGCCGGCCCGGAGGTCA
TCGTAGTAGCTCATAGAGCGTAAACGTGATCAGCTGCGATAAGGTCTGCGACCTC
CGGTGAATCAACAACAATTTCACGGCCTGCGGCAACAATCTCGCGAACGCGACG
CCCGTTCACGTAGTGATAAACGTCCAGCGTGTTACGCAACTTGACGCGGCGCGTA
GCCGGTGCGAGCTCCTGCTCTTGTGACTGTGAAACGATAGCGCTCGCGATTGATT
GAGCGAGTTCATTATCGGTCCCGGACGCAGTATTCACTGTAATCTCGATGCCTCC
CGCGTCACCGGATTGCTCGTTCAGAATATCGAGCTCTGACTCAGCGCCGGCAACG
ATTGATTCCAGTTCCGCAATAGTCCCGCTGCGCGGTAATTCGCGACCGAGCTGAG
CGCTCAGATCGTCAATACGCGCCAGCAATTCTGTTTTTGTGCTCATGTTGATTCCT
TAACAAAAAAGAGCCTTTCGACTCTTACGCGATTTTTACGACAACGAACGCGTCG
GCGTCCGTGAGGACCATTGCTGGCGCGGATTGCGTCATTGTTTGCGTCACTGCCG
GATCACCGGTAGTGGTCCAGACTTTTGGATAGCGCGTCGCTTCGGAAATACCCTC
TTTCAGCGCGTCTTCGTCCTGAATTGCGCCATAAGTGCGGAGCCCGCGGTTTTTT
GTGTTGCCAAGGATCATCGTGTTATCCGGCATATAACGCGTTTCAGTGTCTGTTA
CCGGGTCGAGGTATTGCCCTTTGTAGACAAACAGCGCAGTGTCGCCGTAGTAGCC
TTTAAAGCTGACAACGTCGCCCAGGTTTTTCAGCGCGACCTCGAGCTGGCTGTTT
GAGCCACGGCGCGTATCCAGTGCCGTCCAGAATTTTTTAAAACGCTTCAACTGCT
TCCAGGCTTTACCATCCATGATGATCACGTTAACCGCACCGGAGGCGAGGTCCGC
GTAGGATTCAATATCGTCGCTCGGGTCATACGTTTCCGCGTCCTGAGTCGACCAG
GCTGTCGAACCGGACTGAGTGACGTTATTCGTCGCGCTGCGGCTCATGTCGATCT
CATAGGTCTCGATATTGCTGCCGGAAACAGTGTATTTACCGTACAGAACAGCCTG
TACCGCCTGGAACTCTTCGAGCTGGCTGATGCTCAGTTCCTCATCAAGCAGGTTC
TGCATGATGATTTTTGCACGACGTTCGACCGGCGTTTCCGGCTGGCCAATCTGCT
CACCGGCTGCGCGCTTGATGCTCATATTCGGATTGATCGTGTGCTTGGGTTTCGT
GTAACCCGGTTTGAAATGGTTCGTTGAATAGCCGCGAGTGCGGTCAACTTTGCCG
GTGATCATCGGCGCGCAGTACACGGCCATGTTCACTTTTCCCGGGATTTTATCCA
GAAAAACCTCTTCGCTCGTAAACGTGTAGGTCTCACGGAAAAACAGCTTTAAAA
ACAACGGATTGAACTTAAATACCTGCTGTGTCGCGGTAATCAGTTCTGACGTAGT
AAACGAATCGCTCATTTATATATCCTGTTATAAAAAAACCGCCCGGAGGCGGCTT
GTTTTGAGTTTTTTGCTATCAGCCGACGCTGATCGGCGTACCCGCAAATGCTGAT
TTGCGTTTAGCTACGTCGGTAACCGTACCCCAGTTAATCGCCGTGTAACGGAACG
AGCCTGATTTATAGTACGAGCAGTCTGCAGAACTGGAGCCAGTATTAACTGCGGT
GGCAGTGAGGCCAATAGCTTTACCGACTGAGCCGTCCCAAACCTTAAACGTCCC
GACAGTAGCGTCGAGCATAATCGGTGTCAGCCGGGGTACGTTGATCCCGCCCTG
AAAATGACCGACGGTCGTGACAACAAGGTCAGGCCCAAGAATAAAATCGTCCGG
GCTATACGTTTCTGTAGTCATTATTCATCCCCTAAAATTGAACGCCCTGCAGCAA
CCAGCATCGAGACGTTTGCGGTTGTTCCTGTTGCCGTAGTACTGCCAGCATCCTG
AATAGTCGCTGGTGATTCAGTGCTCATTAGTGTATCGAGCGCCGTTTCTGTCCGC
GCCTGCGCCGTTTTAGGTGCTGCAGCGAGGACTACCTGCGCCTGCCCGACCGTCA
TCCCGGGGACGCCAGCGAGCGCTTTTGCCTGCGCCTCGCGCCCAACAGCTTCGGG
ACAATTGATGATGGCCATCACACGCGCCAGTTCGCTTGATGCGGCGTCCGCGCGA
ATCTGCTCCGTGTTAAACTCAACCTGCGCGACAGTAGTCGTGGTCGCGGTTTGTT
CTGTGGTCGTGGTCTCCGCCGTGGTTTCTGTACCAGACATAAAGCGCTCCATTTTT
GGTTTCAGTGCGTCGGCCATCACTGCGATAGCGTCCGCGTAATTAACGAGTTGAT
CAGCGAGACCAGATTTAATCGCGTCCGCGCCGATAAATACTGCAGCTTCCGTGGC
CAGCACCCTGGATTTTTTCAGCCCGGTATAATCCGAGACTTTTTGCGCGAACTGC
TCGCGCGTGCTGTTAATACTCAGCTGGAATTCGTCGCGAACGTCGCCTGGCAGCT
GGGAATACGGGTTCCCGTCAACTTTGTGCGCGCCGGCGTAAATCAGCGTCACGTC
GACGCCAGCAATCTCCAGCGCTTTTTCGACGCAGCGGTGAGCCATCAGGACACC
GATTGAACCAACTGTGCCTGTCTGCGTTATCAGCCGGCGCGAACACGCTGACGCC
AGCAAATAGGCAGCGCTGCAGGCCGTATCGCTGGCCAGCGCCCACACAGGTTTT
TGCTCTCGCGCCCGGGCGATTAAATCAGCAGTATCAAACGCGCCGGCGACTTCAC
CGCCAGGGGAGTCAATATCAAGCAGGACCCCTCTTACATCGGGATCAGAAATCG
CCTGCTGCAGGCGTTTTGCGATTCCGTTGTAACCGCTCATCCCGCTAACTGGGTT
GATATAACCCAATTTATGAACCAGCGTCCCGGTAACCGGCAGAACGGCTATACC
ACGCTCCACGCGATACGATTTCTGGCGTGTTCGCTCGCTGCTGTCCCACCCCATC
GCGAGCGCGTTCATTTCGTCGCTGTTCATTACCTCTCCAGACGCTGTATCAATCA
GTCGCCCGGTACCGAACCGGTCGCTCAGCGCCGAAAAAAATACCCGCGCGTAGG
TGGGCTCCAGCAAAAGCGGTTGATTGAACGCCCTGGCGGCGAGGTGCGGAAAAT
TGTTCCACGGCATCAGTTACCTCCCCAGCCGTTATCGTTGGTATTTGAGGGGTGA
GATACAGACCATGACGGTTCGCTCAGCCCCATTTCACGCCGGCGCTGAATTTCGT
ATTCCTGCTGCTCCATAACCTCCTCGTAATCCTGTCCCTGCAGCGCCAGTTCGTTC
TGATACGTGCTGAGGCCAGTTGTGATGCGCATGGCGCTCTCCTGAACCTCTTTCA
GCCCATCAATCGCCATGCGACCAGCGCCGATCCAGAGCGCGTTCGTCCAGGAGT
TCCGCGCCTCATAAAACGACCGGACAGCTGAGCGTGGCAGGGTGATAATTCCGC
GTGCCAGCGCCTCTTCAAACCAGCAGCAGAACATCAGCGACGCCTGTCGCGCGG
CGATAAAACGTCGACGCCCCATGAAAAATCGCCAGCTGACGTTAGCACTGGCCC
GGGCGCTGGAGTAACTGACCTGGCTGTAGTCCCGGCTAAGTTCTTCGTATGACGC
GCCAACGCCAGCTGCAACGTAACGCAGGAGTGATTTTTCCAGTGAACTAAAACC
AGCATCAGCATTCTGAGCGGTCTGTAAACTGAGTTTGTCGCCGGGATGCAGGTGG
GGAACTTTCACACCACCGAGCTTGATATTTGCGCCGTTGTAGTACGTCACGTAAC
TCTGAATAAACGAATTCAGCGGGTTCGAGTCGATATCAGTACCCGCGCCGGCGA
TATATTCAAACGCCTGCTGGCTGTCGAGCTCTGATTCGATTGTCGCGGCATACAT
CGCCTTCACAATGGCGCTCTGCAGTTGTGTTTGCTGCAGTGTGTCGAGCATTTTCA
GGCGCTCCATGACGCTGTAAAAAATATTGTCCCCGCGGGTCTGTCCGTCCTCGAG
CGGCTCGAATATGTGGATGAATGCGTGTCTGCCGCTGCTGAGCTGTGCTGGGATA
CGCCGGCATTTACCGACTCCGCCGAGAGGGTAAGTATCCTCGGCTATCCAGTACC
CGACCGCGGCCCCGTTTTTATCGATATCAACACCGGCGCGGCGAAATTGAGTGTC
AGCTGCGTAACCCGGATTTCGAATCCGTTTCGGTGAGACCATTTTGAATCGCGTC
CGAAAAACGCTGCCGGCACTACTCTCCCAGACTGGTTGTACGCAGGTTTCACCGT
TGAAAGCGTGAGTAGCCACGCCCTCGCGGATCATCATTGTGAACGTCCGTTTCCG
CTCGATATCAATCGTGCAGTGAGGGTCCTCGGCGTATTCAGTCCACGCTACCTCG
ACGTCACGCGCCAGCGCCCTGGCGTCCTCGCGAGATATGCCGAGGTAACGCCAG
TTAGGGCGATAACTCAACTTAAACAGATTGCCGACGATATGATCCTGGTGCAGCT
GCACGGCGTTCGACGCGACGCCGTTATTGCGAACGAGATCGTCTGCTCGCGCGTT
ACCGCGATAAAAATTCGGCAACAGTGCTGCGTCGGCGCTCTGCTGTGGTGCGTTC
CAGTCAATCAGCTGGCCACCGAAACCGGGACCGCCGCCGTTATACCCCGCGTAA
CGGCGTAGTGGCGTTTTACCGTCAGGCCCGAGCAGCTGCTGCGGAGCAGTCATA
ATCGAACTCCCACGGGCCGACGGCGTCCCGTACTCAGACCGAGCTGTGATTTCAG
CTCGATAATGTACGTTCGGAGCTGCTCAATACTCGCCTGCGAGTATTGATAGCTC
CGGTTATTTCCTGCTGAATCGCCGTGAGACAGCGACACGGTCGATTTACCCGTTA
ATAATTTGTGCAGAGCGATTTCCGCTTCCAGCAGTCGTTCTGTTAAAACCTCGCG
CGACGTCATTAGCTCCCTCCGAGCATTCTGGCCATCTCCTCGAGAGACAGTTTGT
TTTGTGATTTTTTCCGTTGCTCAGCGAGCGTCTCGAGATTGAGCTGGAAACGAAT
TTTACTGATACGCAGCGCTGCCAGTCCGTACACCCAGCAGTCGAGCGCCTCATTT
CGCCGGCCCTGGTTGTCCCAACGATAAACGACGCGACCGTTAATGAGTTTCGGG
ATCAGAACCTCGGAGACGAGCTGTTTTGCCTCTGTCGTGCTGAATATTTCGTCGT
CGTTGGGAAAATGAATCGCACCTGGTGTCGCCGATTTGGAGTCGGGTTCCAGCTG
CAGGCGCATTGCGAGCAGGTCTTTCGCTGTATCTGTCCCAATCAACGACAAATAC
ACTTTATTCGCGTTACGGGTACGAGGCATATTTACGACCGGCTGGCCGTATGAGC
TCGCGCCCTTGATTGGAATGACCCACAGCGGACCGAGTTTCAGCGAGCGGTTATA
AACGACCTGCGCATCTATACCACCGGTATCCCACGCCCAGCGACTGACGCCGATT
GTGGTCCCGTCGCTGCGCCGGTATTGTTTGCGAATCACTCCGTCGACGCGCTGCA
GCGTGTCCTCCTCGTCGTAGCGACCGAGGACGATTGTTTTATCAATCAGCCAGCA
CTCCTCCTCGGCTCCCCAGCCCCACACATAACACTCGTAGCGCCCGGACGTCTGA
GAGTCGATGCCGCCGGTTATATAAACAACACCGTCAGGAACCTGGCTGGCGTAT
TTTTCCCGGCGATTAACCAGAATATCGTGCTCCAGCTGCTCGCTGGCCACGTCGC
TCCAGAGTTCGCCGAGCGTCGTGTTATGAAACGTCTTTTCCTTGAGCGGGTCGCC
TTTCGCTTTCAGCCACTCGCTGACAATCTCGCCCCATCCATCGAGGTTTAGTGAAT
ACAGGGCGTTTATCACGATCGCAGCGTGTTTCGGCGCGCGAACGACGCCACCGT
CATGATCAAAAAAATGAATGCCGTCTCGGGTCCAGGTGCAGTCCTCGGCTATCCA
GCGCCCCCCGAACTCCATTTTTTCGAGATCGCGATAGTAAAAATGTTCCGGGCAG
TGGCAGCACTGGTAATACGCGGACGACGATTTCGCCTCATTGGTCTGCAGGCTGT
TATCCCATTTGAGGCCGTATTCGATACCGTCGAAACCAAATACCAGGACCTGCTC
CTCGCCACAATGCGGACATGTCAGGTAAAAACGAAACGTCAGATCGGCAGCGTC
TTCGAGCATTTCGATATGACTTTTGCCGGTAACGGTCGGCGTCGAACCAAAAATC
GCCTTCGGGTACGCTGCCCCTTTAATTCGAACGAGCGCCAGTTCGATGGGCGAGC
CCTCGCCTTTGCCTTTTTTGGCAACTTCCAGCGGCCAGCCGTCAACTTCGTCACCC
TCGACAACCTGTTTCGTTAGTCGCCGGAAATTTCCTGGTGCACTCGCGCCGCGAA
AATCAAGAATCGCGCCGCTCATTTCTTTACGCTGGAGATTGTTACGCTCGTTGCTT
TTATCCCAGTCGGGGAAAATTTTCTGAATCACTGGCATTTCGGCGATAGCGGGGT
CAACCTCGTCGGCGACAAATCCATCCGATTCGTCATCGATAGGCTGATAGACCAC
GGCACTACGTTTTTTGTGCTCAGCGAAATAGAGCAGCGCCGCGACGAGTATTTTT
GTATAACCGAGACGAGCTGATTTGCGAACAGACACGATTTTTATCGCGTCGTTCG
TCATCATATTGAGCATCACTACCTGGACCGGCTGAGTCGTCCAGTGCCCAGCGAT
GTGGCTGGAGCCCTCAGGGAGATAAAAATATTTATCAGCCCACTCAACACCCGT
CATCGGTATCGTCACGCGCAAGGGATTCAACCCTGTCGAGATCGCGTTCGATATC
GCTGACATCGTAATCGCTGAAGTCGATTCGTATGTCTGACAACTCATTTAAGGCT
ATCGCCAGCTCCTCTCGTAATACACTGCTCGCCTCTTGCGGCATCTCCGGCCAGA
CCTTTTTCAGGCGTGGTGGCCACGACTCGACGCGGGTACGTAATTCAACGGCGAC
GCGAGATACAGCGACGCTAATTAATTCAATTGGCGCGTAGCGTTTAGCGAGAAT
GCGGCGTTTTACTCGTGCCATCAGGATTCGCTCCTGTCGTTCCTCATTTTTCAGCC
ACTGCTCGCGATTTTTTTCGGGAGAATTGTCCCCCGCTTCCGGTTCGTCATTACTA
GTTGTGTCGCGTCTATTGCTCCGGAGATAACGAATATAAAAATGGCGCCATGCGT
CTAGCTCCCAATCACCACGTCCTTGTGGGACTGGTGCTCCCGGAAGTTTTGCGAG
ATCGCGCAGACGACGATCAGATAGCAGTAAATGTGCTGCAACCTCAGCCTGACT
TGCCATTAAAAATCCTCACCGGAACCGGAAATACCCAAAATGAAAAAATACTAA
AAACGAGCGAGTTTTTGCGCGTCTACCGACCCTCGGTGTTTTGGATTTCGGAAAG
GACCCGCGACCAGGGGGCTATCTCCCTTTCAGGACAGGAATTAGGGGCTCGCAT
CAGGTTCATCAAACTCAACTGTAGCCTCGCGGCGTACTTGTTTGCATACATCAGC
TTTGTGTATCTGATTATCAGCGTAAGCTTTCACTTTGGCAGCGAGCCATCCTTTTG
TGCTGTATCGATAGACTACCGAACAAGTTTTCCATATATGTATATATCTTGAACTT
ATTGATATCATTACAGAAAAACAACAACCATGAGAACGCAAAAAATGAACAGA
GAAAAAATTGAAGCCTTAATGAGCGTAATATTAAAAGAAGCTGACCCAAAAAAG
TTTCCTGAGAAGTTATCAGAGGACGCCATCGTATCTTTTTATCGTGCGAACTCCTC
TTATCTAAATTGTTCAACTTTAAGAGAATGGCTAAGCGGCGAAGCAGAGCCTTCA
GATGAGATACTTCAGAAGATTATGGTTCAGAACAACTGGCGCGCAACAAAATGG
TCAAATCAGTTACCTTAATCTGTTGAATGTGTGCCGCCTTAAAACAAAAAGGCGG
CCTCTCGTTAAAGTAAATTACCCTATTTTCCACCAAGGCATGAAGCCTCTGCCCC
GGTTGAATCAATACAACCACCGTTCGCAGTAGTAATCTTCGTACCCGGCGCGATC
ATCACGTGTTTAATACTGCTGCCGTCAGCGCGAGTCGCTTCGGTGTATGTCGAAT
GTGTGCAGCCTGCCAGCGTGAAGAACGCGACAGCTAAGAGAAGTTTTTTCACGT
TTTACCTACGCTTCTGAGATAGATTTCTCAGCATTCACCAATGGAAGTGATTGTG
GAACATCCGTTTGACCTGCTGGCCAGCGGTAGCCTGTTACGCGGGAACGAGGAA
AAGCTTTGATATTTACTTCATCTGCCTGGTTGCCCCCCAAAATCATTAAATCCCCG
GCGGCATTTTTCCCGACAACAAAGCCGACGTGACCACCGCCAGACCGAGACAGA
ATAGCAATGCATCCATAGGCGGGTTCCTTCAGTTCATTACCCCAATCGAGATAAG
AATTCGCAGACTCAAAACGCGTTGAGCGAATACCCGCACGTTCCAGCATTGCCCC
GGTGAAAGCAGCGCACCACGGTGTTTCGTCATCCTTAATGCCGCCGCGTTTGATA
TCTTTCCAGTACTGCACAATTTCTGCAGCATGTTGCTTCCCTTTCATTTCCCGAAT
GCCGAGGTTTTTTCGAGCCTCGATTAGCCATTTAGGATCCGTCATTACCTGTCTCC
TGTTATTTTTGAAATATTCCCTCGTGCCCGCCAGACAGCAATACAGATGGTGAGG
TTGAGAAAGAACTCTGCGGGATCAACCTGCGTATATTTCCCGTAAAAAATTCGTA
GGGCTATCCACGCAGCAGACAGAATCGCGAGGTAAGCAGTTAGTGAGATGAAAA
AACGATAGCGGCCTGTTTTTTGAAAAAACATCAACCGTAGTGCCAACATCAGAC
AAATAATTGCATTGGCATTGATGAGGACAGAGTGCAGGGTCATTATTTCATCCCC
TTATCTGATTTCCATAAACGAGCTTGTGACATGATCCGCAACAGTGATGTAACGC
TGATTACTGATGCAACCAGTGCACCAATGGCGGGAGAGACTTTTACTGTTACGGG
AGGGTTCAGGTGACTCAGCACCGCGTTCAGAATACCGGTAATGATGGCTGACGC
TGTCTCAGCGCAATACACACCGCCAACAAACGAAATGAGTGCGAAAATAACCTG
CTTCCAGAATCGATGCTGCCCGGACGACAACACATAGAGAGCTGCGCCTGCCAG
CGCACAGATCATCACTGCAGGTGTTGCCTCAGGAAATAACGCAGCAAACGTCAC
CCCCGCCGAGCCAGCAGCAACGCCAGCGGTTAGAGGTTCAGACATGATTTTTCCT
GATTCAGAAACGACAAAACCCCGCCGTGGCGAGGTTCTTAGAAATTTGGCAACA
TACCAAATTAGCGTCAAATATGGCCTATTTTGTTCGTTTTTGCAAGCATTATACGC
AAACGTCGTCTAAATGGTTCTGATAATGATGCAAAGTTGCAAGAAGTGCCACTTC
ATCAAGTGCTCTCACCGCCTCTTTCATTCCTACCCAGTGCAGGGAGTATGTTTCGC
ACCAGGTGGAACGAGAAACCGACAGCATTCCTGCTAGGGTAGATCCGGCCATCT
CCTTAAAATCCTCGCGTTTATTTGCTGCTGCGACCGCCTGCACGGACAACCACAC
CAACGAAAGCAGGCGCTTAACTACTTTTCTCTGAGTTGACGCGGGGATGTTTCCT
TTATAGCGACTCCAGACAGCCTCACAAATAGCTGTTTGGTGTTTGAATGCCAGGT
TACCACCGTAGCAATAACGCAACCACGCTTGCTGACTTGCATCCAGCGAGTTCAC
AGAACGCCGCCAGGATGAATCAGCAAATTCAAAATCACTCATCGGCGGCGCGGG
GCGACGTCTGCTGAAACTTTCCAGAGCGTAAACTGGCGTAACCAGAGCATCAAC
GGTCCGGTCCTCCAGCTGCACACGGTGAATATGTTTGCGTGGGTATGCGTTTTTG
TCTGCCGGTGGGTGCTCACACAATGCTTCCAGTTGCCCCTTAGAACCACCAGACA
AATCCCGCAATGCAGCGCGCAGTTTTATGCGGGTATATTCCAATAAATACTCGTT
ACTCATCAGATAAACTCCTCATCGCGCCAGATAGCCAGTGTTCTGAAAACTCCCT
CAGCGTGCATCAACAACAATGAGTCGTGCTCGTAATCCGTTTTTGTACGTCCGTC
GATGGCATCGTGACACGCACTGCAGGCGATAGCGCCCTGCATATCGTGAGGTTT
GATGGCTGTGCCGCACGTTCCGGCGAGACGATAATGAGCCAGTACGCTGGTTTC
AGGATTGTGATTGCACACACCGGGGATACGCACAGTACAAGCGCGACTGCGTGC
AGCTTTTCTCAGGTCGATTTTTTTCATGATGTGTAATCCAGCAGCTGCGCGGCGG
CATTTTCAGCGGCCTCCTGAGAGGCAAATGTGCGGAACAAAATATAATTCCACA
GCACATCTAAAACGGCTGAATACAGTTGACCGAACTCAAGATCGTCCATCGCTG
CAAACGAGATTGAACGAGGTACGCGCATCAAACTGCCGTCTGGCATCTGATGAA
CGTCATAAAACCCAGCTTCCATAATCGCCCATGCGCGAAACGCCTCGAACGACTT
CACCGCACTGATACTCCCCGCGCGCTTTTCCGACTCATCGAGAAGATACTGATCA
GCCAGCTCTGCCATGACATCACCGTGTCCACCGTAATACGCCAGTAACTTAACGT
AGCCGTGAACCAGTTCGCGCTCCGCAGGAGAAATAGCCCCTCCGGACGGATGCC
AGTAATCAAAGCCCAGGTTGAGAAGTGAGAAAAATTTACGATGAAACGCGGGAT
TACGCGCCTGTTTGAAATCTGCATACACAACAGCACCTGGTTTGACTCGTTCAGT
CAACCAGCGTTGCGTGTCTGGTGTCGCGGGTATTAGCGAACCGCCGGATACCCTG
ATAAATGAATGCTGCGCCATGGTTAACTCCAGGTGGCGCGGCAGTGTTCAGAAA
ATGATGCCGGGTGTTCAATCCAGCACCGATATTATACCGTGTTTTACGCGTCAGG
GGGTGTAATAGTATATCCAGCTATTTCTGCCAACTCGAGAAACGCCATCAGGCTG
GCAATCCGCTCATCATCGGTCAGAACACGCACACCAACGATGTGTCCGTCCTTAC
GCTTGATAACAGCCCGGCATGTTTCCGGTAGTTCGCCTATAGATTCTTGAATATT
CAT
ST131 Region-7 (SEQ ID NO: 18)
TCACAGCAGCACCTCCTGACCTTCCGGCTTCACAAATTCAAAACCTTCGGGTAGT
TCGCCATCAGGTGCTCCGAAAATAATATCCCGGTAAACCTCACGCATCTCTGTGA
CACCAAAAACTGACAGGCGAATATTTTCTGACATAAAGCCTGGTTTAAGCATTTT
TTCCAGTTTCTCTGCGGCCAGTATGCAACCAGCATCAGCCCCAAACTCATTCACC
CACGGCATTTCAATATGGTAAATAATATTACTCACCACTTTTTCGTGGGAGAGTT
CTATTCGTTCGCCATCTTCAATATAAACAGGTTCATTATGAACACGCACCATTAA
GGAAATGAATGCATCAGCAATGTAGTAGCGTAAGACTGCTGTTCGTAAAGCATG
CGCCTTTAAATTTTGATTATTATTCATCGCGCATCACTCCCTTAATTGTGCTGGTT
TCCTTAACAACGCCATCCAGTTGTTCGGCAATACAGGCCACTAAAGCCGCAGCGC
CTTCCGCTGAAACAGTTCTCGCTCCATTCACTGTGGGCACGGACAAGACCTCTGC
AAGAAACTCACTGGCCAGTGCCGCCCGTAACAGACGACTCTCCCCCTGTTCACTC
AATATGCAGCCTTGCTTAAAATTCATGGATTTCATGCAACCACCTCCCCGGCTCT
TTTCACAGGGATACGGGCAGCAAAGCTCAGGATAAATTCAGTGGCAAGTTTAAG
GCGTGCAGCATGCTCGCATTCTGCGATTACACGCACAGGGCGTGGACGGGCATC
ACGGTCTGTACGGCGGACAGCCAGAAAGACAAAGGTAAATTCAGGGTGAGATA
AAGCAGGGACTGTAGCCATAATGGCAACCTCCTTCAGATAGCGGGTAACGCTAC
CACCGGAGTTCCTACGCTCATGGGTGGTAGCCCAGACGGGGGTAGGAATACCGG
CTCTGAAGGATACCGGCCAGCCCGAAAGCTGCCCCGCCTGGACTACCATAATTCT
GATGATGTGGCGTAAAAAATAAAAAAATACAATCAGCCACCACACCATAAATTT
TAGGTGAGTCAAAGCTGCGACACAAAAAAACACGCCTAGCGCGTGTAGTATCGC
CTTCAGATAACACGGGTTCCTACGCCCGGCTGCCGATTTTGCGGCAACCTTTAAA
CTATATCCCGCGCATTCCTGGTGAGGCAAGAAATTTATAACCGAAAACTGATCAT
TGCCTGGATTAATTAACGCTGGCATGCTTAAGCCTCTGCTTAAAAATAGTTCTTC
ATTTTTCCTGATGCTGTAAACGAGACCCGCCTCTGAGCGGGTCTTTCTGTCGCTA
AGTCAATCCGCAAACGTCAGGGTGCACTCATCAACAGCCGCGTACAGTTCAGGT
TCACCGAGTGCATAACCTTCATTGCGCAGGTAGGAATGCACGTATTCACTGCTTT
CATCATCAAACTCAGCACGAAACCAGCGTGTTATCGCCTGAGTGTAAATATGCA
GATTATCCAGCGTCGTTACTGCCAGACGCTTACCCGGCATAAACGGGGGAATAA
ACGCAAAGCGGCCAGCAACAGAACTTGTAGCCATCTGTGCCGCATTCACATCAG
ACGGGCGGTCTGCGGCATTAAATAACTTCAGGCGTTGATGTGCTGCCAGCTCTGC
CCCAACCAGTACAACCAGACGAGGATCTTCACGAAACTGCTCAGCAATAAGCTC
TGTAATCAGATGGTTGGCCAGAGCATCAATATTTTTCCAGGTGCCAGTCTCTCCC
AGAGTGACAGGCTCAGAGATGATTTGCTTTCCGTTCCCGTAGGCTTTCGCCAGTG
CATGCCAGCCAATATTGACATCCTCGCCTTTTTTATTGATTTCCGGGTTTGTAGTA
TTTGCAATCGACACACCGTTGAAACCGACCCGCAGCATGTCCAGCGCATATGCCT
GGGCAAAAAAGCTCTCAACCGTTTTCTCAAACTCATCCGGCACGCCGGTATGGTA
AATATGAGACATATCGGAATAGCTGATCCGGGCGCAGGTATCCGTCTCTGTCAG
AAAGAACTCCGTACCATTGATTGCCATTTTTTTGTGGAAACGGCCACCATCCACA
CGTCCGGTATACAGTTCACTGGCACCAATATTGACGGCATTACCTGACACGGCGT
TAACATCACGTAGGGTAATATTTTTAATAAGCCACCCGGATTCCAGAATTGAGGT
ACGCAGTATATTTTCAGTAGGTTCGCTTAATGCAAAGCGTCTGTTTGATTCTTCCT
GATAGTTAGTGCCAGTGACGAATTTATTATGATAACGACGTGCTGCGTCCGGCCT
TTCAGTTTTGAAGGCATACATAGTTTTTCACCTTTTTAAGTATTTAAGAAAAGATT
TTATTTTTGTACTCAGTAACCAGATTGTATTTCATTACATGCTCAACATAATCCCG
GTCACTTACGACTGAACGTAATACATAATTATCTGCTCGATAGTACTCCCTGTAA
GCCATAGCTCTTGCCATTCCACCGTTATTTAATACATAACGATCAGTTGGAACGC
TACAACGACTAATAATTTCACTCACGGAATCAGATAAAGACTTGAGTGCATCAGT
AATAAATTTAATATCACGGACAACGTCACTGTCATTAAATACAGAAATAAAATC
AGCAGGGATCTGGCCGGATTGAGGAAATATATTCAGTAAACGCTTATCGATTCCC
TTCCCAATGTAGTTTCCTTCATCGGCCAGTACTCCCGGCAAAAGCTTGATCGTGC
TCAATCGACTATGTTGTTCATTCAACCGGGAAACAGCCAGATGGTAATACCGTTC
AGCATCCGCTCGTTCAGCATGCCCCATTTTATTCGTTTTGGCTTCAGCAATGCTAC
TGACGCCGTTTTTTGAGAATGTGCTTTTCCATGCAACTTCCGCATTTGTCACTTCA
CGCTGCCGGAAAGATAACTCTTCCTCTTCCTGACGACACAACTGGGCATAACGGC
TCAGGAGCAGGTTGATCTCATTCGCCACAGTTTCCGTATCAGGTACTGTTAACCC
CGTAAGTGAATACAGCTTTTCACCTTTAGCTCGCAGCTTATTCAGTGAATATATTT
CAATAATACGCACAATACAGGCCGTGTCGCTATGAGTCTCTTTAAATGATCTTAC
TTTGTAACTATCTAACACCGACACATGTTCGTTCTTAATTGATGCCAATTTCTTCT
TAAAAACATTGTCAACCAT
ST131 Region-8 (SEQ ID NO: 19)
AGATAACAGAGGGAAGTTTAACCTCCCTCTTGCAAATAGT
ST131 Region-9 (SEQ ID NO: 20)
GTGATAAGTGAATCAGTGATTTCCGCAGCTGTACGAGCACAACAGTCAGAAGAT
GGTAAAGTGGGAAATTGTGGCCGGATCTGGCATGTCGCTTTCTTTTTTGATGGAG
TGGGACGAAATATCGACCGGGATGCGACAGACTGCCGTTTGAGTAATATTGCCC
GCCTGTTCCGGGCATATCCTGATGAACAAAAAAATACAGGTGATACTTGTTATAA
TAAATTTTATTTCTCCGGAATGGGCACTGCCTACCATGACGATTCTGTCGACAAT
ATTCACGCTCTTATGGATATTGGTTTAGATAACTTTCTGGAGGATATGAAAAGTC
TGCCAGAGGATACTATATCGGATGCAGGGGAGTCTGTTATTAAAGGAGAAAAGA
AATGGACGGATGTGCTTGGTAATCTCCTTGAGGATCTCAATAATCCGGTAGAGTG
GGTTAAAGGGATTGGCAAGATGGCGGCTAAGTCGGTAGGCAAAGCAGGAATTGA
GTCCACACCTTGGTTGCGTGATAGCGAAGCCATGAGCGCCTATTTTATGACGGGA
GAACCCGTACGCCTTGAAGCTGCAAAGCGATTGTTTAAGAAGTTTTATACGGAA
AATATGAAGGGAGATGTTCCCATCAAAAAAATTTCTGTTTCATTATACGGTTTTG
ACCTTGGAGCTACACTGGCACGTAAATTTTTGGATAATTTTCTTAAGGAAGTTTG
CCTGAAAAATAAAGAAAAAAAACACCAATATAAAAACGCGTTGGTAGATATTGC
TTTTATTGGTCTTTTTGACTGTTCTCGGCATTCACCGACTAGTAGTAATAATGGGT
TGGACTATTTTTTTATGTGGTTGGGTTTACCAGGGAAAATGATTGGTTCAGTATTA
GGTGAAAAAGCTATTGATCAGGACTCACCACTGCCGGACGTAGTAAATAACGCA
CTTCACCTTGTAGCAGCTCATGAACGGCGTCCATGGCGTGGTCTGTATCTGTTGG
GGAAAAAAACGGATACAGCGAATAAAAAATACAGGGAAGAACTGCTTCCCGGT
TGTAGTGAAGATATTGGTGGTGGCTTGAAACCGGATGAACAGAAACCCAGTGCA
GAACTGTGCAGGGTTGCACTATACAATATGTATCATGCAGCATGTCAAGCTGGTG
TGCCATTTCCTGATTTTGACACTTTGGATCAATATGATACGAAAGTTGCCAGCTAT
TTTATTATGAATGATGCTGTTCAGGGAGGATCTGTAAATTACTGGGTTAGTCGTT
ATCAAAGAGAGGAAGTGAAGGATAAAGTATTTAGTGAGGCAATGCAGGAGAAG
TATCTAGATAACTATTTTCTCTGGTTGGGTGAGCAATATTATATGTATCAGTATGA
ACGGGAACGTCTGGATAAGGAGTTGACATCGGCTCATCGTGGGAAAATAAGTGA
CTATGGGCCACTTGCAGGTACGGGAATTAATCCAAATACTAACGCTGATGATATA
AAATCGCAGATTTCAGAACTTGACTCACTATGGGGATGGTTGAATGATGTAAAA
AGTGTAGCTATTGGATTACGAAATGACTTTATAGTCAACCCGAGGAAAAATGAT
AGCCGAATAAAACTTAAACCTCAGGTTTATAATGCTGCTGTCAATCGGGCTGTGA
AATTCCTTGAGTTTTCTCGCGCAGTTTATAATGATAAAAATCTGCCATCATCATCA
TCTTTGACGGCAAATACACTATATTCCTTTTTTGTGCATGATATACAAAAGGTTGA
TCACACACCTTCTATATCTGAAGATTTCTTCTTACGTCGCTCATCTGAAATTCCAG
ATGCGGATGAACCTCCAGGAAATAAAGATGATGGAAAGAAACATACTCGCAGG
GATGATTAGAAATTATAAGTAGCTTACTTTAATTAGTTATATTTTTTGCATTTTTC
CCTTTTTAAAGAGAATCATACTTGGATTTATTTTTCATAAATATAAAGGAGTACA
GAGA
ST131 Region-10 (SEQ ID NO: 21)
CTATCTCTTTCCAAAGCTGATCTGAATAACATTATCTGGAGTACCATTATCAGAC
TCAATGATCTCATGCTCAATAAAATCCCCCCATTTCAGAAACAGATTCCGTAATT
TTTCCGCTTGCTGCCCGACCTGGTAGGTTCTGATTAACCTGTCTTTGGGCACATGA
TTTAACAGAAGTTCTATAAGCCGTTCTGGTGCGTCCAGATATTCAGCGCAGCCTG
TAGCAATGCTGCGGCGTAAATCATGTGGTGAGAAAGGAGCGACTTCTGCCATAT
ATTTTGTTTTCAGGCGTTCAAGAGCACGAGTTATTGCGTCACCGGTGACAGGAAA
ACTTGGCTTACTGAGCGACTCGAATACCCAGCCGCCTTCTGAAATAATGCGCTGA
GTCTTCAATAGAGAAAGCGCCAGAGGATGTAGCGTCACGATATGACTTTTACCGT
TTTTGGTTTCAGGTATTGTCCAGCGGTTGCCATTAATCTGCTCCCAACGCATATTC
ACCGCTTCGGAACGTCGCACCCCCGTCAGGATGATGAGGCGTAATGCGTTAGCA
ATCGCTTGTGAAAGAATAACGCTGGACGCGATGCCTCGCCCTCCGGTTGCGACA
GATCCGCCGCCGAACCCCGCTTCATCCAGTGCCTTCCATAACATTTGCAACTCGT
CTTGTGGCAACCAACGCTCACGCGGTTTTCCCATTGAGGCACCAAACATTGCGGG
TTTAAGCAAGCGAGCAGGGTTGTGATCGATCAATCCCTGCAAAGCGGCATGGTC
AAGCGTCATATTGAGAAGAATAAGTCCCTTGCGGACACCTTCGGCGCTGGTCTTT
CTGATCCGTATGAGATGTTCATAAATGTAGGCTCTGGACAGGTCGCAGACGCGTA
CTGAACCTAAACCTTTCTCCAGATGGCGGCGATATGTTCCCTCATAATCAGCCAG
AGTACGCGCCCCAATGGGTTTATTGGTTTCACGGAATGCCAGCCAGTTTTTTCAT
AATTGTGAAAAAATCGGCATTTGTTCATTTTGCTGTTTTTCCGTTTTTCGCGCATA
ACGCGGATCAATACCTTGTTTAACCAGCGCCAGCGCGTCTGCATGTGCTTCACGC
GCCTGAGACAATGTCATTGCAGGGTAGGAGCCTAACGTTATTTTAATCTGTTTTC
CGTTGAGGTAAGGGCGGTAAATCCAGCTCTTCGTTTGACTCCCGGAACGGGATTC
GACAGACAGGCCGGAACCTACTTTTGATTTGACGCTGACGGTTATCCGACTTTTT
CCTTCAGGGGGAGTCAGGTTTGCGACTTGCCTGTCCTGGGTTATCAATATTGCCA
T
ST131 Region-11 (SEQ ID NO: 22)
CTATTTCATCAGACTTTCGATTAAGCCGCTCATTGCCATATAGCCCATGATTAGC
GTAAATACGCTTATGGCAATAAACATGACGTTTTCCCAGCGTGAGCAACGATATT
TGCCCATTAACGAAGGCTTGTTCGCCAGCCAGAACATACATAAAACAGTCAGCG
GCAGAATAATGGCGTTAAAGGCCTGGCTGGCAATCATCACCAATACCGGGCGTG
CGTGGAACAGCGGCACAACCAGACAAAGCAGAGACATAACCGTCATCAGGATA
CGGTAACCTGGGCTAACTTCGTTGCTGGTATCACGCCAGTCAGCCAGCAGCCAGG
GCGTTAACAGCAAATTTGGGAACTGGCTGGAAAGCCCGGCTGCAACAATACCAA
TAACAAAGAGCCCCACGGCAGCTGAGCCAGCAATAGGTTCGAGAATAATGACCA
TTTGCGAGGCATTATTTAAACCTGTGCCAGTTTGGTACAACGTACCCGCAGCCGC
AGCCATCACCGCCGCACTGATGATGAACATCATTCCTGCACTGACAATGGCATCA
CGATTTTGATGAGGCAAATTGTCCACACCCCAACCATTACTTTTTACCAGGGTCG
TGCGCATAAGAAACATTGCTGGTGCTACGGTGGTACCCACCATGCCAGAAATTAT
CAGAAATGGCGTACTGTTATCGCCAGCGACAGGATCGGGAACAGACGGAATAAA
TCCGGCCAGCAGATCTGCGACCGGCGGCATCATAATGAAAAAATTAATCACAAA
CGCCAGCCCCATAATGCCAACCAGCGCGGAGAGGATTTTGTCGAACCCGGCGTT
GCTGTTGGTCCAGAGCATCACGTTAATCATAATGACCATCGCGATAGCCCAGGCG
AGAGCTGAGATACCGCCACTAAACCAGGTCGTGGACCATGTTGCCATTACATCC
GCCAGAATACCCATTACCCCCATAATGGAACCGCTCACGGCAACGGTTAACCCG
ATCAGAAACAACATCGCAACGCCGGGATGGATATGGCGACGAAATGCAGTCAAC
GCGCGTTCACCAGTGACGGAAGCATAACGACCATAAATACGGATTAAAAACCAG
GTAACAATGCAAGAAAGCGCCACGGCCCACATGAGCGAGACACCATAGTTAGCA
CCTGCTTTAGCCATAGTGGTGATGCTGCCTGTTCCGATGCTGTAGCCAATAAGAA
AGATGCCAGGTGCAAAGGCCGCGAGTTTTGTCATTATTCGCCGAAATAAAGAAG
GCGTTTGGGGAACAGAGGTTGTCATATTCAT
ST131 Region-12 (SEQ ID NO: 23)
AAACCACCTTCGGGTGGTTTTTTTGTATCTTCTTTCTCATTTCCGAAGATGCGTCC
GAAGATGATTTTTCTCTGGACGCATCATCATTCATTTTCTCTGGCCACCAACTACG
GGCACTACTTTAACTTTCCGGTCATATGCTGCTGTCTGTCGCGGGTTCTTATGCCC
GGAAATTGCTTGCTTCTCTTCCAGGCTGCCTTCCAGATCAGAGATACCTTTTGCTT
TCAGATCATGAAATGTGAAGTCTATTTGCAGATGAGGATATTTTTCCTGTGCTGC
CGCTTTGACATCACGCCAACGAGAGTTAAAACCATCACGGGTATACTTGCCACCG
GTAGTTTGATGAATCACAAACAGGCTACTGATTCCCGTTTTTAATGGCAGAGAAC
GAGCCAGAGCTATCGCTTTCTGCAGACGAGGCGACCAGGCTTTGATTTGCTTAAC
GCCGGTTTTTCCCTGACGAATGTAGATCCCGGAATCGAACAGCTGGTCTTTCTGC
AATGAAAGTACATCACTCTGTCTGGCCACGCATAAGTAGGCAATCTCCATTGCTA
CGCGAACAACATCAGGAGACACTTCATAAACGGCCTGATATTCTTCATCCGTAAT
ATAACGTTCGCGAGCTTTCTCTTTAAACTGTTTAACTCCCTGACAAGGATTCCTCT
GAACATAACCTCGTTCATAACCCCAACGGAATACCCGGGACATAAAGCTTTTTTC
CCTGTTTGCCTGAGTTCGGCTTGATAAACCGCGTTGATCCATATAGCGCCGAATA
TGTTCAGGTTTGATTTTATCCGGGTCGATCTTCCCGAAGACAGGCAGAACTTTTC
CGGAATATTTTGTGTAGTCTTTCCGGGTTTCGACTGCCAAATCCATAAAATCAGG
GGAGGTCATGAATTGTTCCGTGAGCGCCTGAAAAGTATTTCTTTCTTTTTCGTCAC
CGACCGCTTTTTCATATGCCAGCCATACGGCTGATCGTGGTGCATCCAATGCGCA
CAGGCGTATCGCTTTGTTATCTTTATTCCTGAATTCGTAGGCCGCTTTGCCCTGGT
AAACACGAGGCGGCATCCAGTTATCTGCTGGGTTCTTTCTCTTTCCGGCCATTAA
ATATTGCTCCAAAATCAGGTTCATCATCTTTTCGCGTCGTCACCTCCTGTAAATTG
CGAAATTTTATTGGGTTCATGAAATGGCCCCAGGTTGTTTTTGGGTGTCCGTCAG
CCCTTTCCATGAAGAAAATTCCAGCCCGGCGAAGAGCCTCACATTGCTTTGATTT
AAGTGGGCTTCCCGTCAGCTCGACCATTTCTTCTCTGGTGATTATGTCGTGATCGC
TTTTCATGGTCTTTCCTCATTATGTAGCTTATTGCGTCATCTGCCTTCTGGCAGGC
ACGGGCAATATCAGACTCGGTCAAAGTCTGTTTCCTGACGCTGGCCGACAGTCGA
CCAATTTTGATATCAAATGCGGAGAGCAGAGTTGCTCCCGGTTGCCATCGCAGCA
TTGTGATCCTCCGGTGTCTGGTGAATCGCAATGCTAGCGATGGTGAGTTTTTATTT
CTGATTAGGCGTAATCAGTTCTTGTGGGGTGAATGCCGATTTCTCCCGAGTGACC
TTAATGTTTTCTGGCAGGTGTAGCCCAAGTTCGCAGCGGCTCCGTGCTTCAATAA
TGCCATTGGTACCATCGGCAAGTACCAGGTGAACCGCGTCACCACGCTTTAATGT
GAGTTTAAGCATTAGCGTATCCTCAGTGACCGTTCGCCGCGTTCCAGATGTGCGC
CTGGTACCGGGTTTAATAACTCGGCTGGTGGGGTTTCTCCACGCGCCAGTATTTC
GGCGGCAACCGCCTCCGCAGATTCAATCACTTCTTTAATGGCTCGCTTGTTTGGT
GTAATAACCGTTTCTACATCAACAAGTGACACGCCCTCATACTCATCCGGGATCT
TATCTTTGCTATCGATAATTACCCGGATAGCCCCCTGTGCATCAGTAAATGTGTTT
TTTGCGGTTTTCAGTTTATCCAGGCCAGCAGCTTGCAGGCAGGTGAGCATATGCT
TTCGAATTGCTTTTTCCTTGCCTTCAAAGGACGTCTTACGGGTTGCCAGGCGAGA
CATTTCTTCAGCGCAGGTATTGGCATTGCCCTGTAAATTACGGCAGATGACCATC
ATGGCGTCCAGTTTGTCTCCCAGTTCTCCTTCCATGCCTTCAAGCGTGTCGGCGAT
CATCTCTGGCGTCAGTTCATCGGCGGTTTCTAACAGGTCAATCAGACTGGCATAT
TCTTTTGCAATAGCGATAGCAGTGCTCATGCTGTTTGCTCCTGTGCAGCGGTAAG
GGCGGCGAGGCGTTCAGTTTTAATGTCGGTGATACGGCGCAGACGTGATCCCAG
ATACGATGAATATTCTTTATCGCCTTTGGTTTCCGCTGCTTTCCTGTGTACATCCA
CTTCGCGGGCGATGAGGCCAAATACCTTGTTTACTTCGTTGGCGGTTACCGCGGC
GGCCAGCGTATTGGCAACGTTGATAAGCTTCTCATCCAGTTCCTTACGCAGACGG
GTAGCATCTTCCGCATTCTCGCTGGCATTCTTGATATCAAACTCAGCTTTATTCCT
CTGGCGGTATTCCGGATTGTCGTACAGGCCCATGAAAATATCGGCGCTGAATCCG
AGAGGGGATAGCGCTTTCTTAACAGCATCCGTCCAGGATTTCTTCGGTGCTTCGC
CGTCGCTTGTTGGTCCGTATTTAGTGTCGTAGATATAAGGTGTGCATCCGTAAGC
CTCGGACTCACCGCGTACCCCATTCAATATGTACCAGACTCTCACTTTCATGGTG
TGGTGCATTTCACATAGGTACCCGCCAACACCGTCTTGAATCAGATCCCATTCTA
TTTTTTCACCAACCTGTTTTTTGCGAACTATTGGTGCGCCTTTATCAAAGCGCTCC
TCCAGCACCTCTACCCCCCAGCCAATACCAAATGGCCCAAATTCGCGAGTCGCTT
TCATCGCTATATAAGTGCCGTTGATGGATGTGCCGCCGCCATTCTGAGAGAAAGC
ACTGGTGAAGCGCTCATCGGTTTTGAATACGTTCTTCCACAATGCCAGGTTATCC
GTGTCATCTGAGTCCTTTAACTCCGTTTCGATAGCTGAGATTGCATTCTGCCGCTT
GTGCTCTTCGGCATGGATCCGCTCAACCAGCGCGTCAACGTTCTGCACCAGATCC
TTTACCTGCTCGCTAAGTTGTTCTTGTGGAGGAGTCTCTTCTGCCAGGGTAATAGC
TGCAGGTTCCCTCTGTGATACGGCGTTATTTTCAGGTACGTCATACACATGGCGA
GGCGTGACAAACTGCTCACGCAACTGCTGCAAACTACGTTCTCCAGCATTTGCAG
CTTCGCTTTTTTCGCCCTGATTTGAGGGTGTTTCTGACATCATCCCATCAATTGAG
AATCGTCCGCCGCCGTGGTTGGTGACTTTTACATCATCCTTGGGTGCTGCTTCCTT
TGGCTTCTCTTCAACAGCATTTGCATCACGCTGCCCGTTAGCTTGCAGCCAGTTGT
CGATGTGACTGCGCAGGGATTGTGGGAAGTGGTACGTGTCTTTTGCGGGGGCGCT
CTGAATAACACCGAATACTGATGCGCGGTCATAGGCAAGGATCTGATCCGTTGT
GCGCAAGGCCATTGACCAGCGTTTAAAATCTTCCCTGTCCTGACTGATTATTTCG
TCTGCCGCTTTCAGGATGCTGGGGGTGATTTTTTCCGGGGCAACAGGTAACAGGG
CGCAGGCAATTTCACGATCCAGAGTGGCGTGAGTCTGTTTGTAAGGACGTGCGG
ATGCTGTTTCTGGTTTTACCTCCGGCAGCATTTCATCTCGTTTACCGGGATTCTCT
ACCCATTTTTTTATAAATTTGGAGATAGTCCAACTGGTCGGGTTTTGTTCCTGGGT
ATCGCAACTTGCCAAAATTGCATGTACCAGATTATTCAATCCAGCAATATGCATG
TACTCGATGGGCTTACACGCCACCATGGCATCAAGAACGATGCGGTTAAACGCG
TCAACCTCATCCTCACTGTCAGTATCACTGTTATCGAGCATCTCCAGGTATTCACG
CGTCTGCACAAGCAATTCACTGTCGACTTCGTTGGCGTCGTGGCTGAACAGCAGA
ACGGCGGCGAATCGTTCACGAGCCGGCAGTTTCATCAGGTCGCTAACCTGTGAG
ATATCTGCTGAACTGCAATCTGAATCAACAGGAGTGTTAACTACCCATTTTTCGC
CATCAAAACTGTGTTCTGTGGCAAACGTCTCATCAAACTCACCGACGCCTGGGCG
CGGTTGGCCCGGGGCATCTTCCCAGATTTTAGGTTTGAAATAGTTGTCGCCGTGG
TCAGGGTAGGCTTCCCACAATTTGCCGATGATGATGTTCTCGGCAACTTTTTTGTT
AGGGGCGGCGATGGCGATGGCCAGCGCCGGGATCGCGCCGTTTTTGAGCGCGCC
TTTTTTCGCTTCTAACAAGCCGTTAAAAATGGTCATTGGTCTTTCCTCATTACAGG
TCAGTGGTTTTTAGTAGGGGAGCTCATCGGTATCTGGCGTGTATTCGATGCAAAG
TAACTGCTGGATTTTGTCATCGATAGCAGCAATACGCTGTTGGGCGTCGGCGACT
GTTTTCTGTTTTTGTTCACGCAGTTGCTCGACCTGTAAACCGATGATGTCGATTGG
CTCGGGCTGGTTAACGGTAATTTCAATTTCGCGGCTTTCCAGAAGGATGTACCTG
TCTGGAAAATTTTTTGACATATCAACAGTCGCAACAAGCAGCTTTTCGTGGCTGA
AAGTTGAAGTTGCATAATGAATAAAGAGTGTTACTGGGATGGTGCGCGCTTCCAT
AGCGACTCCTTGGTAATGTATACTCAGAGCCGATCAGCGAATATTGATCAGCTTC
CATAGCGTCAGTGTCGTTGGTCTTTCCTCATCACAAGTTTGGTCACTTGTGATAAA
TCCGAATGGTTTGGTCGCCGTTCGGGGTAACTGGCCCGCCTTGTGCGGGTCTTTT
GCTATCTAAAGGGTGCCGGTTACGTTTCCGGCGTCGTTATGTTTCCATTACGACC
GTACAGGGCTGGTCAGGCCCGGGCTGGTCTTTCCTGTTTTGAGCTGGTCAGGCTC
ATGGTCAACGCTGGTCAGGCGTGGTACTTCCTCCGGTCTTTCCCTGGTGTCACGC
TGCGGGTTTGGCCTCTCCTGTTGGGGACTCAGGCACAGCGATAAAACCTGGCTGT
GTGAAAAAATGGCCCATCGTGCGGACTGGGCAAAGACTAAACACAGCAAATTGA
TGATGGGCGCCCGGAATCGAACCGGGTAACGGGCAGGGAGTTCCCGTTAAACAC
CTGTTCACCACAACCGGGAGCGCACTCCGCCATTTCAAAATTTAACGACAAAGCT
CAAAGTTGAGTCGATGAAGTGCGCTCTCGTGTTGTAGCCAGGACTCTTCCCTGGT
GGTCACACCGTATCGCCTTGATGGTGAATCAATCGCTCATACCTGGCTGTGGCTT
GCACATTCCGGCTACCTGCTATGGAGACAACGCGTTAAGGCTCCGCTTCCATCCA
GACCGCTTCGACACATGTGCCATATGCCGGTGAAGTCTTTCCCTCTGTCACCGTG
TTGGCGCCGACGCGCAAAATTTGGTGCCGTCTTTCCGGCTGTCAGAACGTGTTTC
TGAACAACTGCCGCTGGGTAAGCGTTGTTGATGAAAGTGACTTTACAATTTGTAT
TTGTAATGGTCAATGTGTTTTTAAAAAATAATTTGTAGTGAGGGGCAAAAAAATA
TTCACTGTGTAAAGGTGTTTTTGTGTTAGGGGAAAGTTGCATGCCAGCAGCGGCT
GGCCTGGAAATTTGAACTGAACTATTTTGTCTGGTTTTTGGCTTCAAGTAGTTCTT
TTAGCAGTAGATCGAAGTGCTCTCTTTTTTCTTTTAGCTCATTGATTAATTTATTTT
TTTCGCTCTCAGGCAGTCCTCTGAATAGTTGCAGTAGAGCCTGTTCTTGCGGGGT
TAGTTCTTGATTGTAATTACCCGGGTGTAGTGTGGATTCATCCAAATATTCCCCTT
CATTCATGAAGAAAAAATGAACAGGGTATCCGGTCACTTTTGCTAGCTTGTCTAA
TTTATCTTTTCTTGGAGTTACCCCATTACACCATGCCTGAACTGATTGAGCCGTAA
CCCCAATATGGCGAGCTAACTCAGATTGAGACCAGCCTAAGTCTTTTAGAATCCG
TTGTAGTCGGCTAGCAAAAGCCATGTTTTTCGTTTTTGTGTTCATACCGTAAGGAT
ACAAGGTTTAATTGTAGGGAGCATTGCAAATATAATTTGTAATTACAGTTATTGT
TTGTAATGATGACGCGGACACAAGGAGTCCATATGGATAAACAACTGCAAGACA
AAATTCTTTCCGTAATGAGTCAATCCGAGTTAGGGCGGCGATTAGGGAAAAAAC
CACAAACGGTAAGCCTCTGGTTCAAAGGACGGGTTCCCGGTGAAGAGGTTTTGC
GCACATCTGAAGCTCTTGAATGGCGCGTGACCCCTCATGATCTTAGACCGGATCT
TTACCCAAACCCAGATGACAGCTTGCCTAAAACGGAAGCAGCTTAACTGTTGGT
GAGCCCCAAATCTGATTAAGCGTAATCAATTTTTAGCGACAGGAGACGCTATGG
AAAACCAAGAGGAATTACAAAAAGAGATTTTGACCTGGGCGGCAAGGGCAGGG
CAGGAACTCGTCACGATTGAAATCTGCCGGGCCTGGTTCAGTCAGGGACGCAAT
GATGAGTTGAGACTACATGAATTTGAGGACGCGGACGGCAACGTGGACTGGAGA
GCCATCAACAACAATCGGCAGAAAATCTTTCGCTGGTTACGTGGCGAGACAACG
GCGGCGCGCCGAAAAACTCAGGTGCTGGCCAGTGTGATGAAAGCCGTGCTACCC
GCAGAACGGCGGGCACGTCTGGAGTCGCCGGGCGATCCCGTTTTGCTGGCAACG
CTGGCGGCAAAAGAAGGGGTGGAAGCGATTAACGCTGTACATCTCCACTTCGCG
CCAGAGTTAACTATTCAGGAAATTGACGAAGCAATAGCGGCGCTGGTAGCGACG
CGAGGAGCAGTGATACGCACTGCGCAAGACCACCATGCATGAGCGCCTGACCAG
CGTTTAACTTATACCGAGGAAAGACCGATGTTAAGACATATTGACCGCATTACCT
GGCGTAACGGCTGGCACCTGAATGGACGCCCGGCACATGTTGCAGAGATCCGCC
CCATATTCGATGGCCGCGTCGCAGCTGCACGTTCTGTATGGGAAAAGTACGAAG
AAGAAAAAGCGAAACTGCGTGAGCAAAATCTCTCTGGCGCTGCCTATGAGGCTG
GCTGTCGCGTTCTCTCAGAGGCTTTGGGCATATGAACATCCTCCCGTTACTTGAT
AGACCCATAGCCTTCCAGAGAAGTTTTATCCGTCTTGAAATGGGCGTGACTGCCG
CATTGTTTCTGTCGCAGTTGACGTACTGGACAAACAGAACAACTGACGACGGCTG
GGTATATAAAACCCAGGATGAGTGGGAAGAGGAAACAGGGCTTTCACGATACGA
GCAGGAGGGCGCGCGCAAAAAACTACGTGGTCTCGGCGTCTTGCTGGAACGTAA
ACAGGGCTTGCCTGCCAGGCTGTACTACAAAATCGATAATGATGTGTTGTGCCAG
CTACTTACGTCCGCATACAAGGATGCGGAAAAACCACATACAGGTGAGGGGAAA
ACCACCAGGCCTGTACGTGGAAAACCAACAAACATTCTTACAGAGAATACTACA
GAGATTATTACTGATGGTGAATCGGCTGACGCCGACACCCAACAACCTGCCGAC
CAGAGAATAAATTATCAGGTGATACTCGATACCTATCACGAGATTTTGCCAGAG
ATGCCACCAGTGAAAATCCTCACTGACGGCAGAAAAAAACACCTTCGCACTTTCT
GGAAAAAATTCAATTTCAATGAGTCCCGTTGGCGGGCCTATCTGGAATTTATCGG
TGGCAATTGTCGCTGGATGCTGGAGGACAGGCCAAATGGCAGGGGAGGATTCTG
GCGGCGTAAAAATCTTGATTATCTGATTACCGAACGCTGCTATGTTTCGGTCAAG
GAAGAACGCGCAAATGATAAATAACGCGATTGCACTGTACAGCGTTGACGCAGA
ACAGGCTGTTCTGGGCTGTCTGATGCTGAATACCGACCACGACAGAACCAATGC
GGTATTTGCGTTACTGAAACCGGAAACGTTTTATATCAATGCGCATCAGGTTATC
TACCGGGAGATCAAAGGATTATTTCAAGCTGATAAACCAACTGACCTGATAACA
CTGACCGAACTGATGGAATCGAAGGGGCTGTATGAGCGCGTGGGGGGATTCGCA
TACCTGGCTGAAATGAGTAAGGCGGCGATTCCGGCCTCAATGGTCAACTACGCG
AAAATTGTACGTGAAAAAGCCATTCTTCGTTATGCCGTGGAAAAGTTGAATTCTT
GTCTGGAGATTATGATCCAGCCTGCAGATATGACTGCTACTGACAGGATCACCGC
CGTTCAGCAAGTGATTGGGCAGGTGGCCGAACATTCCCGTACCGGGCATAAGGG
CGGTTTGCGTCCGGCAAGTGAAGTCGTCGATGACTGGATAGATGACCTTGAGCG
CCGTTTCAATGATCCAGCACATGCAGCCGGACTAACGCTCGGAATCGAAAGCCTT
GATCGCTTAATGGCACCAAAGCAGGCGTTAAGAGGTTCATTGGTCGTTATTGGTG
CCCGGCCAAAAATGGGCAAGACAGCCGCGTTTAATAAAATTGCGGTGCATTTTG
CTCTGAATCATCGTTTGCCGACGCTGGTATTTAGCCTGGAAATGACTGACAGAAG
TCTCATTGAACGCATGGTTGCGCAGGAAGCAAAGGTCAATTCGGAGATTTTTTAT
CTTGGCCCTAACGATGAATCTGAATTGAGTCTCGCAATAGCAAAGGCGGGGGAG
ATTGCCGAATCCAACATGATGATTGACAGCACGGCAGGTGTAACCCTTGCGCAT
ATTGTCGCTGAATGTCGAAAAGTTAAACGGATGCGTGGCTCGGTTGGCCTGGTGG
CAATTGACTATTTGACGCTGATGAAGACCGAATCGGCAGAGCGTAGGGATATTG
CGTATGGCGATATCACAACCGGTTTGAAGAACCTGGCAAAAGAGCTGGATTGTG
TTGTGGTCCTGCTGACGCAACTCAACCGAAAGCTTGAAGAACGTGCCGATAAGC
GGCCTAACCCGAGCGACAGTAAAGATACCGGGCAGATCGAACAAGATTGCGATG
TCTGGATTGGCCTCTATCGTGATGCTGTTTATAACGAAAATGCCGACCCAAATCT
CATGGAAATGTTGCTACGTCTGAACCGCGAAGGACCATCGGGAACAGCCTATGC
CCTGATGAAAAATGGCTCAGTTATTGATATCAGCGATGAGGAGGTTTATCGCCTT
ACCAACGACCGCTCAGAAAAAAATAAACGTTATTCCCGTAGCGATAATTCTCGC
ACAAATGAATTTTAACCACGCCTGACCAGCGTGAAATAACCGAGGAAAGACCAA
TGACCACCACTCCAGGCAAATTAGAGTACCCGTCGGACAAAGGACACATAGACG
ACGGTAAAAACTATCTCGACGTCATTTTGTGGAATATGAACGTCGGGCCACGTGC
GCGCACTCGCGCGGTTTTTGTGCCAAGACCTAAAGCAGGCAATTTTTCAACCCCT
GCTAAGCCTGCCCGTCAGGCATCCGTAGCCGCAGCGCCAGCAGTGAAACGTAAG
GGCAAAACGCACACCGGGATCGCGATTCGCAGAAATGGTGAGCGGCAAGTGAA
ACTCCACGAAACAGCGACAACCTGGTGTGCCTCACCGCATGAAACTTACGACAA
GATAACCGGGCAGCGCATTGGCGCGCCTGGTCGCTGCCGCCTGCTGCTGAGTTCC
ATCACTCCGATAGCAAAGAAAGGGGCGTGATATGGCCGGGCAATCAGATTACCT
GCCGCCCGGCTTACCACACAACCGCGCCAAATGGCCCCAGGAATACCAGCTTAA
AGAGCACTACGACATGCGAGCGGCGGCGCTGATCCGCCAACTCTTTGAGAAACG
TATTCCGAGGGGAAGCGTGATTGAGCAAATCGGAATGACGCCAGATACGTACCG
GGAATTTTTCAGAGAGCGTCTGAACTACTGGAGAGGGGTGATGGAACAATGAAA
TACAAACGGTGGGTGCGTGCGGAAGTCGTAATCATCAAACAATGTGCGGGCAGC
ATGACAGTTGAACGTATAGGGCAGCTTATTGGCAGAACCGGTGCAGCGGTACGC
ACAAAAGCGCGCGAGCTGAAAATCTGTATGTATCTGCGGGGTAATTATCACCAG
TCAGTGAAATACCTCCAGGAAGATATCGAGCTGGCAAGGGAATTACATCAGTCG
GGTATTAATCGCCAGGATATCGCAGAAAAACTCGAAATGCCGATCGGAGCGGTG
AATCAGTTTGTTTATTTTGAGCGGAGAATTTCATGAAAGAACTTTTTCTTGCATTT
GTGCCCCGGTTTATTAATGACCAGATCGCACTGACTGATAATGGTGAACAATATG
AAATTGCCTGCAGCATGGTGGATGTGAATCCTGGCGAACGGTATGACGCGATGT
GTGACCTGAAAATATTTACCTGGCTGGGTTGGGCTATTCCGTGTGGAGAACCAAC
CAATATTCGTCCGTTTGAGAGCAGGGAGGCTGTATGA
ST127 Region-1 (SEQ ID NO: 24)
ATGTCACGTAACTGTGAATCTCCAAAAGGAACATTAGTAATAGATCTATCAACA
AAGAACAAGGAGATAATATCGCAAGCTGCTGCAATGGAACAAATCGACTTAAAC
GATTATATAGTCAACCAGCTATTACTTAATTCGCTAAGAGTTATATTTGGAAGTG
GTAATGTTACAAGTGATGACATAAGGAATATTGATAACATTATCAAAAATTCTCC
CTCACCAGACGAAGATATGCTTAATGCGATTTCGATGTATAATGAAACCTTTAAA
GAATTAATTAAACCTGAAAGATTAGATAAAAGAGATAAAGCTTCAAAGAGAAAA
CCATCCTATGGTAAGGCAGCCCGAGATAATTTAGCTAAAGGAGTACCTATTTATT
ACACAACAGGAGGAATACCGAAAGGGCTAATAATAAAAGAATACCCCTCTGGG
AAGAAAGAATTAGTAGATTTCTCCTCTGGTAAAGAGGTATATATCAAGGATTACA
AAGGATGATAGCCATTGAGTAGCCCCATTGTCTATTTCATAACGAGCTTAAGATA
TGGATGTTTCCAGTCACCTTTCTTGAGCTTGCGGCGAGAAACGAATCCATCATGC
TCTAAAGCACAAATACCAGAGTGTGCGGCAACAAAATCATATACAGCCCTACTC
TCTTCGCCTTGTAGCATATGAGCGAGAAGCTTGCGTCGCATGACATCAGAGCGCC
ACCTTATCTTCGCCGCAGGATCTTTGTAAGTACAGTTGAAGATCTGTCCGACTGC
ATTTCTCACACATAGGCCATAGCGGTTTGTTTTACCCGTAGACAAATAATAATCG
ATTAATTCATTAAGATCATATTTAAGCGGTTCCAACAATGTTCGCCAGCGAAGAA
GTATTCGCCGGGTTTTGATTTCACCATAGGACTTATACAGAAGCTGTACAGTCTT
GCTTTTACGGCTTAAATTTACATGACCTGCACTAAACACAGCAGAATATCTAAAT
TGCTTAACTAAACCCTCGCTTATTTTTAATACCTTGGCGATATATTTAGTTTCAAG
GATATGGAGGTTCTCATCTGATACACCAATCTTTGTTAACTCATCGCGCAGAATT
TCAAGCTGACAACTTTTAATGTCATAGTTATATCCTTCTTCAAGGCATCCCCACTT
CATTCCTTTTGGTAATCCCTGAAAGCCTGTACCATTTTCAAAAGAACGCCCTCCT
ACCTTAGCTGTTTTGTAGGGTTGCCTGTATGAGATAACTAGGGGATTATCATCTA
CCTTACGGCAATCAGTCTCTGCAAGATGAGAAATAAAGTTGAAATAATAACTCTT
GTTTTTAGGCGAAGGATTATCGTTACAATAGGTTAAAAGCTTATCTATGTTTATTT
TAATCTCATCGAGATTTGCATAAACATTTTTCACCAACTCTCTGAATTGTTTATCA
GGAATATCTCTTTTGGATGTCAGGTGCTTTAATTTTTTATTATTGAAGAGTAGCTC
ATTTAAGGAATACTCTTTTTTATTATATATATCGCCTAATGATACATAACGTATAT
CCTTGTTAAGTTTGCTCTCTTAGAACAGTTCAGAAAGAAACTTTTTGGACAAGGC
AAATTCTCTGCATTTATGCCTGTTAAAATCATGTCGAAATATCTTAATGAGACCA
GCATTCTCAATCTCTTCCAGGGCACGAGAATAACCAAGGTTCCGCTCTTGATCGA
AAACAGAAGAGGCTATTTTTAAACCTATACTCTTTGGAATTGGGACAGAGTAAGT
CCATTTCTTATCATCTTTCTCCTGATACTTTAGTTTCTTTCTTTTGATAGTTGTACA
GCAGGCAATTAAATGCTGTAGAAATTTGGCAATTTCTTGATAATAAATACGAGAT
CGTCGTCTATCACTTTCATTATCAAATGGGATTGAAAAACCATATTTCTAAACTTA
ATAAAAGTCATCCTATCTGTGAATATTTTTGCTTCTGCAACAGTTGTTCTTTTCAT
AATATTTTGCTTTTTTTATATATTTAAAATAAATTGGAGGTAATTTTTAAATGTTC
TACATGTATAAATCCATCAGGGAAACCTCGATAAACCTTTTAAAAGCTAGTACCA
TCTGAAAAAAATCATCATGATCTTAATATTTCATCCTGTAGTCATAGATACTTATC
TTTATTCAATGAGACAAGATAAAAATAGAGATATAACCTGTTTTGAGCTAACACC
ACTTTTTTGGGGGGTATTTTTGATCTTGAAGTTTCATCTACAACATATTTATCTTA
ATCTAAATAAGGTTGCAATGCATGAATCTTAGCAAATATTTCAAGGATCGCGCTA
TCACTTTTTTGATTTCCCCCTACTATAGCCCATATATCTACTATATATATACATCT
CATCATATATATACATGTCATAGCCTCCATAAAAAAAATCTATATCTATGTTATC
AGCGTGCGTGAGCACGTAGTATGTTGAAGATGTTAAGCTTCAACATACATATCAA
GTAAGGTCCATTATTCTGACTGGATTTTTTATCAGAATAAACGCTGATCTAATGG
GAGTGTTCTCTGAACAAAGGCAATATCTCCATTTATAAATTTCTTGTACTGATTAA
GATCTTTGTAAGTGTCTTCGTGGATTAAGAACACAGGCCTGCTACCTCTAATCTG
AGACATGTTTATTGATAGAAAAAATTCGAACATTTCTCCCTGAACTAAAGGTGTA
GCCTCTGCTTTATTTGGTCCTTCCCACACAATAGCCAGCCCTATGCAGTTATGTTT
ATTGTATTTAACCTTACATACGGTTTCATATATACCCGTGATCATTGAAGATACTT
GATTTTTCAGCAACAGAAGATCA
ST127 Region-2 (SEQ ID NO: 25)
CTATTCTAAGTCTTCCTCATCAACATCACTATCATAAGGAAAGAGTGTAACTTCT
TCCTCTGGTATAATCGGCTCGCAGGCCTGAAGTTTATCGTTATCGACAAAACTGG
TAGCCATAATACTGTTCTTGAAATTATTGTCACTTATCCATTCAGAATTCCAAACA
TGCTCATGAGGCCTATCGTGAACGCCAGAAACGATAGAGCAAAGCGTCTCTTTAT
ATTCAATATCTCTGTTAATATGATAGGAGATACGTTGCAGCCAGATATCTAAAAT
CCCAGTACCTAAGATAGGTTTAAACTTATCTTTGATCTGTTTGAGCAACTCTAATT
TCTCTACGTCATTATCTACAAACTTTAATACATTACTCAACACTAACGCACAAGA
TGAAAATGTTCTTGGATTATTAATCATTAGATCAGAAATAATACTGACTATCGCT
CGAACCTCCTCCTTTATGCTACTCATGCGTTCTAAACGCTTATTTATCTTAGCAAG
ATAGGCTTCAAGCTGACCTGAATTTTTATACTTACGAGAAAAGATAACAAGCTTC
AAAAGAGTCTTTTGAATAGTTGTGGCATTAACACTTCTTCCAAATACCATCAACG
CTTCTTGTTTATCTGGTTTCATTGAACCAAGGACTAAATCTTCAGTAAGTGAAGTT
TTAGAAGCATTAAGCTGTAGCCCTAACCCTTGCAGAATAACAGTTAGATGTCTCG
CTATCGCCTCTGCATCCTCTTTACTATTTGTGAAGATTCTATAATCGTCTCGATAT
CGAATGATATGATAGTCATTGATATTCTGGCTTTCGAGTTTCTGCCCAAGCAACT
CATCAGCATAACCAAGAACGATTTCCGCAATAAAATCCATAAGGGCAGAACCTT
GAGGAATGCCGTTAGTCTGCCCCCATCGCATCTGTCGTAAGGCATAATCAATTTG
GTCTCCAAGATTTCTACCTGCTCCTCTTGCTGCTTTAGCATCTTCTTTTGTGTAAAT
TGCCCAAGGTATGGAATGGGTATAAATTGATGGATAAAAATTTGCGATATCAGT
ACTGAACAGGTATTTAAACTGAAGAGATTTATTGATAGATTCTTGTTCAACATCA
CGCCACCATCCTGATACAGCTTTTGCCTTATCAGAAACTCCATCTTCTTCAGATTC
TCTTGGAAGGCTTGCACACACAATTTTTGTATTGCTTTGGAACTCACCAAACCTTT
CAAGCAAGAGTTGCCATGATTCATCTTCCGTTATTTTATGGACAAGATGAACGTA
AATTGCAGGATGAATTAATTCGAATGGTCGCCAACAATAGTGGCCATCTTTATTC
GTTTGTAAGATATAGTTGTCAACGACGGATGAAAAGTGATCCAC
ST127 Region-3 (SEQ ID NO: 26)
GTGTGGATCACTATTGCACCGTTCGTGACAATAGTTAACATTATCGAAATCGCAT
GCTTTTTTGTAACCGATATCATTAATACCGTTTTGCTTTTGTTCGAGAGCATTTTTC
ACTGCGAGCAGAAGAGGCGCAAAGTCAAAATAAGCAGGAAGTGCAAAACTACA
ATAGCTCTTGTGCTTCAAGAAAAAGCTTCTTGCGCTCTCTGCTGTCATTGCGGTA
ATCAATGTCATGAATACTAACCCTAATTATTACTTTTGAGTCTCTATTAAACACTC
CTTTGAACTTAATGTCTTATGTTGAGCCACTAAAACGCATCAATGGTCACAATTC
TGCTCATCAGTTTTCACTCAATAGTATCAGAGATATACTATTACCCTTCTTACAAC
GCTTATTTATTGGCCAACCATCATCCCATTGATAAATGATCTATCAATGGGATGA
TAGAACCGCTTTTATTCGAAGCGTTGTTCGCGCATCTCATCCTCCTGCTGCTCTTG
CAACTTTTCATGTTCTATCCAATCGTTTAATTTCTCGGAAACAATTCTAGCTGCCG
CAGCAATATCTGTACGCTCATGCTCTATTAATCTACTAATAGTATTTGCTCTGGGT
TGCATTACGTTAACTCGACTTCCCCACCAAGATGTCGGTGTTACGCGATAAGCGA
ATTCTTCCAGTACGGCCTTTGGCACAGGAGCTGCTTCAAGGAAGCGAATCGCTGA
AACCTGCATTGTGACCTTATCCAAATCATCCCCCACAGACCAAAGGCGGATACCC
TTTGCGAGAGGTGCCCATACATTAATATCGTTTCGAGTAAGACACCATTCGATCA
ATACATTCACGTCGATATTCGCGATAGGGGACTCTGTCACATCATCATAATGGAT
AAAATGAATACGTCGTTGTTGCTGTTCTTCTGTCCCTTCAAAAATACGAGAGAGG
AACGCTTCTGGCATTGATGCCGCAGTCATCTTTATAGTGTCTTCAAACGAATGAA
TACAGCCATACTGTTCATCAACAACAGCAAAGATCGTATTTAACCACTCTAACTT
CCCCGCCTCGTTGCCTTCGAGACGCAATATGGCACCAACAACACATTCCATGCCG
TAATCCATTCCCCCAGCGGAATCATTGTGCTCTCTTTTGAACCTCTGGATTGCAGC
TTTCAATCCTATTTGAAGCAGATCAAAGCCAAGAGTACTCGAACCGTCTTTCTGG
TCACTTAACTTCATGCTTAAAGCTTCCAAAATAACATCATCGCCATTTGGCATAC
TTAAAAGCCTGTGAGCAAGATCAACAATGCGCGGCTCTGGCAAATGAGCGTATT
GGTCTCCCCAAAGAATTTGTCCATACATTGACGGCATGATGTCGGGGTCATCTAA
AAGAGCCAAACAGCGATCCAAGTCAGTTTCAGTGAATTCTCGCCAAGGATGTAA
ATAAACAATTCCGTATCGCAACTCAGGATGACGCACACATTGATCGAGAAATTCT
TGGGCTAATATTGAATCAATAGTATCTGTCTCGTCGATAAAGCCACGGATAACTG
CAAAATCCTTTTGAGAACCGGTTTGTATATCCAAATAAGCAACGAGTTGTTGCCA
ACACTCCCGTAGGTCAAGTGCCCCTCTTGCTAAACCTTGACCAAATTTAACACGG
TAGGGCATCCCCCCCACCAAGAACAGATTAGGCCCCAACTCATCAAGCGTATGC
TGAGATGACGCGAAATCCTGCCCCAATTGATAAGCTTTAGAGTCAAGGTATGTCC
TAAGATTAGCGAACTTATTAGGTTCATCAGGAGAAGTATCGGCACTAAATGCCA
AATAATCGCGTCCGTTACTTAATACATAAGCCATTATTTCCGGAACCAAGTCATG
TGGTTCTAACTCACTCTCCAGAGCGACAAGATCGCTTGGCAACGGCTCTAAATCT
TCGATATCTCTGCGCTTGGTGTAATCAAAATATATCGTTGAACGAACGGCCTTCC
ATCCTTCAACCCACGGATAGTAAGAATGTAATTTGTGAGCAGCATCAATGAGCAT
TGTCCTGGCAGCCTCTTGACGCCATATTGATCGAAAAGCCCTCGCTAAAAGCTGC
CGCGCAGTTTCCTCAACCGCAGGAATTCCGCAAGTTCCTGCATTAACCGCAACTT
CAATGAATGCACTACGCCATTCAACAAGTTCTTCATAGGTTGGTTGAAAACCATA
ATCTCTTGGTCGTGCACCAAACTCATTAGCCCCGAACCCCGTCCAAGGTGGGCCG
CTGAGGGCTGTTGAAAGCATCATAAGGCCCAATTCTTTGCGTCCAACGACGTCTG
ATGAAAGGCATTGTTTCAAAATTGCAAGGCGCTGGTCTAAAGAGGCCTGTGTAC
CAGACAGGTATGGCTGATAAAGCCTCTTGATCTTACCCCGTACAGAGTCATGGTT
GTTGTTTTCAACCTCATAATCTACCGCATGAATTAGCAGCCTGACGCAACGTTCA
AAAGTTTCCGGTTCATATGCCAACGATTGCAGGAGATTGAACATAGTTGTCCGTC
GTAGACTGTAACGCCGTTCCATACCTTTAAAGTCAGATGAAGTAAGCTCTTCTTC
AATCCTGTTGAGAAGTGTTTCAGGCGCAACCGGACCGATGTATGCCAACATTTGT
ATTGATGCATCATCCAACTCAGTTCCTCGTCCAAGCAAATCTCCTGGTTGAAGCC
ACGCTTCAACGATTTCTTTCGCTACAGCGTGTTCATGGAGCAATCCTAGCCGGTG
TGCGAATGATATTAATAAACGTTGGCGACCGGGAGCTTCAAAGGTGTCTCTGAGT
TCATGAACGGGGATGCTATCAAGTGCCGATGCAGCTAGCTTGTTTGCAATAGCAT
GAGGTAGAACAGCCCGCCAATTAGCTCTTTTCTGGATAACGTGACGGTTCAGTAA
ATTAGTAACTGTTTTGAATAACTGCCTGTAGGAGTACCCGCCGAGTGAGCCTAAC
GCCTCCAATTCGTTAATCCCGCCTTCACTGGTTGAGATGGAGAAGGAATACACTA
AGGACAATATTTCAGCTTGTTCCCGCAGATCATCATCGGGATCATTACGCTGCTC
AAAAAGGCGGTTAAACAACTGTGCATCAGACAATTGAGCTAGACTTTCACCCTCT
TCTACCCTCTCTGCTATTGCCAAGGATACACGTGCATTCCCATCAGCAAACGCTG
CTATTCTACGGGCATTGTGATGGCCTATACGGGGGAAGCGACGAATCAAAAGCT
GTTCAGCTACCTCCGGTCCAAACGCCTCTATGCGGATTACCTCGGTTGTTTGCGG
TTTATCGTCCCTAATATCGTATTCGATGGTAATAAGTCTTACCTTGTTACCTACGG
CTGATATCTTACCTGCCAATAAAGAGTGTAGTGCTGATGGGCAATTATCGAGGAT
CATAATAGCTCGTCGATCTTCGACAATAAGCCGCTCCAGCATAGCCGTTGCCGAG
GGGGCTGGGTCAGCACCTGTATCAACATAGATTACAGAGGTTCTATCTAGAGGG
TCAGATCCAACCGATTCATCAAAAAGTGCCTGAACGATCCGAGTCTTACCTACAC
CAGACAATCCTGTTATGCGAATAGCTTTTTTAGTAGAACGGATTAGTTCTCGCAT
CGGATTGATAGCATCTTCAATGCTAAGCTTCTGCCCTTGCCCTGATGGAAGAGTG
ACCGTGACTCCAGGAGCTAAGATTAGAGTGTCTACGACGCCATTGGGAGGATTA
CTCCACGCACCGTATGGCTGCCAGCCTGAATATCCCTGTCCGAGTATCCCTTTTA
CCCACAACATAACTGACGGATGCTGGCGTAACCATTGAGTCAGTTTCGAGCGATC
AAAGAAGTCGAGATGGATATTGCCATTATTTGGCTCATCCTTAACTGCTTCCTGC
ATTGCTTGCAGGCGTTTCATCTTAGAAATAGGAGAACAATCATCACTGAGGCAG
ACAATTACGTATCCTCCTCTTTTAGAAACTTGCTCAGAGATTAACGGTAGTAATC
CTTGTTCAGTTTCCATTTCATTCTTGATCGCGGACGCACTCATCGAAGGCTTCTTG
GCCTGAAAGATCGTGTCGGGCCTCTCGATAAACCCTGTACTTAGTTTGTCGATAG
GAACCTGAACATGAACGTCAATTCCTCCGTCTGGTGCATTGATGGAACCAGACCA
ATTAACCCATGCGGGACTGTGGCCATGAGCTGCTACCTCTGCCTCAGCAAGACGA
GCAGTCAACTCTTCAAGTTGACTATCGGTGAGTCGGAGTAATTCATCTTTCTCAA
TGTCAAAGATCGCCACATCCAAACCTTAGTAAAATTATTACGGCCACAGTCAGAT
TTAGCCTCCTAGTCTAACTAAAGTTGAAAGAGTTGTCCGCTGAACTGCCCATAAC
CATAGAAAAACGTTGAAGTTATTGGGAAGCGTCGGCTTAGGCAATATCGTTAATC
ATGCCCGCAACTGGCTTAACGCCAGCATGCAGATGGGATTTAGCTCTAATCTTCA
ATGAATGACGGTCTAAACTGACTCAGGTAAAGGGGGAGGCATCTACCTCCCCTG
TGATTTGCTCTTACTGTGATAGTTCCTTTTCACTCAGAAAACGAGCACGACGGTT
ACAGGTCACTTTTGTTCTGGCTTGCCGAATGCTTGATTTAGCCAACATTGCAGGA
TTAACAAAGTGTTCATCTAAGCATTGTCGGGCAATCGCGAAGTCGTAGGCTAAAC
TCAACTCTCGCATTACATTACCCGCCTGAACTCTTGCTCCTCGTTCAACAATACTG
TTAACCAACTCCACCACATCCTTACGCGTAACAGCCATAGCTTCCATATGCCCCA
GCACTCTGATGGCATCGCCTTCCAGAGTTCTCCGGCACTCTTTCTGGCCTTTAACT
TTGCGTGCGCCACGGATCAGCTTACCATCAGAGTCTTTACGGTCCTCAATCCTCT
CCGTCAGATATAACTCGACTACATCTTTGACTGTTACAGGCTTCTGCTTAACTTTC
TGCTTTTCGAGTTTAAGCTGCTTCTTTTCCTCATCCAGCTCAGTTCTGGGACATCG
CCCCTGCTTTCTGATTATCTTCAGTTCGTGTAACCTAACGCGGGCATCAGCCAGA
GTGGTCACAGGATAATGGCCTATCAAGATCTGTTTATCTTTACCTGTTATTGGACT
GGTATAGCGGTAGAAAAATGTGCAGATTCCCATTTTTCCACGCGTAACACGAAG
GCCTCTATTTTCGCCCGTATCTGCCAGAGTCATGCCGAGCTTCATGGTATCTATAC
CGCGAGCGGTTAGAGGTTTATTAGGTTGTTTTTCAGACACAGAAACTCCATGTAA
CTGGTTGATATTAATGATGTAAATCTTATAAGTGTAGGTTGTTTTCGAATTCAGGT
TATCACCAGACTCTTACATTGAAAACCTACACTTTGACCTACACTTTTTTTTGAGA
TTTGGTGATTTTTCATGAGACAACGTGACACCACAAAAGATTGTAAATCTCTTAA
AAAGTATTGTTTTTCAATGAAATGGAAATCATAAGTCCA
ST127 Region-4 (SEQ ID NO: 27)
TCAGTTTACGCTTACCCATTCATAAATACGCGGTAATGTTAATACTACACTCTCCT
CCGAGTAAACAGTTGCGAACTTCTGCTGTGCGCTCATAGCAGACCTTGCAGGTCT
TCCGACGGTCCGCTTCGTGCCAGGGGCAGACATAGATAAAGCATGCTCTGCATG
AATCATTGAGGAGATAGTCACTCTTGAAATGTAAATCCCCTGGAATTCTCACCAT
TCCATAACTGGTATTTGTAAGCTTTAGTATGATATAAATATTATTTTATGGATTTT
AAGAAAAACCAATAAAAGTTCTATTTTTTTTATAAATGGAGAACAAAGATGAAT
AATGGCGAAGATAAAAAAACAGCTTTCTTGATAAAGGATTAGAAATATCCAAAC
CTATCGCAGGATTATTTTCTTCTGCAATCGGTGGCGGTATAGCGGGGTCTTCAGG
AGGTATGCCTGGAGTCGTTATAGGGGCGGTTGCATATTACCTTGCAGATGTTAGT
AATCGAGGGTTATCTCGGATTCAAGAAAATAGAGCATTAACTGCGCTAAATATC
GCATCTCGAGAAATCGAAAAAAATTAACTTACAGCAGAACAGTAGAGATATGGC
TAGTGACCTCTGCGTATAGACATTTTTTTTAACATAACCACACACATATATTATAT
CAATATATTATTGTTAATCAAAGCACTGCTGAGTGCTTGCATTGTAATACTTTCAG
GTGAATATCTGTGCCAGTAAAGCTTCACCTTTTGCTCAAACTCTGGACTCAGATT
TATAATTTTACCGGAAGCAAGTTCAAAGCTTATTTGTTGTTTAGGTATCATACAA
CATGCCAGGTGTTGTAATAACATTGTGACATATGCTTCTGAGGAAGATACAATAT
GACTTTGTAGCGTTCCTGGCACCAACCCAAAATATTGCTGCAAAAATTTATGATG
CATATCTGTATCATGATTAAAGGATACAATCGGGGCCTTTAATAAATTTTCCTTTG
ATACGCCCTGGCTGAAATATCTGGAATAAAACTCTGGTGTTGCACAAAAAATAT
AATCGAGAGACCCAACATAATCACATAGCCCGCCAATGATTGCCTGCGGCTGAG
AACTGATACAACCTACAACCTCTCCTTTTAACAGACGCTTAAGGTTTTTATTTTCA
TCATCCACTTTTATATCCAGTCTTATAGATGGATTTTTTAAAAAAACACACAAGG
CAGGAATTAGCCATGTTGCCAGCGTGTCAGAATTCACTGCGATCGGAATGGTCAC
AATGCTTTTATTTGCATCAAGGTTCAATACATCATGCTCGAATATATTAACATGAT
ATAGCAGTCCTGCTAATTTCTGACCATAATCAGTTAAATGTGGAGGTGAAGTACG
AGTAATTAACTGAACACCGCACTGATTTTCAAGTTTTTTTATTTTCTGAGATATTG
CCGATTGTGAAATACCAAGAATTCGGGCCGCTCTGTAAAACCCTTTTTGCTTGAT
AACGAGATCAAGATTTTTTAACAATGAATAATCAATAAAGTTTCTCATTTGCAT
ST127 Region-5 (SEQ ID NO: 28)
TAGATATTTGACCCCGGACTGATGATCACCATCAGTCCGGGTTAATAT
ST127 Region-6 (SEQ ID NO: 29)
TATTTCTTCCCCTCCGGTTATTTTGTTTTCATACTGATTATTACCACACATCTGTTA
CAAAGCAAGGGGGACATTATAAGCATGATCTTATAATCAGATTATTATTATTAAT
TTTTAATTATAACATATGTATTCTGTGAACTAGTTGTTTCAATCAATTCATCTGAA
ATCGATAAAGAACTGCATGGAAAGTTGAATCAACCAAGAAAGATATCACACGAA
ATCACCTCGTTCAGGGAGGTGACATTTGCTATAAAAATGAACAGGAAATGCTGT
GAGGAGGGAACATAAATACTATTCATGTACCGCCATACAGGAGATTTTATTGTGT
AGATATTCTTTGATATAATCTCTGGGGCCAATACTAAACTCTTTCTTAAAAGAAT
AATTAAAAGAGGATGATGAACTGTAGCCTAACTCTATTGCAATTTTTTTTATCTTA
TCATTATTTTGTATTTTGCTTATAGCTTTCAATAATTTTATCCAGTTATAAAATTGA
TATAATCCTGTCCCGGTGTACCTCCTGAAAATCCGCTTAAGATAGTCCTCCTGTA
AATAAACACTTGCGGCCAGTTCACGAATCGTTGTAGTATTATTATTCAGCATATT
AAGAACAGTCAGTCTGACTCTGACATCAGTATTATCATCTGCGATAAAATCAATC
AACTTCATCTCCAGTAGTGATAATATAAGTGTAACCGATACTTTCCCATTAATAG
ATGGAGTAATATTTCCAATTGTTATTTTTTCTCCTGTTATTTTAAATATTCTGTCTA
TTGTTTTTTTTATTGTTTTTGAATATGCAAAATAGTGCGTATTTTCAGAAGTATTCT
GAAAAGATGTAATTTCTTTTCTTGTCAGTGTAACACTATAATAAATGTTATTGTCA
TAGCCAAAGTGGTTTAAAACTATTAAACCCGCATCATTAAATCCCAGTTCTTGTG
GAGAATACAATAACCGAATATGAGGGGAAAGGACTTCTTTTATAAAAGAAAATA
CTGTCATATACAACCTCAATATATCTTATACTAACTGTATTTGCTATGTAACTTTA
ACATGCATTCAGTTATCAGAGATATTCTATCAAAATGGTATTATTTTACACACGC
AGATATGCCTGTTAACAATTATTTATGTTTTACACATTAAGCTCGACAAGCTTATT
GCGAGAATAACTTCCTCACAACAAACTTGTCGAATATTACAGAT
ST127 Region-7 (SEQ ID NO: 30)
TAATTTTATCCTCTTTAGCCAAAGATTTATTAATAATTAACTTCCTCAATGACATA
TTTAGTATTTACTTATAGTAATTCGATGATGTGATATTGATCATGAAAATTTTATT
AAGCACGAACCTTTAGTTTTGCATTTTAGATATAGTTATATATTCTGGATAAAGTT
CAGTTTTTTGTTTTGTGATGTATTTACTCAAGAATTTCCTTATGCATAATTTCTTCG
CTATGAAGCATGGAGCGAAGAATACTTATTCTGTAATCTCTTGTTTTATTCCGAA
GATATGTAAGTGTGAAACCTGCCAGCACTGCTTGTTATACCAATTTCTTGCAAGA
CAACTGAGAACCTTTCAAAATCAGATGGAGTTCTAATGAATTCAAACTTCGTA
ST69 Region-1 (SEQ ID NO: 31)
TTAGAGAACCTTAGACTGTTCTGGGAACCAGTTATTAATTATATGTCTTCGACAG
ACTTTAGATATTTCAAATACCTGTCGTGACACTAATGCACCATCAGGATGGCGCT
GGTCAGGTATGTCATTTTCTATGTCATAAAAATTATGCGTTATTTCTGGGGGAAA
GAAAACGTTTTTGTCTAAAGGCACGCTTGCATAGGGATTATAATACACATGCAAA
CCATCAAAATGAGTTTCCTTGTAGTCCCTTGTTCGGCACATTACAATATCCGCGC
CAACAACCTCATTGTTGTAGAATTGTCGCTTCGTAGTAACATAATGGTCAGGACC
AAGTTTTCTCGTATGAGAACCTTCGTTCTTCATACCTTCATTGGCAATAAAAACAT
TCACATCAAGAGCTCGGAATCTCGTATATCGGACATATCGATCGACCCGACTCTG
AACGATAGCCTTACCAAAAGTACCTGTCGTTGAAAACACGATAGCACTGATTTCT
TTGTATGAGTCATTTGTAAAAATACCTAAAGGTATTTCAGTGTCTTCATTTTTTTT
AATCGATAAAACCCTTCTTTGCCTACCATTTACTAAATCTCTCATCGTAGGTTCTT
CAATGCCAAACAATACCCGGTTAATGATTGTATTATTCTGGGTTAAAGATAGATC
TGAATCAAATGGGGCCAATGCTAACACAAAAGGCTTACCGATTACATGTTCCATT
GTTGAGTATGGGAACTTCTTACCATTAATTCCGGTAAAGAGATCGCGCTTATCTT
TAATTTTCCCAAGAAGTTTTAGCGTAGATTGATGCTTAAACTTAGCTTCACTGAA
AATTTCACTGACTGGCGGTTTATGGGGCTTGTCAGAGACCATAGCCTCTACGTTA
AATTTATAACCTAATGGCGTGCTAACACAAAAATCAGGACTGTCTTTTGTATAGT
CAACCGAATACCCCAAACGAATAAACATCTCATTTAGATATAATTCCCACATAGA
AGAATTAAAGGTAGTCTGAAATTCGTTAATAAATTTAGTTTGTTCACCACTGCGT
TCCAGTAATCCGCGTCCCCATGATTGGATCACCGCCCTAACAGGAGCAAGTTTAG
GATCTGTGTAGATTGTTTTAAAATTGTGATTTAGTTTATTTCTAGCTATTTTTGTTG
AGAAGAGATCCATTATTTACCTCTATAAACACTTTGTAAAACTAAACATGTGTAT
TTTACTGCGCTGATTGTCGAACATATGCTTCAAATTGTAATGCGGTATTCTACATC
AGAAAAACCTCTTTTATATTTTCCTCATTAAATTTATTTACACCGAAAAATAGCCG
CATGAATCATGTCTGAACAGATGAACTTCTCTAATATAGCCTAAATACCTACGAC
TGCGCTATAAAGAGGGAACCTCAAGCACTAATAGCACAAAGAAAATATGCGAAC
ACAAATTTGACGCGAAACTATGACTTAGATAGGATGAATCTAGGTGGTGATGGT
CACCTGGCGCCTGTCTCGCAAAGCAATAGCCCATCGTTCAGAAGTATACCAAATA
CTTGGCCGCCATCCATTATGACGCTGCCCGGTTAGCAGGCTATCGGTATCCAAAG
CGTTACTTTATTGGAGGTACAGCCATGTCTGACTATTCATACCTAAGCATTCTTAA
AACGTGCGCTAAAAAGCTGGCTCGCATTCAGAAAATTAAGCTTTCCAAAGCACT
CGAAATCGTTGCCCGTGACGCGAAATTCACCAGCTACCATGAGCTGTCCGAAGTC
GCAAAACGTGCCCCGCTAGAACAACGGCTGGTTTTCGCTGCAATAGGCGAAGAT
ACTCAAGAGGATGTGATTTATCGCGATGAGATTTTTTATGCCATAGACCGCGAGG
TGGAAGATATGCTCTCCGGCGACATAGCGGAAACAAACGCCTATGGTTTTAGCA
TAGAGAATCTGGAGGCAACAAGCATAAATTACGACCCAAAGCGAGGCATTCTGG
TCGTCCAAGTCATATTTGATTATCAGGGTGAACAAGATTCTGAACGCCCTTTCTC
TGGTTCGTCCTTTGAAATTGTTGCGAAAATGGAGCTACTTCTTCGCGACAATGTA
TGGAGCCTCGTAGAAAATAGTCTGGAATTGACCTCTGTTAATTCAAATGTCGATA
ATGACTGGTACGATTAAGACATATAACAGTAGGAGGCATTCCAAATTTTTGGCGG
AGTGCCTTTTTAGTAACTATATCCAGATGAAATCATCGAGTTATCACATGAGCAT
CCATCTTACGTGTACTCAAAACACTTATGGGCTTCTAAATTCAGGAAGTCCCTAC
TCCGCTTGCTGGCGCTTCTTTGAGCCTCGTGGCGTCCATATACACCAATTAGGCC
AGACCATGCTCAAACAACTTGTCGAGAAATGCTATGTGCAAAAACCACTGGACA
TGTTCTCGATGACAGCAGGCAAACTAACTGGGCTTGATCAATCTGGGCCTAAACT
GGCTTCCATCATATGCAGAGGGATAGAGCAGGCCAAAGATGTACAACTCGGAGA
GTTACTTTTTGCCTGCGGTATCTATGGTGTGGAAGAAGAAGAAGCCTGGTTACTA
GCTAAAAGATTCAGTAATCTTGAGGCCTTGTATGGTGCCAGCATAGATTCTCTGA
TGAGTTATAACCTTCTTAATGAGGCAGTAGCTGTCAATACGTATAACTTCTTCCGT
CATCCGCTCAATGTGTCGGCTCTTAACGAGTTACAAACAGAAGGTGGATTGAAG
GTGCGCCATGGATAGAATTAACACCAAGGCTGTGCCCGGCCTTTTATCTATGCAA
CGTAGCCGATGACAGTTAACGGTGAGACAGCAACACCTGTTGTAACATGTTTGTA
CTCGTGCATTGCCATCCGTAGGCCTAAACATATCTGACTCCCACAGGATGGGGAT
AAAATACAACATTTGCATTTTCAATTTGTTATCGAAAAATCATTAGTCCTGATGG
GAAATAATATCTTCCCGGACTAATGATTTTCTATCCGGACTCCACTTTTTTATCCG
GGCCTATACCTCCCCTCATCGTTCCGCGCTATAACCGCTCCGACACTCTGTTCCCG
TCCCGTGAGGTGCAATCATGAAAACCAGTGCAGACAGTAACGATGCTTTTCCCG
AAAGCGGCAACGTGCGTATGCGGCAGGTAGTCCAGTTTCTGGCGATGAGTGAAT
CGTCGGTTTACCGCCTGATTAAAAACACCGACTTTCCCCGCCCCGTCCACCTCTCT
TCCCGACTGGTGGTCTTCGATGCTGCTGAAATCCGTCAGTGGCAACAGCGCCGCG
CCGCCATCCGTTAATCCACGCTCAGGGATGAGCCGACTGATACCCCGTCATGAAA
CGCGCCCCTTTTCTCTGTAAGCAGTCTCCGGACCGCACGCTCGAAGTGGTGATCC
TTGCCGGAAGCCTGGCATGGGAGACCTCACACGTGTGGCGAAAAGATCCCGACC
GGGAAGATGATGTTCCCCCGATGGTACTCGGCCCGGATGAACTGGCCGATCTGA
GCAATCTGACCATTATCAGGCCCGACACGCTCTACGTCCGGGTACTTCGCACCGG
CGATATCAGTGAAGAAGAGCTGCTGAAAATTGCCGTAAAACTGGCGCACGCGGG
CGTGCAGATGGCCCGGCTGATGACCCCTGACGGTGAGTTACTGGAGAACTGGAC
CGGACAGCTTGAACGCCTGCGACAGGAAAGACCTTCTGACATCCTGCCGGATCA
CTTCCGCCTCGATGAAGAAGCACTGTGGTTTGATAAGCTCACCGAACGGCGCGA
CGGTGAAAGCGATGTCCAGCCCCAGCGCATCTGTTCCCCTCTGCGCGTCACTGCC
ATCACCTGCGACAGCCATGACGGCAGCTATGGCCGCCTGCTGGAATGGCACACC
ACCACCGGGCAATTGCGGCGCTGGGCCATGCCGATGGCGATGCTCAGCGGCAAC
GGCGAGGAGCTGAGGCGCATTCTGCTGGAGAACGGCCTGACCTATATCTCCACC
CGCCCTGCCCTGCGCAGTCTGCTGTGCGAATATATCTCGCGGTCACTCCCCGGAC
GCCGGGTCACCTGCGCGGAGAAAACCGGCTGGCACAACGGCGTGTACGTGCTGC
CGGATGAGGTTATCGGTCCCGATGGCGACAACGTCATTCTCCAGGGCAGCCACT
ACCTGACTGGCGGCTTTGCACAATCCGGTACGCTGGCAGAGTGGCAGGAGCAGG
TCGCCGCGCTCTGTGCAGGCAATTCACGGCTGGTCTTTGCCGTCTGCTGTGCGCT
GGCCGCCCCGCTGCTGCGCCTGACAGGAACGGGCGGCGGAGGCTTTCATCTGCG
CGGTGAGTCCACTGACGGTAAGACCACGGTGATGAAAGTGGCCGCTTCCGTCTG
TGGCGGGACGGATTACTGGCACTCCTGGCGGGCCACCGGGAATGCGCTGGAAGG
AATTGCCAGCCGCCACAACGATGCCCTGCTGCCACTTGATGAACTGCGCGAAGT
GGACCCGCGTGAGGCCGGGATGATTGCCTATATGCTGGCGAACGGACAGGGCAA
GGGGCGCGCCCGCACCGATGGCGAAGTGCGTAACCGCAGGCACTGGACGCTGCT
GCTGTTCTCCACCGGGGAGCTGTCGCTGGCGGAACACACCGAATGCGCCGGGGA
GCGCCTCTACGCCGGGATGGACGTGCGCATGGTACAAATCCCCAGCGACACCGG
GCAGCATGGTTCGTTTGAGCAGTTGCACGGGTTTGCCAGCGGCCAGCAGTTCGCC
GATACTCTCTGCGACCGGGTGGCCCGCTTCCACGGCACCGCCTTTCGTGCCTGGC
TGGCCTTCCTCACCAGCGACCTGGACGCCAGCACCACGCTGGCGAGAGAGTTAC
TTCGCCGCTACCAGACCGCCCTGATGCCGGACAACGCCGGAAACCAGGTGCAGC
GCATCGTGGCCCGTTTTGCCCTGCTGGCGGTGGCCGGGGAGATAGCGACACTGA
ACGGTATCACGGGCTGGCAGGAAGGCTCGGCGTATGGGGCGGTGCAGATCTGCC
TTCACGCCTGGCTGAACGAGCGCGGCCATATTGCCAACCAGGAAGATGAAGGCG
TGCTGGCGCAGATCAAGCGGTTCATTACCGCGCACCAGTACAGCCGCTTTGCCTC
GTGGGATGGCCCGGACCGCCCGCTGAATATGGCAGGCTTTCGCCGGGTGGAAAA
AGATCCGCTGACGGGCGAGGAACACACGCTGTTCTTCATCCTGCCTGAGGGCTG
GCGGGAAATATGCCGGGGATTCAGTCCTGCCAGAGCGGCCAGATTATGCCAGGA
AGCGGAATGCCTGCTGCCCGGCAGCGACGGGAAATACCAGTCGCAGGTTCGCCT
GCCGGAGATCGGCAAAGCCAGAGTCTACCGCCTCACCTCCCGCATCCTCAGTATC
TGAGTGCCATCTCGCGTGTCTTAAAAAAAGGTGGTACAGGGTGGGACAAGTGGT
ACACGCAGAGCTGGCGCGGGTTTCAGCGTCCCACTTTAGATAATTTGGTTTAAAA
AAAGTGGTACAGAGTGGGACAGCCGTTGCTGAATCTGTCCCACTTTTCTGACTGG
TATTTTTCAGGTGGTACAGCGCAAAGCCACGGCTGGCGCGGTTGTACCACTTGTC
CCACCTGTACCACCTTAAAAACAGAAGAAATGAAAATGGCGAAAAGGGACTGCA
AAAGCAAAGGCTGAAAGGAAGAGAGAATAAGGGGAACGGACTTACTCTTAAAT
GGATAACCTATATTTTTATATTCGTACCATTTACATGATGGAGTGCAAAGTTACC
ATTCAACATGATTTGGATTAGATAGCAAAATTCTCTATAGAATGTTTTCTAGATTT
CAGAGGGATTTAACGATAGCTTATCTCACAAATCACAAATCGGTCAAAAAATAT
ACAGAAAACCAAGTTTTGCCCAGAAATCCTTTTAAAATTTTTGAGTTAAAAATCA
TTGTGTTGTATTAATTTCAGGACGCCACATGTCAAATATCGACACCCAATATAGT
GTATAAAAACACAGGAGGACTCATTGTGATTTATATAATAAAATAACCTTATACA
TCTTTCTGGAAACCAAGTAATGGTTAAAATTCACATTTTAGATGCCGGACATGGT
GATTGCTTGTTAGTAGACTGCGATGGTGTCAAATTGTTAATTGACGCTGGCCCAA
GTACATTTAGGTATAGAAAAAAAATTTCAGCCAAGTTAGCTGAACTTCTGAATGG
CGAGTCTGTTGATATCGCTTTTGTAACTCACAATGATGATGACCATATCGGTGGT
TTCAAATACCTAATTGAAAATAAAATAAACATCAAGAGATTTGTATTCAATAGTT
TATCTAACATTAAACATGTAATAAAGAATTCAAGCAATAAAATATCGACCAAAC
AAGATATAAATCTTGATAGAATAGTGAAAGATGGAAGTTTTGTCTTTTCGACACT
AACTTCAGATGACTCTCCTATTTTAATCAGAAATATAAAAATAACACCTATTACT
CCTTCAAAAAATATCCTTTTAAAATATTTAGAACAACAAGAGCGTAAAAACACA
GAAATTAAAATATCATCTTCATCTGAAAAATATAGTATTAAAGAAGCACTGCAGT
TGCTAAGCAATGGAAATGATATGTTTGTAAAGGATCCATCGGCAACTAATAAAA
CCAGTTTATCATTTATGATTGAGTATGGGAATTTTTGTGGATTATTCCTTGGTGAT
GCACACGAAGAAGACATCTACTCATATATGGCTAATCATTGTGCCAATAGAAAA
ATATCATTTAACGTTGTTAAGTTAGCACATCATGGTAGTGAAAAAAACACGAGTT
CACGGTTATTGAAGTTAATTGGTACAACAGAATATATCATATGTGCGAATAAAAC
CAAGCATAATCATCCAAATAATTTAACTCTTGCAAGAATTTTAATACATGAACAA
GAACCTAAAATTCATATGAGTAGCAGCAATCAGGAGATAATAAATTTACTCAAT
CAAATAAATTTATTAGGCTTCGGCATTACTGCAACCCATTCTGAAAATGGAGTTA
ATACTTTAATCTATGAGTAGAAAAAATGCTGTAAAAATAAAAGTCAATGATTCG
GAATCAAGTGGCTATTTTTTCAAAGCAAGCTCCAAGGATGATTCTTTTGTAATTTC
ATCCAAGCACGGCCTTTGTTCTAGACAGTCAGATTGTGAAGAATTTCTTGATAAT
GTTCAGAATTGTTGCCGTTTATGCACTCAAGATTTAAATATTGACAATATATCTTT
TGAAATTGAAGGAAATAAGAAGCTAAAGCCAATAAGTTATTTCAGTCTTGAAAA
CAAAGATATTGTCATCACCAAAGTGGATGGGGTATCTAATTATCCTTTAAGGATA
GGAAAGATAGAAAAAGAAAAGTATTATACTTATGGTTACAAGAGTAAATGTGAT
AAGCCTGGCAGAATTTTATTAAATGAGCCAGATTTATTAGATGGGATTTGTTATT
TCAATATATGTTCCGATGCAACACCTGAGTTGATTGAGAAAAGTGAAGAATATTA
TGGAATTTCAGGTTCATTAGTTTTTGATTCTCCAGAAAAAGATGTTTTAACCGCAC
ATGCAGTAATAACAACTAATGAAACCAATAATGACTTAGGTTCAGAAATATTAC
ATGATATTGATTTCAAAGAAATTAATAACTTTTTCGGCTGTGAAGTATTCTTGGGT
AAAAAACTCCAATTTCAACTAGATGATACTTTTAGTAAAAATTTTTCAAAGATTG
GAGAAGTGCTTGTTAGCGACAAACTCCAAATTGAAATATTTATTTCATCTTATAA
AGGTATACCATATTTTAATTTGACACCAATTGCTGATGCATTAATTCAAAGTAAG
TTTTATTATGTTTTTGGAAATGTTAGTAGAAGTGATCATATAAATATAATCGCTGC
ATCAAACATTATACTCAACAAATCTCATTTAGAACCAGCTAATAAATTATTAACT
AGCAAATTAACTGAATCGATATTATCAGCACCTCATATTTATTCCACTAGCATTG
ATGATAATAATTATCATCATATTCATTTTAAACAAATGACTTCTGGTGATATTGA
GCTCATAATATCATGTTATGGTGGAGGAGATGATCTTCCTAAAGATATAAATGGC
ACTTTAACTGAGTTGTTACACAATATAAATACTTATAACTTAAATAAAAATTCAA
TAATTGAGAGGTCTTTTCTTAATCAAAAATTTACAGATGCACAATGCGAAATTTT
ATATGAGATTTTTTTGAGCGATTAACTGATTCAATCGACAGAATTTCATTATTGCA
TGTAATTCCTTTGGACCATTATTTAATAAAAGATAAATCAGAAATTAAAAATGAA
ATTATTACCATTGTTGAGAAAGCGTGTCAAGAATTAGACAGCAACATTTTAGATT
ATCTTGAGCATGGAATATCTATAAATCTATTAATATTTCCAACTAATAATAAAGA
TGAATTATCAGGGTTAATGCAAAGGTTGATAAATAATTATGAATGATGCTATTAA
TTATATCTGCAATAGAATAAAAAATAGTGACGGATTTAGTTACTGTTATGAAAAA
CTATTGATATCTATTTCAAAGAATCTATTCAATCCAACTAGCAGCAACCCCATTA
CTCAGCGTGAATATCAACAATTGCTAAAATTCAGCGATTTTTTATCTAATTCCGA
AAAAGAAGAAGATCGAAACCTAGCGTTAAAAATAATATCTGCAATATATGATCT
TTATAAAGAAGATCATAGTTGCCAATTACTGACAAAATCCATCCTTAGCAAACTT
GGACTTTTTGCCGCTGAGGAAGTATTTACTGATAGTGATATAAAACTACCATTAT
CCTATGAAATTTCTAGCAAATACAGAAAAATAAAAAATCGTATAAATGGTTCTG
AGTACATATTCACTAATCGACAATGTGATGTGTACAGTGAGATCATGCAGAATGA
TTATTTTAGTTTTTCTGGCCCGACATCCCTCGGCAAATCATTCTTGATTAAACATG
CTGCCGTTGATCTTATTGAAAATAATAAACTAATTATTTTCATCCTTCCAACAAAA
GCTTTATTAGAAGAATATCTAATTGACTTAAAATCTATACTCAATGAAAAAGGCG
TGAAAGATATAAATGTAAGTAAATCCGTATCACAAGTTGACAAAGAAAGTAAAA
ATATATTGGTGTTTACACAAGAAAGATATAACTCATTTTTATATGAAAAGTCTTA
TGATAATATGGATGTTGACTTTTTATTTATTGATGAAGCTCATAAAGTACTTGACA
AAAAGAATAATAGAGCTATAACCCTATATAAAGTAATAAATAACTCCATAGAAA
AATATGGTAAATTAAAAGTCATTTTTTCCTGCCCAGTAATTTCCAATCCTGATGTG
TTTTTCAAAACATTTAATATACCGAATAGCAAGAAAACAAGAAGTTTATGTATTA
AAGAATCTCCGGTAAATCAAGATTTGTACTTTGCAAATTATCAAGATAATAATTT
TGTCTATTTTGATTCTATTTTAAATAAAAAAATTAATTTCAATGTAAATAATGCTT
ATCAAAATGACTTTGAAATAATCAGAGGATTAGCACAACAATCCAAGTCAAACC
TTATCTACGTATCAAGTAAAACAGAATGTATAAGGAAATGTAACGAATTTCTAGA
GTATTTAATTCGGGAAAAAAAGTATCGATAACAACAGACGATGAACTTATTTCCG
AGTCAAAAATAATCATGGAGTATATTCATAAAGATTTTAACTTAGCTCATGTATT
AAAATATGGTATAGCATTTCACCATGGTTCACTACCTCTATTTATAAGAAAAAGA
ATAGAGGACTTATATTCCAAAAAGAAAATAAATTTTATCTTTTGCACATCAACTT
TACTTGAAGGGGTAAACTTACCCACAAAGAATGTTTTTATTTATCCATTCCCTAA
AAAGACTGTTAACGATGAAAAAAAATGCCGATTAGATTTTTGGAATCTGGCGGG
GCGAGCTGGAAGATACAGAAGTGAATTGACTGGTAACATAGTATGCCTGAATAC
CGCTGATAATAACTGGAGCAATGCAGATGCTAAAATTAGCCTAGGTAATAATGTT
ATTATAGAGGATGAAATAAACTCAACATTACTACGCCATCAAAAAATATTAAATT
ATTTTGATGGTAAAGTTAAATCTCCAACTAATGACATTATTCAACTCAGCTCACTT
ATTCTATCAGAAATATTAACTTATTTAGATAAAGGTTATATAGGGCGAATTTTAA
ATACCTTTAATGAAAAGATTCGGTTTATGCTTATTGATTCAGGAAAAAAACATCT
TGATCGTAATAGAATTATTGAAATAGATAAAGTGACCTTTTCAGAAAATCATATA
TTCTCAACATCCATACACGCTAAAGCCCAACTCAATGCTAAAAATAAAGAAAAA
TTATTGCGTTCTTATAGCAGAGAGGATGTTTTCAATTATTTGGAAACAATAAATG
AAATATATGGCCTGCGTAACACCCCTGATTCATTAAACCAACTGTGCATTGTGAC
TTATTCGTGGCTCATGGGTAATACGCTGAATTTATTAATATCAAACGCTATTAAA
TATAGCAATTCAGTTAGAGATCCTATTAGCTATAGATGGGTTAAATTTGATAAAA
CCAATCCTGATCATATAAACGCAAAAATAATGGAGGCAATACAATGCATTGAAT
CTGAAGTTACTTTTAAACTTGAAACATGTATTGCTCATTTTTATCAGCTATGTCAA
TCAATTCACGGCGATGAAAATGCAGGCATTAATCTTTCACCTTATTTAGAATATG
GAACATTAGATACTAACATCATAGAACTTCAAGAGTTTGGTTTCTCTCGTTTAGC
CGCAATTGAAATTATAGCAAAGCACAAAGAGTGTGTTACATTCAAAACAAATGA
GACATCATTGCAAATAAACACTCAGAAATTAAGAGCTAAGATTGAAAAGCACAG
TGTAATTGATAGAGAACTATCATGGTTGAATTTATAATTCAGCCATGATAAATAA
TAATTTGAAAGATAAATAGGTTGTAATGATATAACCTATTTATCTTCTCGTTACAT
GAATTTCATAAGGATTTTTATACGGGGTATAAAATATCATTTTATATTTAACGCG
GTATACTGGTAACCTAACCGAATGATTAATCATCATCTAAATTATAATCATGGTA
AATAGCCAGTTGCTTGTTGATTGGTAATTTATTGATTTTTAAGGCCAACTCGATAG
AGGATGCCGATAACATCTTTCTTTCTTCATCTGAAGTTCGTTCAACACCTTCAACT
TGGTAGAGCAAAGCATTTAGCATCTTTTTTATAAATATACCTCGCTGTTCATCAG
ACAAATGTTCAGCCAAAGTCATTAGTAGACCTAATTCCCTTTTTACTCGTTCGCTA
TCTTGAGATTGACCTGTTTCCTCACCTAGTAACCAAGCCGGACTAACTCCAGTAG
CATGAGCGATCTTCTGAAGTTGCTCAATTCCATTCGGAAGCGAATCTCCGGCAAC
ATATCCACGGATCGTAGAGTACGGGATATCACATTTTTGTGCCAGATCACGCATG
GAATCGCCTGTCATAGCGAGAAGTTTCTCTAATCTGGCACTAAATTCACTCTTAA
TCATCATACTCCGATTTTGTAACCATATGAATGATAAGAAAAACAGATGAACTAT
CGAAAATTAGATATTTATCACTTTAATTTGTCTAATTTTCGATCTAATCTATCTGT
ACACATTGCAGACAATCACCCTCTGCAAACCCAACTGCTGAGCATCGCATACTCA
GGGCAGAATTGCCACGCATCCGCTCGTCCTGAAAACCGCTCAGTTCACAAAAAC
ACGCCACAAGCGGGGATAACAAAGAAAGCAGTATGAGCCATAGCTGTTTATCGC
CATTACCGGAGCCCCAGACGCTTACGCCTGTCATGTCTTAACCTCTGTCCGATAA
GTCTGTGCGCTCCGGGCCTTTTACCCGGAGGACTCCTTATGTGGACAGTCAGCCT
CGCTGAGGCGTTTATTCATGTATTCCAGGTCGCAGGCAAAATCTTCCTCGACATG
ATGACCGGGCTTATCCCGATGCTCATCTGCCTGCTGCTGGCCATCAACTTCCTGA
TGAAGCTGGTTGGTACCGTGCGGATGGAGAAAGTTGCCGCCCTGCTCGGCCGCTC
GCGCATTCTCACCTACGGCGTGCTGCCGGTGCTCGGCTGGTTCTTTATGAGCAGC
CCCGGCGCGCTCACTTTAGGCAAACTCCTGCCCGAAAAGAGCAAGCCCGGCTAT
GAGGATGCGCTCGGCACCCCCGCCCATCCCCTCACCAGCCTGTTTCCCCACGTAG
TGCCATCAGAGCTGTTTGTCTGGCTCGGCGTGGCTGCCGGGGTTAAGGCGCTCGG
CCTGCCCGTCACCTCGCTGGCGCTGCGCTATATCGCCGCCGCCATGCTGATTGGT
TTAATCCGCGGGATTATCACCGAGTACCTGTTCCTGCGGCTCAGCAAGCGGGGGA
ATGCGCCATGATGATTTACTGAGCACGTATCGTCGCCATCGGGCTGTTTGTCGCC
GATGGTCTCACCGACAAAATGCTTATCACCTTCGACAGCAACGGGCCGAAGGAC
TGTCTCGATTACTCGCTATCGCTGGAGCCGTCGTTCCGGGAAGAGAGCCTGATGA
TACTCCCCGGCGACCGCCTGCTGCTGGCGGGCCACGATTTTCTGGTGACTGCCGT
CGGCAAAGGGGCACAGCAGGCGCTGTTTGAACTCGGCCACCTGACGCTGGTGTT
CAATGGTGACCTGAATCCCTGCCATGTCGGCGCGGTTCATCTCTCCGGCCCGGTG
CCGAACCTGCGCGACCTCCACGGCAATCTGGTGATTGAGGAGGGGCGGCCATGA
ACACGGTGCTCATTCCCCCCGGTCCCGGCGGCTATGGTAAGGGGCTTTGGCTACC
CACAGGGAGCGGCAAAAAGGTACTGTCGCTGACCGGGCGCGAGATTCATCCGGT
GGCGATAGAAATCGGCGCACTTACCGAATCGGAAGTGGTGAATGGCTTTGCCGA
TATCCCGCCGGATAACGACATCCTGTGCGTGGTGATTAACTGTGCCGGGTCCCTG
CGCTGCGGCCTGTATCCGCAGAAGGGTATCCCGACCATCAACGTGCTGCCTACCT
GGCGGGCCGGACCGCTGGCGCACTTTATTAGCGAAGAGAACTACGTCTCCGGTG
TGACGATAGAGCAACTGGTTCTGGTGGATACCGCCGAAGCGCCGGACTGTCAGC
CTGAGTCAGTTCCAGCTCCGGAGCCAGTCATTACCACCGCTCCTCCACCTCCGCG
CGGTGCGGAGAAACTGGTGCGCATGGTCGAGCAGACCGGCACCGCCGTCGGCCA
CGTTATCGCCCTGCTGTTCGCCGCCAGCCGTGAGGCGGTGGACGTGTCGCTGCGC
AACGTTATCCCGTTTATGGCCTTTGTCTCGCTGCTGATTGCCCTGGTGCAGGAGA
CCGCGCTCGGCAGCCTGATTGCCCACGCCCTGACGCCGCTGGCGAACTCACTGTG
GGGACTGGTGCTGCTGTCGCTCATCCGCGGCATTCCGTTTCTCTCCCCGGTGCTA
GGGCCGGGTGCGGCTATCTCGCAGGTGATTGGCGTGATGATTGGCACACAGATT
GGCGCAGGGGCGATATCTCCGGCCTTCGCCCTGCCCGCCCTGTTTGCGATCAATG
TGCAGGTCGGCTGCGACTTCGTGCCTGTTGGCCTTTCGATGCAGGAGGCAAAGCC
GGAGACCATCGCAAAAGGTGTACCCGCCTTTCTGCTGTCGAGACAACTGACCGG
ACCGCTTGCGGTAATAGTCGGCTGGCTGTTTTCCCTGGGCTTATTCTGAGGACAT
AACGATGAACAAAATCAGAGCCGCCGTGGTCGGCGCAGGTATCTACGGCAAACA
CCATATGAATGCGTACCTCCATAACCCGGACACCGTGCTGGTCGCCATCTGCGAC
ACCGATACAGAACGCTGCGACGACCTCGCCATGACCTGTGGCGTGATGGGTTAT
ACCCGGCTGGATGTCCTGTTACAGGAGGAAGCCATCGACGTGGTCTCGGTGGCG
ACACCTGACCCGTACCACACTGAATCCATCCTCACCGCCCTGCGCCACGGCAAAC
ATGTGCTGGCGGAGAAACCGCTCGCCACCTCAGTACGCGAGTGCGAGCTTATCG
TGGAGATGGCGCAACAGCGCGGCCTGCTGGTCGGCGTCGATTTCCACAAACGCT
GGGACCCGGCGGTGATGCGCATCAAAGCCGAACTGGAGAAACCGGAGACCGGG
CGGATCCTGCGCGGCCATATCAGTATGGACGACGTGATTACCGTGCCGACGGAC
TGGCTGGACTGGGCGGGCGCAAGCTCCCCTGTGTGGTTTCTCGGCTCACACTGCT
TTGACCTGGTGCGCCACCTTTCCGGTCAGGAAGTGGTGTCGGTGTATGCCGCCGG
GCAGAAACGGCTGATGACCGAATGCGGCCTCGACACCTTCGACAGCGTGCAGAG
CCTTCTGACGATGGCCGACGGCAGTTCGTGGGTGGTGGAAAACTCGTGGGTGCT
GCCGGAGGGCTTCCCGAAGGACAACGACGGGCGCATTGATATTCTCTGTGAGGC
AACGTATATCCGCAGCACCTCGCAGCATCGCGGGCTGGAAATCACCACGCCAGG
CATGACGCTCACGCCCAACAGCTACTTCATCAACTACCGCAACGGTGTCGCCAGC
GGTTTCGGTATCGATCCGATAAACGACTTTGTTCGCGCGGTCAGGAACCGTTCGC
CGTACCCGGTGACGGCAGAGGACGGGCTGGCGGTCAGCCGGATTTGCGAGGCGG
TGCACCGCAGTCTGGAGAGCGGGAAACCTGAAGATGTTTAAACAAACGGCGCAG
GGTAGCTCCCTGTGCCAGTTCCATATGATTTTGCTAATTCTACTCACTGGTATATT
CACTCGTTAGGCTTTGGCCCGCAGATATATTAATGTTTTCCATCCTGCCGCAACA
AAACCCCACACACGATTGTATGTTTTTTAGTCATCAAGGTGAATAGAGGTATTTT
ATGGCTATGACCTTTTTCACCTCCCTGCCAATAGTATCATGCTAAACCTCTCATCT
ATGTTTATTGCGCCATAATAGTCAACAAGTATTTCAAAAAAGTCCCAAGCAAATT
ATAATGCTGGAGTTTCATAATGTCCCTTTAAAAACCATTTTAAGAAGAGCTATTA
TGTCCCTCCCTACAAATTTTAATGATATTCTTCGTCTTTTTGAAAAAGATTACGAT
ACAGCCAAAGAAGATAATGCCTTAAGTGCACGCGGACAATTTCTGCAACTTTAC
CCCCTTAATCGCCTAAAAAAAATGACGCTTGATGACTATGTCATCGGCAAAGGC
ACGGCCTCATTTTGTGCTTGTGTTGAAGTAAAAACCAGAACATGGGCAAATATGC
AAGGTGCGACGGCGCTCAAATTTGGTATTTATTATGGAAAATCAAAATCAGATCC
AGCCGTCCGCTATCGTTTTACTCAGAAATTTGGCGATGATGATAGTACTAATAAA
GAAGTTTTCGCTAATGTTAAAGACGCTTTACTAGACCTAATACAGTCAGGGAAAG
AATTAGATTTTAGAGCGATTGACGAGAACCCCCTATCCCAAATGTTTAAGGCTAA
AATATTGAGCCTTTACTTTCCAGAACACTTTATAAACATTTGCAGCAAAGATCAT
CTTAAAGAAATTGCTATGAAAATGGGTATAAAAGAGCAACAGTTTATTAGTAAA
TATCAACATTTGTTATTCAAGAAAAAACTAGAGCATAAAATCACCCGAAACTGG
AGCAATCCAAAATATATGTCCTTCCTTTATGCCCAGTTCATACGTAAGGATCTTA
GCAGCGCTCCTGCTGTGATAGTTAAAAAACCACAAAAAAGAAACCATCCCGAAG
TCAATTTCGAAGAAATAACGGACAATCGTGATTTAATAGGCAAAAAAAGCGAAG
AATATGCATTAAACTGGGAAAAAAACCGCCTAATCGGTCTCGGCTATTCAAAAC
TAGCTGAGGAAATAGATGATCGCCGTAATCGTCCAACTTATGGTTACGACTTTCT
TTCTTTTAATGCCCCAGGTGATGAGCGATACATCGAAGTAAAATCAATTGGCCGA
GATGGAAAAGAGGGAGCATTCCGTTTTTTCCTCTCAGGAAATGAACTCACCGTTT
CTAATTTAAGTAACCACCGTAAAAACTATTATTTTTATCTTGTACAGTATGGGAA
AGATGGAGAGCCATGCAATCTATATGTAAAACATGCTCAAGATCTTTACACTAAT
AGTGAAATGACCCCTTGTGCTTACGTTGTTAGGTTCGATCTGGAAGAACCTGCTT
AATAGTTTCGACAACCTCCGACTCATAACTTTAAAAGTGTGAAATCAGGCGCAG
AGTCACCTGAGCCTTCATCTCAGTGATACTGCAACAACACCTGCGAAATCTTCCT
CGCTTCATCAGCAGGATCCGCTGCATGCGTGATCGCTGATCCCACAATCACCACA
TCCGGGCCTAACAGCGCATAGTCTTTCACCGTCTGGCTACTGATGCCGCCTGCTA
CGGCAATCCGTGCTTTCCGGCGCGCCTTCAGCATTGTTATCAAATCATCAATCGG
CTTGCGCCCTGCCGCCTGCTGGTCGGTGCCTGTGTGTACCGCCAGCATATCCGCG
CCCGCCTCTTCCAGGAGGCGGACCCGCGCAGGGAGATCGTCGACGCAGATCATA
TCCACCACCACCTGCTTTCCAGCCTCCTTCGCCGCACAGATGCATGACTGGATCG
TCAGCAGGTCGGTCACACCTAACACCGTGACATAGTCTGCTCCGGCGTCGAAAA
GCAGCTGCGATTCAAAATGGCCGCCATCCATAATTTTCGCATCCACCAGTACTTC
CTTATGCGGGTATTTCTCTTTAATGACTTTAATCGCGTTCACGCCTTCCCGAATAA
GAAAAGGTGTACCCACTTCAATAATATCAACGTCGTCAACCACCTTATCGATAAA
TACCAGCGCTTCAGGCAGGGTTAACTCGTCCAGGGCAAGCTGTAATTTCATGTTG
GTTTCCTTGTGTCGAATGACTATTCGAGGTTGGCGTGTAGCGCAAAACCTTTGGT
TAAGCGATATCCCGTCGACTGCGCCAGATTCACAATCATGGCATCGCCCAGCACC
ATGACCGCCTCTTCAAACAGGCTGCCGCCGGGCAGAATGCCTTTCACGTTATCGA
GTCCATCCGGCAGTTTGTCGGTATAGGCCGGGATCCTGACCACTACCTCCGCCAG
ATTCCCCAGCGTGGACTCGGGGAAAATGGTCAGCAAGGCCACAGTGCCACCCAG
TTGCTTCGCTTTCGTTGCCACATTCACCAGCGAAGCCGTTTCACCCGATGCGCTG
GCCAGTAACAGCAGGTCGCCATTTTTCAGCGCAGGCGTGACCACATCGCCGACC
ACATGCACCTTCAGGCCGATATGCATCAGGCGCATCGCAAAAGCTTTCAGCATTA
ATAGCGAACGCCCTGCGCCAAAAACAAACACCGTGTTCGCTTCAGCGATGGCCT
GCTCCAGACGTGCCAGCGCAGCGCCATCAATCCGCGACATCGCCTGATGCAGGT
CAGTACAGGCGACACCTGCAACAGATTGCGTTTCCATCATGCGTCCTCCTGACAG
AGCGGGAATAAATCATCCTCATACTGGAGCATCCCCATGATTTCCCGGACATAGT
TATCGACATGCCCCAGAAAGTGAAAACGTTCCGGTGCGGCGCGATAGCGTTGAT
AAAGCGGCGTATCGAAGGTGATGCCAAGATGCGGGTTCCCTTTGACGGTGGCGA
ATTTGATGCGCGGAATATGCGGGTAGTAATGATTCTTGCGCACGCAGAGCTGCCC
GTTCTCTTCCACGATGTAGCAGGCCATACCGTGATAAAACTGCACCGTGTCGTAG
TGAACCGCGCAGGGTTTATAGGCCAGATTGCTGAACACCAGCGGCCCGTCGCCA
ATATTGATGGAGCAGTGGCCGTAATTCGGCGGCACGATAATGCTTTCCCCCTCCT
TCACCACCGCCACGATAAGATCGTCCACCAGCAGATCTTTTGGCGCGACTGCAAA
ATCCGGCGACTTTTGCAGGATATATGCCGCAGTCCCCTTAATCACTTCATATAAT
TCCGGATGGGTGTTGCGCCGCGTGTCGTTATAGCCGTGATAATGACCGCTGGTTT
TCTTGCGCTCCCTACCAATCGTTCCCGACATGATGATGGTGATGTCGTACTGATA
CTGGTCGGCAGCAAGCCGTTCCTGATCCTCCGCAAAACTGAGCCCGCGATAAAC
ATCGTAAGCCGGTTCATCACGCTGCGCTTCGGTGAACTCCGGTAAGACCACCGCC
ATCTGCCCGGCGCTTTTGCGGCCAAAGCCGAGATAGTTCAGCGGGGGTTTCAGCA
CCATCACGCCGTCGTCATCGAGATACAGCGGTAAGCCGCTGTGGTGTAGCTGTTT
CATTTTGCCTCCTGTGTATACGCGTTAGCTGCGCGGATCGCCTCTTTCAGCCGATC
GGATTTCATGCCAAAGACGCTTTGAATGTTGTTCCCGACCTCGACGACACCCGCC
GCACCAAGCTGTTTCAGCAGCGCTTTGTCCACCAGCGACTTATCCGTCACGTCAA
TGCGCAGACGCGACATGCAGGCTTCGATGCGGGTCATATTCCCCAGCCCGCCATA
GGCCAGAATGATGCCGCTCACCAGGTCGTTCTGTTCGTTTTCCTGCGCCACAGTG
CTTTCTTCGACTTCACACCCCGGCGTCAGCAAATTCCAGCGGCGGATGGCAAAGG
TAAACAGGGCGTAGTACACCGCTCCCATCACCAGCCCGACCGGGAACACCATGT
ACCAGTGAGGCGCGCGCGGCAGGACGCCAAAGAACAAATAGTCGATAAGCCCG
CCGGAGAAAGAGAGCCCGATGTGCACGCTGAACCACTCCATCAGCAGGAACACC
AGCCCCGCCAGGACGGCGTGAATGCCATATAGCACGGGCGCAACAAACAGGAA
GGAGAACTCAATCGGCTCGGTGATGCCGCAGACGATGGAGGTGATCGCGCCAGA
GAGCATAATGCCCTTCACCCGCGCTTTGTTTTCCGGGCTGGCGCAGCGGTAGATC
GCCACCGCAATCGCCGGAATGCAGAACATCTTGATCGGCGTCAGCCCGGCCATA
AAGGTTCCGGCGGTAATCGGCACCTGATCTTTCAACTGGGCAAAGAAGATAAGC
TGGTCGCCATGCACCACCTGCCCGGCTTTGTTCACGTACTCGCCAAACTGCAGCC
AGAACGTCGGCCACCAGACATGGTTAAGACCAAACGGAATCAGCAGCCGCTCGA
CAAAACCGAACAGGAAGGCGGACACGCCCGGCCCCTGCACCGTCATAGTATTCG
ACAGGCCATTAATCAGATGCTGAACCGGCGGCCAGACCACCGTCATCACCAGCC
CGACAAACAGCGCGGCGAAGGAGGTCACTATCGGCACAAATCGCTTGCCGGAGA
AGAACTCCAGCCATGAAGGAAGCTGGATGCGGTAATAGCGTTTATACAGCCAGG
CGGCGATAATCCCGATAATGATGCCGCCAAACACGCCGGTCTGGAGCGAAGGGA
TGCCCAGCACCATCGTGTAGTCACGTACCTGCGCCACCGACTCGGGCGTGATCCC
CAGAAACTGGCCGATGGTGACGTTCATAATCAGAAAACCGGCGATCGCCGACAA
CCCGGCAATCCCCTGATCGTCACTCAGTCCTACCGCCACGCCGACCGCAAACAGC
AGCGGCAGGTTAGCGAAAATCGCACTGCCCGCTTCAGCCATCAGCTTCAGCACG
TGAACCAGCCAGTCAGCGCCGAGAAACGGCAGGCTGGCGACAATATTGGGATCC
TGAAAGCTGACGCCGAAGGCCAGCAAAATACCGGCGGCAGGCAGCATGGCAAT
GGGGATCATTAACGATCGCCCGATGCTTTGTAACATTTGCAGAAGGTTGAATCTT
TTCATCTCGATCTCCTGAGCGCGTCTTTATGACAGGGATTCGCACAACAGCAGGT
TCTGCTCCGAACACCTTCAGTATCCGAACCGGAGATGATTAAAAAAACAGCAAC
CGACAACGAAAGTTATTTCCGGTTTTCGCTTAAAACCGGAAAATTTATGAGGACA
ATCACGCCGGAAACCGTGAGAAAGGGATAAAAGAGGAAATCGCGTTCGGTGGG
AGGCCATCATATGAGTATCTTCGAGGCGCATTTTCGCAGGCTGCACGCCCGCTAC
GGCGCAGGTCAGACGCACGAATTACAGATGCAGGAGATCGCCGCCATCTTCGGC
TGTTCAGTGCGTAATTGCCGGATTGCTTTGAAAAAGATGCATCAGGAAAAATGGC
TCGACTGGCAGCCCCAGCGCGGGCGTGGCAAGCGCTCACGGCTCCACCTGTTAA
CCTCGCCGGAAAAGCTGTTCAGCCAGAACGTCAATAAGCTACTGGAGAAGCAGG
ATTACGGCAACGTGCTGCGGTTTATCGGCAACGACAAGTATCTGCTGGATCGCCT
GAGCCTGTGGCGCTTTGGGGTACAGGATAAAAGCAGTGAAACGCGGGTGCGCAT
CCCCTACTATCGCAATCTGGATCCGCTTAACCCGCTCGTCCCCCTGCGGCGGACC
GAACGACATCTCCTGCGACAGTGCCTCAGCGGGCTGACGCGCTATGACGCCGTTC
AGGGCAGGATCATCCCCGATATCGCCCACTACTGGACCCACAACGACGACTTTA
CCCGCTGGGAGTTCTGGCTGAAATCCACCGCCCGCTTTGCCGATGGCAGCGAACT
GAATGCCAGCGCCGTTCAGCGCTGCCTGCTCGCCGCCAGCCAGAGCCCGCAGTTC
GCGCCGCTTTTCAGCCCAATCAAAACCATCACCGCTGACGCCCCCTGGCATCTGG
TGATTGAAACGTACCATCCGGTCAGACGGCTCGACTGCCTGCTCGCCACTCAGCC
GACGATGCTGTTCGATTACCAGCACGGACACATCCGCTGTACCGGCGCTTTCCAC
CTGCAGGAACACAGTGACAACTTTATGGTTTTGCGGCGCAATCAACACTGGCACC
AGGCGAGGCCCGGACTGGATGAGATCACCATTTTCACCTGGGCTCCGGAGCATA
TCAGCATGAGCTTTATTCCCCTGCTGCGGGGCGAAGAGGCGCAGGATGACCGCC
CGCTCAACGAGCGTAGTCTGGAGCAGGGCTGCTGTTTTGTGCTGCTCGACGGTGA
AGGTGCCTTTGCGGATGAGGCCGGAAGACGGTTTATCAATTACCTGCTGCAACCT
GTCGAACTCCTCAGCCAGACGCAGCTTCCTGACGAATACGCACGCATCCTCTCCG
TTGCTCAGGGCATGCTGCCGCAGTGGAATCACCGCCCGGTGGATTTCGGCGGGAT
CACCGCGCCGTTTAACCTGCGTCAGCCGGTTATCATCAGCACCTTTCAGCAGCCG
GAACTGGTGGAGCTTGCCGGAGCGATTCGCATCCTGCTTGAACGCTGGCATATTC
GCGCCGAGATACGGATCGACGCATTTGACAGCTTCAACAGCCAGCCGCGCCCTC
CTGCGGATATCTGGCTCAGCAACTTTATGCTCGATACCCTTTCGGTTCCGGCTTTT
CTGGAATGGCTGGCTTCCACCACGCTGTTTACGCGACTGCCTGAAACCCAGCGAC
AAAACCTGAACGCCCTGCTGCCGACCATTCTGAACAGCGATAATGAACAGGCGT
TCGCAACCATTGCCGCCTTTTTCCATGAGATGACCCACCAGCGATATGTCATTCC
TTTGCTACATCACTGGATGGAATTTGCGACCGAGAAGTCATTTACCTGGCGCGAT
TTAAATACGCTGGGATGGCCGGATTTTAGCCAACTTTGGCTCGAATAATGCTACG
CCCGCAAAAGCTCGCTAAAGGGCCTTTAAAGGCCTTTAAGGGTAAGGGAGGGAA
GTGCGGGATTTGGGGCGGGGAACGGTAGACAAATTCAGTCCTCACGACGGAGAC
TGTTCAAATGTCTCTGTACTGACGCCCCAATATGGCTTTACCACCGTCTCCGATTG
TTGAACACGTCAATGTCATCGAAGATATCTGCACGATCCAGATCCTGGGTTTTGT
AGGCCCGTTTTCTGATGCACTGCTTTTTCAGCGAACTGAAGCAAGGCCATAGAAG
TAAAACAGTACCGCTGGCAACTGGGCATTTTCTCGCTTTAGCGGAGACCAACAGT
ATGCTTAGATTCAGTTTATGCCGTCAGCCGATAGTGCGACTGCACCAGACCCGAA
GGGAAGCTGCGGCTGGATAATAGCGTCAAATCGATATCATGGGGTAATACTCCG
AATAGCGGTTTTCCCGAACCGAGCAGAACAGGAACAGTTGTGATAACGATATCG
GCAACCAGACCTTCGCGCAGGAACGACTGTATTAACTGCCCGCCATCGATATAA
ACCCGGTGCACGTTCTTCTCCATAAGGTCAGCAAGAGCCTCCCTTGGCGTGCGGC
ATGAAAACTGTACTTTACCCTTCAGCGCCTTGGGCACGGGGGTATCGGTTAGCTG
CCGGGAAAGCACCAGCACGGGCCGATCATAAGGCCATTCGTCAAAAGTCAGCAC
CTTCTCATAGGTGCCTCGTCCCATAATGATCCAATCTTTGTCCGCGATGAATGCG
GCATAGCCATGATCTTCTGTCGGGTCATCGCGCTTTAACAGCCAGTCGATATCGC
CATCCTGTCGGGCGATATAACCATCAAGACTGACCGCGATGAAAACATGTGTGG
TGACCATCAAGTGCTCCGTTTTCAGGTTGCATATATCTCGTTGAGTTTAGCAATGT
TCGCTCCAGGCACGAAGCGAGCAAGTTACCTAAGTTGAAGGTCCGCAGGGAGCG
AGGAGCAGAAGTTCGAAGCACATCATGAAGTAAGTTTTAAAATGGGAGATAACT
TGCTTTTACATATTTTCCAATGCAATATCATACCCTTTTAAAAATAAATCAGATAT
ATGAGGTGAAAATGTCTAGTCTTAATTTATTCGATGATGTCGTACCTTTAGAAAA
ACAGCATCCAATATATATAATGATGAAAAAGGAACGCTATGAACCGGAGAGAGA
AGTAATTAACCAATGGGCAAAAGGATTTCTAGACAGGGATAATAAGTTTACAAA
AGAATTTCAAACAAGTTTTGAACCTTGCCTATGGGAGTTATATCTTTTTGCTTATC
TTAAAGAATTGGGTTTAAGGAACGACTTTTCATACGATGCACCTGACTTCATCGT
CAATGAACCTGGGTTTTGCATTGAGGCAACCATAGCCCTTCCGGCACAAGGAGC
CCCTGGAGCGCATGGTTTCAGTACGGAAGATATGCCAAGAGATTTTAATAAGTTT
AATTCTGAGGCATCTATTCGACTTAGCAATAGTTTTATATCAAAGGTAAAAAAAT
TAAGATCGAGATATTCTCAACTTCCTCAATGTAAAGAAAAACCTTTTGTTATCGC
CATAGCTTCCTTCGACCGACCTTTTGCGCACTTTGCCGCAGCCAGACCCATACTT
GCAACATTATATGGTTTATATCATGATGAAGAAGCAACCATTGAATCAGGATCAA
AGAGCATAATTTCTTATAACGTTGAGGCTGCGGTAAAAAAAGAAAATGTTAATA
TAGCCATGGGGTTATTTTGCACGCCTGATTATTCGGATGTAAGTGCTGTGATTTAT
AGTAGTTTAGCTACTTGGGGAAAAGTAAGAGCTTTGGCGGACAATCCTTCAGCTC
TAACAATTTATACAACCTTTACCCCAAGAGAAAATTCATTATATCCTAAAGTTCA
TCAAGCCAAAAAAAATGATTATATAGAGCATCTGGCAGATGGATTGTATATTTTA
CATAACCCATTTGCCAAGTATCCGTTGCCTAAAGAGACGCTAAGCCACCCTAGAG
TAGCACAAGGTTATGTTGAATCTGATGGATACGTCAATTTTGTAGCGCCAGAGGA
TTTTCTTTTATTGCGTTTTTTACAATCATTCAATCTTAAAGATTAGTAAATATGGT
CATATTTATTTACTTTACATAGATAAAGTCTAAAGATGGCACTTCCTTTAAACTCA
ACTACCTATGATTTAGAGTGGTTGTCCGGCCTGCTTCCGTAACCGTTAGTTCGACT
GACGAGATTCCTGTTTCCCTCTAAAAGACTTGGTTGATTAAAACAGGTCACCGAA
TAGCCATTTAGTCCACTTTGACCACGAAGCGACCGAACTAACTGAGCAGAAGGT
CCGTTATGAGCGAGGAACGGAAGTTCACAGTTGATCTCACACCCAAATAGTCGC
ATTACTAACATTGCTGCGTGTTAATCAACAGGCAGCAGGTCACTCTTATTTTCTTG
ATAGGCTTTGTATAGTATGAATAAACCTTACAAAGTTATGAAGAAGCTCGTGATT
TTCCTCTGATATACCATCTGGGTCAAAGTGCATCACGTCGTTACGAATTATTCTG
ACCTTGTCGAGCTCTTTAGTAAATATTTTCTTATCTATTTGAAGTTCGGTTTTTTTC
CACAATTCTGGATTTTCAAAAAGTCGTATATATTCCCCAAAGGTTAAATCTGCGA
CCGACTCTATAACCCGTTCGCTGTCAAATGGATTAACGATGCTAACAAGCTCATC
TTTCGTAAATTTTCCATCTATCAGCTTACGAATATGATTTTCGATTTCAGATAGTA
AAAGAAAAGGTTCTGACAACTGTCTGAACTGAGAACTAAGATCGGTAGTAGTTA
TAATCCCACTAATTCTTTGATCAGAAGATCGCACTAATACGTAAGCATACTCAAT
AATTCTAGGTAGTGCAGAAAAAATAGAGTCACTGGCGTTTATTTCCCTGTGTGGC
TTCATATAATCACGCACAACCGCAGATTCTGATTGGGTCAACGCTAGTATTGGAG
CAATGCTTTCCCAACTAATAACACCTTTAACATCTCGCTCGCTGGTCATAACGGG
TAGTTGAGAGTAGTCATGCCGCAACATCAATGTGATGGCTTCTGTTAATGTAGCG
TCAGGTTTCACGGTCACTAGTTTAACATCCGATGCTTCGAGTCGACTAACACGGA
AGGCTGGCTCGGAAACTGCACCGGAAATTAGTTCATCATGCGGCTGAAATACTG
TTTCAGGTGTAGCCAATTCGGCGTTACTTGATTCGGTTTCTTCATGTAAACATTCA
CCTGCTTCATGGGGCGTTGCGTCATTCCCCGGAGGTGCATAGATAGTAATTTTTG
CATCCAGCGGCACACTATCAAAATCAGGTTCGGTTTTAACTCCTAAGCTTTCTAA
TGCTTTGCGAATTTTGTACGCAACCATGTAGCCTCGACGCTCTGCGCCAAACCAG
CTTATGAATTCTCGCACTGATACTGGGTTTGAGGACATACCGTCTTTTAGTTCACG
AAGGATTTCGTTAAGTCTTTCTCGATTTTCTACTGCCATAAAATTTTCTCCATGTA
GGTTGTCGGCTACTGTTAGGTTTCAATCTTGATTCAGGCTTCTCACGCTTTTTTTA
AAATCTGGACAACGAGGCTGCACTGACTTTTTCAGCTCACCTCAGATTGCACCAT
CCCATACACATAGCTCAGATTCCTAATGTTCAGTATAAGGAGGCCAGAATACGA
ATTGCCCTTACCGCTAGTGCCAGCGCGTTGAGTTTAGTAGATGCCTTATTCATTTA
GAATTTGATAAAAATACAAGACTCGGACAACAAGCTGCAATATTAATCGTTATTT
TTCCACTCGAGGCCCACCGTCCGTTTCTGGCACAGGGCTGCTGTTGCAGCCCTGA
AAACCTTAGACTTCAATCGATTCAAAGATCTCTAATGCATCATCTACTTCAATAC
CCAGATAACGGACTGTGCTTTCCAGTTTCTTATGGCCCAACAGAAGTTGGATCAC
CCGAAGATTCTTAGTTTTCTTGTAGATAAAGTAGGGTTTTGTTCTTCTCATGGAAC
GTGTGCTGTAAAGCGAATCTTCGAGACCAAGCTTTCCTGCCCACCCATGAAAGAT
TCGGTTGTATTGTAGGGTTGATATGTGCTGATTAGTTCCGATACGAGACCGGAAC
AAGAAGTCTTTATTGTGCAAATAATCAAGCTTTATCAATGCAGCAATAGCTTCTC
TTGTTCCTTTGGTTATCTCAAATTGCACAGGGCTACCGGTTTTCTGTTGCAACACC
GTTGCTCTGCTTGAAACCAAGCGACCATATGCCACATCAGATACTTTGAGTTTGA
CCAGATCACAGCCTCGAAGCTTACTGTCCAGGGCCATATTGAACAGAGCTAAAT
CGCGCGTTTTGCCTTCCAGTTCAAGCCGGATTCGGGTCCCCCAGATATGAGATAT
CTGAAGTGGTCTTTTTTGGCCGATGATACGGTCTTTGTTCCACGGTGACGTGTTCA
TACTCAAATCTCCTGCAATGTGGGAGATTTGAGTATGGTTGTCCCTTATGAGCGC
AGGCTGTGTGAAAACACTCACCAA
ST69 Region-2 (SEQ ID NO: 32)
TTACAAATCTGCTTGATGAGGACGATATACTGGATACCAAATATTACTGCAACAA
AGAACTTCACTGTTTTTGTCAGAAATAAAAATCGTAATTTCCATATCATCCCAAC
CTTTAACATCTCTGACTACCATCCATAATGGAATATCAAGGCCGTATGATTTCAT
ACCATCAGGAAGAAATTTAATAGCAGTGATTTTGTATGCCAGCGAGCCATTAAG
ATTATACTTTTTACAATATTTTTCTGCTATCGAATTCACTTTGTTTTCAGTAAGAAT
AGGAGCGATTATTTTATTATTCATGAGACTCCACATTGCTGTAAATAAAGAATAA
TTTCAACTTGCATTTCCACTAACTCACCAAAATTTTCAAAGCATAACTCACAAGG
GAAATCACCGACATCATCGGAAAATATACCAAAATCATGAATGAGAATATAAAT
TTTTAGGCTGTTGCGGTCAAGAAAAGCTACACTCCCATCGCCAAAACTGCCAACA
GGAAAAGGATCAATGTTGTTCAATAATGGATATTCTTTTTTATAATCTTCCCATTG
TTGCAAAAATAGAGGGTAATATCTGTCCAGATCCTTCAAAGTATAAATTTGAGGA
TCGACAAAATGACATGCGCCAAAATGAGACAATAACTGCCGATAGTCTTTTGGA
ATATGTGTTGCGTATTTATCCTCAAATAAAAGAAGGTTATCTTCAGTTTCCGGAG
CGGTTACGCAACCTGTAAGTCCTGTTGTTTTTACGGCAGGCCAGGCGGTTTCTAA
AATTTGAAACATTTTTTCCAT

1. A method for detecting the presence or absence of one or more infection-causing E. coli in a sample, said method comprising applying said sample to one or more wells, wherein each of said one or more wells comprises at least one of:
a. a first probe that binds to a nucleic acid target sequence, wherein said nucleic acid target sequence is defined by E. coli ST 69 region 1 (SEQ ID NO: 31) plus region 2 (SEQ ID NO: 32) nucleic acid sequences, and wherein the first probe comprises at least 10 contiguous nucleotides having at least 80% complementarity to a corresponding 10 contiguous nucleotide sequence of said target sequence;
b. a second probe that binds to a nucleic acid target sequence, wherein said nucleic acid target sequence is defined by E. coli ST 73 region 1 (SEQ ID NO: 1) plus region 2 (SEQ ID NO: 2) nucleic acid sequences, and wherein the second probe comprises at least 10 contiguous nucleotides having at least 80% complementarity to a corresponding 10 contiguous nucleotide sequence of said target sequence;
c. a third probe that binds to a nucleic acid target sequence, wherein said nucleic acid target sequence is defined by E. coli ST 95 regions 1-9 (SEQ ID NOs: 3-11) nucleic acid sequences, and wherein the third probe comprises at least 10 contiguous nucleotides having at least 80% complementarity to a corresponding 10 contiguous nucleotide sequence of said target sequence;
d. a fourth probe that binds to a nucleic acid target sequence, wherein said nucleic acid target sequence is defined by E. coli ST 127 regions 1-7 (SEQ ID NOs: 24-30) nucleic acid sequences, and wherein the fourth probe comprises at least 10 contiguous nucleotides having at least 80% complementarity to a corresponding 10 contiguous nucleotide sequence of said target sequence;
e. a fifth probe that binds to a nucleic acid target sequence, wherein said nucleic acid target sequence is defined by E. coli ST 131 regions 1-12 (SEQ ID NOs: 12-23) nucleic acid sequences, and wherein the fifth probe comprises at least 10 contiguous nucleotides having at least 80% complementarity to a corresponding 10 contiguous nucleotide sequence of said target sequence;
allowing nucleic acid present in the sample to contact with the probe within said well;
detecting the presence or absence of sample nucleic acid that has bound to one or more of said probes;
wherein the presence of sample nucleic acid bound to one or more of said probes confirms that nucleic acid from one or more of said infection-causing E. coli is present within the sample, and wherein the absence of sample nucleic acid bound to one or more of said probes confirms that nucleic acid from said infection-causing E. coli is absent from the sample.

2. The method of claim 1, wherein:
a. the first well comprises the first probe and wherein the second to fifth probes are substantially absent from the first well;
b. the second well comprises the second probe and wherein the first, third to fifth probes are substantially absent from the second well;
c. the third well comprises the third probe and wherein the first to second and fourth to fifth probes are substantially absent from the third well;
d. the fourth well comprises the fourth probe and wherein the first to third and fifth probes are substantially absent from the fourth well; and
e. the fifth well comprises the fifth probe and wherein the first to fourth probes are substantially absent from the fifth well.

3. The method according to claim 1, wherein the probes comprise a tag and/or a label.

4. The method according to claim 3, wherein said tag and/or label is incorporated during extension of the probe(s) such that the amplification product(s) become tagged/labelled.

5. The method according to claim 3, wherein the probes are labelled with different labels or tags.

6. The method according to claim 1, wherein each probe is immobilised within its respective well.

7. The method according to claim 6, wherein said immobilisation is permanent or transient.

8. An array of target nucleic acid sequences as defined by:
a. region 1 plus region 2 nucleic acid target sequence of E. coli ST 69 (SEQ ID NOs: 31 and 32), wherein said target nucleic acid sequence comprises at least 10 contiguous nucleotides thereof; and
b. region 1 plus region 2 nucleic acid target sequence of E. coli ST 73 (SEQ ID NOs: 1 and 2), wherein said target nucleic acid sequence comprises at least 10 contiguous nucleotides thereof; and
c. regions 1-9 nucleic acid target sequence of E. coli ST 95 (SEQ ID NOs: 3-11), wherein said target nucleic acid sequence comprises at least 10 contiguous nucleotides thereof; and
d. regions 1-7 nucleic acid target sequence of E. coli ST 127 (SEQ ID NOs: 24-30), wherein said target nucleic acid sequence comprises at least 10 contiguous nucleotides thereof; and
e. regions 1-12 nucleic acid target sequence of E. coli ST 131 (SEQ ID NOs: 12-23), wherein said target nucleic acid sequence comprises at least 10 contiguous nucleotides thereof.

9. The array of target nucleic acid sequences of claim 8 for use in detecting the presence or absence of an infection-causing E. coli.

10. A set of nucleic acid probe sequences comprising at least 10 contiguous nucleotides having at least 80% complementarity to a corresponding 10 contiguous nucleotide sequence of a target sequence as defined by:
a. region 1 plus region 2 nucleic acid target sequence of E. coli ST 69 (SEQ ID NOs: 31 and 32); and
b. region 1 plus region 2 nucleic acid target sequence of E. coli ST 73 (SEQ ID NOs: 1 and 2); and
c. regions 1-9 nucleic acid target sequence of E. coli ST 95 (SEQ ID NOs: 3-11); and
d. regions 1-7 nucleic acid target sequence of E. coli ST 127 (SEQ ID NOs: 24-30); and
e. regions 1-12 nucleic acid target sequence of E. coli ST 131 (SEQ ID NOs: 12-23).

11. (canceled)

12. A set of nucleic acid probe sequences of claim 10, wherein at least 20 contiguous nucleotides having at least 80% complementarity to a corresponding 20 contiguous nucleotide sequence of a target sequence as defined by:
a. region 1 plus region 2 nucleic acid target sequence of E. coli ST 69 (SEQ ID NOs: 31 and 32); and
b. region 1 plus region 2 nucleic acid target sequence of E. coli ST 73 (SEQ ID NOs: 1 and 2); and
c. regions 1-9 nucleic acid target sequence of E. coli ST 95 (SEQ ID NOs: 3-11); and
d. regions 1-7 nucleic acid target sequence of E. coli ST 127 (SEQ ID NOs: 24-30); and
e. regions 1-12 nucleic acid target sequence of E. coli ST 131 (SEQ ID NOs: 12-23).

13-15. (canceled)

16. A test card for use in a method according to claim 1, wherein said test card comprises at least five wells, wherein the first well includes the first probe, the second well includes the second probe, the third well includes the third probe, the fourth well includes the fourth probe, and the fifth well includes the fifth probe.

17. The test card according to claim 16, wherein:
a. the first well comprises the first probe and wherein the second to fifth probes are substantially absent from the first well;
b. the second well comprises the second probe and wherein the first, third to fifth probes are substantially absent from the second well;
c. the third well comprises the third probe and wherein the first to second and fourth to fifth probes are substantially absent from the third well;
d. the fourth well comprises the fourth probe and wherein the first to third and fifth probes are substantially absent from the fourth well; and
e. the fifth well comprises the fifth probe and wherein the first to fourth probes are substantially absent from the fifth well.

18. The test card according to claim 16, wherein the probes are immobilized on the surface of the respective wells; preferably wherein the probes are present in lyophilized form adsorbed to the surface of the respective wells.

19. The set of nucleic acid probe sequences or probe nucleic acid sequence of claim 10 for use in detecting the presence or absence of an infection-causing E. coli.

20. The set of nucleic acid probe sequences or probe nucleic acid sequence of claim 12 for use in detecting the presence or absence of an infection-causing E. coli.
